(19) United States
(12) Patent Application Publication
(10) Pub. No.: US 2026/0048047 A1
(43) Pub. Date: Feb. 19, 2026

(54) SMALL-MOLECULE ACTIVATORS OF MYCOBACTERIUM TUBERCULOSIS ADENYLYL CYCLASE

(71) Applicants: THE SCRIPPS RESEARCH INSTITUTE, La Jolla, CA (US); CORNELL UNIVERSITY, Ithaca, NY (US)

(72) Inventors: Bo QIN, La Jolla, CA (US); Case MCNAMARA, San Marcos, CA (US); Arnab CHATTERJEE, San Diego, CA (US); Michael H. PETRASSI, San Diego, CA (US); Melissa LOVE, La Jolla, CA (US); Sean JOSEPH, San Diego, CA (US); Ashley WOODS, La Jolla, CA (US); Brian C. VANDERVEN, Ithaca, NY (US); David G. RUSSELL, Ithaca, NY (US); Paridhi Sukheja, San Diego, CA (US)

(21) Appl. No.: 19/100,359

(22) PCT Filed: Aug. 2, 2022
(86) PCT No.: PCT/US2022/039160

§ 371 Date: Jan. 31, 2025

(51) Int. Cl. A61K 31/4439; A61K 9/00; A61K 31/439; A61K 45/06; A61P 31/06; C07D 413/14

(52) U.S. Cl. CPC A61K 31/4439; A61K 9/0053; A61K 31/439; A61K 45/06; A61P 31/06; C07D 413/14

(57) ABSTRACT

The present application discloses novel compounds, and compositions comprising said compounds, that are effective small-molecule agonists that stimulate overproduction of cytosolic cyclic AMP (cAMP) in Mycobacterium tuberculosis (Mtb) which inhibits cholesterol metabolism. These small-molecule agonists demonstrate potentiation of current tuberculosis (TB) therapeutics, exhibiting potential as a promising new class of agents for the treatment of TB. The application further discloses methods of treatment of TB using compositions comprising the disclosed novel compounds in combination therapy with current TB therapeutics.

### FIELD OF THE INVENTION

The present application relates to novel compounds, and compositions comprising said compounds, that are effective small-molecule agonists that stimulate overproduction of cytosolic cyclic AMP (cAMP) in Mycobacterium tuberculosis (Mtb) which inhibits cholesterol metabolism. The application further relates to methods of treatment of tuberculosis (TB) using compositions comprising said novel compounds as a new class of therapeutic agents useful in combination therapy with current TB therapeutics.

### BACKGROUND OF THE INVENTION

Tuberculosis (TB), a respiratory and sometimes systemic infectious disease caused by Mycobacterium tuberculosis (Mtb), is a major global health problem [1-4]. It has been almost 150 years since TB has been clinically recognized and more than half a century since chemotherapy for TB became available; however, TB still kills almost two million people every year [5]. Although high-income countries have largely been moving towards TB elimination, high incidences of TB persist in many middle- and low-income countries. India, China, Indonesia, Philippines, Pakistan, Nigeria, Bangladesh, South Africa, DP Congo, and Myanmar have the 10 highest total TB incidences. While South Africa has the highest incidence of TB at 520 per 100,000, India and China have the greatest overall burden of TB due to their large populations, with 2,740,000 and 889,000 cases reported, respectively, in 2017.

Drug-susceptible TB is usually treated with four antibiotics [Isoniazid (INH), Rifampicin (RIF), Pyrazinamide (PZA), and Ethambutol (EMB)] for two months, followed by INH and RIF for an additional 4 months [1, 6, 7]. The requirement for prolonged treatment creates many opportunities for non-compliance and places an enormous burden on TB control programs, especially in low-income countries [1, 7, 8]. Incomplete treatment due to poor adherence paves the way for emergence of multidrug-resistant (MDR; resistant to INH and RIF) and extensively drug-resistant (XDR; MDR strains that are also resistant to Fluoroquinolones and the second-line injectable drugs amikacin, caperomycin, and kanamycin) forms of TB which pose a greater threat to humanity [1, 9, 10]. In addition, recent global reports indicate that ˜3.3% percent (480,000 cases) of newly notified TB cases are MDR-TB positive and only ˜20% of these patients previously received treatment. The treatment of these drug-resistant TB cases requires the administration of more toxic drugs, sometimes for prolonged periods, and is often associated with poor treatment outcomes.

In addition, one-quarter of the world's population is reported to be latently infected with TB. These latently infected individuals asymptomatically harbor dormant TB infection and have a 10% chance of re-activation and developing active disease in their lifetime. In low-income countries, a majority of TB cases occur due to the progression of latent TB infection (LTBI) to active TB disease [11]. Therefore, the treatment of LTBI is important for controlling TB around the world. Latent TB infection is widely treated with INH for 9 months [12]. However, shorter course therapies have recently been developed that require either the administration of INH and Rifapentine (RPT) once weekly for three months or monotherapy with RIF daily for four months [11]. This huge reservoir of latently infected individuals, the long treatment duration required for treating TB, and the global increase in drug-resistant cases present substantial obstacles for TB eradication goals. Many of these problems are likely due to the existence of non- or slow-replicating and differentially drug-susceptible persistent heterogeneous Mtb sub-populations in these disease states that are difficult to treat with currently available drugs [7, 13, 14].

Delamanid and PA-824 are promising new anti-TB drugs that are now in advanced stages of clinical development. Delamanid was conditionally approved for MDR treatment in 2013 and is a prominent part of endTB and MDR-END clinical trials [15]. PA-824 is a major part of the recently described NixTB treatment [16] which involves the administration of three oral drugs (PA-824, Linezolid (Lin) and BDQ) for six months for MDR TB [17]. Unfortunately, BDQ approval had concerns over potential cardiac toxicities and was soon followed by clinical resistance [18]. Both Delamanid and PA-824 are nitroheterocyclic compounds and belong to the class of bioreducible nitroimidazoles. They are prodrugs that are reductively activated by a specific deazflavin (F₄₂₀H₂) dependent nitroreductase (ddn) [19]. Activation releases nitric oxide which causes respiratory poisoning, inhibition of keto mycolates [19] and cidality against both actively growing and persistent Mtb populations. While clinically effective against TB, this class of compounds has a very low barrier to genetic resistance, with resistance frequency of 10⁻⁵-10⁻⁶. Despite their recent introduction, cases of acquired clinical resistance have already been reported due to mutations in the biosynthetic pathway for F₄₂₀H₂ production and recycling [20]. Furthermore, Delamanid and PA-824 resistant isolates with mutations in ddn have been recovered from MDR-TB patients who never received these drugs, raising concerns for formally approved regimens [21, 22].

After infection, Mtb is engulfed by alveolar macrophages in the lungs of a host. Inside these nutritionally limited environments of macrophages, Mtb utilizes host lipids such as fatty acids and cholesterol for energy production. Pro inflammatory cytokines such as TNF-α secreted by the invaded macrophage attract neutrophils, monocytes, T cells, and B cells which aggregate around the site of infection, forming granulomas. Granuloma formation walls off the infecting Mtb bacilli and slows bacterial replication [7, 14]. However, these granulomas are a major pathologic barrier that limit immune cell recruitment and antibiotic diffusion [23]. While depletion of TNF-α exacerbates TB disease [24], high levels of TNF-α can also promote excess inflammation, pathology, and tissue damage [24, 25]. Paradoxically, TNF-α depletion synergizes with TB antibiotic treatment when co-administered in infection models of TB [25, 26]. It has long been known that TNF-α production by macrophages can be down regulated in response to high levels of cytosolic cyclic AMP (cAMP) and it is also known that Mtb-derived cAMP down modulates TNF-α production in macrophages. Thus, stimulating cAMP overproduction in Mtb could be a novel strategy to reduce TNF-α levels specifically in the infected macrophages to enhance activity of current TB drugs [27, 28]. Moreover, stimulating cAMP production in Mtb can perturb multiple aspects of bacterial physiological condition such their ability to utilize cholesterol as carbon source [29], transcription [30], pathogenicity [31], dormancy and stress responses [32].

The recalcitrance of Mycobacterium tuberculosis (Mtb) to conventional antibiotics has created a need to identify novel pharmacological mechanisms to inhibit Mtb pathogenesis. There is a growing understanding of the metabolic adaptations Mtb adopts during infection to support its survival and pathogenesis. This has generated interest in identifying small molecule compounds that effectively inhibit these in vivo metabolic adaptations, while overcoming challenges like poor pharmacokinetic properties or redundancy in target pathways. The Mtb cholesterol utilization pathway has repeatedly been speculated to be a desirable antibiotic target, but compounds that successfully inhibit this complex pathway and are suitable for use in vivo are lacking. (See: Wilburn, K. M., et al. (2022). PLoS pathogens, 18(2), e1009862. https://doi.org/i0.1371/journal.ppat.1009862 and references cited therein).

Thus, there exists a need in the field for more effective therapeutics for the treatment of TB.

As disclosed herein it is established that stimulating cAMP synthesis in Mtb is a mechanism that is sufficient to block cholesterol utilization by the bacterium, preventing the release of key metabolic intermediates that are derived from breakdown of the cholesterol molecule. This work also identifies small molecule agonists of the Mtb adenylyl cyclase Rv1625c that have promising pharmacological properties and are suitable for use during in vivo studies. These Rv1625c agonists increase cAMP synthesis, inhibit cholesterol utilization by Mtb, and disrupt Mtb pathogenesis in mouse models of chronic infection.
1. Organization, W. H., Global tuberculosis report 2014. 2014.
2. Rao, S. P., et al., The protonmotive force is required for maintaining ATP homeostasis and viability of hypoxic, nonreplicating Mycobacterium tuberculosis. Proc Natl Acad Sci USA, 2008. 105(33): p. 11945-50.
3. Geneva: World Health Organization, (WHO). Tuberculosis Fact Sheets. World Health Organization, Geneva. 2018.
4. Geneva: World Health Organization. Global Tuberculosis Report 2018. World Health Organization, Geneva. 2018. 2018.
5. Keren, I., et al., Characterization and transcriptome analysis of Mycobacterium tuberculosis persisters. MBio, 2011. 2(3): p. e00100-11.
6. Burki, T., Tuberculosis post-2015: looking to the future with optimism. Lancet Infect Dis, 2015. 15(1): p. 21-2.
7. Fattorini, L., et al., Targeting dormant bacilli to fight tuberculosis. Mediterr J Hematol Infect Dis, 2013. 5(1): p. e2013072.
8. Bald, D. and A. Koul, Respiratory ATP synthesis: the new generation of mycobacterial drug targets? FEMS microbiology letters, 2010. 308(1): p. 1-7.
9. Riccardi, G. and M. R. Pasca, Trends in discovery of new drugs for tuberculosis therapy. J Antibiot (Tokyo), 2014. 67(9): p. 655-9.
10. Kint, C. I., et al., New-found fundamentals of bacterial persistence. Trends Microbiol, 2012. 20(12): p. 577-85.
11. Rosales-Klintz, S., et al., Guidance for programmatic management of latent tuberculosis infection in the European Union European Economic Area. Eur Respir J, 2019. 53(1).
12. Chee, C. B. E., et al., Latent tuberculosis infection: Opportunities and challenges. Respirology, 2018. 23(10): p. 893-900.
13. Walter, N. D., et al., Transcriptional Adaptation of Drug-tolerant Mycobacterium tuberculosis During Treatment of Human Tuberculosis. Journal of Infectious Diseases, 2015.
14. Sacchettini, J. C., E. J. Rubin, and J. S. Freundlich, Drugs versus bugs: in pursuit of the persistent predator Mycobacterium tuberculosis. Nat Rev Microbiol, 2008. 6(1): p. 41-52.
15. Lee, M., et al., Delamanid, linezolid, levofloxacin, and pyrazinamide for the treatment of patients with fluoroquinolone-sensitive multidrug-resistant tuberculosis (Treatment Shortening of MDR-TB Using Existing and New Drugs, MDR-END): study protocol for a phase II/III, multicenter, randomized, open-label clinical trial. Trials, 2019. 20(1): p. 57.
16. Lienhardt, C., et al., Development of new TB regimens: Harmonizing trial design, product registration requirements, and public health guidance. PLoS Med, 2019. 16(9): p. e1002915.
17. Li, H., et al., Long-Term Effects on QT prolongation of Pretomanid, Alone and in Combinations, in Patients with Tuberculosis. Antimicrob Agents Chemother, 2019.
18. Pontali, E., et al., Cardiac safety of bedaquiline: a systematic and critical analysis of the evidence. Eur Respir J, 2017. 50(5).
19. Singh, R., et al., PA-824 kills nonreplicating Mycobacterium tuberculosis by intracellular NO release. Science, 2008. 322(5906): p. 1392-5.
20. Hoffmann, H., et al., Delamanid and Bedaquiline Resistance in Mycobacterium tuberculosis Ancestral Beijing Genotype Causing Extensively Drug-Resistant Tuberculosis in a Tibetan Refugee. Am J Respir Crit Care Med, 2016. 193(3): p. 337-40.
21. Fujiwara, M., et al., Corrigendum to “Mechanisms of resistance to delamanid, a drug for Mycobacterium tuberculosis” [Tuberculosis 108 (January 2018) 186-194]. Tuberculosis (Edinb), 2018. 110: p. 122.
22. Schena, E., et al., Delamanid susceptibility testing of Mycobacterium tuberculosis using the resazurin microtitre assay and the BACTEC MGIT 960 system. J Antimicrob Chemother, 2016. 71(6): p. 1532-9.
23. Dorhoi, A. and S. H. Kaufmann, Perspectives on host adaptation in response to Mycobacterium tuberculosis: modulation of inflammation. Semin Immunol, 2014. 26(6): p. 533-42.
24. Lin, P. L., et al., Tumor necrosis factor neutralization results in disseminated disease in acute and latent Mycobacterium tuberculosis infection with normal granuloma structure in a cynomolgus macaque model. Arthritis Rheum, 2010. 62(2): p. 340-50.
25. Skerry, C., et al., Adjunctive TNF inhibition with standard treatment enhances bacterial clearance in a murine model of necrotic TB granulomas. PLoS One, 2012. 7(6): p. e39680.
26. Subbian, S., et al., Phosphodiesterase-4 inhibition alters gene expression and improves isoniazid-mediated clearance of Mycobacterium tuberculosis in rabbit lungs. PLoS Pathog, 2011. 7(9): p. e1002262.
27. Katakami, Y., et al., Regulation of tumour necrosis factor production by mouse peritoneal macrophages: the role of cellular cyclic AMP. Immunology, 1988. 64(4): p. 719-24.
28. Roach, S. K., S. B. Lee, and J. S. Schorey, Differential activation of the transcription factor cyclic AMP response element binding protein (CREB) in macrophages following infection with pathogenic and nonpathogenic mycobacteria and role for CREB in tumor necrosis factor alpha production. Infect Immun, 2005. 73(1): p. 514-22.
29. Wilburn, K. M., et al., Pharmacological and genetic activation of cAMP synthesis disrupts cholesterol utilization in Mycobacterium tuberculosis. PLOS Pathogens, 2022. 18(2): p. e1009862.
30. Kahramanoglou, C., et al., Genomic mapping of cAMP receptor protein (CRP Mt) in Mycobacterium tuberculosis: relation to transcriptional start sites and the role of CRPMt as a transcription factor. Nucleic Acids Res, 2014. 42(13): p. 8320-9.
31. Shleeva, M., et al., Cyclic AMP-dependent resuscitation of dormant Mycobacteria by exogenous free fatty acids. PLoS One, 2013. 8(12): p. e82914.
32. Choudhary, E., W. Bishai, and N. Agarwal, Expression of a subset of heat stress induced genes of Mycobacterium tuberculosis is regulated by 3′,5′-cyclic AMP. PLoS One, 2014. 9(2): p. e89759.

### SUMMARY OF THE INVENTION

The present application generally discloses the use of novel small-molecule agonists that stimulate the overproduction of cAMP in Mtb via the putative activation of the mycobacterial adenylyl cyclase, Rv1625. In turn, the increase in cAMP inhibits cholesterol metabolism.

The present application specifically relates to use of mCLB073, one of the small-molecule agonists disclosed herein that stimulates the overproduction of cAMP in Mtb and accordingly inhibits cholesterol metabolism. As described herein, mCLB073, in particular, demonstrates surprising potentiation of the approved Nix TB regimen of Bedaquiline, Pretomanid and Linezolid or the reduced backbone of just BDQ and PA-824, and is viewed as a promising agent for the treatment of TB. Given the impressive efficacy when dosed in combination, there is promise that mCLB073 could lead to treatment shortening of Nix TB or other drug combinations to import increased compliance, reduced resistance potential and a reduced cost of treatment for significantly shortening treatment duration.

In summary, the advantages associated with treatment of TB using mCLB073 in combination with approved TB therapies include, but are not limited to: treatment shortening from the standard 6-month regimen to potentially a ≤3-month regimen; low predicted human dose (30 mg once daily), which may further reduce the cost of treatment; higher compliance and reduced potential for drug resistance; and a novel mechanism of action with no pre-existing resistance having yet been reported. (See: Wilburn, K. M., et al. (2022). PLoS pathogens, 18(2), e1009862. https://doi.org/i0.1371/journal.ppat.1009862, Supplemental Tables and Figures, and references cited therein).

The present application provides a compound of Formula I or Formula II,

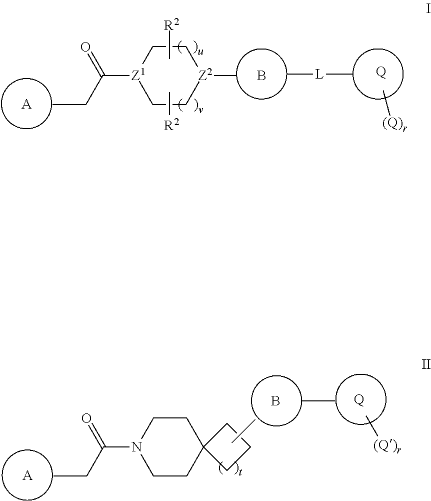

wherein:
A is selected from the group consisting of

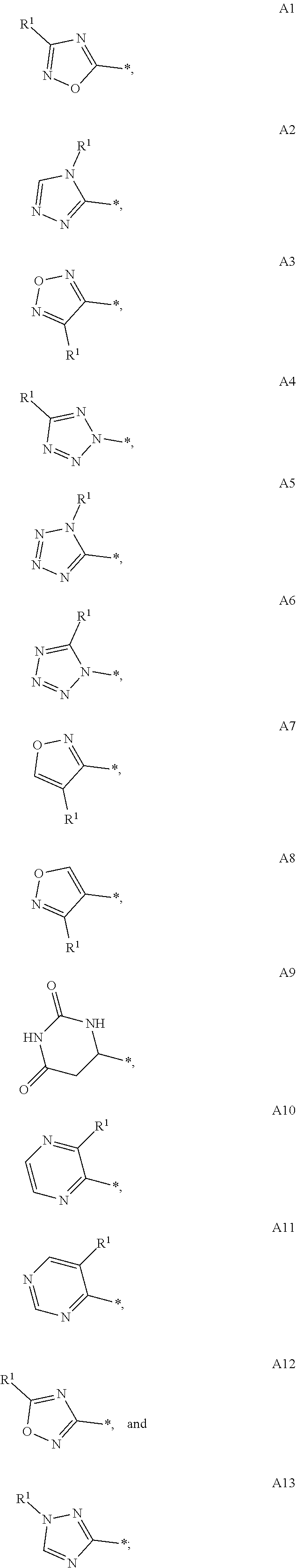

each R¹ is independently H or (C₁-C₆)alkyl;
Z¹ is CH or N;
u is 0 to 2;
v is 0 to 2;
both R² are absent or both R² together form a (C₁-C₂)alkylenyl bridge between two ring carbon atoms, provided that u and v are not both 0;
Z² is CH, CR³, or N;
   R³ is OH, CF₃, or (C₁-C₆)alkyl;
t is 0 or 1;
B is selected from the group consisting of:

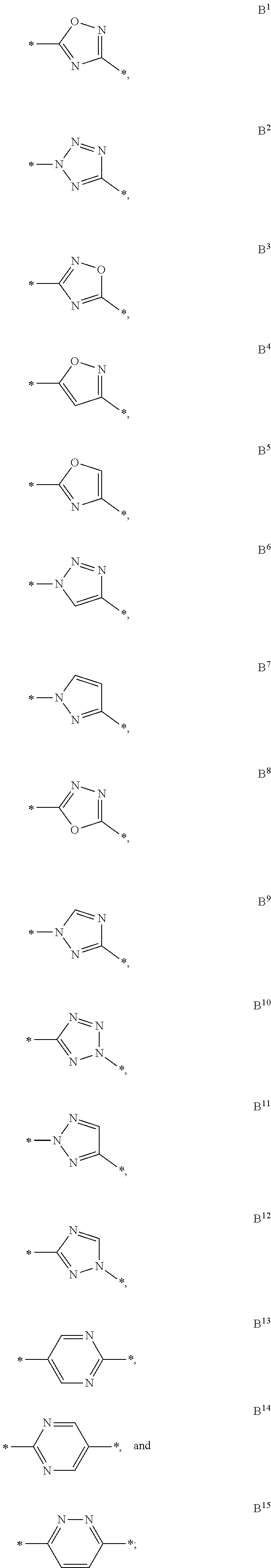

L is a bond;
   or if A is A³ and B is B¹, B², or B⁶, L may be —C(═O)—, —C(═O)N(R⁹)—, —CH₂—, or —O—; R⁹ is H, (C₁-C₆)alkyl, or (C₁-C₆)heteroalkyl;
Q is 3- to 8-membered monocyclic, 6- to 16-membered bicyclic, 4- to 14-membered fused bicyclic, or 5- to 11-membered spirocyclic cycloalkyl or heterocycloalkyl, Ph, or 5- to 8-membered heteroaryl, each optionally substituted with (Q′)r;
   or if A is A³ and B is B¹, B², or B⁶, Q may be (C₁-C₆)alkyl or (C₁-C₆)heteroalkyl;
   each Q′ is independently halo, halo (C₁-C₆) alkyl, haloalkoxy, —CN, (C₁-C₆)alkyl, (C₂-C₆)alkenyl, (C₂-C₆)alkynyl, (C₁-C₆)heteroalkyl, OH, oxo, amino, amido, imino, (C₁-C₆)alkyl ester, carboxyl, —SF₅, 3- to 8-membered cycloalkyl, 3- to 8-membered heterocycloalkyl, 3- to 7-membered spirocyclic cycloalkyl or heterocycloalkyl, aryl, or 5- to 6-membered heteroaryl; and
r is 0-5;
   with the proviso that the compound of Formula I is not 2-(4-methyl-1,2,5-oxadiazol-3-yl)-1-(4-(5-(p-tolyl)-2H-tetrazol-2-yl)piperidin-1-yl)ethan-1-one; 2-(4-methyl-1,2,5-oxadiazol-3-yl)-1-(4-(5-(pyridin-4-yl)-1,2,4-oxadiazol-3-yl)piperidin-1-yl)ethan-1-one; 2-(4-methyl-1,2,5-oxadiazol-3-yl)-1-(4-(5-(6-methylpyridin-3-yl)-1,2,4-oxadiazol-3-yl)piperidin-1-yl)ethan-1-one; 2-(4-methyl-1,2,5-oxadiazol-3-yl)-1-(3-(3-phenyl-1,2,4-oxadiazol-5-yl)piperidin-1-yl)ethan-1-one; 1-(3-(5-cyclopropylpyrimidin-2-yl)azetidin-1-yl)-2-(4-methyl-1,2,5-oxadiazol-3-yl)ethan-1-one; 8) 1-(4-(5-(furan-3-yl)-1,3,4-oxadiazol-2-yl)piperidin-1-yl)-2-(2H-tetrazol-2-yl)ethan-1-one; 2-(2H-tetrazol-2-yl)-1-(3-(5-(thiophen-3-yl)-1,3,4-oxadiazol-2-yl)piperidin-1-yl)ethan-1-one; 2-(1H-tetrazol-5-yl)-1-(4-(3-(thiophen-2-yl)-1,2,4-oxadiazol-5-yl)piperidin-1-yl)ethan-1-one; 2-(5-isopropyl-1H-tetrazol-1-yl)-1-(4-(3-phenyl-1,2,4-oxadiazol-5-yl)piperidin-1-yl)ethan-1-one; 1-(3-(3-phenyl-1,2,4-oxadiazol-5-yl)piperidin-1-yl)-2-(1H-tetrazol-1-yl)ethan-1-one; 1-(3-(5-(1-(cyclopropylmethyl)pyrrolidin-2-yl)-1,2,4-oxadiazol-3-yl)azetidin-1-yl)-2-(1H-tetrazol-1-yl)ethan-1-one; 1-(3-(4-phenyl-1H-1,2,3-triazol-1-yl)azetidin-1-yl)-2-(1H-tetrazol-1-yl)ethan-1-one; 1-(3-(4-phenyl-1H-1,2,3-triazol-1-yl)pyrrolidin-1-yl)-2-(1H-tetrazol-1-yl)ethan-1-one; 1-(3-(5-cyclopropylpyrimidin-2-yl)azetidin-1-yl)-2-(1H-tetrazol-1-yl)ethan-1-one; 1-(4-(6-cyclopropylpyridazin-3-yl)piperazin-1-yl)-2-(1H-tetrazol-1-yl)ethan-1-one; 1-(4-(6-(1H-1,2,4-triazol-1-yl)pyridazin-3-yl)piperazin-1-yl)-2-(1H-tetrazol-1-yl)ethan-1-one; 1-(3-(3-cyclopropyl-1,2,4-oxadiazol-5-yl)pyrrolidin-1-yl)-2-(isoxazol-3-yl)ethan-1-one; 2-(isoxazol-3-yl)-1-(3-(3-phenyl-1,2,4-oxadiazol-5-yl)azetidin-1-yl)ethan-1-one; or 1-(3-(3-isobutyl-1,2,4-oxadiazol-5-yl)piperidin-1-yl)-2-(4-methyl-1,2,5-oxadiazol-3-yl)ethan-1-one;
or any pharmaceutically acceptable salts or any stereochemically isomeric forms thereof.

The application further provides a composition comprising the above compounds of Formulae I-II, further comprising one or more therapeutic compounds or compositions used for the treatment of a TB infection.

The application further provides a method of preventing, ameliorating, or treating a TB infection, comprising administering to a subject in need thereof a therapeutically effective amount of a composition comprising the above compounds of Formulae I-II, further comprising one or more therapeutic compounds or compositions used for the treatment of a TB infection.

### BRIEF DESCRIPTION OF THE FIGURES

FIG. 1. V-59 inhibits Mtb growth in an Rv1625c-dependent mechanism.

FIG. 2. Structures and activities of compounds.

FIG. 3. V-59 stimulates Rv1625c to produce cAMP.

FIG. 4. The transmembrane domain of Rv1625c is essential for complete degradation of cholesterol and the catalytic domain of Rv1625c is required for V-59 activity.

FIG. 5A-C. Inducing cAMP synthesis independent of V-59 and Rv1625c is sufficient to block cholesterol utilization.

FIG. 6. V-59 treatment and induction of TetOn-cAMP are associated with shared transcriptional changes to cholesterol utilization genes.

FIG. 7. Activating cAMP synthesis decreases liberation of propionyl-CoA from cholesterol.

FIG. 8. V-59 treatment and induction of TetOn-cAMP are associated with transcriptional. changes in select CRPMt regulon genes.

FIG. 9A-G. Chemically activating Rv1625c reduces Mtb pathogenesis in vivo.

FIG. 10. In vitro potency of mCLB073 in differentiated THP-1 cells.

FIG. 11. In vitro potency of mCLB073 in J774.1 cells.

FIG. 12. In vitro potency of mCLB073 in Mtb H37Rv.

FIG. 13. In vitro potency of mCLB073 in Mtb H37Ra.

FIG. 14 A-C. Determining Extracellular Activity Against M. tuberculosis in Cholesterol Media.

FIG. 15A-C. Determining activity of mCLB073 in a Hu Coats 100-day old culture model.

FIG. 16A-D. Combination testing of mCLB073 with known anti-TB drugs.

FIG. 17A-E. The inhibitory activity of V-59 in cholesterol media is dependent on Rv1625c.

FIG. 18A-C. The activity of mCLB073 is dependent on Rv1625c.

FIG. 19A-C. An intact cyclase domain of Rv1625c is required for V-59 to inhibit cholesterol degradation.

FIG. 20A-F. Construction and validation of TetOn-cAMP constructs.

FIG. 21A-F. Results of activation of cAMP synthesis.

### DETAILED DESCRIPTION OF THE INVENTION

An important aspect of Mtb pathogenesis is that the bacterium persists in the human lung within lipid-rich phagocytes and/or tissue lesions while promoting pathology that is required for dissemination and transmission. Mtb primarily lives within macrophages and stimulates the formation of lipid-loaded cells, but the bacterium can also survive in the acellular core of necrotic granulomas that are rich in cholesterol, cholesterol ester, and triacylglycerols. It is generally understood that Mtb utilizes host-derived lipids, including cholesterol, as key nutrients to survive during persistent infection. Mtb completely degrades cholesterol into two- and three-carbon intermediates that are metabolized for energy production or serve as biosynthetic precursors of cell wall or virulence lipids. In animal models, Mtb requires cholesterol metabolism to maintain optimal chronic lung infection and cholesterol utilization was recently found to belong to a set of “core virulence functions” required for Mtb survival in vivo across a genetically diverse panel of mice. Furthermore, it was recently demonstrated that a multi-drug resistant strain of Mtb is more dependent on cholesterol for growth than the H37Rv reference strain. Thus, cholesterol metabolism in Mtb represents a novel, genetically validated target for drug discovery. However, tools to pharmacologically inhibit this pathway during infection in vivo have yet to be developed.

Signaling through the universal second-messenger 3′,5′-cyclic adenosine monophosphate (cAMP) has long been studied in a variety of prokaryotic and eukaryotic systems. In pathogenic bacteria, cAMP is essential in regulating functions such as carbon metabolism, virulence gene expression, biofilm formation, drug tolerance, and manipulation of host cell signaling. How cAMP signaling regulates Mtb physiology during infection is not well understood, partly due to the limited tools available for investigating this and the myriad of pathway components present in Mtb. The Mtb genome encodes an unusually large repertoire of at least ten biochemically active class III adenylyl cyclase (AC) enzymes, which catalyze the intramolecular cyclization of ATP to form cAMP upon activation. These ACs are structurally diverse, and the majority of these proteins are composed of a catalytic domain along with other accessory domains, which are thought to participate in regulatory or effector functions. Studies using recombinant expression systems have proposed environmental stimuli (e.g., pH, fatty acids, or CO₂) for five Mtb ACs. Additionally, Mtb possesses twelve predicted downstream cAMP-binding effector proteins, only four of which have been functionally characterized. Thus, the understanding of how individual ACs and downstream cAMP-dependent effector proteins regulate specific aspects of Mtb physiology is extremely limited. To date, no individual AC enzyme has been directly linked to the regulation of a specific biological or metabolic process in Mtb.

A series of compounds was previously identified that was found to inhibit Mtb growth in macrophages and in cholesterol media. The activity of a subset of these compounds is dependent on the AC Rv1625c, and compound treatment increased cAMP production in Mtb. The Rv1625c protein is composed of at least four structural elements: an N-terminal cytoplasmic tail, a six-helical transmembrane domain, a cytoplasmic helical domain, and a C-terminal cyclase domain. Based on its topology and sequence homology, Rv1625c is comparable to ‘one-half’ of a mammalian membrane-associated AC. Rv1625c forms a homodimer to generate two active sites composed of complementary residues, conserved active site residues, as well as the cytoplasmic tail and helical domain have been linked to its catalytic activity. Although it has been proposed that Rv1625c may be activated by binding CO₂ or lipophilic ligands, it remains unclear what the native role of Rv1625c is in Mtb during infection. The possibility that chemical tools comparable to forskolin in the Mtb system had been identified led us to investigate the mechanism of these Rv1625c-dependent compounds and their impact on Mtb carbon metabolism and pathogenesis. It then became important to test the hypothesis that activating cAMP synthesis in Mtb through an Rv1625c agonist could disable cholesterol utilization and undermine Mtb persistence during infection in mice.

To carry out these studies, previously identified screening hits for Rv1625c-dependent compounds with favorable pharmacokinetic properties were re-examined. From these, selected was a potent compound (V-59) that permitted both in vitro and in vivo studies to examine the impact that chemically activating cAMP signaling has on Mtb metabolism. In this work, it was determined that V-59 is an Rv1625c agonist, and its ability to inhibit Mtb growth in macrophages and cholesterol is dependent on Rv1625c and an associated increase in cAMP synthesis. Additionally, it was found that the transmembrane domain of Rv1625c is necessary for the complete metabolism of cholesterol, linking the protein target of V-59 directly to the cholesterol utilization pathway. This finding connects a single AC to the regulation of a downstream metabolic pathway in Mtb. Using a complementary genetic approach, an inducible system to activate cAMP synthesis independent of V-59 and Rv1625c was developed, and it was determined that upregulating cAMP synthesis is sufficient to inhibit cholesterol utilization in Mtb. V-59 was optimized through medicinal chemistry, which produced a lead compound (mCLB073, Table 1: Compound 164) with improved potency and in vivo activity against Mtb when delivered orally to infected mice. Collectively, these results reveal a novel cAMP signaling mechanism in Mtb that inhibits cholesterol utilization and may represent an improvement over developing conventional single-step inhibitors against this complex pathway. Using a small molecule AC agonist as an antimicrobial compound is an unconventional approach, and this study explores this as a mechanism of action to inhibit growth of a bacterial pathogen during infection.

Previously identified compounds have been shown to inhibit Mtb replication in macrophages (29) and determined that one of these compounds (V-58) preferentially inhibits Mtb growth in cholesterol media in an Rv1625c dependent manner. Unfortunately, these previously identified compounds have poor potency and pharmacological properties. For example, a resynthesized analog of V-58 (sCEB942) displayed sub-optimal intramacrophage potency that could not be improved (Table S1).

TABLE S1
V-59 is structurally distinct from other
cholesterol utilization inhibitors.

| | | | Analog of | Analog of |
|---|---|---|---|---|
| | Rv1625c- | Rv1625c- | Previously | Previously |
| | Independent | Independent | Published | Published |
| | Cholesterol | Cholesterol | Rv1625c- | Rv1625c- |
| | Breakdown | Breakdown | Dependent | Dependent |
| | Inhibitor | Inhibitor | Inhibitors | Inhibitors |
| | V-13-012725 | V-13-011503 | sCEB942 | mCCY224 |
| MW | 224 | 276 | 426 | 468 |
| Intramacrophage | 25 | 25 | 4.16 | 2.96 |
| EC₅₀ (μM) | | | | |
| Cholesterol | 1.4 | 5 | 0.67 | 0.35 |
| EC₅₀ (μM) | | | | |
| 7H9 OADC | >50 | >50 | >20 | 9.56 |
| EC₅₀ (μM) | | | | |
| hERG (%) | — | — | — | 89.09 |
| HepG2 CC₅₀ | — | — | — | 20 |
| (μM) | | | | |
| Cyp IC₅₀ | — | — | — | 9.55/23.29 |
| (μM, 2C19/2C9) | | | | |
| Mouse PPB (%) | — | — | 89.61 | 99.96 |
| Caseum binding | — | — | 3.34 | <<0.01 |
| (% unbound) | | | | |
| ER | — | — | 0.79/0.56 | 0.48/0.43 |
| (mouse/human) | | | | |
| Solubility | — | — | 183.09 | 1 |
| (mM, pH 6.8) | | | | |
| Time over EC₅₀ | — | — | — | >12 h |
| CLogP | — | — | 3.42 | 4.04 |
| PSA | — | — | 109.7 | 59.3 |

Chemical structure of previously published single-step cholesterol breakdown inhibitors, and resynthesized analogs of previously described Rv1625c-dependent inhibitors. MW, molecular weight; —, not determined; EC₅₀, half-maximal effective concentration; hERG, human ether-\dot{a}-go-go-related gene; CC₅₀, 50% cytotoxic concentration; PPB, plasma protein binding; IC₅₀, half-maximal inhibitory concentration; Cyp, cytochrome P450; ER, extraction ratio; PSA, polar surface area.

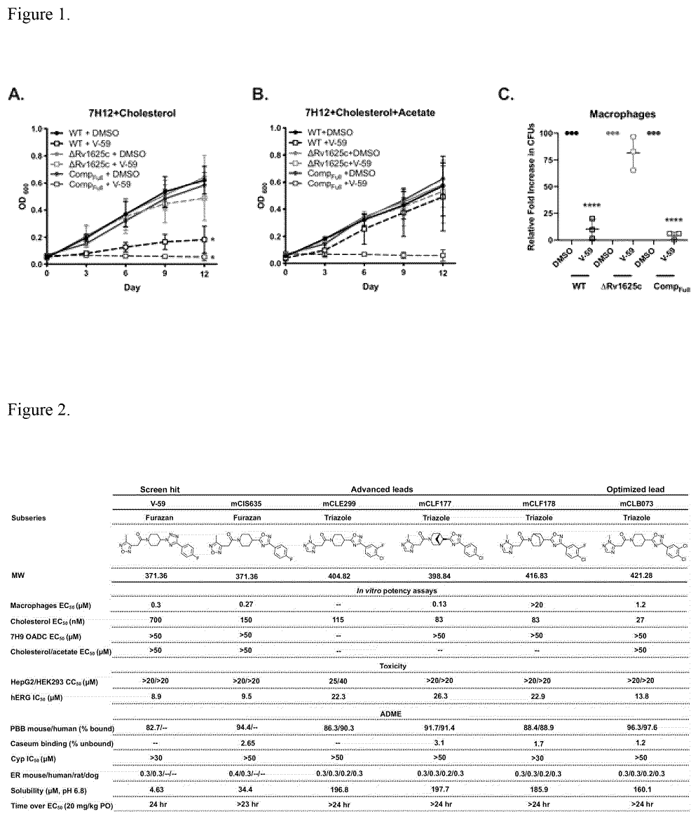

Similarly, the previously identified compound (mCCY224) displayed poor solubility, high plasma protein binding, and high levels of caseum binding (Table S1). These properties precluded the use of these Rv1625c-dependent compounds in mice. Since a primary goal of this work was to investigate the impact that activating cAMP synthesis has on Mtb physiology during infection in mice, the screening hits were re-examined to identify candidate Rv1625c-dependent compounds with more favorable pharmacological properties that are permissible for both in vivo and in vitro studies. This effort revealed a small molecule 1-(4-(5-(4-fluorophenyl)-2H-tetrazol-2-yl)piperidin-1-yl)-2-(4-methyl-1,2,5-oxadiazol-3-yl)ethan-1-one, named V-59, that inhibits Mtb replication in macrophages (half maximal effective concentration (EC₅₀) 0.30 μM. Because the availability of carbon sources can potentially impact activity of chemical inhibitors against Mtb, V-59 was evaluated in different in vitro culture conditions. Similar to a previously characterized Rv1625c agonist, V-59 inhibits Mtb growth in cholesterol media (EC₅₀ 0.70 μM) (FIGS. 1A and 2) but not in media containing the two-carbon fatty acid acetate, or in standard rich growth media (FIGS. 1B and 2). V-59 also displayed a promising pharmacokinetic profile (FIG. 2) and was therefore selected for further investigation as a potential Rv1625c agonist.

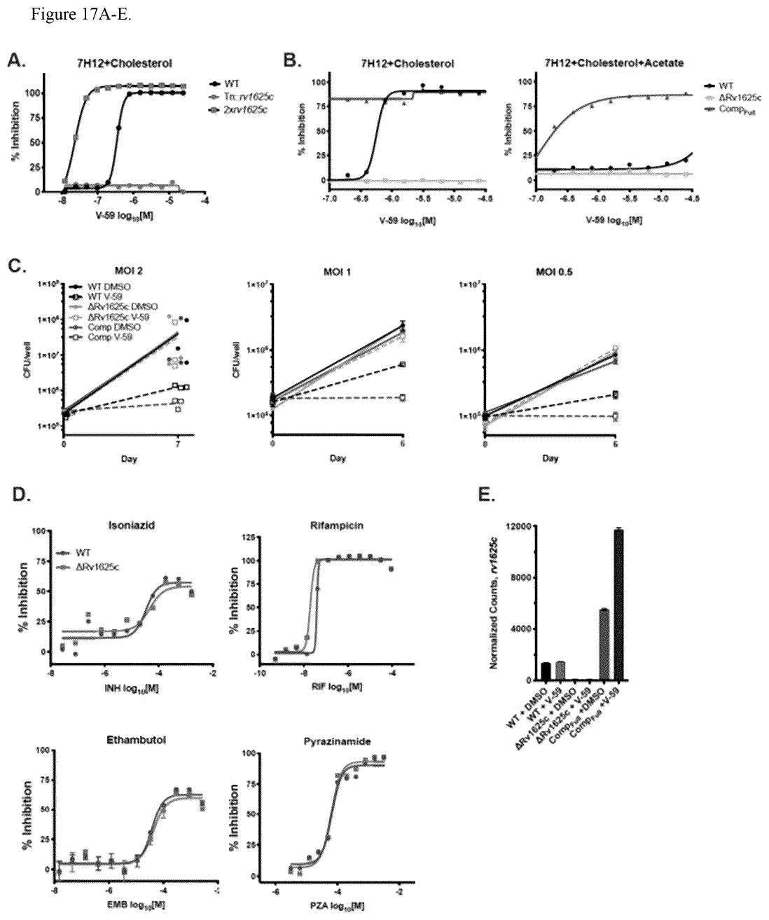

V-59 is structurally distinct from previous cholesterol utilization inhibitor candidates (Table S1). Similar to a subset of other cholesterol-dependent Mtb growth inhibitors that were identified, a transposon insertion in the rv1625c gene (Tn::rv1625c) confers resistance to V-59 (FIG. 17A). Inversely, WT Mtb transformed with an rv1625c overexpression plasmid (2×rv1625c) was ˜15-fold more susceptible to V-59 than WT (FIG. 17A). This heightened susceptibility suggests a mechanism in which V-59 activates Rv1625c, and growth inhibition scales with Rv1625c enzyme levels. To test this further, the gene encoding Rv1625c (ΔRv1625c) was deleted and this mutation was complemented with the entire rv1625c gene (Comp_{Full}). The ΔRv1625c mutant is refractory to V-59 inhibition in cholesterol media (FIGS. 1A and 17B). Because macrophages contain various nutrients that can support Mtb growth it was determined that V-59 inhibits Mtb growth in murine macrophages in vitro and confirmed that Rv1625c is required for V-59 activity during macrophage infection (FIGS. 1C and 17C). Importantly, the ΔRv1625c strain does not have a pan-drug resistance profile (FIG. 17D). Across all of these assays, the Comp_{Full} strain was more susceptible to V-59 treatment relative to WT, even in media containing acetate. This is likely because rv1625c is overexpressed in the Comp_{Full} strain relative to its native expression levels in WT (FIG. 17E). It was concluded that a functional Rv1625c enzyme is required for V-59 activity, and that this compound inhibits Mtb growth in cholesterol media and macrophages.

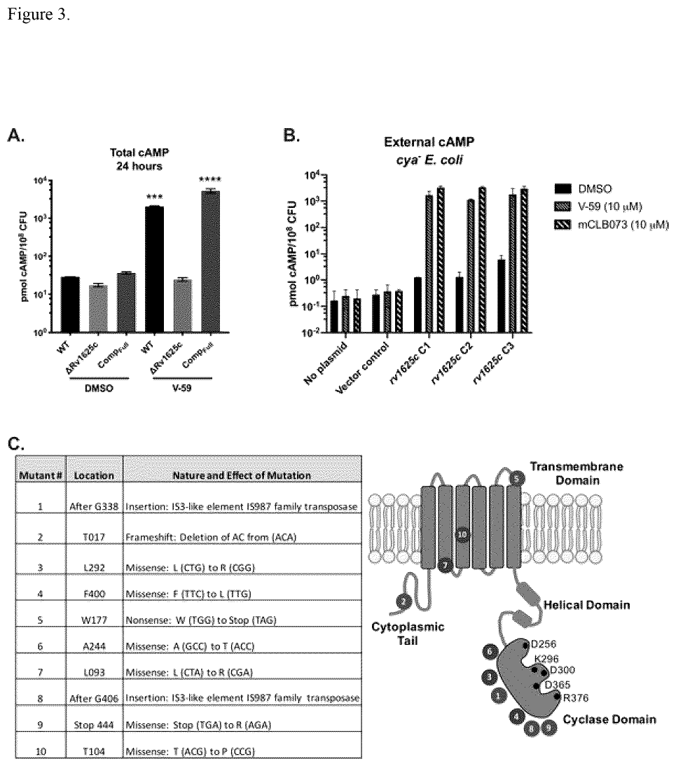

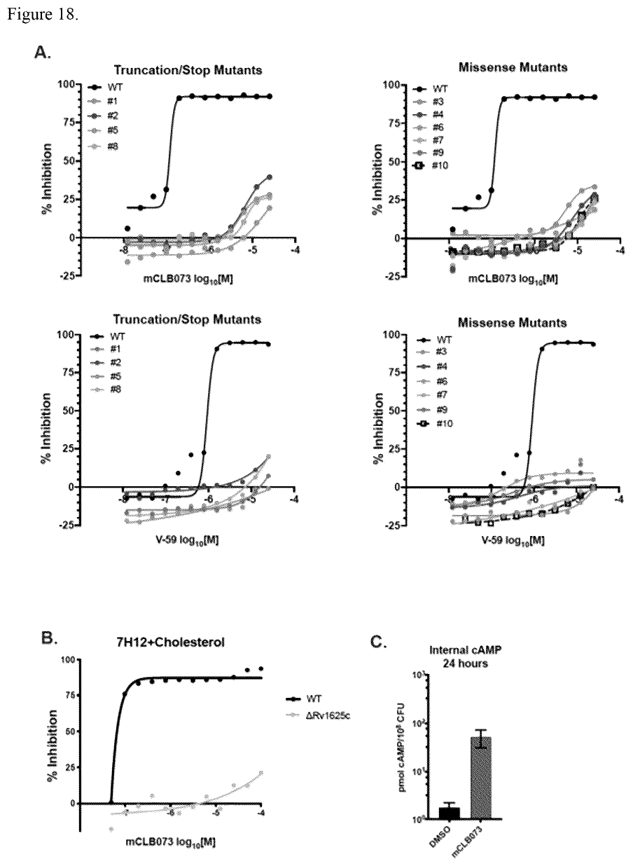

Rv1625c is a biochemically confirmed AC enzyme that catalyzes the intramolecular cyclization of ATP into cAMP. Therefore, whether V-59 increases cAMP production in whole bacteria in an Rv1625c-dependent manner was determined. V-59 induced cAMP by ˜70-fold in WT and ˜140-fold in Comp_{Full}, but did not affect the ΔRv1625c mutant (FIG. 3A). To determine whether Rv1625c is sufficient for V-59 to stimulate cAMP production, the rv1625c gene in an AC-deficient strain of E. coli was heterologously expressed. This strain is deficient in its own single AC (cya⁻ E. coli), ensuring that the cAMP produced in this experiment is due to Rv1625c activity. V-59 treatment increased cAMP levels in cya⁻ E. coli transformed with the Rv1625c expression plasmid (FIG. 3B). Similar results were obtained during treatment with an optimized analog of V-59, named mCLB073, which is described later in more detail. In Mtb, it was found that spontaneous mutations in rv1625c confer resistance to V-59 (FIG. 3C). Mutations predicted to truncate the Rv1625c protein and inactivate its cyclase domain resulted in resistance (FIG. 18A). Also identified were missense mutations within the transmembrane and cyclase domains of Rv1625c that confer resistance; without further biochemical characterization, it is ambiguous whether these mutations generate resistance by preventing V-59 binding to Rv1625c, or by disabling Rv1625c enzyme activity. Together these results indicate that V-59 activates Rv1625c selectively in Mtb, and that Rv1625c expression is sufficient for V-59 to activate cAMP synthesis, which is necessary for V-59 to inhibit Mtb growth.

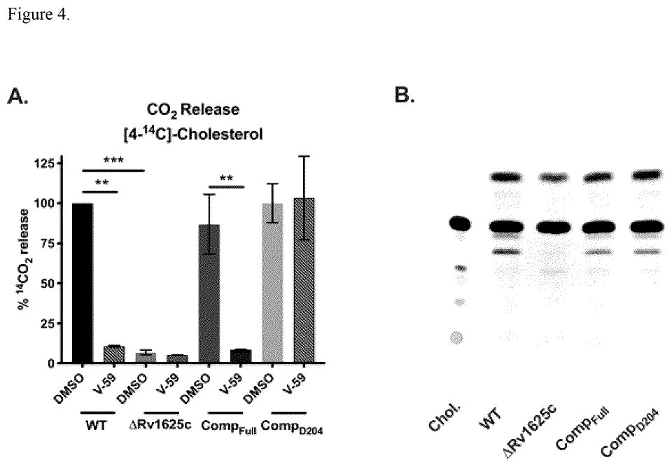

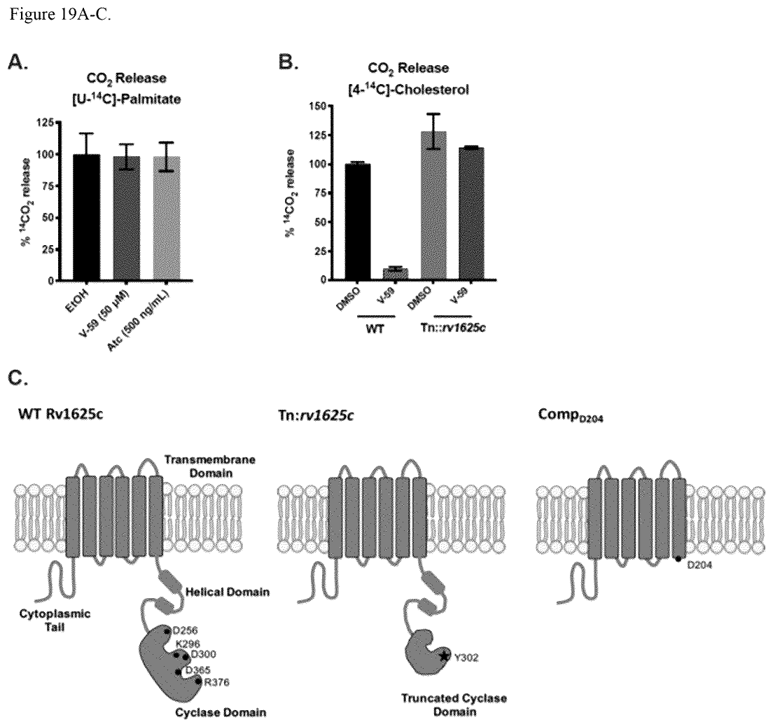

Because V-59 impairs growth of Mtb in cholesterol media, whether using V-59 to chemically activate Rv1625c inhibits the bacterium's ability to break down cholesterol was tested. When Mtb degrades the A-ring of [4-¹⁴C]-cholesterol, [1-¹⁴C]-pyruvate is released; subsequently, pyruvate dehydrogenase activity mediates the conversion of [1-¹⁴C]-pyruvate into acetyl-CoA and ¹⁴CO₂. Therefore, to quantify cholesterol degradation in Mtb, ¹⁴CO₂ released following [4-¹⁴C]-cholesterol breakdown by the bacteria was captured. It was found that V-59 decreased ¹⁴CO₂ release in WT by ˜89% (FIG. 4A). By contrast, V-59 had no measurable effect on ¹⁴CO₂ released from breakdown of the fatty acid [U-¹⁴C]-palmitate (FIG. 19A). This suggests that chemically activating Rv1625c preferentially inhibits cholesterol utilization in WT Mtb, rather than equally inhibiting all lipid utilization by the bacterium.

Unexpectedly, it was found that the ΔRv1625c mutant has an intrinsic defect in cholesterol degradation (FIG. 4A). In contrast to ΔRv1625c, the Rv1625c transposon mutant strain (Tn::rv1625c) had no defect in ¹⁴CO₂ release from [4-¹⁴C]-cholesterol (FIG. 19B). The Tn::rv1625c strain has a transposon insertion within the coding sequence located after the last exit of Rv1625c's six-helical transmembrane domain (amino acid Y302) (FIG. 19C). This likely truncates the protein, eliminating more than half of the C-terminal cyclase domain, while leaving the N-terminal cytoplasmic tail and six-helical transmembrane domain intact. Thus, the ΔRv1625c strain was complemented with a construct that expresses only the N-terminal cytoplasmic tail and six-helical transmembrane domain of Rv1625c (Comp_{D204}) (FIG. 19C). Cholesterol degradation was restored in the Comp_{D204} strain (FIG. 4A), indicating that the transmembrane domain of Rv1625c is required for the complete degradation of cholesterol. Importantly, V-59 inhibited ¹⁴CO₂ release in the Comp_{Full} strain; however, V-59 did not prevent ¹⁴CO₂ release in the Comp_{D204} strain which lacks the Rv1625c cyclase domain (FIG. 4A).

To further examine whether cholesterol degradation is blocked in ΔRv1625c Mtb, thin-layer chromatography (TLC) to track accumulation of [4-¹⁴C]-cholesterol-derived metabolites was used. Compared to WT Mtb, the culture supernatant of ΔRv1625c was deficient in at least one cholesterol-derived degradation intermediate, and the production of this intermediate was restored in the Comp_{Full} strain (FIG. 4B). Collectively, these results indicate that the cyclase domain of Rv1625c must be present for V-59 to inhibit cholesterol catabolism, and the transmembrane domain of Rv1625c is required for complete cholesterol breakdown, thereby establishing a direct link between the target of V-59 and the cholesterol pathway in Mtb. To our knowledge, this is the first AC that has been linked to modulation of a downstream metabolic pathway in Mtb.

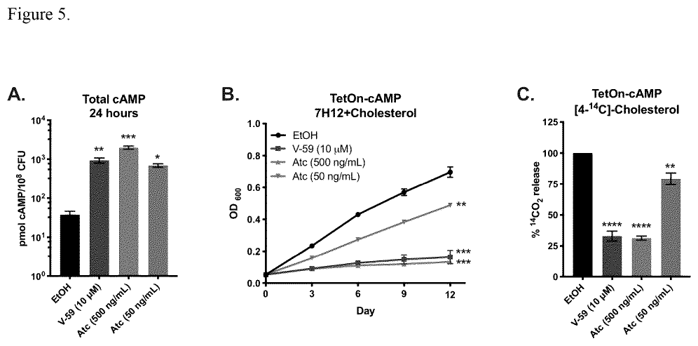

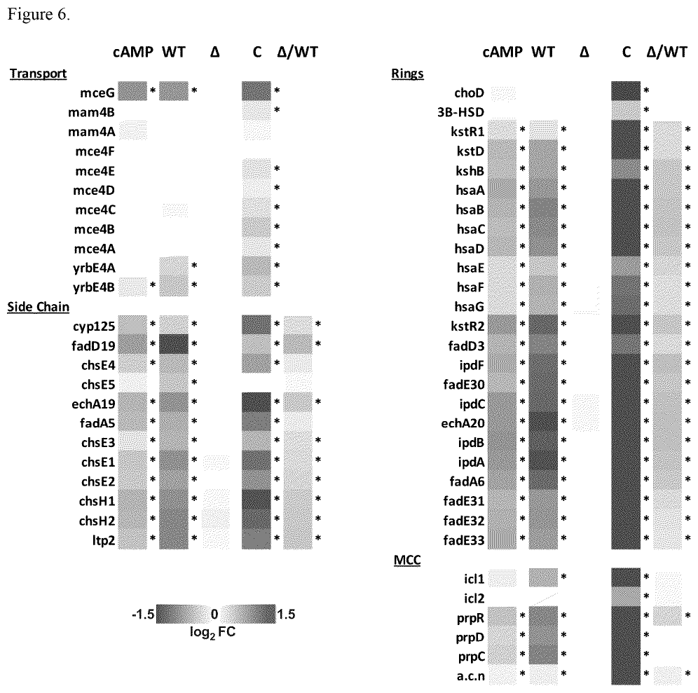

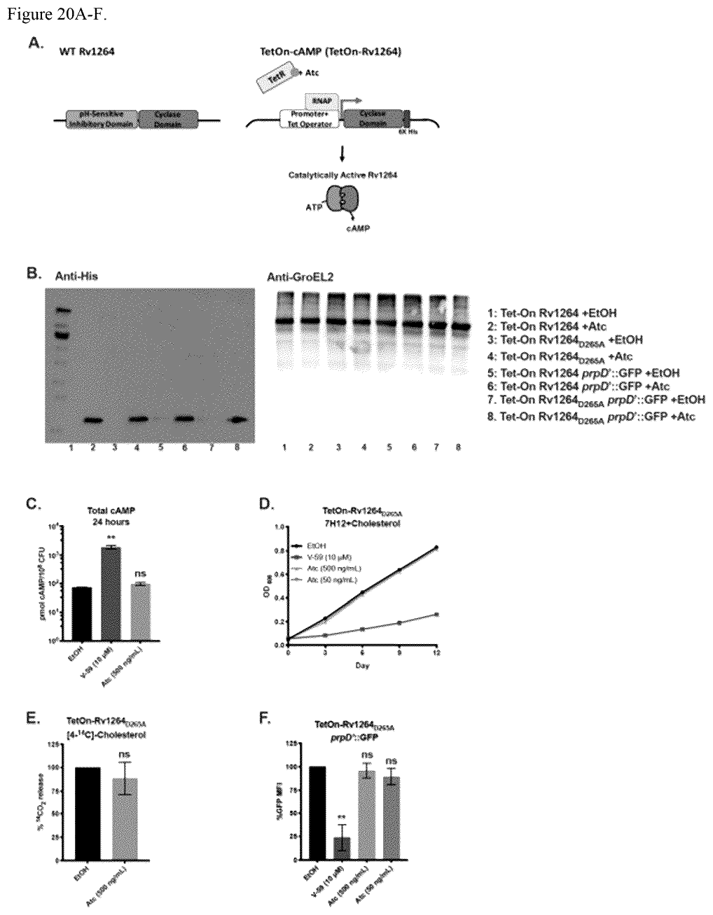

Next, it was investigated whether cAMP signaling can modulate cholesterol metabolism in an Rv1625c-independent manner by using a novel inducible construct (TetOn-cAMP) to increase cAMP synthesis in Mtb. This TetOn-cAMP construct carries an anhydrotetracycline (Atc) inducible promoter that controls expression of the catalytic domain of the mycobacterial AC Rv1264 (FIG. 20A). Atc induced cAMP synthesis in WT Mtb carrying the TetOn-cAMP construct in a dose-dependent manner, reaching levels comparable with V-59 treatment (FIG. 5A). This tool is an advancement over previous approaches for several reasons: it does not rely on diffusion of an external cAMP analog into the bacteria, it requires the bacteria to synthesize cAMP from ATP which more closely models the dynamics of AC signaling, and it increases cAMP by 24 hours post-induction in a dose-dependent fashion. Atc treatment inhibited growth of WT bacteria carrying the TetOn-cAMP construct in cholesterol media (FIG. 5B) and also decreased [4-¹⁴C]-cholesterol degradation to ¹⁴CO₂ (FIG. 5C). Similar to V-59 treatment, activating cAMP synthesis with Atc did not inhibit degradation of the fatty acid [U-¹⁴C]-palmitate to ¹⁴CO₂ (FIG. 19C). As a control, the TetOn-cAMP construct by mutating a catalytic residue of Rv1264 (TetOn-Rv1264_{D265A}) to render it catalytically inactive was modified. Atc induced expression of the Rv1264_{D265A} protein in WT Mtb carrying the TetOn-Rv1264_{D265A} construct (FIG. 20B), but this strain did not produce increased cAMP in response to Atc (FIG. 20C). Inducing Rv1264_{D265A} expression did not inhibit bacterial growth in cholesterol media (FIG. 20D) or cholesterol degradation (FIGS. 20E-F). These results demonstrate that activating cAMP synthesis through a mechanism that is independent of Rv1625c is sufficient to regulate cholesterol utilization in Mtb in a dose-dependent manner. Next, transcriptional responses of Mtb following V-59 treatment, or upon induction of the TetOn-cAMP construct, during growth in cholesterol media were characterized. The RNA-seq datasets revealed a shared pattern of differential gene expression that is consistent with an early blockade in the cholesterol degradation pathway. In Mtb, the side-chain and A-B rings of cholesterol are degraded by enzymes encoded in the Rv3574/KstR1 regulon. KstR1 is a TetR-like transcriptional repressor that binds the second cholesterol degradation intermediate, 3-hydroxy-cholest-5-ene-26-oyl-CoA, which de-represses the KstR1 regulon and permits cholesterol degradation to occur. Thus, increased expression of the KstR1 regulon is an indicator of cholesterol degradation in Mtb. Inducing cAMP synthesis, with V-59 or by activating TetOn-cAMP, prevented transcriptional induction of the KstR1 regulon in WT Mtb (FIG. 6). This included key genes required for cholesterol transport (rv0655/mceG and rv35021/yrbE4B) and cholesterol catabolism. Cholesterol degradation releases propionyl-CoA, and Mtb primarily assimilates this intermediate into central metabolism via the methylcitrate cycle (MCC). As propionyl-CoA pools increase, Mtb upregulates expression of the genes encoding MCC enzymes (rv0467/icl1, rv11301/prpD, rv1131/prpC). V-59 treatment and induction of the TetOn-cAMP construct in WT Mtb each prevented upregulation of MCC genes (FIG. 6). Paralleling previous experiments, V-59 induced more pronounced changes in the transcriptional signature in the Comp_{Full} strain. Notably, genes required for cholesterol transport and genes (hsaEFG) necessary for conversion of the cholesterol-derived catabolic intermediate 2-hydroxy-hexa-2,4-dienoic acid to pyruvate and propionyl-CoA were upregulated in the Comp_{Full} strain following V-59 treatment. It is plausible that these expression profiles reflect a compensatory response to inhibition of cholesterol degradation by V-59, and a concomitant decrease in availability of MCC or tricarboxylic acid cycle intermediates. Consistent with our previous observations (FIG. 4), expression of cholesterol side-chain and ring degradation genes, but not transport or MCC genes, was intrinsically blocked in the ΔRv1625c strain relative to WT (FIG. 6). These observations further support the conclusion that Rv1625c is involved in downstream cholesterol metabolism in Mtb. Importantly, V-59 treatment did not alter the transcriptional signature of the ΔRv1625c strain.

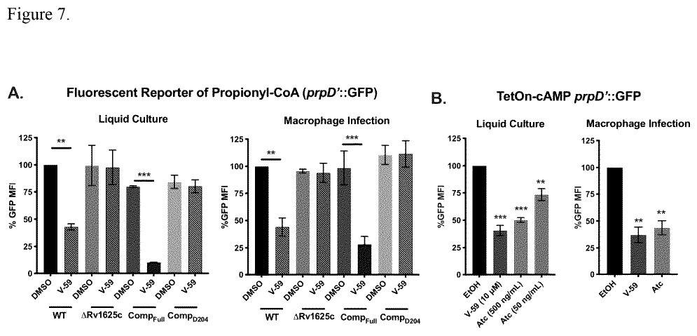

To validate these findings, a reporter (prpD′::GFP) that expresses GFP under control of a MCC gene promoter (prpD), which indicates cellular levels of propionyl-CoA was used. V-59 decreased GFP signal in WT by ˜50%, but did not impact the ΔRv1625c or Comp_{D204} strains in cholesterol media or during macrophage infection (FIG. 7A). V-59 also dampened GFP signal by ˜90% in the Comp_{Full} strain (FIG. 7A). Similarly, inducing cAMP synthesis in WT Mtb carrying the TetOn-cAMP construct was sufficient to inhibit GFP signal during growth in cholesterol media and during macrophage infection (FIG. 7B). Inducing expression of the inactive Rv1264_{D265A} protein (FIG. 20B) did not change the GFP signal (FIG. 20F). Overall, these data demonstrate that inducing cAMP synthesis in Mtb, via V-59 treatment or TetOn-cAMP activation, impairs cholesterol degradation and the release of key metabolic intermediates including propionyl-CoA in Mtb. During macrophage infection, additional signals not present in cholesterol media (e.g. low pH, additional carbon sources) that can regulate Mtb metabolism may also influence propionyl-CoA levels, contributing to overall prpD′::GFP expression. Importantly, the effects of V-59 treatment require the catalytic domain of Rv1625c, and cAMP synthesis is a dominant signal in the mechanism by which V-59 inhibits Mtb growth.

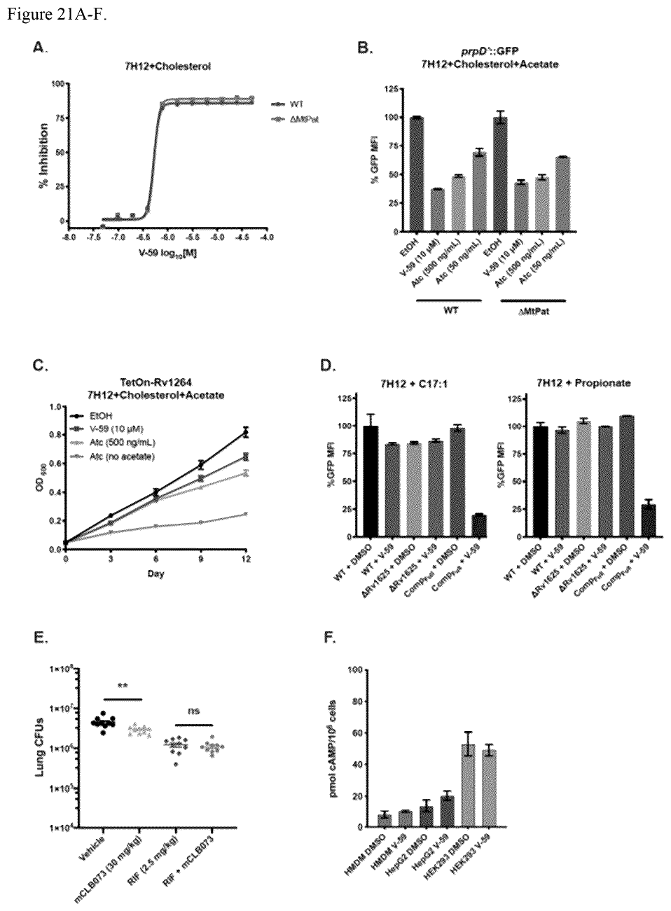

Inducing cAMP synthesis blocks cholesterol utilization in Mtb, but the mechanism mediating this is unknown. Because fatty acid metabolism can be modulated by the cAMP-binding protein Rv0998/Mt-Pat, whether Mt-Pat also mediates V-59-dependent inhibition of cholesterol utilization was investigated. However, inhibition of growth (FIG. 21A) and inhibition of MCC gene induction (FIG. 21B) by V-59 treatment were not altered in an Mt-Pat mutant. Notably, the Comp_{Full} strain was uniquely susceptible to V-59 in cholesterol media supplemented with the short chain fatty acid acetate (FIGS. 1B and 21C), and V-59 was also found to block MCC gene induction in the Comp_{Full} strain during growth with odd-chain fatty acids (FIG. 21D). This suggests that additional metabolic defects, possibly in fatty acid utilization or central metabolism, are induced under these conditions. These observations correlated with a higher threshold of cAMP induction (FIG. 3A).

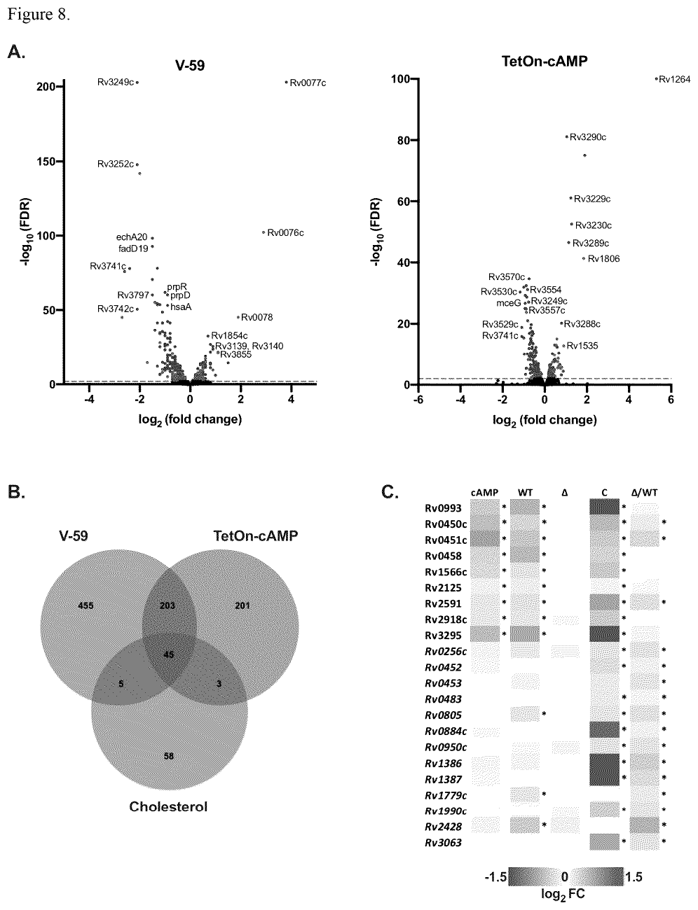

To define a set of commonly regulated cAMP-dependent genes using an unbiased analysis, all of the statistically significant differentially expressed genes associated with V-59 treatment or TetOn-cAMP induction in WT Mtb were compared (FIG. 8A). A shared set of 248 genes were identified (FIG. 8B). Because the selected growth condition was cholesterol media, 45 of these genes are associated with cholesterol utilization. As discussed above, those with biochemically confirmed roles in cholesterol utilization were noted (FIG. 6). Many of the remaining 203 genes do not have well defined functions in Mtb, and it was chosen to focus on a small subset that were previously predicted to be regulated by the cAMP-binding transcription factor Rv3676/CRPMT.

Aside from Mt-Pat, CRPMt is the best-studied cAMP-binding effector protein in Mtb. CRPMt is designated as a potential cAMP-responsive transcription factor, with a predicted regulon of ˜100 genes in Mtb. CRPMt may also be required to maintain Mtb fitness in macrophages and during chronic infection in mice. It was found that activating cAMP via V-59 treatment or TetOn-cAMP induction was associated with transcriptional changes to a shared set of only 9 CRPMt regulon genes during growth in cholesterol media, which have unknown functions except for rv0450c/mmpL4 and rv0451c/mmpS4 (FIG. 8C). Importantly, among the predicted CRPMT regulon genes, rv0805 is upregulated during V-59 treatment. Rv0805 is the only known phosphodiesterase in Mtb, and these enzymes contribute to cAMP signaling pathway homeostasis by hydrolyzing cAMP to AMP. Given that rv0805 mutant Mtb also has a growth defect in cholesterol media, it is reasonable to speculate that rv0805 is upregulated during V-59 treatment in the presence of cholesterol as a compensatory response to help decrease cAMP levels and restore cholesterol utilization. Taken together, this is consistent with our other results indicating that a threshold of increased cAMP is inhibitory during cholesterol utilization in Mtb. Surprisingly, an additional set of 13 predicted CRPMt regulon genes displayed intrinsic differential expression in the ΔRv1625c strain relative to WT (FIG. 8C) but were not universally differentially expressed in response to cAMP induction. This suggests that Rv1625c might play a native role in regulating some CRPMT operon genes during cholesterol utilization. Overall, these findings are significant because they demonstrate that only a subset of CRPMt operon genes are altered either in response to induction of cAMP synthesis, or through loss of Rv1625c, in the presence of cholesterol. While this study does not explain the native role of CRPMT during infection, V-59 and TetOn-cAMP can be used as tools in future studies to examine regulation of this operon under different growth conditions or during infection which may provide insight into its function in Mtb pathogenesis.

Next, it was sought to identify chemical features that are essential in a potent Rv1625c agonist, and to develop an optimized compound for use during in vivo studies. The screening hit (V-59) was relatively potent against Mtb in macrophages and had several satisfactory pharmacological features including plasma exposure above the EC₅₀ (as determined in cholesterol media) for approximately 24 hours following oral dosing in mice at 20 mg/kg (FIG. 2). It was sought to improve the properties of the V-59 compound series with medicinal chemistry. Structure activity relationship studies determined that replacing the tetrazole ring in V-59 with the oxadiazole ring in mCIS635 improved potency and slightly improved solubility. Replacing the 4-methyl-1,2,5-oxadiazole ring in V-59 with a 1-methyl-1H-1,2,4-triazole ring addressed the liability of the oxadiazole ring and generated the lead compounds mCLE299, mCLF177, mCLF178, and mCLB073 that had improved properties including better potency and extended plasma exposure following oral administration in mice. Constraining the piperidine ring was explored in order to increase compound solubility by lowering its lattice energy, through an azabicyclic ring and a chiral center in several molecules of this series (FIG. 2 and Table S2). Interestingly, the cis isomer (mCLF024) displayed potency similar to the advanced lead compounds in this series, while the trans isomer (mCLF025) was inactive (Table S2).

TABLE S2
Structures and activities of isomer compounds.

| | 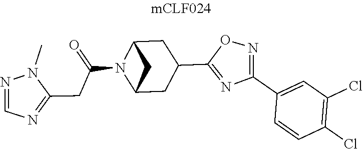 | 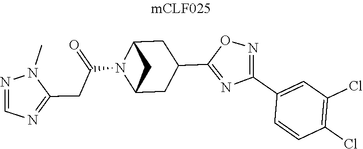 |
|---|---|---|
| MW | 433.29 | 422.29 |
| | In vitro potency assays | |
| Macrophages EC₅₀ (nM) | 97 | >30,000 |
| Cholesterol EC₅₀ (nM) | 49 | >30,000 |
| | Toxicity | |
| hERG IC₅₀ (nM) | 11.8 | — |
| | ADME | |
| PBB mouse | 97.8 | — |
| (% bound) | | |
| Caseum binding | <0.01 | — |
| (% unbound) | | |
| ER mouse/human | <0.3/<0.3 | — |
| Solubility (μM, pH 6.8) | 94.0 | — |

MW, molecular weight; —, not determined; EC₅₀, half-maximal effective concentration; IC₅₀, half-maximal inhibitory concentration; ADME, absorption,
distribution, metabolism, excretion; PPB, plasma protein binding; ER, extraction ratio; PO, per os.

Among the optimized analogs, the lead compound (1-(4-(3-(3,4-dichlorophenyl)-1,2,4-oxadiazol-5-yl)-1-piperidinyl)-2-(2-methyl-2H-1,2,4-triazol-3-yl)-1-ethanone), named mCLB073, exhibited a ˜-17-fold potency improvement against Mtb in cholesterol media relative to V-59 while maintaining excellent pharmacokinetic properties and a good safety profile. It was then verified that mCLB073 retained on-target activity. The ΔRv1625c strain was refractory to mCLB073 treatment (FIG. 18B), and mCLB073 activates cAMP synthesis (FIGS. 3B and 18C). Additionally, spontaneous resistant mutants in Mtb cultured with mCLB073 were isolated. All spontaneous resistant mutants that were isolated contained mutations in rv1625c that conferred resistance to mCLB073 and cross-resistance to V-59 (FIG. 3C). These results indicate that mCLB073 is a genuine Rv1625c agonist. When dosed orally in mice, mCLB073 maintained plasma exposure over the EC₅₀ identified in cholesterol media for at least 24 hours (FIG. 2). This demonstrated that mCLB073 is suitable for once-daily oral dosing in mouse models of infection and justified using this compound as a chemical probe during in vivo studies.

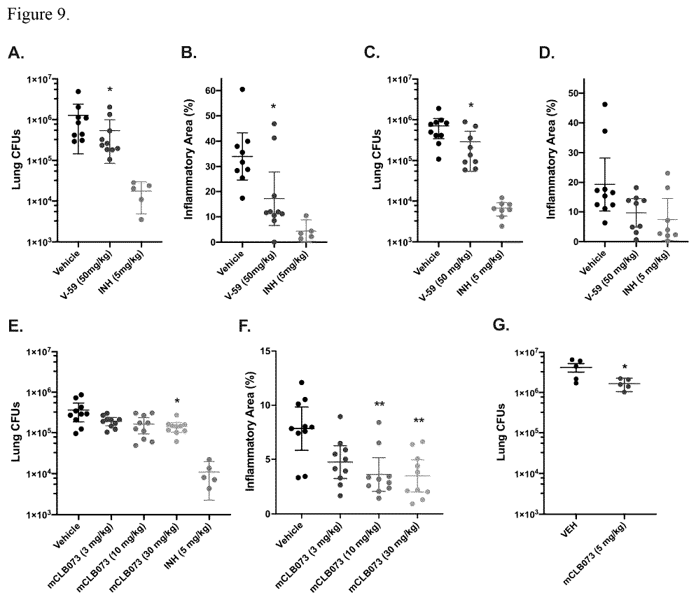

Next, examined was whether treatment with Rv1625c agonists would alter Mtb survival in a mouse model of infection. BALB/c mice were infected with WT Mtb via the intranasal route, and administered a vehicle control, V-59, or isoniazid by oral gavage once-daily during weeks 4 through 8 post-infection. V-59 (50 mg/kg) caused a ˜0.4-log₁₀ reduction in lung CFUs and reduced the extent of lung inflammation by ˜50% (FIGS. 9A and 9B). Similar results were obtained in CFU counts (˜0.5-log₁₀ reduction) in the lungs of C3HeB/FeJ mice infected and treated in the same manner (FIGS. 9C and 9D). The C3HeB/FeJ Mtb infection model was used because these mice produce type I IFN- and neutrophil-driven pathology that results in well-organized necrotic granulomas containing high numbers of extracellular bacterial. Thus, the finding that an Rv1625c agonist inhibits bacterial growth and limits lung pathology in both a relatively resistant and a susceptible model of TB suggests that this compound series could be effective despite the heterogeneous host response mounted in Mtb infections. To verify the improved potency of mCLB073 against Mtb in vivo, BALB/c mice were infected with WT Mtb via the intranasal route, and administered a vehicle control, mCLB073, or isoniazid by oral gavage once-daily during weeks 4 through 8 post-infection. Treatment with mCLB073 (30 mg/kg) reduced Mtb CFUs in the lungs of mice significantly (˜0.4-log₁₀ reduction) (FIG. 9E) and decreased the extent of lung pathology by ˜45% (FIG. 9F). In a separate study of BALB/c mice that were aerosol infected and treated in the same manner, a significant reduction in lung CFUs (˜0.4-log₁₀ reduction) at a lower dose of mCLB073 (5 mg/kg) which further confirms the improved pharmacological properties of mCLB073 was observed (FIG. 9G).

Because the mechanism of action of mCLB073 is novel, whether mCLB073 treatment would lead to increased tolerance to a frontline TB drug (rifampicin) during infection was tested. It was found that the addition of mCLB073 (30 mg/kg) to a sub-optimal dose of rifampicin did not increase the bacterial burden in the lungs of BALB/c mice, suggesting this mechanism of action does not promote tolerance to other TB antibiotics (FIG. 21E). Finally, the potential concern that this chemotype would activate off-target, mammalian AC enzymes was addressed. No evidence was found that V-59 activates ACs in mammalian cells (FIG. 21F), and the low toxicity profile of the Rv1625c-activating compounds (FIG. 2) suggests limited off-target activation of mammalian ACs. These results demonstrate the increased potency of mCLB073 relative to V-59 in vivo, and suggest that chemically activating cAMP synthesis in Mtb during chronic infection confers a fitness cost to the bacterium.

Mtb possesses an expanded repertoire of cAMP signaling pathway components compared to other bacteria, suggesting this is an important mechanism to coordinate physiological functions in response to environmental cues. However, how Mtb physiology can be regulated through cAMP signaling, particularly through activation of specific AC enzymes, is not well understood. It was also not previously established whether this signaling pathway could be manipulated pharmacologically to disrupt Mtb pathogenesis. This gap in knowledge is partly explained by the lack of chemical and genetic tools that are equivalent to the eukaryotic AC agonist forskolin in the Mtb system. In this study a chemical AC agonist that is suitable for in vitro and in vivo studies was identified. And in a complementary approach, a TetOn-cAMP construct that permits dose-dependent induction of cAMP synthesis in Mtb was created and validated. These tools allowed us to establish a link between induction of cAMP synthesis, downregulation of cholesterol utilization, and inhibition of Mtb pathogenesis during infection.

Here, a collection of compounds that were identified in a high-throughput screen as inhibitors of Mtb growth in macrophages and cholesterol media was re-examined. Based on a previous study it was known that this collection contained at least three Rv1625c-dependent compounds, and sought to identify an additional Rv1625c agonist with improved potency and acceptable properties for use in in vivo studies. From the screening hits, a candidate small molecule named V-59 that displayed promising pharmacological properties was identified (FIG. 2). It was then determined that growth inhibition by V-59 in cholesterol media and in macrophages requires a functional Rv1625c enzyme (FIGS. 1A, 1C and 3C), and V-59 induces cAMP synthesis in an Rv1625c-dependent manner (FIGS. 3A and 3B). By quantifying degradation of the A-ring of cholesterol, it was found that V-59 indeed blocks cholesterol utilization, in a mechanism that requires the cyclase domain of Rv1625c (FIG. 4). Our combined results strongly suggested that V-59 binds to the Rv1625c enzyme to activate AC activity, which artificially increases cAMP synthesis in Mtb to inhibit cholesterol utilization and impairs bacterial growth in cholesterol media and macrophages.

To study the impact of cAMP induction independent of V-59 and Rv1625c, a TetOn-cAMP construct that increases cAMP synthesis in a dose-dependent manner was developed (FIG. 5A). It was found that inducing cAMP synthesis is sufficient to decrease growth of Mtb in cholesterol media (FIG. 5B) and to block cholesterol degradation (FIG. 5C). Transcriptional studies revealed hallmarks indicating that cholesterol degradation is inhibited early in the breakdown process following V-59 treatment, or induction of cAMP synthesis via the TetOn-cAMP construct (FIGS. 6 and 7). Together, this demonstrates that inducing cAMP through a different AC is sufficient to mimic the effects of an Rv1625c agonist, which suggests that AC activation is a general mechanism that can be leveraged to inhibit cholesterol utilization in Mtb. However, the experiments presented here did not address how native activation of the many individual ACs present in Mtb may work together to differentially regulate distinct physiological pathways, and this is a question that warrants future investigation. Collectively, our findings indicate that chemically or genetically inducing cAMP synthesis in Mtb above a certain threshold inhibits cholesterol degradation, blocks transcriptional activation of hallmark cholesterol utilization genes, and decreases propionyl-CoA pools in proportion to the amount of cAMP induced. Cholesterol breakdown is a many-stage process, and side chain and ring degradation can occur in tandem. Considering this, one interpretation consistent across these results is that robust activation of cAMP synthesis prevents cholesterol side chain and ring degradation simultaneously, and this decreases the breakdown of cholesterol to an early intermediate that is required to de-repress the KstR1 regulon.

While investigating the effect of V-59 on Rv1625c activity and cholesterol utilization, it was unexpectedly discovered that the six-helical transmembrane domain of Rv1625c and the associated N-terminal cytoplasmic tail is intrinsically required for complete cholesterol degradation in Mtb (FIGS. 4A and 4B). Given that Rv1625c had no previously predicted role in cholesterol utilization, this is a surprising connection between the AC that V-59 activates, and the metabolic pathway it inhibits. The native function of Rv1625c signaling during infection is not established, and Rv1625c is the only AC that has been linked to a specific downstream metabolic pathway in Mtb thus far. Our finding that the Rv1625c transmembrane domain is required for cholesterol ring catabolism expands on work by others showing that the catalytic domain is not the only functionally relevant component of this AC, but the mechanism that mediates its involvement in cholesterol catabolism remains to be determined. It is not known if the transmembrane domain of Rv1625c mediates protein-protein interactions that are required to complete cholesterol catabolism and whether this, or an alternative mechanism, is involved in maximizing regulation of cholesterol utilization by Rv1625c agonists. It is notable that the cholesterol utilization defect in the ΔRv1625 strain was likely limited to ring catabolism (FIG. 4A), and was not sufficient to impact bacterial growth in cholesterol media or macrophages (FIGS. 1A and 1C), or to inhibit transcriptional activation of methylcitrate cycle genes by liberation of propionyl-CoA (FIGS. 6 and 7A). Because degradation of the cholesterol side chain and rings can proceed in tandem, it is conceivable that a blockage in ring catabolism could be present in ΔRv1625 Mtb, while growth on cholesterol and liberation of propionyl-CoA and acetyl-CoA from the side chain are maintained. Two genes neighboring Rv1625c (rv1626/pdtaR and rv1627c) have been predicted to be required during cholesterol utilization. It will be helpful to determine whether Rv1625c interacts with these proteins, or others capable of regulating lipid metabolism in Mtb. Moreover, it would be interesting to examine whether the binding of V-59 to Rv1625c not only activates cAMP synthesis but also alters an interaction between Rv1625c and a relevant protein. This would help explain the distinct but overlapping cholesterol utilization defects that were observed in WT Mtb treated with V-59, the ΔRv1625 strain, and TetOn-cAMP Mtb treated with different doses of Atc. The amount of cAMP produced by the TetOn-cAMP strain was most similar to V-59 treatment at the lower dose of Atc (50 ng/mL) tested (FIG. 5A). This dose was also correlated with less severe defects in growth in cholesterol media, ¹⁴CO₂ release from [4-¹⁴C]-cholesterol, and propionyl-CoA liberation from cholesterol than the defects observed with V-59 treatment (FIGS. 5B, 5C and 7B). Based on this and our observations in the ΔRv1625 strain, it is interesting to speculate that V-59 interacts with the Rv1625c protein, altering both a relevant protein interaction and increasing cAMP synthesis, both of which contribute to V-59's total effect on cholesterol utilization. However, the mechanism and kinetics of inducing cAMP synthesis with the TetOn-cAMP system are distinct from V-59 treatment, limiting our ability to conclude that a particular dose of Atc mimics V-59 treatment. Future experiments to identify cholesterol degradation intermediates that accumulate in WT Mtb treated with V-59 could help clarify this by allowing comparison of step(s) of the cholesterol degradation pathway that are blocked by V-59 treatment versus TetOn-cAMP induction or loss of the Rv1625c protein alone.

These findings expand our limited understanding of how cAMP signaling can alter metabolism in Mtb, and it remains to be determined whether a downstream cAMP-binding protein is required for V-59 or TetOn-cAMP induction to inhibit cholesterol utilization in Mtb. Rv0998/Mt-Pat was investigated because it is a cAMP-binding lysine acetyltransferase that was previously shown to acetylate and inactive the acetyl-CoA/propionyl-CoA ligase (Rv3667/Acs) and various FadD enzymes in Mtb, which can regulate incorporation of 2- and 3-carbon precursors into central metabolism. Mt-Pat was not required for V-59 to inhibit Mtb growth (FIG. 21A) or induction of MCC genes (FIG. 21B) in cholesterol media. This is consistent with data showing that acetate rescues growth during both V-59 treatment and TetOn-cAMP activation (FIGS. 1B and 21C), suggesting ACs has not been inactivated by Mt-Pat in either condition. The FadD enzyme Rv3515c/FadD19 initiates cholesterol side chain degradation but FadD19 is not a confirmed target of inactivation by Mt-Pat, and other FadD enzymes that are known to be acetylated by Mt-Pat are not established steps in cholesterol breakdown.

Although Mt-Pat likely does not mediate inhibition of cholesterol utilization downstream of V-59 treatment or TetOn-cAMP induction, it is unclear whether any of the other eleven predicted cAMP-binding proteins in Mtb are involved in this mechanism. The predicted operon of one other cAMP-binding protein, the transcription factor CRPMT, was examined and it was found that only a handful of these genes were differentially expressed in a cAMP-dependent and/or Rv1625c-dependent manner during growth in cholesterol media (FIG. 8). Notably, three of these genes are required for optimal growth of Mtb in cholesterol media and/or in mouse models of TB, but are not directly involved in cholesterol side chain or ring breakdown. Most of the remaining genes do not have established functions, making it difficult to predict how changes in their expression could impact specific aspects of Mtb physiology. Further experiments are needed to determine whether these transcriptional changes are indeed mediated through CRPMt activity, and whether this contributes significantly to the cholesterol utilization defects observed in Mtb during V-59 treatment or in the ΔRv1625c strain. Alternatively, because cholesterol uptake by the bacterium and initiation of cholesterol side chain breakdown by FadD19 are both ATP-dependent processes, it is possible that ATP depletion occurring during increased cAMP synthesis mediates inhibition of cholesterol utilization. Considering the large number of cAMP signaling pathway components present in Mtb, the mechanisms by which distinct ACs and downstream cAMP-responsive proteins can coordinate different physiological responses under native inducing conditions requires further investigation.

Compared to the previously-published Rv1625c agonist V-58, V-59 represents a significant advance because it is an Rv1625c agonist that is suitable for use in an in vivo model of TB. V-59 also provided a basis for understanding how to improve Rv1625c agonists through medicinal chemistry. A medicinal chemistry effort was completed that focused on addressing the structural liabilities of V-59 and improving the potency of its activity against Mtb. This identified mCLB073, a stable molecule with improved potency and desirable chemical, pharmacological, pharmacokinetics and safety properties, which makes it a good drug candidate for clinical testing and a useful compound for further in vivo studies of this pathway in Mtb. While investigating the potency of compounds in this series with constrained piperidine rings, also identified were molecules whose potency differed widely based on the chirality of their azabicyclic rings. In the future, it would be interesting to identify the underlying explanation for the differential potency of these compounds on Rv1625c activity through structural biology.

Chemical tools like V-59/mCLB073 that can be used to modulate distinct pathways in Mtb in infection models are valuable because it is difficult to predict the in vivo metabolic state and vulnerabilities Mtb experiences during infection. Then developed were V-59/mCLB073 as compounds that can effectively modulate cAMP synthesis and block cholesterol utilization in Mtb, while being suitable for in vivo studies. Single-agent studies using V-59/mCLB073 established that cAMP induction modestly impairs bacterial growth and lung pathology in mouse models of TB (FIG. 9). A recent study also found that the “MKR superspreader” strain of Mtb, an emergent multidrug-resistant strain of the modern Beijing lineage, displayed enhanced upregulation of cholesterol utilization genes during macrophage infection relative to the H37Rv reference strain. V-59 was used to show that activating Rv1625c to inhibit cholesterol utilization in the MKR strain selectively reduced intracellular survival of the bacteria in infected macrophages. This suggests that efficacy of Rv1625c agonists may be potentiated by cholesterol-related metabolic adaptations that are especially crucial to the intracellular survival of at least one multi-drug resistant strain of Mtb. Identifying this link between cAMP signaling, cholesterol utilization, and Mtb fitness during infection is important because it is challenging to target metabolic pathways in Mtb that are not redundant and are sufficiently distinct from human pathways to limit side-effects. Numerous studies have suggested that cholesterol utilization is a key metabolic adaptation that supports Mtb survival during chronic infection, but the efficacy of single-step inhibitors of cholesterol degradation may be limited, unless they are able to cause accumulation of toxic metabolites in the bacterium. Inhibitors that block this pathway early and/or shut down multiple steps present one desirable alternative. This study revealed that the Rv1625c agonists V-59/mCLB073 are an improvement over the single-step cholesterol degradation inhibitors that were reported previously; these compounds inhibit cholesterol catabolism early and/or at both side chain and ring degradation steps and display excellent pharmacokinetic properties. The optimized compound mCLB073 will facilitate future studies examining how Rv1625c agonists alter Mtb fitness in animal models that more faithfully recapitulate the complexities of the granuloma microenvironment present in human TB. Much remains to be discovered about the restrictions Mtb encounters in vivo when the bacterium can co-catabolize multiple complex substrates. Future infection studies that combine V-59/mCLB073 treatment with chemical or genetic modulation of other pathways in Mtb may provide interesting insights on this topic and reveal how to optimize disruption of Mtb fitness during V-59/mCLB073 treatment.

In summary, it is disclosed herein that activating cAMP synthesis in Mtb, either by activating Rv1625c AC activity with a small molecule agonist or by inducing expression of the minimal catalytic subunit of Rv1264, blocks cholesterol degradation. Rv1625c is also the first AC in Mtb to be linked directly to a particular downstream pathway. Mtb has a cadre of structurally-diverse ACs and predicted cAMP-binding proteins with mostly uncharacterized functions, which may represent potential to alter pathways beyond cholesterol utilization in this bacterium. However, it is unknown whether agonists for ACs other than Rv1625c would have comparable downstream effects in Mtb. In other pathogenic bacteria, cAMP signaling is known not only for coordinating changes to carbon metabolism, but also for mediating diverse functions including biofilm formation, virulence gene expression, and secretion systems. This study identified AC agonists suitable for in vivo studies of cAMP signaling in Mtb, which revealed that chemically activating cAMP synthesis may be an untapped mechanism for manipulating bacterial fitness during infection, redefining the traditional mechanism for an anti-virulence compound. In Mtb, AC activation is able to stall at least one metabolic pathway that supports in vivo survival, with the potential to yield a new antibiotic. (See: Wilburn, K. M., et al. (2022). PLoS pathogens, 18(2), e1009862)

### REFERENCES

1. World Health Organization. Global Tuberculosis Report 2019. (World Health Organization, Geneva, 2019).
2. Sarathy P. J, Dartois V. Caseum: a Niche for Mycobacterium tuberculosis Drug-Tolerant Persisters. Clinical microbiology reviews 33, (2020). https://doi.org/10.1128/CMR.00159-19 PMID: 32238365
3. Hunter L. R., Jagannath C., Actor K, J. Pathology of postprimary tuberculosis in humans and mice: contradiction of long-held beliefs. Tuberculosis 87, 267-278 (2007). https://doi.org/10.1016/j.tube.2006.11.003 PMID: 17369095
4. Peyron P., Vaubourgeix J., Poquet Y., Levillain F., Botanch C., Bardou F. et al. Foamy macrophages from tuberculous patients' granulomas constitute a nutrient-rich reservoir for M. tuberculosis persistence. PLoS pathogens 4, e1000204 (2008). https://doi.org/10.1371/journal.ppat.1000204 PMID:19002241
5. Kim J, M., Wainwright C, H., Locketz M. Bekker G, L., Walther B, G., Dittrich C. et al. Caseation of human tuberculosis granulomas correlates with elevated host lipid metabolism. EMBO Molecular Medicine 2, 258-274 (2010). https://doi.org/10.1002/emmm.201000079 PMID: 20597103
6. Wilburn M, K. Fieweger A, R., VanderVen C, B. Cholesterol and fatty acids grease the wheels of Mycobacterium tuberculosis pathogenesis. Pathogens and Disease 76, (2018). https://doi.org/10.1093/femspd/fty021 PMID: 29718271
7. Pandey K, A., Sassetti M, C. Mycobacterial persistence requires the utilization of host cholesterol. Proceedings of the National Academy of Sciences of the United States of America 105, 4376-4380 (2008). https://doi.org/10.1073/pnas.0711159105 PMID: 18334639
8. Chang C, J., Miner D, M., Pandey K, A., Gill P, W., Harik S, N., Sassetti M, C. et al. igr Genes and Mycobacterium tuberculosis Cholesterol Metabolism. Journal of Bacteriology 191, 5232-5239 (2009). https://doi.org/10.1128/JB.00452-09 PMID: 19542286
9. Nesbitt N, M., Yang X., Fontan P., Kolesnikova I., Smith I., Sampson S, N. et al. A thiolase of Mycobacterium tuberculosis is required for virulence and production of androstenedione and androstadienedione from cholesterol Infection and Immunity 78, 275-282 (2010). https://doi.org/10.1128/IAI.00893-09 PMID: 19822655
10. Yam C, K., D'Angelo I., Kalscheuer R., Zhu H., Wang X, J, Snieckus V. et al. Studies of a Ring-Cleaving Dioxygenase Illuminate the Role of Cholesterol Metabolism in the Pathogenesis of Mycobacterium tuberculosis. PLoS Pathogens 5, e1000344 (2009). https://doi.org/10.1371/journal.ppat.1000344 PMID: 19300498
11. Hu Y., van der Geize R., Besra S, G., Gurcha S, S., Liu A., Rohde M. et al. 3-Ketosteroid 9alpha-hydroxylase is an essential factor in the pathogenesis of Mycobacterium tuberculosis. Molecular Microbiology 75, 107-121 (2010). https://doi.org/10.1111/j.1365-2958.2009.06957.x PMID: 19906176
12. Smith M, C., Baker E, R., Proulx K, M., Mishra B, B., Long E, J., Park S. et al., Host-pathogen genetic interactions underlie tuberculosis susceptibility. bioRxiv 2020.12.01.405514, (2021).
13. Aiewsakun P., Prombutara P., Siregar P, A, T., Laopanupong T., Kanjanasirirat P., Khumpanied T. et al. Transcriptional response to the host cell environment of a multidrug-resistant Mycobacterium tuberculosis clonal outbreak Beijing strain reveals its pathogenic features. Scientific Reports 11, 3199 (2021). https://doi.org/10.1038/s41598-021-82905-x PMID: 33542438
14. McDonough A, K., Rodriguez A. The myriad roles of cyclic AMP in microbial pathogens: from signal to sword. Nature Reviews Microbiology 10, 27-38 (2011). https://doi.org/10.1038/nrmicro2688 PMID: 22080930
15. Chubukov V., Gerosa L., Kochanowski K., Sauer U. Coordination of microbial metabolism. Nat Rev Microbiol 12, 327-340 (2014). https://doi.org/10.1038/nrmicro3238 PMID: 24658329
16. Molina-Quiroz C, R., Silva-Valenzuela C., Brewster J., Castro-Nallar E., Levy B, S., Camilli A. Cyclic AMP Regulates Bacterial Persistence through Repression of the Oxidative Stress Response and SOS-Dependent DNA Repair in Uropathogenic Escherichia coli. mBio 9, (2018). https://doi.org/10.1128/mBio.02144-17 PMID: 29317513
17. Shenoy R, A., Visweswariah S, S. Mycobacterial adenylyl cyclases: biochemical diversity and structural plasticity. FEBS Lett 580, 3344-3352 (2006). https://doi.org/10.1016/j.febslet.2006.05.034 PMID:16730005
18. Tews I., Findeisen F., Sinning I., Schultz A., Schultz E, J., Linder U, J. The structure of a pH-sensing mycobacterial adenylyl cyclase holoenzyme. Science 308, 1020-1023 (2005). https://doi.org/10.1126/science.1107642 PMID: 15890882
19. Townsend D, P.Holliday M, P., Fenyk S., Hess C, K., Gray A, M., Hodgson R, D. et al. Stimulation of mammalian G-protein-responsive adenylyl cyclases by carbon dioxide. J Biol Chem 284, 784-791 (2009). https://doi.org/10.1074/jbc.M807239200 PMID: 19008230
20. Cann J, M., Hammer A., Zhou J., Kanacher T. A defined subset of adenylyl cyclases is regulated by bicarbonate ion. J Biol Chem 278, 35033-35038 (2003). https://doi.org/10.1074/jbc.M303025200 PMID: 12829712
21. Abdel Motaal A., Tews I., Schultz E, J., Linder U, J., Fatty acid regulation of adenylyl cyclase Rv2212 from Mycobacterium tuberculosis H37Rv. FEBS J 273, 4219-4228 (2006). https://doi.org/10.1111/j. 1742-4658.2006.05420.x PMID: 16925585
22. Shenoy R, A., Sreenath P, N., Mahalingam M., Visweswariah S, S. Characterization of phylogenetically distant members of the adenylate cyclase family from mycobacteria: Rv1647 from Mycobacterium tuberculosis and its orthologue ML1399 from M. leprae. The Biochemical journal 387, 541-551 (2005). https://doi.org/10.1042/BJ20041040 PMID: 15500449
23. Johnson M, R. McDonough A, K. Cyclic nucleotide signaling in Mycobacterium tuberculosis: an expanding repertoire. Pathogens and Disease 76, (2018).
24. Gazdik A, M., Bai G., Wu Y., McDonough A, K. Rv1675c (cmr) regulates intramacrophage and cyclic AMP-induced gene expression in Mycobacterium tuberculosis-complex mycobacteria. Molecular microbiology 71, 434-448 (2009). https://doi.org/10.1111/j.1365-2958.2008.06541.xPMID: 19040643
25. Bai G., McCue A, L., McDonough A, K. Characterization of Mycobacterium tuberculosis Rv3676 (CRPMt), a cyclic AMP receptor protein-like DNA binding protein. J Bacteriol 187, 7795-7804 (2005). https://doi.org/10.1128/JB.187.22.7795-7804.2005 PMID: 16267303
26. Rickman L., Scott C., Hunt M, D., Hutchinson T., Menendez C, M., Whalan R. et al. A member of the cAMP receptor protein family of transcription regulators in Mycobacterium tuberculosis is required for virulence in mice and controls transcription of the rpfA gene coding for a resuscitation promoting factor. Molecular microbiology 56, 1274-1286 (2005). https://doi.org/10.1111/j.1365-2958.2005.04609.xPMID: 15882420
27. Rittershaus C, S, E., Baek H, S., Krieger V, I., Nelson J, S., Cheng S, Y., Nambi S. et al. A Lysine Acetyltransferase Contributes to the Metabolic Adaptation to Hypoxia in Mycobacterium tuberculosis. Cell Chemical Biology 25, 1495-1505 e1493 (2018). https://doi.org/10.1016/j.chembiol.2018.09.009 PMID: 30318462
28. Nambi S., Gupta K., Bhattacharyya M., Ramakrishnan P., Ravikumar V., Siddiqui N. et al. Cyclic AMP-dependent protein lysine acylation in mycobacteria regulates fatty acid and propionate metabolism. Journal of Biological Chemistry 288, 14114-14124 (2013). https://doi.org/10.1074/jbc.M113.463992 PMID: 23553634
29. VanderVen C, B., Fahey J, R., Lee W., Liu Y., Abramovitch B, R., Memmott C. et al. Novel inhibitors of cholesterol degradation in Mycobacterium tuberculosis reveal how the bacterium's metabolism is constrained by the intracellular environment. PLoS Pathogens 11, e1004679 (2015). https://doi.org/10.1371/journal.ppat.1004679 PMID: 25675247
30. Guo L, Y., Seebacher T., Kurz U., Linder U, J., Schultz E, J. Adenylyl cyclase Rv1625c of Mycobacterium tuberculosis: a progenitor of mammalian adenylyl cyclases. EMBO Journal 20, 3667-3675 (2001). https://doi.org/10.1093/emboj/20.14.3667 PMID: 11447108
31. Guo L, Y., U. Kurz A., Schultz A., Linder U, J., Dittrich D., Keller C. et al. Interaction of Rv1625c, a mycobacterial class IIIa adenylyl cyclase, with a mammalian congener. Molecular microbiology 57, 667-677 (2005). https://doi.org/10.1111/j.1365-2958.2005.04675.x PMID: 16045612
32. Reddy K, S., Kamireddi M., Dhanireddy K., Young L., Davis A., Reddy T, P. Eukaryotic-like adenylyl cyclases in Mycobacterium tuberculosis H37Rv: cloning and characterization. Journal of Biological Chemistry 276, 35141-35149 (2001). https://doi.org/10.1074/jbc.M104108200 PMID: 11431477
33. Vercellino I., Rezabkova L., Olieric V., Polyhach Y., Weinert T., Kammerer A, et al R. Role of the nucleotidyl cyclase helical domain in catalytically active dimer formation. Proceedings of the National Academy of Sciences of the United States of America 114, E9821-E9828 (2017). https://doi.org/10.1073/pnas.1712621114 PMID: 29087332
34. Beltz S., Bassler J., Schultz E, J., Regulation by the quorum sensor from Vibrio indicates a receptor function for the membrane anchors of adenylate cyclases. eLife 5, (2016). https://doi.org/10.7554/eLife.13098 PMID: 26920221
35. Ziegler M., Bassler J., Beltz S., Schultz A., Lupas N, A., Schultz J, E. Characterization of a novel signal transducer element intrinsic to class IIIa/b adenylate cyclases and guanylate cyclases. FEBS Journal 284, 1204-1217 (2017). https://doi.org/10.1111/febs.14047 PMID:
36. Johnson M, R., Bai G., DeMott M, C., Banavali K, N., Montague R, C., Moon C. et al. McDonough, Chemical activation of adenylyl cyclase Rv1625c inhibits growth of Mycobacterium tuberculosis on cholesterol and modulates intramacrophage signaling. Molecular Microbiology (2017). https://doi.org/10.1111/mmi.13701 PMID: 28464471
37. Russell G, D., Huang L., VanderVen C, B. Immunometabolism at the interface between macrophages and pathogens. Nature Reviews Immunology 19, 291-304 (2019). https://doi.org/10.1038/s41577-019-0124-9 PMID: 30679807
38. Nazarova V, E., Montague R, C., La T., Wilburn M, K., Sukumar N., Lee W. et al. Rv3723/LucA coordinates fatty acid and cholesterol uptake in Mycobacterium tuberculosis. eLife 6, (2017). https://doi.org/10.7554/eLife.26969 PMID: 28708968
39. Gazdik A, M., McDonough A, K. Identification of cyclic AMP-regulated genes in Mycobacterium tuberculosis complex bacteria under low-oxygen conditions. J Bacteriol 187, 2681-2692 (2005). https://doi.org/10.1128/JB.187.8.2681-2692.2005 PMID: 15805514
40. Griffin E, J., Pandey K, A., Gilmore A, S., Mizrahi V., McKinney D, J., Bertozzi R, C. et al. Cholesterol catabolism by Mycobacterium tuberculosis requires transcriptional and metabolic adaptations. Chemistry & Biology 19, 218-227 (2012). https://doi.org/10.1016/j.chembiol.2011.12.016 PMID: 22365605
41. Baker J, J., Dechow J, S., Abramovitch B, R Acid Fasting: Modulation of Mycobacterium tuberculosis Metabolism at Acidic pH. Trends Microbiol 27, 942-953 (2019). https://doi.org/10.1016/j.tim.2019.06.005 PMID: 31324436
42. Serafini A., Tan L., Horswell S., Howell S., Greenwood J, D., Hunt M, D. et al. Mycobacterium tuberculosis requires glyoxylate shunt and reverse methylcitrate cycle for lactate and pyruvate metabolism. Molecular microbiology 112, 1284-1307 (2019). https://doi.org/10.1111/mmi.14362 PMID: 31389636
43. Akhter Y., Yellaboina S., Farhana A., Ranjan A., Ahmed N., Hasnain E, S. Genome scale portrait of cAMP-receptor protein (CRP) regulons in mycobacteria points to their role in pathogenesis. Gene 407, 148-158 (2008). https://doi.org/10.1016/j.gene.2007.10.017 PMID: 18022770
44. Wells M, R., Jones M, C., Xi Z., Speer A., Danilchanka O. Doornbos S, K., et al. Discovery of a siderophore export system essential for virulence of Mycobacterium tuberculosis. PLoS Pathog 9, e1003120 (2013). https://doi.org/10.1371/journal.ppat.1003120 PMID: 23431276
45. Griffin E, J., Gawronski D, J., DeJesus A, M., Ioerger R, T., Akerley J, B., Sassetti M, C. High-Resolution Phenotypic Profiling Defines Genes Essential for Mycobacterial Growth and Cholesterol Catabolism. PLoS Pathog 7, (2011). https://doi.org/10.1371/journal.ppat.1002251 PMID: 21980284
46. Driver R, E., Ryan J, G., Hoff R, D., Irwin M, S., Basaraba J, R., Kramnik I. et al. Evaluation of a mouse model of necrotic granuloma formation using C3HeB/FeJ mice for testing of drugs against Mycobacterium tuberculosis. Antimicrobial agents and chemotherapy 56, 3181-3195 (2012). https://doi.org/10.1128/AAC.00217-12 PMID: 22470120
47. Ji X, D., Yamashiro H, L., Chen J, K., Mukaida N., Kramnik I., Darwin H, K. et al. Type I interferon-driven susceptibility to Mycobacterium tuberculosis is mediated by IL-1Ra. Nature microbiology 4, 2128-2135 (2019). https://doi.org/10.1038/s41564-019-0578-3 PMID: 31611644
48. Moreira-Teixeira L., Tabone O., Graham M, C., Singhania A., Stavropoulos E., Redford S, P. et al. Mouse transcriptome reveals potential signatures of protection and pathogenesis in human tuberculosis. Nature immunology 21, 464-476 (2020). https://doi.org/10.1038/s41590-020-0610-z PMID:32205882
49. Moreira-Teixeira L., Stimpson J, P., Stavropoulos E., Hadebe S., Chakravarty P., Ioannou M. et al. Type I IFN exacerbates disease in tuberculosis-susceptible mice by inducing neutrophil-mediated lung inflammation and NETosis. Nat Commun 11, 5566 (2020). https://doi.org/10.1038/s41467-020-19412-6 PMID: 33149141
50. Lenaerts A., Barry 3rd, E, C., Dartois V. Heterogeneity in tuberculosis pathology, microenvironments and therapeutic responses. Immunological Reviews 264, 288-307 (2015). https://doi.org/10.1111/imr.12252 PMID: 25703567
51. Bassler J., Schultz E, J., Lupas N, A. Adenylate cyclases: Receivers, transducers, and generators of signals. Cellular Signaling 46, 135-144 (2018).
52. Beites T., O'Brien K., Tiwari D., Engelhart A, C., Walters S., Andrews J. et al. Plasticity of the Mycobacterium tuberculosis respiratory chain and its impact on tuberculosis drug development. Nature Communications 10, 4970 (2019). https://doi.org/10.1038/s41467-019-12956-2 PMID: 31672993
53. Warner D, F., Mizrahi V. Shortening treatment for tuberculosis-back to basics. New England Journal of Medicine 371, 1642-1643 (2014).
54. Zhang Y., Mitchison D. The curious characteristics of pyrazinamide: a review. International Journal of Tuberculosis and Lung Disease 7, 6-21 (2003). PMID: 12701830
55. Howard C, N., Khader A, S. Immunometabolism during Mycobacterium tuberculosis Infection. Trends Microbiol 28, 832-850 (2020). https://doi.org/10.1016/j.tim.2020.04.010 PMID: 32409147
56. Pethe K., Sequeira C, P., Agarwalla S., Rhee K., Kuhen K., Phong Y, W. et al. A chemical genetic screen in Mycobacterium tuberculosis identifies carbon-source-dependent growth inhibitors devoid of in vivo efficacy. Nature Communications 1, 57 (2010). https://doi.org/10.1038/ncomms1060 PMID:20975714
57. Ganapathy U., J. Marrero J., Calhoun S., Eoh H., de Carvalho S, P, L., Rhee K. et al. Two enzymes with redundant fructose bisphosphatase activity sustain gluconeogenesis and virulence in Mycobacterium tuberculosis. Nature Communications 6, 7912 (2015). https://doi.org/10.1038/ncomms8912 PMID:26258286
58. Crowe M, A., Casabon I., Brown L, K., Liu J., Lian J., Rogalski C, J. et al. Catabolism of the Last Two Steroid Rings in Mycobacterium tuberculosis and Other Bacteria. mBio 8, (2017). https://doi.org/10.1128/mBio.00321-17 PMID: 28377529

### EMBODIMENTS

The application provides the following embodiments of the invention:

Embodiment 1. A compound of Formula I or Formula II,

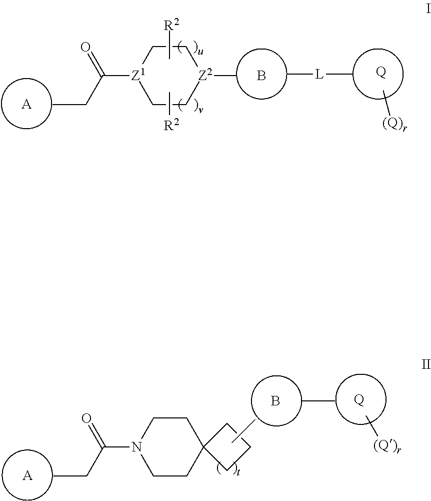

wherein:
A is selected from the group consisting of:

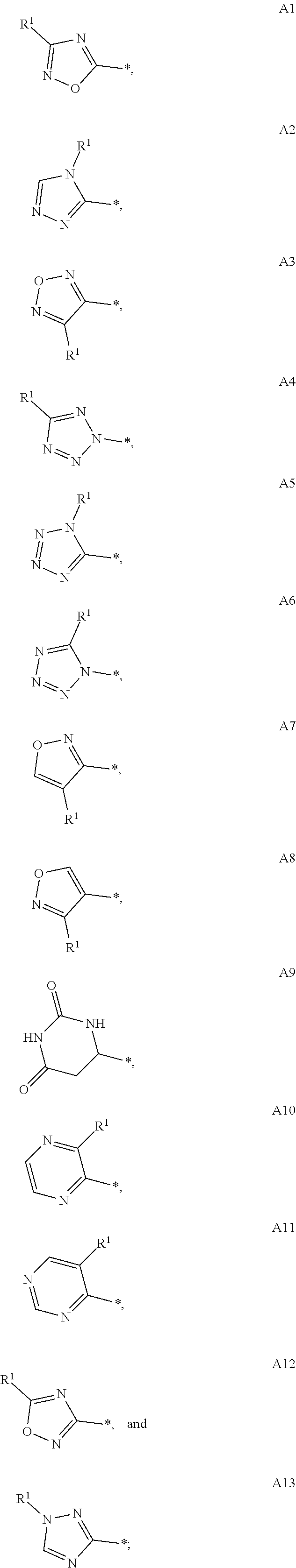

each R¹ is independently H or (C₁-C₆)alkyl;
Z¹ is CH or N;
u is 0 to 2;
v is 0 to 2;
both R² are absent or both R² together form a (C₁-C₂)alkylenyl bridge between two ring carbon atoms, provided that u and v are not both 0;
Z² is CH, CR³, or N;
   R³ is OH, CF₃, or (C₁-C₆)alkyl;
t is 0 or 1;
B is selected from the group consisting of:

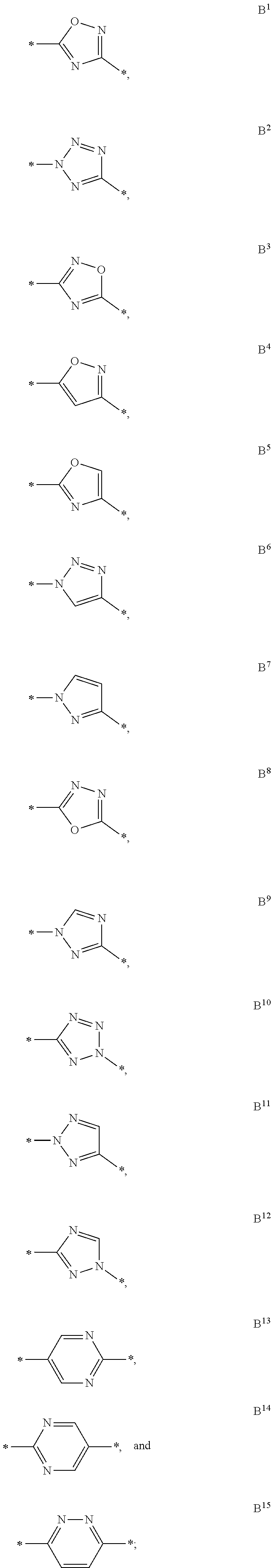

L is a bond;
   or if A is A³ and B is B¹, B², or B⁶, L may be —C(═O)—, —C(═O)N(R⁹)—, —CH₂—, or —O—; R⁹ is H, (C₁-C₆)alkyl, or (C₁-C₆)heteroalkyl;
Q is 3- to 8-membered monocyclic, 6- to 16-membered bicyclic, 4- to 14-membered fused bicyclic, or 5- to 11-membered spirocyclic cycloalkyl or heterocycloalkyl, Ph, or 5- to 8-membered heteroaryl, each optionally substituted with (Q′)r;
   or if A is A³ and B is B¹, B², or B⁶, Q may be (C₁-C₆)alkyl or (C₁-C₆)heteroalkyl;
   each Q′ is independently halo, halo (C₁-C₆) alkyl, haloalkoxy, —CN, (C₁-C₆)alkyl, (C₂-C₆)alkenyl, (C₂-C₆)alkynyl, (C₁-C₆)heteroalkyl, OH, oxo, amino, amido, imino, (C₁-C₆)alkyl ester, carboxyl, —SF₅, 3- to 8-membered cycloalkyl, 3- to 8-membered heterocycloalkyl, 3- to 7-membered spirocyclic cycloalkyl or heterocycloalkyl, aryl, or 5- to 6-membered heteroaryl; and
r is 0-5;
   with the proviso that the compound of Formula I is not 2-(4-methyl-1,2,5-oxadiazol-3-yl)-1-(4-(5-(p-tolyl)-2H-tetrazol-2-yl)piperidin-1-yl)ethan-1-one; 2-(4-methyl-1,2,5-oxadiazol-3-yl)-1-(4-(5-(pyridin-4-yl)-1,2,4-oxadiazol-3-yl)piperidin-1-yl)ethan-1-one; 2-(4-methyl-1,2,5-oxadiazol-3-yl)-1-(4-(5-(6-methylpyridin-3-yl)-1,2,4-oxadiazol-3-yl)piperidin-1-yl)ethan-1-one; 2-(4-methyl-1,2,5-oxadiazol-3-yl)-1-(3-(3-phenyl-1,2,4-oxadiazol-5-yl)piperidin-1-yl)ethan-1-one; 1-(3-(5-cyclopropylpyrimidin-2-yl)azetidin-1-yl)-2-(4-methyl-1,2,5-oxadiazol-3-yl)ethan-1-one; 8) 1-(4-(5-(furan-3-yl)-1,3,4-oxadiazol-2-yl)piperidin-1-yl)-2-(2H-tetrazol-2-yl)ethan-1-one; 2-(2H-tetrazol-2-yl)-1-(3-(5-(thiophen-3-yl)-1,3,4-oxadiazol-2-yl)piperidin-1-yl)ethan-1-one; 2-(1H-tetrazol-5-yl)-1-(4-(3-(thiophen-2-yl)-1,2,4-oxadiazol-5-yl)piperidin-1-yl)ethan-1-one; 2-(5-isopropyl-1H-tetrazol-1-yl)-1-(4-(3-phenyl-1,2,4-oxadiazol-5-yl)piperidin-1-yl)ethan-1-one; 1-(3-(3-phenyl-1,2,4-oxadiazol-5-yl)piperidin-1-yl)-2-(1H-tetrazol-1-yl)ethan-1-one; 1-(3-(5-(1-(cyclopropylmethyl)pyrrolidin-2-yl)-1,2,4-oxadiazol-3-yl)azetidin-1-yl)-2-(1H-tetrazol-1-yl)ethan-1-one; 1-(3-(4-phenyl-1H-1,2,3-triazol-1-yl)azetidin-1-yl)-2-(1H-tetrazol-1-yl)ethan-1-one; 1-(3-(4-phenyl-1H-1,2,3-triazol-1-yl)pyrrolidin-1-yl)-2-(1H-tetrazol-1-yl)ethan-1-one; 1-(3-(5-cyclopropylpyrimidin-2-yl)azetidin-1-yl)-2-(1H-tetrazol-1-yl)ethan-1-one; 1-(4-(6-cyclopropylpyridazin-3-yl)piperazin-1-yl)-2-(1H-tetrazol-1-yl)ethan-1-one; 1-(4-(6-(1H-1,2,4-triazol-1-yl)pyridazin-3-yl)piperazin-1-yl)-2-(1H-tetrazol-1-yl)ethan-1-one; 1-(3-(3-cyclopropyl-1,2,4-oxadiazol-5-yl)pyrrolidin-1-yl)-2-(isoxazol-3-yl)ethan-1-one; 2-(isoxazol-3-yl)-1-(3-(3-phenyl-1,2,4-oxadiazol-5-yl)azetidin-1-yl)ethan-1-one; or 1-(3-(3-isobutyl-1,2,4-oxadiazol-5-yl)piperidin-1-yl)-2-(4-methyl-1,2,5-oxadiazol-3-yl)ethan-1-one;
or any pharmaceutically acceptable salts or any stereochemically isomeric forms thereof.
   Embodiment 2. The compound of Embodiment 1, wherein A is A¹.
   Embodiment 3. The compound of Embodiment 1, wherein A is A².
   Embodiment 4. The compound of Embodiment 1, wherein A is A³.
   Embodiment 5. The compound of Embodiment 1, wherein A is A⁴.
   Embodiment 6. The compound of Embodiment 1, wherein A is A⁵.
   Embodiment 7. The compound of Embodiment 1, wherein A is A⁶.
   Embodiment 8. The compound of Embodiment 1, wherein A is A⁷.
   Embodiment 9. The compound of Embodiment 1, wherein A is A⁸.
   Embodiment 10. The compound of Embodiment 1, wherein A is A⁹.
   Embodiment 11. The compound of Embodiment 1, wherein A is A¹⁰.
   Embodiment 12. The compound of Embodiment 1, wherein A is A¹¹.
   Embodiment 13. The compound of Embodiment 1, wherein A is A¹².
   Embodiment 14. The compound of Embodiment 1, wherein A is A¹³.
   Embodiment 15. The compound of any one of Embodiments 1-14, wherein B is Bi.
   Embodiment 16. The compound of any one of Embodiments 1-14, wherein B is B².
   Embodiment 17. The compound of any one of Embodiments 1-14, wherein B is B³.
   Embodiment 18. The compound of any one of Embodiments 1-14, wherein B is B⁴.
   Embodiment 19. The compound of any one of Embodiments 1-14, wherein B is B⁵.
   Embodiment 20. The compound of any one of Embodiments 1-14, wherein B is B⁶.
   Embodiment 21. The compound of any one of Embodiments 1-14, wherein B is B⁷.
   Embodiment 22. The compound of any one of Embodiments 1-14, wherein B is B⁸.
   Embodiment 23. The compound of any one of Embodiments 1-14, wherein B is B⁹.
   Embodiment 24. The compound of any one of Embodiments 1-14, wherein B is B¹⁰.
   Embodiment 25. The compound of any one of Embodiments 1-14, wherein B is B¹¹.
   Embodiment 26. The compound of any one of Embodiments 1-14, wherein B is B¹².
   Embodiment 27. The compound of any one of Embodiments 1-14, wherein B is B¹³.
   Embodiment 28. The compound of any one of Embodiments 1-14, wherein B is B¹⁴.
   Embodiment 29. The compound of any one of Embodiments 1-14, wherein B is B¹⁵.
   Embodiment 30. The compound of any one of Embodiments 1-29, wherein Z¹ is N.
   Embodiment 31. The compound of any one of Embodiments 1-30, wherein Z² is CH.
   Embodiment 32. The compound of any one of Embodiments 1-31, wherein both u and v are 1.
   Embodiment 33. The compound of any one of Embodiments 1-32, wherein both R² are absent.
   Embodiment 34. The compound of any one of Embodiments 1-32, wherein both R² together form a (C₁-C₂)alkylenyl bridge.
   Embodiment 35. The compound of Embodiment 34, wherein both R² together form —CH₂—.
   Embodiment 36. The compound of Embodiment 34, wherein both R² together form —CH₂—CH₂—.
   Embodiment 37. The compound of any one of Embodiments 1-36, wherein Q is Ph.
   Embodiment 38. The compound of Embodiment 37, wherein r is 1.
   Embodiment 39. The compound of Embodiment 38, wherein Q′ is halo.
   Embodiment 40. The compound of Embodiment 39, wherein Q′ is p-F.
   Embodiment 41. The compound of Embodiment 39, wherein Q′ is p-Cl.
   Embodiment 42. The compound of Embodiment 37, wherein r is 2.
   Embodiment 43. The compound of Embodiment 42, wherein at least one Q′ is halo.
   Embodiment 44. The compound of Embodiment 43, wherein at least one halo is p-F.
   Embodiment 45. The compound of Embodiment 43, wherein at least one halo is p-Cl.
   Embodiment 46. The compound of Embodiment 43, wherein both Q′ are halo.
   Embodiment 47. The compound of Embodiment 46, wherein at least one halo is p-F.
   Embodiment 48. The compound of Embodiment 47, wherein both halo are F.
   Embodiment 49. The compound of Embodiment 47, wherein the second halo is Cl.
   Embodiment 50. The compound of Embodiment 46, wherein at least one halo is p-Cl.
   Embodiment 51. The compound of Embodiment 50, wherein both halo are Cl.
   Embodiment 52. The compound of Embodiment 50, wherein the second halo is F.
   Embodiment 53. The compound of any one of Embodiments 1-36, wherein Q is cycloalkyl.
   Embodiment 54. The compound of Embodiment 53, wherein Q is cyclohexyl.
   Embodiment 55. The compound of either Embodiment 53 or Embodiment 54, wherein r is 0.
   Embodiment 56. The compound of either Embodiment 53 or Embodiment 54, wherein r is 2.
   Embodiment 57. The compound of Embodiments 56, wherein one Q′ is 4-F.
   Embodiment 58. The compound of either Embodiment 56 or Embodiment 57, wherein both Q′ are 4-F.
   Embodiment 59. The compound of either Embodiment 56 or Embodiment 57, wherein one (is 4-Me.
   Embodiment 60. The compound of Embodiment 56, wherein both Q′ are 4-Me.
   Embodiment 61. A compound having any one of the formulae selected from the group consisting of:
6-(2-(4-(3-(4-fluorophenyl)-1,2,4-oxadiazol-5-yl)piperidin-1-yl)-2-oxoethyl)pyrimidine-2,4(1H,3H)-dione;
1-(4-(5-(4-fluorophenyl)-2H-tetrazol-2-yl)piperidin-1-yl)-2-(pyrazin-2-yl)ethan-1-one;
6-((4-(3-(3,4-dichlorophenyl)-1,2,4-oxadiazol-5-yl)piperidin-1-yl)methyl)pyrimidine-2,4(1H,3H)-dione;
1-(4-(5-(4-fluorophenyl)-2H-tetrazol-2-yl)piperidin-1-yl)-2-(pyrazin-2-yl)ethan-1-one;
1-(4-(3-(3,4-dichlorophenyl)-1,2,4-oxadiazol-5-yl)piperidin-1-yl)-2-(3-isopropyl-1,2,4-oxadiazol-5-yl)ethan-1-one;
1-(4-(3-(4-fluorophenyl)-1,2,4-oxadiazol-5-yl)piperidin-1-yl)-2-(3-isopropyl-1,2,4-oxadiazol-5-yl)ethan-1-one;
1-(4-(3-cyclohexyl-1,2,4-oxadiazol-5-yl)piperidin-1-yl)-2-(5-methylpyrimidin-4-yl)ethan-1-one;
1-(4-(3-(3,4-dichlorophenyl)-1,2,4-oxadiazol-5-yl)piperidin-1-yl)-2-(5-methylpyrimidin-4-yl)ethan-1-one;
1-(4-(3-(4-fluorophenyl)-1,2,4-oxadiazol-5-yl)piperidin-1-yl)-2-(5-methylpyrimidin-4-yl)ethan-1-one;
1-(4-(3-(3,4-dichlorophenyl)-1,2,4-oxadiazol-5-yl)piperidin-1-yl)-2-(4-methylisoxazol-3-yl)ethan-1-one;
1-(4-(3-(4-fluorophenyl)-1,2,4-oxadiazol-5-yl)piperidin-1-yl)-2-(4-methylisoxazol-3-yl)ethan-1-one;
1-(4-(3-(4-fluorophenyl)-1,2,4-oxadiazol-5-yl)piperidin-1-yl)-2-(5-methyl-1,2,4-oxadiazol-3-yl)ethan-1-one;
1-(4-(3-cyclohexyl-1,2,4-oxadiazol-5-yl)piperidin-1-yl)-2-(3-methylpyrazin-2-yl)ethan-1-one;
1-(4-(3-(3,4-dichlorophenyl)-1,2,4-oxadiazol-5-yl)piperidin-1-yl)-2-(3-methylpyrazin-2-yl)ethan-1-one;
1-(4-(3-(4-fluorophenyl)-1,2,4-oxadiazol-5-yl)piperidin-1-yl)-2-(3-methylpyrazin-2-yl)ethan-1-one;
1-(4-(3-(4-chloro-3-fluorophenyl)-1,2,4-oxadiazol-5-yl)piperidin-1-yl)-2-(5-methyl-2H-tetrazol-2-yl)ethan-1-one;
1-(4-(3-(4-chloro-3-fluorophenyl)-1,2,4-oxadiazol-5-yl)piperidin-1-yl)-2-(5-methyl-1H-tetrazol-1-yl)ethan-1-one;
1-(4-(3-(bicyclo[2.2.1]heptan-2-yl)-1,2,4-oxadiazol-5-yl)piperidin-1-yl)-2-(3-methyl-1,2,4-oxadiazol-5-yl)ethan-1-one;
1-(4-(3-(1,4-dioxaspiro[4.5]decan-8-yl)-1,2,4-oxadiazol-5-yl)piperidin-1-yl)-2-(3-methyl-1,2,4-oxadiazol-5-yl)ethan-1-one;
1-(4-(3-cyclopentyl-1,2,4-oxadiazol-5-yl)piperidin-1-yl)-2-(3-methyl-1,2,4-oxadiazol-5-yl)ethan-1-one;
1-(4-(5-cyclopentyl-2H-tetrazol-2-yl)piperidin-1-yl)-2-(3-methyl-1,2,4-oxadiazol-5-yl)ethan-1-one;
1-(4-(3-(bicyclo[4.1.0]heptan-2-yl)-1,2,4-oxadiazol-5-yl)piperidin-1-yl)-2-(3-methyl-1,2,4-oxadiazol-5-yl)ethan-1-one;
1-(4-(3-cyclohexyl-1,2,4-oxadiazol-5-yl)piperidin-1-yl)-2-(3-methyl-1,2,4-oxadiazol-5-yl)ethan-1-one;
1-(4-(5-cyclohexyl-2H-tetrazol-2-yl)piperidin-1-yl)-2-(3-methyl-1,2,4-oxadiazol-5-yl)ethan-1-one;
1-(4-(3-cyclohexyl-1,2,4-oxadiazol-5-yl)-4-hydroxypiperidin-1-yl)-2-(3-methyl-1,2,4-oxadiazol-5-yl)ethan-1-one;
1-(4-(3-(bicyclo[2.2.1]heptan-1-yl)-1,2,4-oxadiazol-5-yl)piperidin-1-yl)-2-(3-methyl-1,2,4-oxadiazol-5-yl)ethan-1-one;
2-(3-methyl-1,2,4-oxadiazol-5-yl)-1-(4-(3-(1-methylcyclohexyl)-1,2,4-oxadiazol-5-yl)piperidin-1-yl)ethan-1-one;
1-(4-(3-(2,2-dimethylcyclopropyl)-1,2,4-oxadiazol-5-yl)piperidin-1-yl)-2-(3-methyl-1,2,4-oxadiazol-5-yl)ethan-1-one;
1-(4-(3-(3,3-dimethylcyclopentyl)-1,2,4-oxadiazol-5-yl)piperidin-1-yl)-2-(3-methyl-1,2,4-oxadiazol-5-yl)ethan-1-one;
1-(4-(3-(4,4-dimethylcyclohexyl)-1,2,4-oxadiazol-5-yl)piperidin-1-yl)-2-(3-methyl-1,2,4-oxadiazol-5-yl)ethan-1-one;
1-(4-(3-((3R,5S)-3,5-dimethylcyclohexyl)-1,2,4-oxadiazol-5-yl)piperidin-1-yl)-2-(3-methyl-1,2,4-oxadiazol-5-yl)ethan-1-one;
1-(4-(3-(2,4-dichlorophenyl)-1,2,4-oxadiazol-5-yl)piperidin-1-yl)-2-(3-methyl-1,2,4-oxadiazol-5-yl)ethan-1-one;
1-(4-(3-(3,4-dichlorophenyl)-1,2,4-oxadiazol-5-yl)piperidin-1-yl)-2-(3-methyl-1,2,4-oxadiazol-5-yl)ethan-1-one;
1-(4-(3-(4-chlorophenyl)-1,2,4-oxadiazol-5-yl)piperidin-1-yl)-2-(3-methyl-1,2,4-oxadiazol-5-yl)ethan-1-one;
1-(4-(5-(4-chlorophenyl)-2H-tetrazol-2-yl)piperidin-1-yl)-2-(3-methyl-1,2,4-oxadiazol-5-yl)ethan-1-one;
1-(4-(3-(3-chlorophenyl)-1,2,4-oxadiazol-5-yl)piperidin-1-yl)-2-(3-methyl-1,2,4-oxadiazol-5-yl)ethan-1-one;
2-(3-methyl-1,2,4-oxadiazol-5-yl)-1-(4-(3-(4-(trifluoromethyl)cyclohexyl)-1,2,4-oxadiazol-5-yl)piperidin-1-yl)ethan-1-one;
1-(4-(3-(4-chloro-3-(trifluoromethoxy)phenyl)-1,2,4-oxadiazol-5-yl)piperidin-1-yl)-2-(3-methyl-1,2,4-oxadiazol-5-yl)ethan-1-one;
2-(3-methyl-1,2,4-oxadiazol-5-yl)-1-(4-(3-(4-(trifluoromethoxy)phenyl)-1,2,4-oxadiazol-5-yl)piperidin-1-yl)ethan-1-one;
1-(4-(3-(3,3-difluorocyclopentyl)-1,2,4-oxadiazol-5-yl)piperidin-1-yl)-2-(3-methyl-1,2,4-oxadiazol-5-yl)ethan-1-one;
1-(4-(3-(4,4-difluorocyclohexyl)-1,2,4-oxadiazol-5-yl)piperidin-1-yl)-2-(3-methyl-1,2,4-oxadiazol-5-yl)ethan-1-one;
1-(4-(3-(4,4-difluorocyclohexyl)-1,2,4-oxadiazol-5-yl)-4-hydroxypiperidin-1-yl)-2-(3-methyl-1,2,4-oxadiazol-5-yl)ethan-1-one;
1-(4-(5-(3-fluorobicyclo[1.1.1]pentan-1-yl)-1,2,4-oxadiazol-3-yl)piperidin-1-yl)-2-(3-methyl-1,2,4-oxadiazol-5-yl)ethan-1-one;
1-(4-(3-(4-chloro-3-fluorophenyl)-1,2,4-oxadiazol-5-yl)piperidin-1-yl)-2-(3-methyl-1,2,4-oxadiazol-5-yl)ethan-1-one;
1-(4-(3-(3-chloro-5-fluorophenyl)-1,2,4-oxadiazol-5-yl)piperidin-1-yl)-2-(3-methyl-1,2,4-oxadiazol-5-yl)ethan-1-one;
1-(4-(3-(2-chloro-4-fluorophenyl)-1,2,4-oxadiazol-5-yl)piperidin-1-yl)-2-(3-methyl-1,2,4-oxadiazol-5-yl)ethan-1-one;
1-(4-(3-(3,4-difluorophenyl)-1,2,4-oxadiazol-5-yl)piperidin-1-yl)-2-(3-methyl-1,2,4-oxadiazol-5-yl)ethan-1-one;
1-(4-(3-(4-fluoro-3-(trifluoromethoxy)phenyl)-1,2,4-oxadiazol-5-yl)piperidin-1-yl)-2-(3-methyl-1,2,4-oxadiazol-5-yl)ethan-1-one;
1-(4-(3-(4-fluorophenyl)-1,2,4-oxadiazol-5-yl)piperidin-1-yl)-2-(3-methyl-1,2,4-oxadiazol-5-yl)ethan-1-one;
1-(4-(3-(4-fluorophenyl)isoxazol-5-yl)piperidin-1-yl)-2-(3-methyl-1,2,4-oxadiazol-5-yl)ethan-1-one;
1-(4-(5-(4-fluorophenyl)-2H-tetrazol-2-yl)piperidin-1-yl)-2-(3-methyl-1,2,4-oxadiazol-5-yl)ethan-1-one;
1-(4-(4-(4-fluorophenyl)oxazol-2-yl)piperidin-1-yl)-2-(3-methyl-1,2,4-oxadiazol-5-yl)ethan-1-one;
1-(4-(5-(4-fluorophenyl)-1,2,4-oxadiazol-3-yl)piperidin-1-yl)-2-(3-methyl-1,2,4-oxadiazol-5-yl)ethan-1-one;
1-(4-(3-(4-fluorophenyl)-1,2,4-oxadiazol-5-yl)-4-hydroxypiperidin-1-yl)-2-(3-methyl-1,2,4-oxadiazol-5-yl)ethan-1-one;
1-(4-(3-cyclohexyl-1,2,4-oxadiazol-5-yl)piperidin-1-yl)-2-(3-methylisoxazol-4-yl)ethan-1-one;
1-(4-(5-cyclohexyl-2H-tetrazol-2-yl)piperidin-1-yl)-2-(3-methylisoxazol-4-yl)ethan-1-one;
1-(4-(3-(3,4-dichlorophenyl)-1,2,4-oxadiazol-5-yl)piperidin-1-yl)-2-(3-methylisoxazol-4-yl)ethan-1-one;
1-(4-(3-(4-fluorophenyl)-1,2,4-oxadiazol-5-yl)piperidin-1-yl)-2-(3-methylisoxazol-4-yl)ethan-1-one;
1-(4-(5-(4-fluorophenyl)-2H-tetrazol-2-yl)piperidin-1-yl)-2-(3-methylisoxazol-4-yl)ethan-1-one;
2-(4-methyl-1,2,5-oxadiazol-3-yl)-1-(4-(4-(morpholine-4-carbonyl)-1H-1,2,3-triazol-1-yl)piperidin-1-yl)ethan-1-one;
1-(4-(3-cyclopropyl-1,2,4-oxadiazol-5-yl)piperidin-1-yl)-2-(4-methyl-1,2,5-oxadiazol-3-yl)ethan-1-one;
1-(4-(5-cyclopropyl-2H-tetrazol-2-yl)piperidin-1-yl)-2-(4-methyl-1,2,5-oxadiazol-3-yl)ethan-1-one;
2-(4-methyl-1,2,5-oxadiazol-3-yl)-1-(4-(5-(spiro[3.3]heptan-2-yl)-1,2,4-oxadiazol-3-yl)piperidin-1-yl)ethan-1-one;
1-(4-(3-(bicyclo[2.2.1]heptan-2-yl)-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(5-(spiro[2.3]hexan-1-yl)-1,2,4-oxadiazol-3-yl)piperidin-1-yl)ethan-1-one;
1-(4-(3-cyclobutyl-1,2,4-oxadiazol-5-yl)piperidin-1-yl)-2-(4-methyl-1,2,5-oxadiazol-3-yl)ethan-1-one;
1-(4-(3-(1,4-dioxaspiro[4.5]decan-8-yl)-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(3-(spiro[2.5]octan-6-yl)-1,2,4-oxadiazol-5-yl)piperidin-1-yl)ethan-1-one;
2-(4-methyl-1,2,5-oxadiazol-3-yl)-1-(4-(5-(spiro[2.5]octan-6-yl)-1,2,4-oxadiazol-3-yl)piperidin-1-yl)ethan-1-one;
1-(4-(3-cyclopentyl-1,2,4-oxadiazol-5-yl)piperidin-1-yl)-2-(4-methyl-1,2,5-oxadiazol-3-yl)ethan-1-one;
1-(4-(5-cyclopentyl-2H-tetrazol-2-yl)piperidin-1-yl)-2-(4-methyl-1,2,5-oxadiazol-3-yl)ethan-1-one;
1-(4-(3-(bicyclo[4.1.0]heptan-2-yl)-1,2,4-oxadiazol-5-yl)piperidin-1-yl)-2-(4-methyl-1,2,5-oxadiazol-3-yl)ethan-1-one;
1-(4-(3-cyclohexyl-1,2,4-oxadiazol-5-yl)piperidin-1-yl)-2-(4-methyl-1,2,5-oxadiazol-3-yl)ethan-1-one;
1-(4-(5-cyclohexyl-2H-tetrazol-2-yl)piperidin-1-yl)-2-(4-methyl-1,2,5-oxadiazol-3-yl)ethan-1-one;
1-(4-(3-cyclohexyl-1,2,4-oxadiazol-5-yl)-4-hydroxypiperidin-1-yl)-2-(4-methyl-1,2,5-oxadiazol-3-yl)ethan-1-one;
2-(4-methyl-1,2,5-oxadiazol-3-yl)-1-(4-(3-(tetrahydro-2H-pyran-4-yl)-1,2,4-oxadiazol-5-yl)piperidin-1-yl)ethan-1-one;
1-(4-(5-(bicyclo[2.2.1]heptan-1-yl)-1,2,4-oxadiazol-3-yl)piperidin-1-yl)-2-(4-methyl-1,2,5-oxadiazol-3-yl)ethan-1-one;
1-(4-(3-(bicyclo[2.2.1]heptan-1-yl)-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(3-phenyl-1H-pyrazol-1-yl)piperidin-1-yl)ethan-1-one;
2-(4-methyl-1,2,5-oxadiazol-3-yl)-1-(4-(5-methyl-2H-tetrazol-2-yl)piperidin-1-yl)ethan-1-one;
1-(4-(3-methyl-1,2,4-oxadiazol-5-yl)piperidin-1-yl)-2-(4-methyl-1,2,5-oxadiazol-3-yl)ethan-1-one;
1-(4-(3-isopropyl-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(3-(1-methylcyclohexyl)-1,2,4-oxadiazol-5-yl)piperidin-1-yl)ethan-1-one;
2-(4-methyl-1,2,5-oxadiazol-3-yl)-1-(4-(3-(3-methyloxetan-3-yl)-1,2,4-oxadiazol-5-yl)piperidin-1-yl)ethan-1-one;
1-(4-(3-(2,2-dimethylcyclopropyl)-1,2,4-oxadiazol-5-yl)piperidin-1-yl)-2-(4-methyl-1,2,5-oxadiazol-3-yl)ethan-1-one;
1-(4-(3-(3,3-dimethylcyclopentyl)-1,2,4-oxadiazol-5-yl)piperidin-1-yl)-2-(4-methyl-1,2,5-oxadiazol-3-yl)ethan-1-one;
1-(4-(3-(4,4-dimethylcyclohexyl)-1,2,4-oxadiazol-5-yl)piperidin-1-yl)-2-(4-methyl-1,2,5-oxadiazol-3-yl)ethan-1-one;
1-(4-(3-(3,3-dimethylcyclohexyl)-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(5-(3-methylbicyclo[1.1.1]pentan-1-yl)-1,2,4-oxadiazol-3-yl)piperidin-1-yl)ethan-1-one;
1-(4-(3-(1-methoxyethyl)-1,2,4-oxadiazol-5-yl)piperidin-1-yl)-2-(4-methyl-1,2,5-oxadiazol-3-yl)ethan-1-one;
1-(4-(3-(methoxymethyl)-1,2,4-oxadiazol-5-yl)piperidin-1-yl)-2-(4-methyl-1,2,5-oxadiazol-3-yl)ethan-1-one;
1-(4-(3-((3R,5S)-3,5-dimethylcyclohexyl)-1,2,4-oxadiazol-5-yl)piperidin-1-yl)-2-(4-methyl-1,2,5-oxadiazol-3-yl)ethan-1-one;
1-(4-(3-(2,4-dichlorophenyl)-1,2,4-oxadiazol-5-yl)piperidin-1-yl)-2-(4-methyl-1,2,5-oxadiazol-3-yl)ethan-1-one;
1-(4-(3-(3,4-dichlorophenyl)-1,2,4-oxadiazol-5-yl)piperidin-1-yl)-2-(4-methyl-1,2,5-oxadiazol-3-yl)ethan-1-one;
1-(4-(3-(3,4-dichlorophenyl)-1,2,4-oxadiazol-5-yl)-4-hydroxypiperidin-1-yl)-2-(4-methyl-1,2,5-oxadiazol-3-yl)ethan-1-one;
1-(4-(5-(4-chlorophenyl)-2H-tetrazol-2-yl)piperidin-1-yl)-2-(4-methyl-1,2,5-oxadiazol-3-yl)ethan-1-one;
1-(4-(3-(4-chlorophenyl)-1,2,4-oxadiazol-5-yl)piperidin-1-yl)-2-(4-methyl-1,2,5-oxadiazol-3-yl)ethan-1-one;
1-(4-(3-(3-chlorophenyl)-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(3-(4-(trifluoromethyl)cyclohexyl)-1,2,4-oxadiazol-5-yl)piperidin-1-yl)ethan-1-one;
1-(4-(3-(4-chloro-3-(trifluoromethoxy)phenyl)-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(3-(4-(trifluoromethoxy)phenyl)-1,2,4-oxadiazol-5-yl)piperidin-1-yl)ethan-1-one;
2-(4-methyl-1,2,5-oxadiazol-3-yl)-1-(4-(3-(3-(trifluoromethoxy)phenyl)-1,2,4-oxadiazol-5-yl)piperidin-1-yl)ethan-1-one;
2-(4-methyl-1,2,5-oxadiazol-3-yl)-1-(4-(3-(2-(trifluoromethoxy)phenyl)-1,2,4-oxadiazol-5-yl)piperidin-1-yl)ethan-1-one;
1-(4-(3-(4-chloro-3-(trifluoromethyl)phenyl)-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(3-(4-(trifluoromethyl)phenyl)-1,2,4-oxadiazol-5-yl)piperidin-1-yl)ethan-1-one;
2-(4-methyl-1,2,5-oxadiazol-3-yl)-1-(4-(5-(4-(trifluoromethyl)phenyl)-2H-tetrazol-2-yl)piperidin-1-yl)ethan-1-one;
1-(4-(3-(4-(difluoromethoxy)phenyl)-1,2,4-oxadiazol-5-yl)piperidin-1-yl)-2-(4-methyl-1,2,5-oxadiazol-3-yl)ethan-1-one;
1-(4-(3-(3,3-difluorocyclopentyl)-1,2,4-oxadiazol-5-yl)piperidin-1-yl)-2-(4-methyl-1,2,5-oxadiazol-3-yl)ethan-1-one;
1-(4-(3-(4,4-difluorocyclohexyl)-1,2,4-oxadiazol-5-yl)piperidin-1-yl)-2-(4-methyl-1,2,5-oxadiazol-3-yl)ethan-1-one;
1-(4-(3-(4,4-difluorocyclohexyl)-1,2,4-oxadiazol-5-yl)-4-hydroxypiperidin-1-yl)-2-(4-methyl-1,2,5-oxadiazol-3-yl)ethan-1-one;
1-(4-(3-(3,3-difluorocyclohexyl)-1,2,4-oxadiazol-5-yl)piperidin-1-yl)-2-(4-methyl-1,2,5-oxadiazol-3-yl)ethan-1-one;
1-(4-(3-((1R,3 S)-3-fluorocyclopentyl)-1,2,4-oxadiazol-5-yl)piperidin-1-yl)-2-(4-methyl-1,2,5-oxadiazol-3-yl)ethan-1-one;
1-(4-(3-((1R,3R)-3-fluorocyclopentyl)-1,2,4-oxadiazol-5-yl)piperidin-1-yl)-2-(4-methyl-1,2,5-oxadiazol-3-yl)ethan-1-one;
1-(4-(3-(4-fluorocyclohexyl)-1,2,4-oxadiazol-5-yl)piperidin-1-yl)-2-(4-methyl-1,2,5-oxadiazol-3-yl)ethan-1-one;
1-(4-(5-(3-fluorobicyclo[1.1.1]pentan-1-yl)-1,2,4-oxadiazol-3-yl)piperidin-1-yl)-2-(4-methyl-1,2,5-oxadiazol-3-yl)ethan-1-one;
1-(4-(3-(3-fluoro-4-(trifluoromethyl)phenyl)-1,2,4-oxadiazol-5-yl)piperidin-1-yl)-2-(4-methyl-1,2,5-oxadiazol-3-yl)ethan-1-one;
1-(4-(3-(4-chloro-3-fluorophenyl)-1,2,4-oxadiazol-5-yl)piperidin-1-yl)-2-(4-methyl-1,2,5-oxadiazol-3-yl)ethan-1-one;
1-(4-(3-(3-chloro-5-fluorophenyl)-1,2,4-oxadiazol-5-yl)piperidin-1-yl)-2-(4-methyl-1,2,5-oxadiazol-3-yl)ethan-1-one;
1-(4-(3-(4-chloro-2-fluorophenyl)-1,2,4-oxadiazol-5-yl)piperidin-1-yl)-2-(4-methyl-1,2,5-oxadiazol-3-yl)ethan-1-one;
1-(4-(3-(2-chloro-4-fluorophenyl)-1,2,4-oxadiazol-5-yl)piperidin-1-yl)-2-(4-methyl-1,2,5-oxadiazol-3-yl)ethan-1-one;
1-(4-(3-(2,4-difluorophenyl)-1,2,4-oxadiazol-5-yl)piperidin-1-yl)-2-(4-methyl-1,2,5-oxadiazol-3-yl)ethan-1-one;
1-(4-(3-(4-fluoro-2-(trifluoromethyl)phenyl)-1,2,4-oxadiazol-5-yl)piperidin-1-yl)-2-(4-methyl-1,2,5-oxadiazol-3-yl)ethan-1-one;
1-(4-(3-(4-fluoro-3-(trifluoromethyl)phenyl)-1,2,4-oxadiazol-5-yl)piperidin-1-yl)-2-(4-methyl-1,2,5-oxadiazol-3-yl)ethan-1-one;
1-(4-(3-(4-fluoro-3-(trifluoromethoxy)phenyl)-1,2,4-oxadiazol-5-yl)piperidin-1-yl)-2-(4-methyl-1,2,5-oxadiazol-3-yl)ethan-1-one;
1-(4-(3-(4-fluorophenyl)-1H-pyrazol-1-yl)piperidin-1-yl)-2-(4-methyl-1,2,5-oxadiazol-3-yl)ethan-1-one;
1-(4-(4-(4-fluorophenyl)-1H-1,2,3-triazol-1-yl)piperidin-1-yl)-2-(4-methyl-1,2,5-oxadiazol-3-yl)ethan-1-one;
1-(4-(5-(4-fluorophenyl)-1,3,4-oxadiazol-2-yl)piperidin-1-yl)-2-(4-methyl-1,2,5-oxadiazol-3-yl)ethan-1-one;
1-(4-(3-(4-fluorophenyl)-1H-1,2,4-triazol-1-yl)piperidin-1-yl)-2-(4-methyl-1,2,5-oxadiazol-3-yl)ethan-1-one;
1-(4-(5-(4-fluorophenyl)-1,2,4-oxadiazol-3-yl)piperidin-1-yl)-2-(4-methyl-1,2,5-oxadiazol-3-yl)ethan-1-one;
1-(4-(2-(4-fluorophenyl)-2H-tetrazol-5-yl)piperidin-1-yl)-2-(4-methyl-1,2,5-oxadiazol-3-yl)ethan-1-one;
1-(4-(4-(4-fluorophenyl)oxazol-2-yl)piperidin-1-yl)-2-(4-methyl-1,2,5-oxadiazol-3-yl)ethan-1-one;
1-(4-(3-(4-fluorophenyl)isoxazol-5-yl)piperidin-1-yl)-2-(4-methyl-1,2,5-oxadiazol-3-yl)ethan-1-one;
1-(4-(4-(4-fluorophenyl)-2H-1,2,3-triazol-2-yl)piperidin-1-yl)-2-(4-methyl-1,2,5-oxadiazol-3-yl)ethan-1-one;
1-(4-(4-(4-fluorophenyl)-1H-1,2,3-triazol-1-yl)piperidin-1-yl)-2-(4-methyl-1,2,5-oxadiazol-3-yl)ethan-1-one;
1-(4-(3-(4-fluorophenyl)-1,2,4-oxadiazol-5-yl)-4-methylpiperidin-1-yl)-2-(4-methyl-1,2,5-oxadiazol-3-yl)ethan-1-one;
1-(4-(3-(4-fluorophenyl)-1,2,4-oxadiazol-5-yl)-4-(trifluoromethyl)piperidin-1-yl)-2-(4-methyl-1,2,5-oxadiazol-3-yl)ethan-1-one;
1-(4-(3-(4-fluorophenyl)-1,2,4-oxadiazol-5-yl)-4-hydroxypiperidin-1-yl)-2-(4-methyl-1,2,5-oxadiazol-3-yl)ethan-1-one;
1-(4-(3-(5-fluoropyridin-2-yl)-1,2,4-oxadiazol-5-yl)piperidin-1-yl)-2-(4-methyl-1,2,5-oxadiazol-3-yl)ethan-1-one;
1-(4-(3-(4-fluorobenzyl)-1,2,4-oxadiazol-5-yl)piperidin-1-yl)-2-(4-methyl-1,2,5-oxadiazol-3-yl)ethan-1-one;
1-(4-(3-(2,3-difluorophenyl)-1,2,4-oxadiazol-5-yl)piperidin-1-yl)-2-(4-methyl-1,2,5-oxadiazol-3-yl)ethan-1-one;
1-(4-(3-(3-fluorophenyl)-1,2,4-oxadiazol-5-yl)piperidin-1-yl)-2-(4-methyl-1,2,5-oxadiazol-3-yl)ethan-1-one;
1-(4-(5-(3-fluorophenyl)-2H-tetrazol-2-yl)piperidin-1-yl)-2-(4-methyl-1,2,5-oxadiazol-3-yl)ethan-1-one;
1-(4-(3-(2-fluorophenyl)-1,2,4-oxadiazol-5-yl)piperidin-1-yl)-2-(4-methyl-1,2,5-oxadiazol-3-yl)ethan-1-one;
1-(4-(5-(2-fluorophenyl)-2H-tetrazol-2-yl)piperidin-1-yl)-2-(4-methyl-1,2,5-oxadiazol-3-yl)ethan-1-one;
1-(4-(3-(5-fluoropyrimidin-2-yl)-1,2,4-oxadiazol-5-yl)piperidin-1-yl)-2-(4-methyl-1,2,5-oxadiazol-3-yl)ethan-1-one;
4-(2-(1-(2-(4-methyl-1,2,5-oxadiazol-3-yl)acetyl)piperidin-4-yl)-2H-tetrazol-5-yl)benzamide;
N-(2-hydroxyethyl)-1-(1-(2-(4-methyl-1,2,5-oxadiazol-3-yl)acetyl)piperidin-4-yl)-1H-1,2,3-triazole-4-carboxamide;
3-(4-fluorophenyl)-5-(1-(((4-methyl-1,2,5-oxadiazol-3-yl)methyl)sulfonyl)piperidin-4-yl)-1,2,4-oxadiazole;
4-(3-(4-fluorophenyl)-1,2,4-oxadiazol-5-yl)-N-(4-methyl-1,2,5-oxadiazol-3-yl)piperidine-1-carboxamide;
1-(4-(3-(3,4-dichlorophenyl)-1,2,4-oxadiazol-5-yl)piperidin-1-yl)-2-(1-methyl-1H-1,2,4-triazol-3-yl)ethan-1-one;
1-(4-(3-(4-fluorophenyl)-1,2,4-oxadiazol-5-yl)piperidin-1-yl)-2-(1-methyl-1H-1,2,4-triazol-3-yl)ethan-1-one;
1-(4-(3-(4-fluorophenyl)-1,2,4-oxadiazol-5-yl)piperidin-1-yl)-2-(4-methyl-4H-1,2,4-triazol-3-yl)ethan-1-one;
1-(4-(5-(4-fluorophenyl)-2H-tetrazol-2-yl)piperidin-1-yl)-2-(4-methyl-4H-1,2,4-triazol-3-yl)ethan-1-one;
1-(4-(3-(bicyclo[2.2.1]heptan-2-yl)-1,2,4-oxadiazol-5-yl)piperidin-1-yl)-2-(1-methyl-1H-1,2,4-triazol-5-yl)ethan-1-one;
1-(4-(3-((1R,3R)-3-fluoro-3-methylcyclopentyl)-1,2,4-oxadiazol-5-yl)piperidin-1-yl)-2-(1-methyl-1H-1,2,4-triazol-5-yl)ethan-1-one;
1-(4-(3-((1R,3 S)-3-fluoro-3-methylcyclopentyl)-1,2,4-oxadiazol-5-yl)piperidin-1-yl)-2-(1-methyl-1H-1,2,4-triazol-5-yl)ethan-1-one;
1-(4-(3-(4-fluoro-4-methylcyclohexyl)-1,2,4-oxadiazol-5-yl)piperidin-1-yl)-2-(1-methyl-1H-1,2,4-triazol-5-yl)ethan-1-one;
2-(1-methyl-1H-1,2,4-triazol-5-yl)-1-(4-(5-(3-methylbicyclo[1.1.1]pentan-1-yl)-1,2,4-oxadiazol-3-yl)piperidin-1-yl)ethan-1-one;
2-(1-methyl-1H-1,2,4-triazol-5-yl)-1-(4-(3-(3-methylbicyclo[1.1.1]pentan-1-yl)-1,2,4-oxadiazol-5-yl)piperidin-1-yl)ethan-1-one;
1-(4-(3-(2,4-dichlorophenyl)-1,2,4-oxadiazol-5-yl)piperidin-1-yl)-2-(1-methyl-1H-1,2,4-triazol-5-yl)ethan-1-one;
1-(4-(5-(3,4-dichlorophenyl)-2H-tetrazol-2-yl)piperidin-1-yl)-2-(1-methyl-1H-1,2,4-triazol-5-yl)ethan-1-one;
1-(4-(5-(3,4-dichlorophenyl)-1,2,4-oxadiazol-3-yl)piperidin-1-yl)-2-(1-methyl-1H-1,2,4-triazol-5-yl)ethan-1-one;
1-(4-(3-(4-chlorophenyl)-1,2,4-oxadiazol-5-yl)piperidin-1-yl)-2-(1-methyl-1H-1,2,4-triazol-5-yl)ethan-1-one;
1-(4-(5-(4-chlorophenyl)-2H-tetrazol-2-yl)piperidin-1-yl)-2-(1-methyl-1H-1,2,4-triazol-5-yl)ethan-1-one;
1-(4-(3-(4-chloro-3-(trifluoromethoxy)phenyl)-1,2,4-oxadiazol-5-yl)piperidin-1-yl)-2-(1-methyl-1H-1,2,4-triazol-5-yl)ethan-1-one;
2-(1-methyl-1H-1,2,4-triazol-5-yl)-1-(4-(3-(4-(trifluoromethoxy)phenyl)-1,2,4-oxadiazol-5-yl)piperidin-1-yl)ethan-1-one;
2-(1-methyl-1H-1,2,4-triazol-5-yl)-1-(4-(5-(4-(trifluoromethyl)phenyl)-2H-tetrazol-2-yl)piperidin-1-yl)ethan-1-one;
1-(4-(3-(4-(difluoromethyl)phenyl)-1,2,4-oxadiazol-5-yl)piperidin-1-yl)-2-(1-methyl-1H-1,2,4-triazol-5-yl)ethan-1-one;
1-(4-(5-(3-fluorobicyclo[1.1.1]pentan-1-yl)-1,2,4-oxadiazol-3-yl)piperidin-1-yl)-2-(1-methyl-1H-1,2,4-triazol-5-yl)ethan-1-one;
1-(4-(3-(4-(fluoromethyl)phenyl)-1,2,4-oxadiazol-5-yl)piperidin-1-yl)-2-(1-methyl-1H-1,2,4-triazol-5-yl)ethan-1-one;
2-(1-methyl-1H-1,2,4-triazol-5-yl)-1-(4-(3-(4-(pentafluoro-16-sulfaneyl)phenyl)-1,2,4-oxadiazol-5-yl)piperidin-1-yl)ethan-1-one;
1-(4-(5-(4-chloro-3-fluorophenyl)-2H-tetrazol-2-yl)piperidin-1-yl)-2-(1-methyl-1H-1,2,4-triazol-5-yl)ethan-1-one;
1-(4-(3-(4-chloro-2-fluorophenyl)-1,2,4-oxadiazol-5-yl)piperidin-1-yl)-2-(1-methyl-1H-1,2,4-triazol-5-yl)ethan-1-one;
1-(4-(3-(2-chloro-4-fluorophenyl)-1,2,4-oxadiazol-5-yl)piperidin-1-yl)-2-(1-methyl-1H-1,2,4-triazol-5-yl)ethan-1-one;
1-(4-(3-(2,4-difluorophenyl)-1,2,4-oxadiazol-5-yl)piperidin-1-yl)-2-(1-methyl-1H-1,2,4-triazol-5-yl)ethan-1-one;
1-(4-(3-(4-fluoro-3-(trifluoromethyl)phenyl)-1,2,4-oxadiazol-5-yl)piperidin-1-yl)-2-(1-methyl-1H-1,2,4-triazol-5-yl)ethan-1-one;
1-(4-(5-(3-chloro-4-fluorophenyl)-2H-tetrazol-2-yl)piperidin-1-yl)-2-(1-methyl-1H-1,2,4-triazol-5-yl)ethan-1-one;
1-(4-(3-(3-chloro-4-fluorophenyl)-1,2,4-oxadiazol-5-yl)piperidin-1-yl)-2-(1-methyl-1H-1,2,4-triazol-5-yl)ethan-1-one;
1-(4-(3-(3,4-difluorophenyl)-1,2,4-oxadiazol-5-yl)piperidin-1-yl)-2-(1-methyl-1H-1,2,4-triazol-5-yl)ethan-1-one;
1-(4-(5-(3,4-difluorophenyl)-2H-tetrazol-2-yl)piperidin-1-yl)-2-(1-methyl-1H-1,2,4-triazol-5-yl)ethan-1-one;
1-(4-(3-(4-fluorophenyl)-1,2,4-oxadiazol-5-yl)piperidin-1-yl)-2-(1-methyl-1H-1,2,4-triazol-5-yl)ethan-1-one;
1-(4-(5-(4-fluorophenyl)-2H-tetrazol-2-yl)piperidin-1-yl)-2-(1-methyl-1H-1,2,4-triazol-5-yl)ethan-1-one;
1-(4-(3-(4-fluorophenyl)-1,2,4-oxadiazol-5-yl)piperidin-1-yl)-2-(1-methyl-1H-tetrazol-5-yl)ethan-1-one;
1-(5-(3-cyclohexyl-1,2,4-oxadiazol-5-yl)-2-azabicyclo[2.2.1]heptan-2-yl)-2-(3-methyl-1,2,4-oxadiazol-5-yl)ethan-1-one;
1-(5-(3-(3,4-dichlorophenyl)-1,2,4-oxadiazol-5-yl)-2-azabicyclo[2.2.1]heptan-2-yl)-2-(3-methyl-1,2,4-oxadiazol-5-yl)ethan-1-one;
1-(5-(3-(4,4-difluorocyclohexyl)-1,2,4-oxadiazol-5-yl)-2-azabicyclo[2.2.1]heptan-2-yl)-2-(3-methyl-1,2,4-oxadiazol-5-yl)ethan-1-one;
1-(5-(3-(3,4-difluorophenyl)-1,2,4-oxadiazol-5-yl)-2-azabicyclo[2.2.1]heptan-2-yl)-2-(3-methyl-1,2,4-oxadiazol-5-yl)ethan-1-one;
1-(5-(3-(4-fluorophenyl)-1,2,4-oxadiazol-5-yl)-2-azabicyclo[2.2.1]heptan-2-yl)-2-(3-methyl-1,2,4-oxadiazol-5-yl)ethan-1-one;
1-(5-(3-cyclohexyl-1,2,4-oxadiazol-5-yl)-2-azabicyclo[2.2.1]heptan-2-yl)-2-(4-methyl-1,2,5-oxadiazol-3-yl)ethan-1-one;
1-(5-(3-(3,4-dichlorophenyl)-1,2,4-oxadiazol-5-yl)-2-azabicyclo[2.2.1]heptan-2-yl)-2-(4-methyl-1,2,5-oxadiazol-3-yl)ethan-1-one;
1-(5-(3-(4,4-difluorocyclohexyl)-1,2,4-oxadiazol-5-yl)-2-azabicyclo[2.2.1]heptan-2-yl)-2-(4-methyl-1,2,5-oxadiazol-3-yl)ethan-1-one;
1-(5-(3-(3-fluorocyclopentyl)-1,2,4-oxadiazol-5-yl)-2-azabicyclo[2.2.1]heptan-2-yl)-2-(4-methyl-1,2,5-oxadiazol-3-yl)ethan-1-one;
1-(5-(3-(3,4-difluorophenyl)-1,2,4-oxadiazol-5-yl)-2-azabicyclo[2.2.1]heptan-2-yl)-2-(4-methyl-1,2,5-oxadiazol-3-yl)ethan-1-one;
1-(5-(3-(4-fluorophenyl)-1,2,4-oxadiazol-5-yl)-2-azabicyclo[2.2.1]heptan-2-yl)-2-(4-methyl-1,2,5-oxadiazol-3-yl)ethan-1-one;
1-(5-(3-(4-chlorophenyl)-1,2,4-oxadiazol-5-yl)-2-azabicyclo[2.2.1]heptan-2-yl)-2-(1-methyl-1H-1,2,4-triazol-5-yl)ethan-1-one;
1-((1R,4S)-5-(3-(4-chloro-3-fluorophenyl)-1,2,4-oxadiazol-5-yl)-2-azabicyclo[2.2.1]heptan-2-yl)-2-(1-methyl-1H-1,2,4-triazol-5-yl)ethan-1-one;
1-((1S,4R)-5-(3-(4-chloro-3-fluorophenyl)-1,2,4-oxadiazol-5-yl)-2-azabicyclo[2.2.1]heptan-2-yl)-2-(1-methyl-1H-1,2,4-triazol-5-yl)ethan-1-one;
1-(5-(3-(4-chloro-2-fluorophenyl)-1,2,4-oxadiazol-5-yl)-2-azabicyclo[2.2.1]heptan-2-yl)-2-(1-methyl-1H-1,2,4-triazol-5-yl)ethan-1-one;
1-(5-(3-(4-fluorophenyl)-1,2,4-oxadiazol-5-yl)-2-azabicyclo[2.2.1]heptan-2-yl)-2-(1-methyl-1H-1,2,4-triazol-5-yl)ethan-1-one;
1-(6-(3-(3,4-dichlorophenyl)-1,2,4-oxadiazol-5-yl)-3-azabicyclo[3.1.1]heptan-3-yl)-2-(3-methyl-1,2,4-oxadiazol-5-yl)ethan-1-one;
1-(6-(3-(4,4-difluorocyclohexyl)-1,2,4-oxadiazol-5-yl)-3-azabicyclo[3.1.1]heptan-3-yl)-2-(3-methyl-1,2,4-oxadiazol-5-yl)ethan-1-one;
1-(6-(3-(3,4-difluorophenyl)-1,2,4-oxadiazol-5-yl)-3-azabicyclo[3.1.1]heptan-3-yl)-2-(3-methyl-1,2,4-oxadiazol-5-yl)ethan-1-one;
1-(6-(3-(4-fluorophenyl)-1,2,4-oxadiazol-5-yl)-3-azabicyclo[3.1.1]heptan-3-yl)-2-(3-methyl-1,2,4-oxadiazol-5-yl)ethan-1-one;
1-(6-(3-(4-fluorophenyl)-1,2,4-oxadiazol-5-yl)-3-azabicyclo[3.1.1]heptan-3-yl)-2-(3-methylisoxazol-4-yl)ethan-1-one;
1-(6-(3-(4-fluoro-4-methylcyclohexyl)-1,2,4-oxadiazol-5-yl)-3-azabicyclo[3.1.1]heptan-3-yl)-2-(4-methyl-1,2,5-oxadiazol-3-yl)ethan-1-one;
1-(6-(3-(3,4-dichlorophenyl)-1,2,4-oxadiazol-5-yl)-3-azabicyclo[3.1.1]heptan-3-yl)-2-(4-methyl-1,2,5-oxadiazol-3-yl)ethan-1-one;
1-(6-(3-(4,4-difluorocyclohexyl)-1,2,4-oxadiazol-5-yl)-3-azabicyclo[3.1.1]heptan-3-yl)-2-(4-methyl-1,2,5-oxadiazol-3-yl)ethan-1-one;
1-(6-(3-(3,4-difluorophenyl)-1,2,4-oxadiazol-5-yl)-3-azabicyclo[3.1.1]heptan-3-yl)-2-(4-methyl-1,2,5-oxadiazol-3-yl)ethan-1-one;
1-(6-(3-(4-fluorophenyl)-1,2,4-oxadiazol-5-yl)-3-azabicyclo[3.1.1]heptan-3-yl)-2-(4-methyl-1,2,5-oxadiazol-3-yl)ethan-1-one;
1-(6-(3-(3,4-dichlorophenyl)-1,2,4-oxadiazol-5-yl)-3-azabicyclo[3.1.1]heptan-3-yl)-2-(1-methyl-1H-1,2,4-triazol-5-yl)ethan-1-one;
1-((1R,5S,6r)-6-(3-(3,4-dichlorophenyl)-1,2,4-oxadiazol-5-yl)-3-azabicyclo[3.1.1]heptan-3-yl)-2-(1-methyl-1H-1,2,4-triazol-5-yl)ethan-1-one;
1-((1R,5S,6s)-6-(3-(3,4-dichlorophenyl)-1,2,4-oxadiazol-5-yl)-3-azabicyclo[3.1.1]heptan-3-yl)-2-(1-methyl-1H-1,2,4-triazol-5-yl)ethan-1-one;
1-((1R,5S,6s)-6-(3-(4-chlorophenyl)-1,2,4-oxadiazol-5-yl)-3-azabicyclo[3.1.1]heptan-3-yl)-2-(1-methyl-1H-1,2,4-triazol-5-yl)ethan-1-one;
1-((1R,5S,6r)-6-(3-(4-chloro-3-fluorophenyl)-1,2,4-oxadiazol-5-yl)-3-azabicyclo[3.1.1]heptan-3-yl)-2-(1-methyl-1H-1,2,4-triazol-5-yl)ethan-1-one;
1-((1R,5S,6s)-6-(3-(4-chloro-3-fluorophenyl)-1,2,4-oxadiazol-5-yl)-3-azabicyclo[3.1.1]heptan-3-yl)-2-(1-methyl-1H-1,2,4-triazol-5-yl)ethan-1-one;
1-((1R,5S,6r)-6-(3-(4-chloro-2-fluorophenyl)-1,2,4-oxadiazol-5-yl)-3-azabicyclo[3.1.1]heptan-3-yl)-2-(1-methyl-1H-1,2,4-triazol-5-yl)ethan-1-one;
1-((1R,5S,6s)-6-(3-(4-chloro-2-fluorophenyl)-1,2,4-oxadiazol-5-yl)-3-azabicyclo[3.1.1]heptan-3-yl)-2-(1-methyl-1H-1,2,4-triazol-5-yl)ethan-1-one;
1-(2-(3-cyclohexyl-1,2,4-oxadiazol-5-yl)-7-azaspiro[3.5]nonan-7-yl)-2-(3-methyl-1,2,4-oxadiazol-5-yl)ethan-1-one;
1-(2-(3-(3,4-dichlorophenyl)-1,2,4-oxadiazol-5-yl)-7-azaspiro[3.5]nonan-7-yl)-2-(3-methyl-1,2,4-oxadiazol-5-yl)ethan-1-one;
1-(2-(3-(4,4-difluorocyclohexyl)-1,2,4-oxadiazol-5-yl)-7-azaspiro[3.5]nonan-7-yl)-2-(3-methyl-1,2,4-oxadiazol-5-yl)ethan-1-one;
1-(2-(3-(3,4-difluorophenyl)-1,2,4-oxadiazol-5-yl)-7-azaspiro[3.5]nonan-7-yl)-2-(3-methyl-1,2,4-oxadiazol-5-yl)ethan-1-one;
1-(2-(3-cyclohexyl-1,2,4-oxadiazol-5-yl)-7-azaspiro[3.5]nonan-7-yl)-2-(4-methyl-1,2,5-oxadiazol-3-yl)ethan-1-one;
1-(2-(3-(3,4-dichlorophenyl)-1,2,4-oxadiazol-5-yl)-7-azaspiro[3.5]nonan-7-yl)-2-(4-methyl-1,2,5-oxadiazol-3-yl)ethan-1-one;
1-(2-(3-(4,4-difluorocyclohexyl)-1,2,4-oxadiazol-5-yl)-7-azaspiro[3.5]nonan-7-yl)-2-(4-methyl-1,2,5-oxadiazol-3-yl)ethan-1-one;
1-(2-(3-(3,4-difluorophenyl)-1,2,4-oxadiazol-5-yl)-7-azaspiro[3.5]nonan-7-yl)-2-(4-methyl-1,2,5-oxadiazol-3-yl)ethan-1-one;
1-(2-(3-(4-fluorophenyl)-1,2,4-oxadiazol-5-yl)-7-azaspiro[3.5]nonan-7-yl)-2-(4-methyl-1,2,5-oxadiazol-3-yl)ethan-1-one;
1-(4-(3-(3,4-dichlorophenyl)-1,2,4-oxadiazol-5-yl)piperazin-1-yl)-2-(3-methyl-1,2,4-oxadiazol-5-yl)ethan-1-one;
1-(4-(3-(3,4-dichlorophenyl)-1,2,4-oxadiazol-5-yl)piperazin-1-yl)-2-(4-methyl-1,2,5-oxadiazol-3-yl)ethan-1-one;
1-(4-(3-(4-fluorophenyl)-1,2,4-oxadiazol-5-yl)piperazin-1-yl)-2-(4-methyl-1,2,5-oxadiazol-3-yl)ethan-1-one;
1-(1-(3-(4-fluorophenyl)-1,2,4-oxadiazol-5-yl)-6-azaspiro[2.5]octan-6-yl)-2-(4-methyl-1,2,5-oxadiazol-3-yl)ethan-1-one;
1-(1-(3-(4-fluorophenyl)-1,2,4-oxadiazol-5-yl)-6-azaspiro[2.5]octan-6-yl)-2-(3-methyl-1,2,4-oxadiazol-5-yl)ethan-1-one;
1-(1-(3-(3,4-dichlorophenyl)-1,2,4-oxadiazol-5-yl)-7-azaspiro[3.5]nonan-7-yl)-2-(3-methyl-1,2,4-oxadiazol-5-yl)ethan-1-one;
1-(1-(3-(4-fluorophenyl)-1,2,4-oxadiazol-5-yl)-7-azaspiro[3.5]nonan-7-yl)-2-(3-methyl-1,2,4-oxadiazol-5-yl)ethan-1-one;
1-(1-(3-(4-fluorophenyl)-1,2,4-oxadiazol-5-yl)-7-azaspiro[3.5]nonan-7-yl)-2-(4-methyl-1,2,5-oxadiazol-3-yl)ethan-1-one;
1-(6-(5-(4-fluorophenyl)-2H-tetrazol-2-yl)-3-azabicyclo[3.1.1]heptan-3-yl)-2-(3-methyl-1,2,4-oxadiazol-5-yl)ethan-1-one;
1-(6-(5-(4-fluorophenyl)-2H-tetrazol-2-yl)-3-azabicyclo[3.1.1]heptan-3-yl)-2-(4-methyl-1,2,5-oxadiazol-3-yl)ethan-1-one;
2-(4-methyl-1,2,5-oxadiazol-3-yl)-1-(3-(4-(morpholine-4-carbonyl)-1H-1,2,3-triazol-1-yl)piperidin-1-yl)ethan-1-one;
N,N-diethyl-1-(1-(2-(4-methyl-1,2,5-oxadiazol-3-yl)acetyl)piperidin-3-yl)-1H-1,2,3-triazole-4-carboxamide;
N-(2-hydroxyethyl)-1-(1-(2-(4-methyl-1,2,5-oxadiazol-3-yl)acetyl)piperidin-3-yl)-1H-1,2,3-triazole-4-carboxamide;
1-(2-(5-(4-fluorophenyl)-2H-tetrazol-2-yl)-7-azaspiro[3.5]nonan-7-yl)-2-(4-methyl-1,2,5-oxadiazol-3-yl)ethan-1-one;
1-(3-(5-(4-fluorophenyl)-2H-tetrazol-2-yl)piperidin-1-yl)-2-(4-methyl-1,2,5-oxadiazol-3-yl)ethan-1-one;
1-(8-(3-(4-fluorophenyl)-1,2,4-oxadiazol-5-yl)-3-azabicyclo[3.2.1]octan-3-yl)-2-(4-methyl-1,2,5-oxadiazol-3-yl)ethan-1-one;
1-(4-(1-(4-fluorophenyl)-1H-1,2,4-triazol-3-yl)piperazin-1-yl)-2-(4-methyl-1,2,5-oxadiazol-3-yl)ethan-1-one;
4-(3-(4-fluorophenyl)-1,2,4-oxadiazol-5-yl)-N-(4-methyl-1,2,5-oxadiazol-3-yl)cyclohexane-1-carboxamide;
1-(4-(2-(4-fluorophenyl)pyrimidin-5-yl)piperidin-1-yl)-2-(4-methyl-1,2,5-oxadiazol-3-yl)ethan-1-one;
1-(4-(5-(4-fluorophenyl)pyrimidin-2-yl)piperidin-1-yl)-2-(4-methyl-1,2,5-oxadiazol-3-yl)ethan-1-one;
1-(4-(2,3-dimethylphenoxy)piperidin-1-yl)-2-(4-methyl-1,2,5-oxadiazol-3-yl)ethan-1-one;
1-(4-(6-(4-fluorophenyl)pyridazin-3-yl)piperidin-1-yl)-2-(4-methyl-1,2,5-oxadiazol-3-yl)ethan-1-one;
1-(3-(3-(4-fluorophenyl)-1,2,4-oxadiazol-5-yl)piperidin-1-yl)-2-(4-methyl-1,2,5-oxadiazol-3-yl)ethan-1-one;
1-(3-(3-(4-fluorophenyl)-1,2,4-oxadiazol-5-yl)azetidin-1-yl)-2-(4-methyl-1,2,5-oxadiazol-3-yl)ethan-1-one; and
1-(1-(5-(4-fluorophenyl)-2H-tetrazol-2-yl)-7-azaspiro[3.5]nonan-7-yl)-2-(4-methyl-1,2,5-oxadiazol-3-yl)ethan-1-one.
   Embodiment 62. The compound of Embodiment 61 having the formula:
1-(4-(3-(3,4-dichlorophenyl)-1,2,4-oxadiazol-5-yl)piperidin-1-yl)-2-(1-methyl-1H-1,2,4-triazol-5-yl)ethan-1-one.
   Embodiment 63. The compound of Embodiment 61 having the formula:
1-(4-(3-(4-fluorophenyl)-1,2,4-oxadiazol-5-yl)piperidin-1-yl)-2-(4-methyl-1,2,5-oxadiazol-3-yl)ethan-1-one.
   Embodiment 64. The compound of Embodiment 61 having the formula:
1-(4-(3-(4-chloro-3-fluorophenyl)-1,2,4-oxadiazol-5-yl)piperidin-1-yl)-2-(1-methyl-1H-1,2,4-triazol-5-yl)ethan-1-one.
   Embodiment 65. The compound of Embodiment 61 having the formula:
1-((1R,5S,6r)-6-(3-(4-chlorophenyl)-1,2,4-oxadiazol-5-yl)-3-azabicyclo[3.1.1]heptan-3-yl)-2-(1-methyl-1H-1,2,4-triazol-5-yl)ethan-1-one.
   Embodiment 66. The compound of Embodiment 61 having the formula:
1-(5-(3-(4-chloro-3-fluorophenyl)-1,2,4-oxadiazol-5-yl)-2-azabicyclo[2.2.1]heptan-2-yl)-2-(1-methyl-1H-1,2,4-triazol-5-yl)ethan-1-one.
   Embodiment 67. A composition comprising the compound of any one of Embodiments 1-66, admixed with a pharmaceutically acceptable carrier, diluent, or excipient.
   Embodiment 68. The composition of Embodiment 67, further comprising one or more therapeutic compounds or compositions used for the treatment of a mycobacterial disease.
   Embodiment 69. The composition of Embodiment 67, further comprising one or more therapeutic compounds or compositions used for the treatment of a TB infection.
   Embodiment 70. The composition of Embodiment 69, wherein the one or more therapeutic compounds or compositions is selected from bedaquiline, pretomanid and linezolid.
   Embodiment 71. The composition of Embodiment 69, wherein the one or more therapeutic compounds or compositions is Nix-TB.
   Embodiment 72. The composition of Embodiment 69, wherein the one or more therapeutic compounds or compositions is selected from bedaquiline, pretomanid, linezolid, isoniazid, rifampin, rifabutin, rifapentine, pyrazinamide, ethambutol, ethionamide, moxifloxacin, tedizolid, radezolid, sutezolid, posizolid clofazimine, gatifloxacin, kanamycin, nitroimidazo-oxazine, delamanid, OPC-167832, streptomycin, prednisolone, an oxazolidinone, a fluoroquinolone, a corticosteroid, EMB analogue SQ109, a benzothiazinone, a dinitrobenzamide, and an antiviral agent including an antiretroviral agent.
   Embodiment 73. The composition of Embodiment 69, wherein the one or more therapeutic compounds or compositions is a therapeutic agent approved or recommended for the treatment of tuberculosis.
   Embodiment 74. The composition of any one of Embodiments 67-74, wherein the composition is suitable for oral administration.
   Embodiment 75. A method of activating Mycobacterium tuberculosis (Mtb) adenylyl cyclase encoded by Rv1625c, comprising treating intracellular and/or extracellular Mtb with the compound of any one of Embodiments 1-66 or the composition of any one of Embodiments 67-74.
   Embodiment 76. A method of inhibiting the cholesterol degradation pathway during intramacrophage Mtb infection or non-replicating extracellular Mtb, comprising treating Mtb with the compound of any one of Embodiments 1-66 or the composition of any one of Embodiments 67-74.
   Embodiment 77. A method of preventing, ameliorating, or treating a mycobacterial disease, comprising administering to a subject in need thereof a therapeutically effective amount the composition of any one of Embodiments 67-74.
   Embodiment 78. A method of preventing, ameliorating, or treating a TB infection, comprising administering to a subject in need thereof a therapeutically effective amount of the composition of any one of Embodiments 67-74.
   Embodiment 79. Any compound, composition, or method as described herein.

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention, the Detailed Description of the Invention, the Experimentals, or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line drawn through a bond or “˜˜˜˜” drawn through a bond each refer to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent as defined herein.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds disclosed herein may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH— —C(—OH)═CH—), amide/imidic acid (—C(═O)—NH— —C(—OH)═N—) and amidine (—C(═NR)—NH— —C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 12 carbon atoms. The term “lower alkyl” or “C₁-C₆ alkyl” as used herein denotes a straight or branched chain hydrocarbon residue containing 1 to 6 carbon atoms. “C₁₋₁₂ alkyl” as used herein refers to an alkyl composed of 1 to 12 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like. As used herein the term “heteroalkyl” refers to any alkyl group wherein at least one carbon atom is replaced by —N—, —O—, —S—, —S(═O)—, or —S(═O)₂—.

“Alkenyl” or “olefin” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₅), octatrienyl (C₅), and the like. As used herein the term “heteroalkenyl” refers to any alkenyl group wherein at least one carbon atom is replaced by —N—, —O—, —S—, —S(═O)—, or —S(═O)₂—.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. As used herein the term “heteroalkynyl” refers to any alkynyl group wherein at least one carbon atom is replaced by —N—, —O—, —S—, —S(═O)—, or —S(═O)₂—.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an-O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR′S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N,O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocycloalkyl”, “heterocyclyl”, or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetra-hydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —S(═O)R^{aa}, —OS(═O)R^{aa}, —B(OR^{cc})₂, C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O; each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR^{aa}, —N(R^{cc})₂, —CN, —C(═O)Ra, —C(═O)N(R^{cc})₂, —CO₂R^{a}, —SO₂R^{a}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂,C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd}substituents on a carbon atom may be joined to form ═O.

“Halo” or “halogen” refers to fluorine (fluoro, —F), chlorine (chloro, —Cl), bromine (bromo, —Br), or iodine (iodo, —I).

As used herein, the term “composition” is intended to encompass a product comprising the specified ingredients, as well as any product which results, directly or indirectly, from combination of the specified ingredients.

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, replacement of a carbon by a ¹³C- or ¹⁴C-enriched carbon, and/or replacement of an oxygen atom with ¹⁸O, are within the scope of the disclosure. Other examples of isotopes include ¹⁵N, ¹⁸O, ¹⁷O, ³¹P, ³²P, ³⁵S, ¹⁸F, ³⁶Cl and ¹²³I. Compounds with such isotopically enriched atoms are useful, for example, as analytical tools or probes in biological assays.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability.

Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present application may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

As used herein, and unless otherwise specified to the contrary, the term “compound” is inclusive in that it encompasses a compound or a pharmaceutically acceptable salt, stereoisomer, and/or tautomer thereof. Thus, for instance, a compound of the present application includes a pharmaceutically acceptable salt of a tautomer of the compound.

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.
The terms “prevent,” “preventing,” and “prevention” refer to the prevention of the onset, recurrence, or spread of the disease in a patient resulting from the administration of a prophylactic or therapeutic agent.

The term “pharmaceutically effective amount” or “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present application, the terms “patient” and “subject” are used interchangeably.

“Inhibitor” means a compound which prevents or reduces the expression, catalytic activity, and/or localization (i.e., local concentration) of mtb.

### DETAILED DESCRIPTION OF THE FIGURES

FIG. 1. V-59 inhibits Mtb growth in an Rv1625c-dependent mechanism. (A and B) Impact of V-59 on Mtb growth in cholesterol media (A) and in media containing cholesterol and acetate (B). V-59 (10 μM) was added to the cultures every three days, and DMSO is the vehicle control. Data are from two experiments, with three technical replicates each (*P<0.05, One-way ANOVA with Sidak's multiple comparisons test). (C) Effect of V-59 on growth of Mtb in murine macrophages. Macrophages were infected at an MOI of 2 and treated with V-59 (25 μM) or DMSO. Data are from three experiments with two or more technical replicates each (****P<0,0001, One-way ANOVA with Sidak's multiple comparisons test on fold-change in CFU's normalized to DMSO). All data are means i SD.

FIG. 2. Structures and activities of compounds. EC₅₀, half-maximal effective concentration; CC₅₀, 50% cytotoxic concentration; hERG, human ether-à-go-go-related gene; IC₅₀, half-maximal inhibitory concentration; ADME, absorption, distribution, metabolism, excretion; PPB, plasma protein binding; Cyp, cytochrome P450; ER, extraction ratio; PO, per oral.

FIG. 3. V-59 stimulates Rv1625c to produce cAMP.

FIG. 4. The transmembrane domain of Rv1625c is essential for complete degradation of cholesterol and the catalytic domain of Rv1625c is required for V-59 activity.

FIGS. 5A-C. (A) Total cAMP induced in TetOn-cAMP Mtb. Cultures were treated with V-59 (10 μM), Atc (500 ng/mL or 50 ng/mL), or EtOH and samples were collected after 24 hours. Data are normalized as total cAMP per 108 Mtb and are from two experiments with two technical replicates each. (B) Impact of inducing TetOn-cAMP on the growth of Mtb in cholesterol media. Cultures were treated with V-59 (10 μM) or Atc for the duration of the experiment. Data are from two experiments with three technical replicates. (C) Catabolic release of ¹⁴CO₂ from [4-¹⁴C]-cholesterol in the TetOn-cAMP strain treated with V-59, Atc, or EtOH. Data are from two experiments with three technical replicates, normalized to OD and quantified relative to EtOH EtOH is the vehicle control throughout. All data are means SD (*P<0.05, **P<0.01, ***P<0.001, ****P<0.0001, One-way ANOVA with Dunnet's multiple comparisons test).

FIG. 6. V-59 treatment and induction of TetOn-cAMP are associated with shared transcriptional changes to cholesterol utilization genes.

RNA-seq analysis quantifying differentially expressed genes from Mtb grown in cholesterol media, following V-59 treatment, or induction of TetOn-cAMP with Atc. Genes depicted are in the KstR regulons and involved in cholesterol utilization. MCC=methylcitrate cycle. Data are displayed as log 2 fold change in gene expression in response to cAMP-inducing vs. control treatment (“cAMP”=Tet-On cAMP Atc vs. EtOH, “WT”=WT V-59 vs. DMSO, “Δ”=ΔRv1625 V-59 vs. DMSO, “C”=Comp_{Full} V-59 vs. DMSO). Also shown are differentially expressed genes intrinsic to ΔRv1625 (“Δ/WT”=ΔRv1625 DMSO vs. WT DMSO). Data are from two technical replicate samples from one experiment (*adjusted P-value≤0.05).

FIG. 7. Activating cAMP synthesis decreases liberation of propionyl-CoA from cholesterol.

(A) Relative GFP signal from the prpD′::GFP reporter in response to V-59 (10 μM) or DMSO treatment in murine macrophages or cholesterol media. Data are normalized to WT treated with DMSO (**P<0.01, ***P<0.001, Two-way ANOVA with Tukey's multiple comparisons test). (B) Relative GFP signal from the prpD′::GFP reporter in response to inducing TetOn-cAMP with Atc treatment in murine macrophages or cholesterol media. Data are normalized to EtOH vehicle control (**P<0.01, ***P<0.001, One-way ANOVA with Dunnett's multiple comparisons test). GFP MFI was quantified from 10,000 mCherry⁺ Mtb. Data are from two experiments with two technical replicates, shown as means±SD.

FIG. 8. V-59 treatment and induction of TetOn-cAMP are associated with transcriptional changes in select CRPMt regulon genes.

(A) Volcano plots displaying differentially expressed genes following V-59 treatment of WT Mtb relative to DMSO control (left), or following Atc treatment of TetOn-cAMP Mtb relative to EtOH control (right), based on RNA-seq. Each dot represents a single gene, genes in blue are significant (FDR <0.05) in both data sets and genes in red are unique to their respective data set. Dashed line indicates FDR cutoff <0.01. (B) Venn diagram showing the number of significantly differentially expressed genes shared by the V-59 and TetOn-cAMP conditions, and how many of these belong to the KstR cholesterol-related regulon. (C) RNA-seq analysis quantifying differentially expressed genes from Mtb grown in cholesterol media, following V-59 treatment, or induction of TetOn-cAMP with Atc. Genes depicted are predicted members of the CRPMT regulon. Only genes with significant differential expression in both the V-59 and TetOn-cAMP conditions, or genes with intrinsic changes in the ΔRv1625 strain (in italics), are shown. “cAMP”=Tet-On cAMP Atc vs. EtOH, “WT”=WT V-59 vs. DMSO, “Δ”=ΔRv1625 V-59 vs. DMSO, “C”=Comp_{Full} V-59 vs. DMSO, “Δ/WT”=ΔRv1625 DMSO vs. WT DMSO.

FIG. 9A-G. Chemically activating Rv1625c reduces Mtb pathogenesis in vivo.

Effect of V-59 treatment on bacterial burden and pathology in the lungs of BALB/c (A and B) or C₃HeB/FeJ mice (C and D). In (A-D) mice were infected and treated with V-59, INH, or vehicle control. Data are from two independent experiments with 5 mice (A and B), or one experiment with 10 mice (C and D) per group. Outliers with CFUs below the infectious dose were excluded from the analyses (*P<0.05, Mann-Whitney test). (E and F) Impact of mCLB073 treatment on bacterial burden (E) and pathology (F) in BALB/c mice infected and treated with the indicated doses of mCLB073, INH, or vehicle control (*P<0.05, **P<0.01, Kruskal-Wallis test and Dunn's multiple comparisons test). Infections in (A-F) were by the intranasal route. Data are from one experiment with 10 mice per group. (G) Impact of mCLB073 treatment on bacterial burden in BALB/c mice infected by aerosol and treated with 5 mg/kg mCLB073 or vehicle control. Data are from one experiment with 5 mice per group (*P<0.05, Kruskal-Wallis test and Dunn's multiple comparisons test). All data are shown as means±SEM.

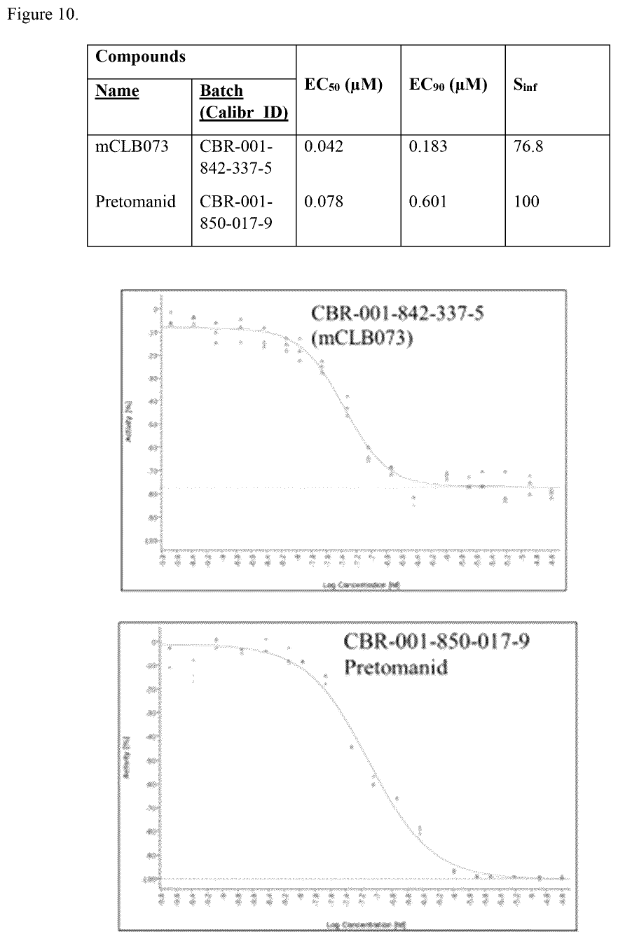

FIG. 10. In vitro potency of mCLB073 in differentiated THP-1 cells. mCLB073 was tested for its ability to inhibit growth of Mtb residing inside PMA-differentiated THP-1 cells. An EC₅₀ value of 0.04 μM was determined for mCLB073. There was no significant variation in activity between replicates (FIG. 10). The recently FDA-approved anti-TB drug Pretomanid was used as a positive control. Representative curves are shown in FIG. 10 of the non-linear regression curve fit of the drug concentration versus the normalized luminescence signal. Of note, mCLB073 demonstrated a partial response based on S_{inf} values (indicating maximum inhibition). The S_{inf} for mCLB073 was 76.8%, whereas Pretomanid showed the maximal response.

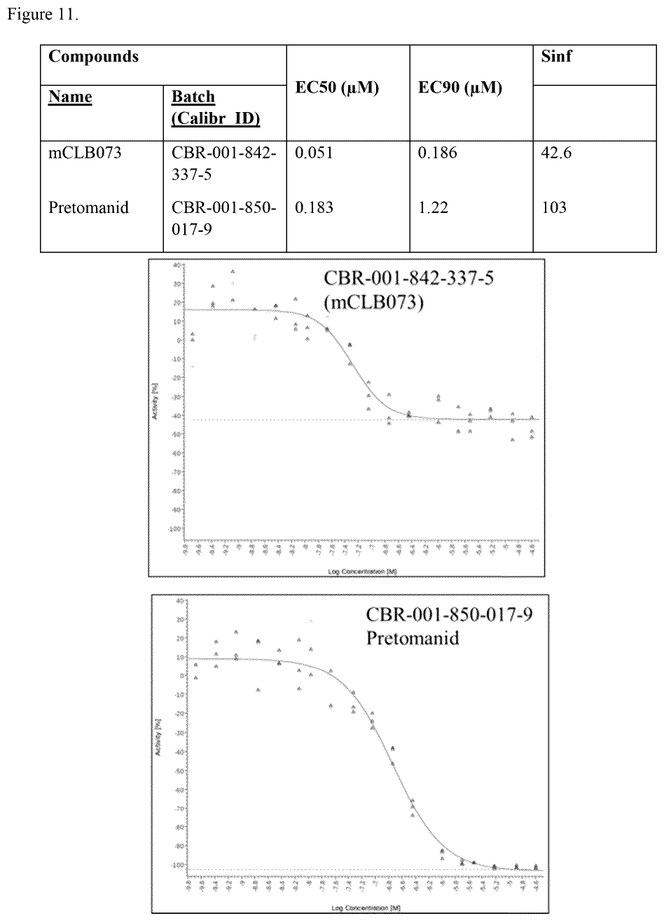

FIG. 11. In vitro potency of mCLB073 in J774.1 cells. mCLB073 was tested for its ability to inhibit growth of Mtb residing inside J774.1 macrophages. An EC₅₀ value of 0.05 μM was determined for mCLB073. There was no significant variation in activity between replicates (Table 2). The recently FDA-approved anti-TB drug Pretomanid was used as a positive control. Representative curves are shown in FIG. 11 of the non-linear regression curve fit of the drug concentration versus the normalized luminescence signal. Of note, mCLB073 demonstrated a partial response based on S_{inf} values (indicating maximum inhibition). The S_{inf} for mCLB073 was 42.1%, whereas Pretomanid showed the maximal response.

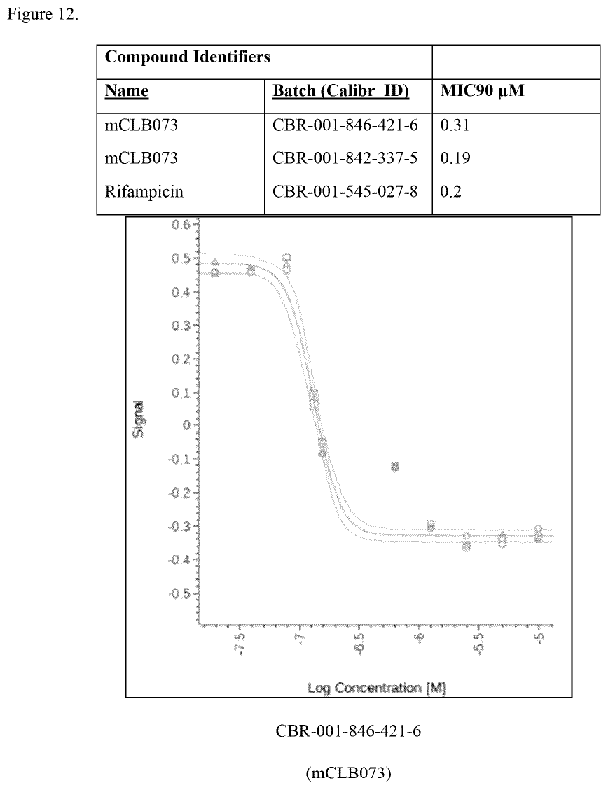

FIG. 12. In vitro potency of mCLB073 in Mtb H37Rv.

Two batches of mCLB073 were tested for in vitro inhibition of Mtb in cholesterol media. MIC90 values ranged from 0.19 μM to 0.31 μM with an average value at 0.28 μM. There was no significant variation in activity between replicates (FIG. 12). Rifampicin, the most potent first-line anti-TB drug, was used as a positive control. A representative dose-response curve is shown in FIG. 12.

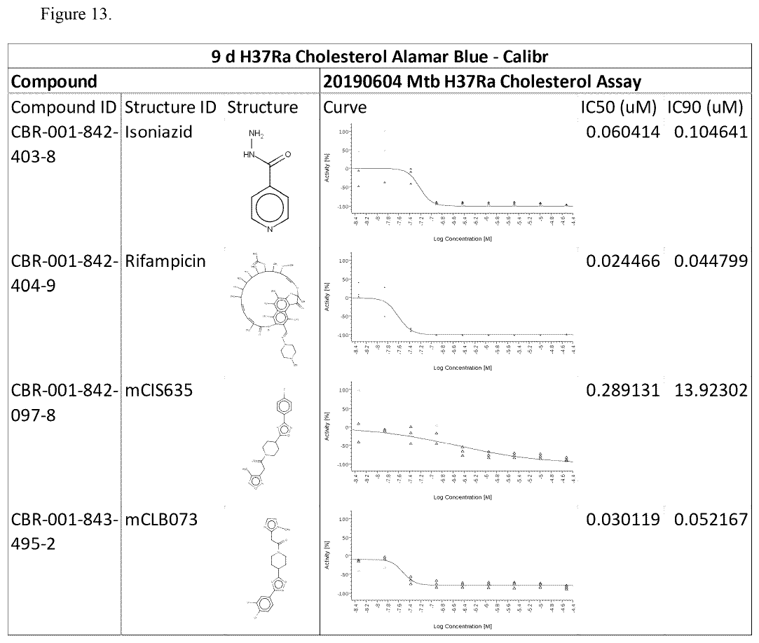

FIG. 13. In vitro potency of mCLB073 in Mtb H37Ra. mCLB073, related compound mCIS635, and control compounds Rifampicin and Isoniazid were tested against the attenuated BSL2 Mtb strain H37Ra in cholesterol media. The measured half maximal concentration (IC50) of mCLB073 was 30.1 nM.

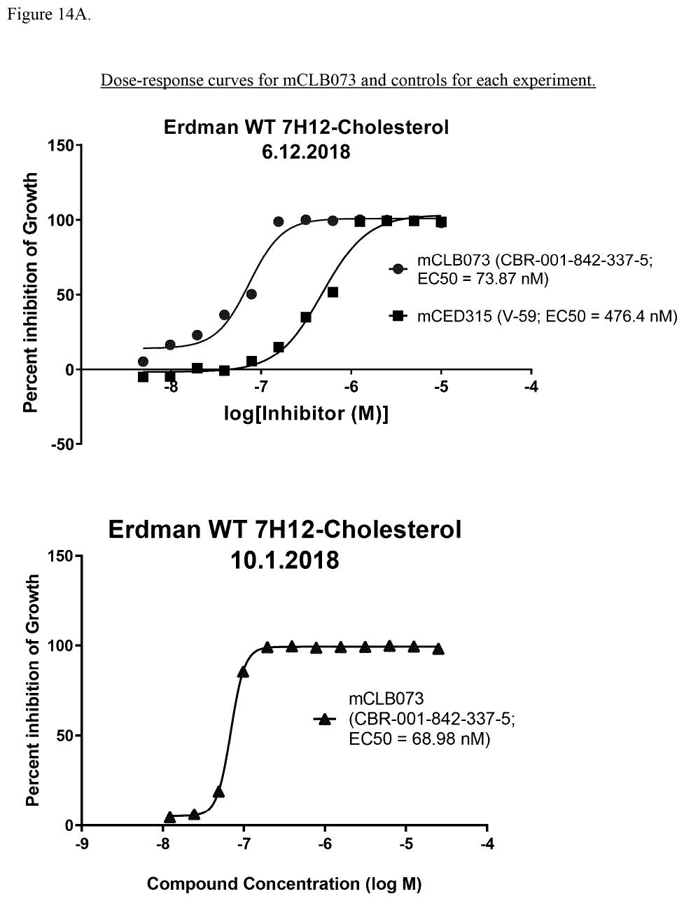

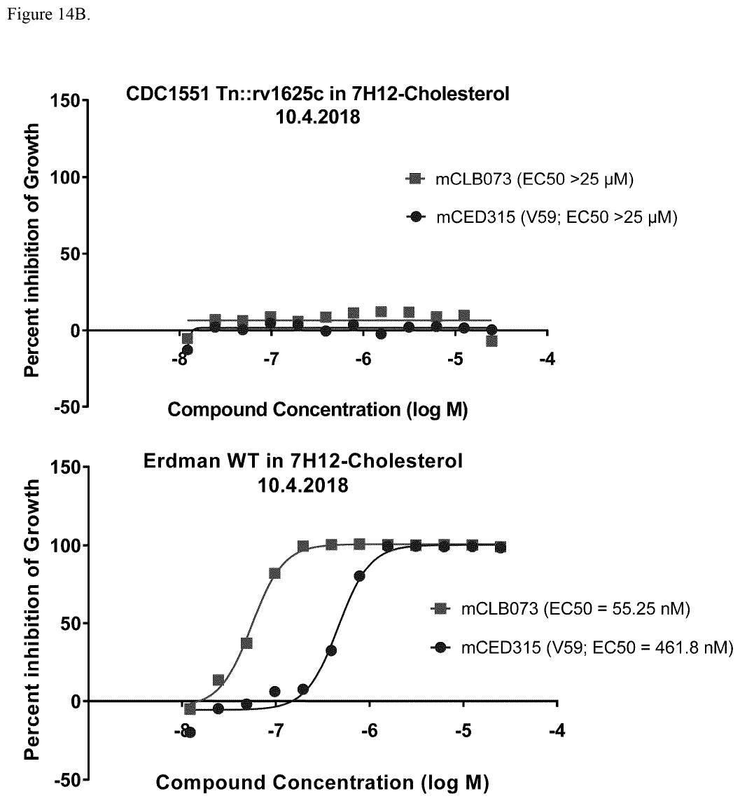

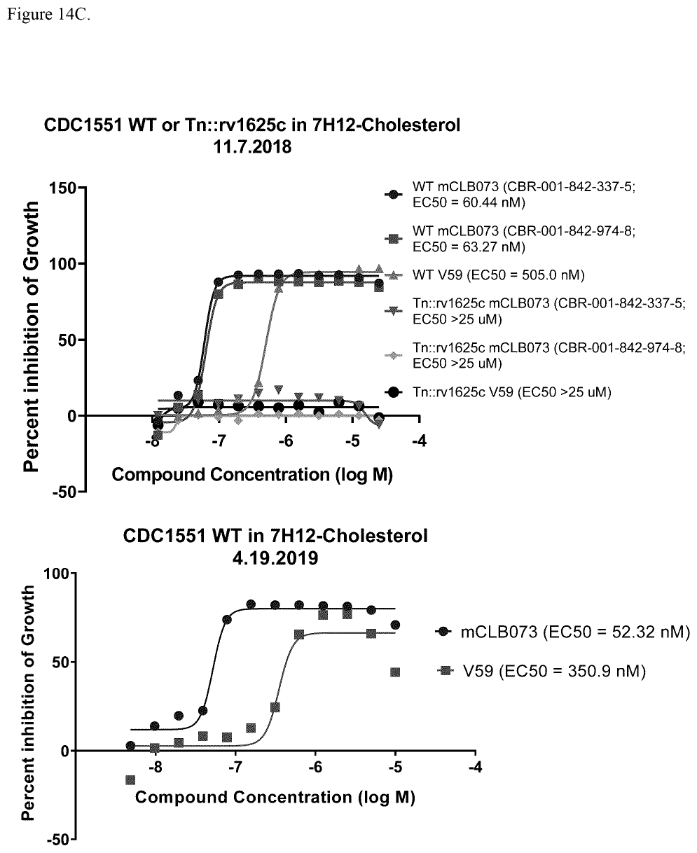

FIGS. 14A-C. Determining Extracellular Activity Against M. tuberculosis in Cholesterol Media.

Summary of activity of mCLB073 in 7H12-Cholesterol media against Mtb Erdman wild-type, CDCl551 wild-type, or the Rv1625c transposon mutant (CDCl551 Tn::rv1625c) is shown in FIG. 14A-C. For each experiment, the compound batch is identified, and n=1 for all values.

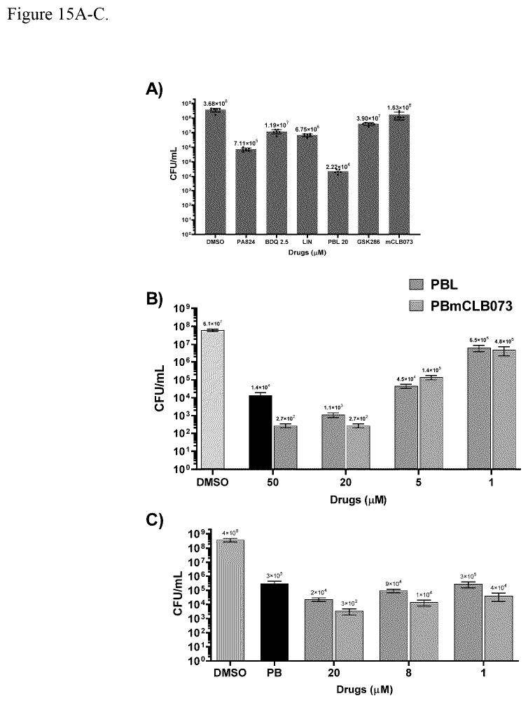

FIGS. 15A-C. Determining activity of mCLB073 in a Hu Coats 100-day old culture model.

Efficacy of NixTB combination (Pretomanid, P; Bedaquiline, B; Linezolid, L) either alone or by replacing Linezolid with mCLB073 at 20 μM and 50 μM was tested in the Hu Coats 100-day-old culture model. Rv1625c activator mCLB073 had no efficacy when tested individually in this model (FIG. 1A). However, combination of mCLB073 with Pretomanid and Bedaquiline (with all compounds at 20 μM) produced a one-log improvement in cidal activity compared to the NixTB combination. Moreover, when mCLB073 combination with Pretomanid and Bedaquiline was increased to 50 μM, it led to complete sterilization of non-replicating Mtb, which was two log units better than the NixTB combination (FIG. 1B).

Testing was repeated using a fixed concentrations of Pretomanid and Bedaquiline at their estimated physiological concentration in human dosing (Pretomanid=20.87 μM and Bedaquiline=2.25 μM). The PB combination was tested either with mCLB073 or Linezolid at three concentrations (20 μM, 8 μM and 1 μM). It was observed that mCLB073 in combination was comparable in activity to NixTB combination (FIG. 1C).

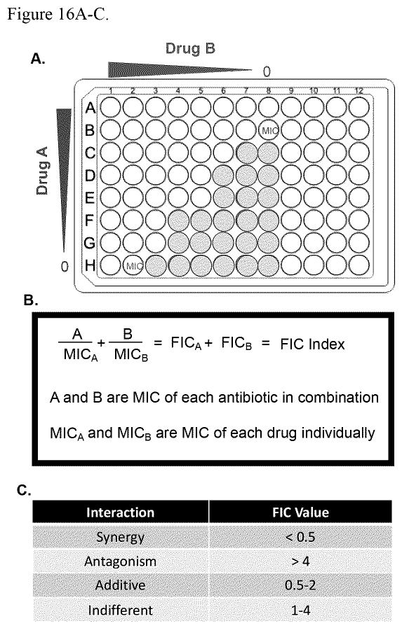

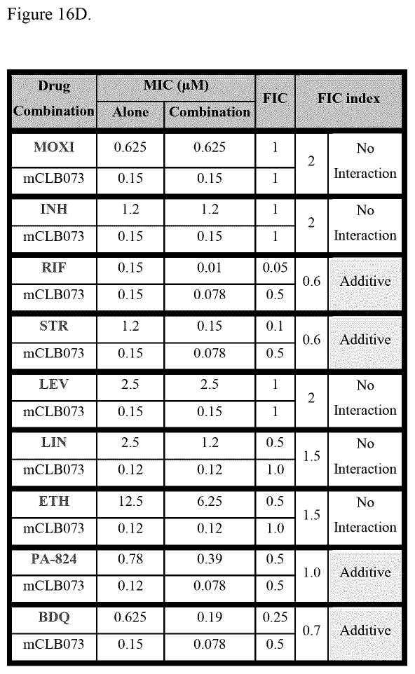

FIGS. 16A-D. Combination testing of mCLB073 with known anti-TB drugs.

A checkerboard assay to test for interactions between mCLB073 and drugs with known anti-TB activity using Alamar Blue as a viability marker (Lechartier et al., 2012) was used. Drug combinations were categorized based on fractional inhibitory concentration (FIC) index of FIC≤0.5 as synergistic, 0.5<FIC≤1 as additive, 1<FIC<4 as no interaction and FIC ≥4 as antagonism (Hall et al., 1983). FIGS. 16A-C shows the FIC index and interpretation of each combination. FIG. 16 D shows the synergy between mCLB073 and anti-tubercular drugs.

FIG. 17A-E. The inhibitory activity of V-59 in cholesterol media is dependent on Rv1625c.

(A) Inhibitory activity of V-59 against WT, the Rv1625c transposon mutant (Tn::rv1625c), and WT transformed with an overexpression plasmid expressing the rv1625c gene (2xrv1625c) in 7H12+cholesterol media. (B) Inhibitory activity of V-59 against WT, ΔRv1625c, and Compfull in 7H12+cholesterol media (left) or 7H12+cholesterol+acetate media (right). Data shown are representative, from one experiment with two technical replicates. Symbols are mean data points, and curves display nonlinear fit of dose-response. (C) Effect of V-59 on growth of Mtb in murine macrophages. Macrophages were infected at an MOI of 2 (left), 1 (middle), or 0.5 (right) and treated with V-59 (25 μM) or DMSO. Data for MOI of 2 are from three experiments, with two or three technical replicates each. Data for MOI of 1 and 0.5 are from one experiments with two or three technical replicates each. Data are shown as means±SD. Each symbol indicates one replicate. (D) Effect of frontline antibiotics on WT and ΔRv1625 in 7H12+cholesterol (INH, RIF, EMB) or in MES-buffered 7H9OADC+glycerol, pH 5.9 (PZA). Data shown are representative, from one experiment with two technical replicates. Symbols are mean data points, and curves display nonlinear fit of dose-response. (E) RNA-seq derived normalized counts of rv1625c reads in WT, ΔRv1625c, and CompFull strains in 7H12+cholesterol media.

FIG. 18A-C. The activity of mCLB073 is dependent on Rv1625c.

(A) Inhibitory activity of mCLB073 or V-59 against spontaneous resistant mutants that were generated during culture with mCLB073. (B) Inhibitory activity of mCLB073 against WT or ΔRv1625c in 7H12+cholesterol media. Data shown are representative, from one experiment with two technical replicates. Symbols are mean data points, and curves display nonlinear fit of dose-response. (C) Impact of mCLB073 on cAMP production in WT Mtb. ELISA was used to quantify cAMP from lysed cells 24 hours after addition of mCLB073, or vehicle control (DMSO). Data is from one experiment, with two technical replicates. Data are shown as means±SD.

FIG. 19A-C. An intact cyclase domain of Rv1625c is required for V-59 to inhibit cholesterol degradation.

(A) Catabolic release of 14CO2 from [U14C]-palmitate in media containing fatty acid. EtOH (control), V-59, or Atc were added to the TetOn-cAMP Mtb cultures 24 hours prior to the start of the experiment. Data are from one experiment with three technical replicates, normalized to OD and quantified relative to EtOH control. Data are shown as means±SD (B) Catabolic release of 14CO2 from [4-14C]-cholesterol in media containing cholesterol and acetate. V-59 (10 μM) was added to the cultures once at the beginning of the experiments and DMSO was used as a vehicle control. Data are from one experiment with three technical replicates, normalized to OD and quantified relative to WT treated with DMSO. Data are shown as means±SD. (C) Schematic illustrating the topology of the N-terminal transmembrane domain and essential residues of the C-terminal cyclase domain of Rv1625c (left). Schematics illustrating modified Rv1625c constructs used in these studies (center and right).

FIG. 20A-F. Construction and validation of TetOn-cAMP constructs.

(A) Schematic illustrating the domains of the native Rv1264 adenylyl cyclase (left) and the design of the TetOn-cAMP construct (right). The TetOn-cAMP construct contains the minimum-necessary cyclase domain of Rv1264, and lacks the pH-sensitive inhibitory domain of the native Rv1264 protein. Expression of the cyclase domain is under control of a TetOn promoter. Upon treatment with Atc, release of the tetracycline repressor (TetR) causes initiation of transcription of the Rv1264 catalytic domain. The C-terminal end of the cyclase domain is His-tagged to allow immunoblotting. (B) Immunoblots of bacterial lysates confirm that the TetOn constructs are expressed in the presence of Atc and not the vehicle control EtOH. The anti-His blot detects the Rv1264 cyclase domain and the anti-GroEL2 blot is the loading control. (C) Impact of inducing the TetOn-Rv1264D265A construct in media containing cholesterol and acetate. Cultures were treated with one dose of EtOH, V-59 (10 μM), or Atc at the indicated concentrations and samples were collected 24 hours later for ELISA. Data are displayed as total cAMP per 108 Mtb. Data are from two independent experiments with two technical replicates each, shown as means±SD (**P<0.01, One-way ANOVA with Dunnett's multiple comparisons test). (D) Effect of inducing TetOn-Rv1264D265A on the growth of Mtb in 7H12+cholesterol media, monitored by serial OD measurements. EtOH, V-59 (10 μM), or Atc at the indicated concentrations were added initially and every three days for the duration of the experiment. Data are from one experiment with three technical replicates, shown as means±SD. (E) Catabolic release of 14CO2 from [4-14C]-cholesterol in media containing cholesterol and acetate. The TetOn-Rv1264D265A strain was treated with EtOH, V-59 (10 μM), or Atc at the indicated concentrations overnight and again one hour prior to the beginning of the experiments. Data are from two independent experiments with three technical replicates each, normalized to OD and quantified relative to EtOH vehicle control. Shown as means±SD (not significant, Student's t test). (F) Relative GFP signal from the prpD′::GFP reporter in response to inducing TetOn-Rv1264D265A in media containing cholesterol and acetate. Cultures were treated with EtOH, V-59 (10 μM), or Atc at the indicated concentrations. Data are normalized to EtOH vehicle control (**P<0.01, One-way ANOVA with Dunnett's multiple comparisons test). GFP MFI was quantified from 10,000 mCherry+Mtb. Data are from two independent experiments with two technical replicates each, shown as means±SD.

FIG. 21A-F. Activating cAMP synthesis inhibits lipid metabolism in an Mt-Pat independent mechanism, and can inhibit fatty acid utilization without increasing antibiotic tolerance or increasing mammalian cAMP synthesis.

(A) Inhibitory activity of V-59 against WT or ΔMtPat in 7H12+cholesterol media. Data shown are from one experiment with two technical replicates. Symbols are mean data points, and curves display nonlinear fit of dose-response. (B) Relative GFP signal from the prpD′::GFP reporter in WT versus ΔMt-Pat strains carrying the TetOn-cAMP construct in media containing cholesterol and acetate. Cultures were treated in parallel with EtOH, V-59 (10 μM), or Atc at the indicated concentrations. Data are normalized to EtOH vehicle control in each strain. GFP MFI was quantified from 10,000 mCherry+Mtb. Data are from one experiment with two technical replicates, shown as means±SD. (C) Effect of inducing TetOn-cAMP on the growth of Mtb in 7H12+cholesterol+acetate media. EtOH, V-59 (10 μM), or Atc at the indicated concentrations were added initially and every three days for the duration of the experiment. Data are from one experiment with three technical replicates, shown as means±SD. (D) Relative GFP signal from the prpD′::GFP reporter in Mtb treated with V-59 or DMSO, in 71H12 media supplemented with C17:1 or propionate. Data shown normalized to WT+DMSO. GFP MFI was quantified from 10,000 mCherry positive Mtb. Data are from one experiment with two technical replicates, shown as means±SD. (E) Effect of mCLB073 treatment combined with a sub-optimal dose of rifampicin (RIF) in BALB/c mice infected by the aerosol route. Data are from one experiment with 10 mice per group (**P<0.01, Mann-Whitney test). Data are shown as means±SEM. (F) Quantification of cAMVP in human cell lines treated with V-59 (10 μM) or DMSO control. Data are from one experiment with two technical replicates, and are shown as means±SD.

In the various embodiments described herein, the mtb inhibitor of any one of Formulae I or II or pharmaceutically acceptable salt and/or stereoisomer thereof, is one selected from Compounds 1-258 in Table 1 shown below. Compounds 80 and 126 are reference compounds.

TABLE 1
Chemical Characterization Data and Assay Results

| | | Cholesterol | |
|---|---|---|---|
| | | broth: alamar | |
| | | blue--EC50) | |
| # | Structure | [μM] | Compound Name |
| 1 | 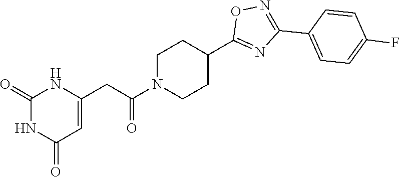 [SMILES: O=C(Cc1cc(=O)[nH]c(=O)[nH]1)N1CCC(c2nc(-c3ccc(F)cc3)no2)CC1] | 0.784 | 6-(2-(4-(3-(4-fluorophenyl)- 1,2,4-oxadiazol-5-yl)piperi- din-1-yl)-2-oxoethyl)pyr- imidine-2,4(1H,3H)-dione |
| 2 | 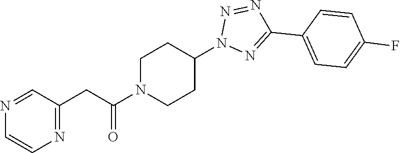 [SMILES: O=C(Cc1cnccn1)N1CCC(n2nnc(-c3ccc(F)cc3)n2)CC1] | >10 | 1-(4-(5-(4-fluorophenyl)- 2H-tetrazol-2-yl)piperidin- 1-yl)-2-(pyrazin-2-yl)ethan- 1-one |
| 3 | 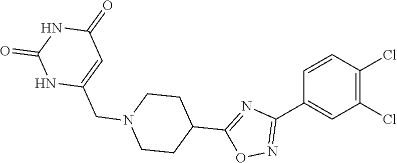 [SMILES: O=c1cc(CN2CCC(c3nc(-c4ccc(Cl)c(Cl)c4)no3)CC2)[nH]c(=O)[nH]1] | 0.179 | 6-((4-(3-(3,4-dichlorophen- yl)-1,2,4-oxadiazol-5-yl)- piperidin-1-yl)methyl)pyr- imidine-2,4(1H,3H)-dione |
| 4 | 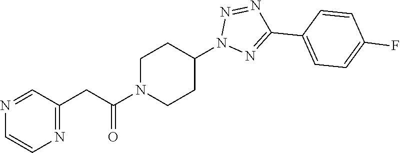 [SMILES: O=C(Cc1cnccn1)N1CCC(n2nnc(-c3ccc(F)cc3)n2)CC1] | 0.122 | 1-(4-(5-(4-fluorophenyl)- 2H-tetrazol-2-yl)piperidin- 1-yl)-2-(pyrazin-2-yl)ethan- 1-one |
| 5 | 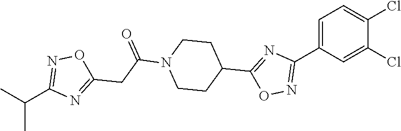 [SMILES: CC(C)c1noc(CC(=O)N2CCC(c3nc(-c4ccc(Cl)c(Cl)c4)no3)CC2)n1] | 1.43 | 1-(4-(3-(3,4-dichlorophen- yl)-1,2,4-oxadiazol-5-yl)- piperidin-1-yl)-2-(3-isopro- pyl-1,2,4-oxadiazol-5-yl)- ethan-1-one |
| 6 | 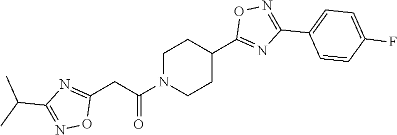 [SMILES: CC(C)c1noc(CC(=O)N2CCC(c3nc(-c4ccc(F)cc4)no3)CC2)n1] | 2.98 | 1-(4-(3-(4-fluorophenyl)- 1,2,4-oxadiazol-5-yl)piperi- din-1-yl)-2-(3-isopropyl- 1,2,4-oxadiazol-5-yl)ethan- 1-one |
| 7 | 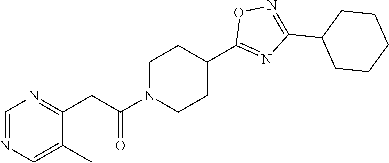 [SMILES: Cc1cncnc1CC(=O)N1CCC(c2nc(C3CCCCC3)no2)CC1] | >25 | 1-(4-(3-cyclohexyl-1,2,4- oxadiazol-5-yl)piperidin-1- yl)-2-(5-methylpyrimidin-4- yl)ethan-1-one |
| 8 | 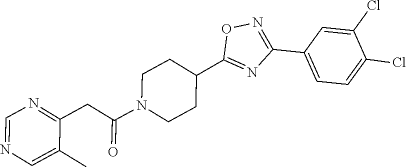 [SMILES: Cc1cncnc1CC(=O)N1CCC(c2nc(-c3ccc(Cl)c(Cl)c3)no2)CC1] | 0.479 | 1-(4-(3-(3,4-dichlorophen- yl)-1,2,4-oxadiazol-5-yl)- piperidin-1-yl)-2-(5-methyl- pyrimidin-4-yl)ethan-1-one |
| 9 | 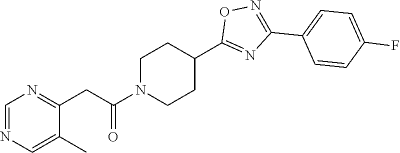 [SMILES: Cc1cncnc1CC(=O)N1CCC(c2nc(-c3ccc(F)cc3)no2)CC1] | 3.68 | 1-(4-(3-(4-fluorophenyl)- 1,2,4-oxadiazol-5-yl)piperi- din-1-yl)-2-(5-methylpyr- imidin-4-yl)ethan-1-one |
| 10 | 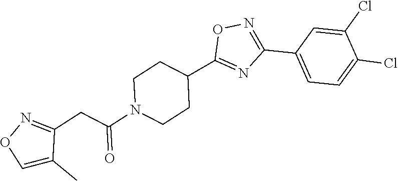 [SMILES: Cc1conc1CC(=O)N1CCC(c2nc(-c3ccc(Cl)c(Cl)c3)no2)CC1] | 0.284 | 1-(4-(3-(3,4-dichlorophen- yl)-1,2,4-oxadiazol-5-yl)- piperidin-1-yl)-2-(4-methyl- isoxazol-3-yl)ethan-1-one |
| 11 | 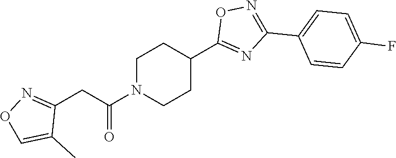 [SMILES: Cc1conc1CC(=O)N1CCC(c2nc(-c3ccc(F)cc3)no2)CC1] | 1.43 | 1-(4-(3-(4-fluorophenyl)- 1,2,4-oxadiazol-5-yl)piperi- din-1-yl)-2-(4-methylisox- azol-3-yl)ethan-1-one |
| 12 | 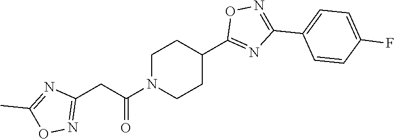 [SMILES: Cc1nc(CC(=O)N2CCC(c3nc(-c4ccc(F)cc4)no3)CC2)no1] | >10 | 1-(4-(3-(4-fluorophenyl)- 1,2,4-oxadiazol-5-yl)piperi- din-1-yl)-2-(5-methyl-1,2,4- oxadiazol-3-yl)ethan-1-one |
| 13 | 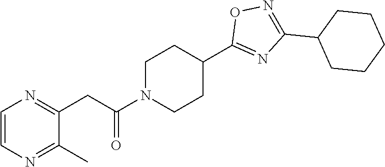 [SMILES: Cc1nccnc1CC(=O)N1CCC(c2nc(C3CCCCC3)no2)CC1] | >25 | 1-(4-(3-cyclohexyl-1,2,4- oxadiazol-5-yl)piperidin-1- yl)-2-(3-methylpyrazin-2- yl)ethan-1-one |
| 14 | 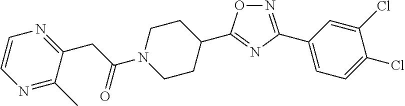 [SMILES: Cc1nccnc1CC(=O)N1CCC(c2nc(-c3ccc(Cl)c(Cl)c3)no2)CC1] | 8.32 | 1-(4-(3-(3,4-dichlorophen- yl)-1,2,4-oxadiazol-5-yl)- piperidin-1-yl)-2-(3-methyl- pyrazin-2-yl)ethan-1-one |
| 15 | 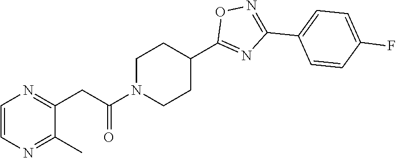 [SMILES: Cc1nccnc1CC(=O)N1CCC(c2nc(-c3ccc(F)cc3)no2)CC1] | 1.22 | 1-(4-(3-(4-fluorophenyl)- 1,2,4-oxadiazol-5-yl)piperi- din-1-yl)-2-(3-methylpyr- azin-2-yl)ethan-1-one |
| 16 | 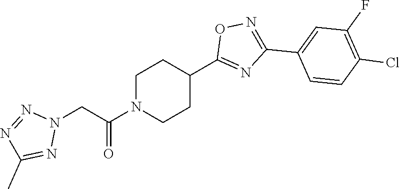 [SMILES: Cc1nnn(CC(=O)N2CCC(c3nc(-c4ccc(Cl)c(F)c4)no3)CC2)n1] | 0.79 | 1-(4-(3-(4-chloro-3-fluoro- phenyl)-1,2,4-oxadiazol-5- yl)piperidin-1-yl)-2-(5- methyl-2H-tetrazol-2-yl)- ethan-1-one |
| 17 | 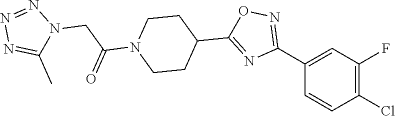 [SMILES: Cc1nnnn1CC(=O)N1CCC(c2nc(-c3ccc(Cl)c(F)c3)no2)CC1] | 0.16 | 1-(4-(3-(4-chloro-3-fluoro- phenyl)-1,2,4-oxadiazol-5- yl)piperidin-1-yl)-2-(5- methyl-1H-tetrazol-1-yl)- ethan-1-one |
| 18 | 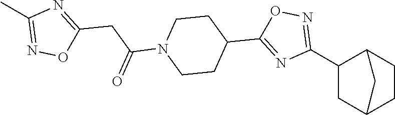 [SMILES: Cc1noc(CC(=O)N2CCC(c3nc(C4CC5CCC4C5)no3)CC2)n1] | >10 | 1-(4-(3-(bicyclo[2.2.1] heptan-2-yl)-1,2,4-oxadia- zol-5-yl)piperidin-1-yl)-2- (3-methyl-1,2,4-oxadiazol- 5-yl)ethan-1-one |
| 19 | 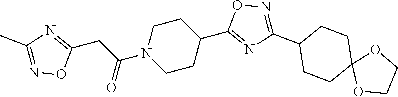 [SMILES: Cc1noc(CC(=O)N2CCC(c3nc(C4CCC5(CC4)OCCO5)no3)CC2)n1] | >10 | 1-(4-(3-(1,4-dioxaspiro[4.5] decan-8-yl)-1,2,4-oxadia- zol-5-yl)piperidin-1-yl)-2- (3-methyl-1,2,4-oxadiazol- 5-yl)ethan-1-one |
| 20 | 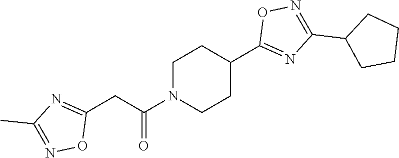 [SMILES: Cc1noc(CC(=O)N2CCC(c3nc(C4CCCC4)no3)CC2)n1] | >25 | 1-(4-(3-cyclopentyl-1,2,4- oxadiazol-5-yl)piperidin-1- yl)-2-(3-methyl-1,2,4-oxa- diazol-5-yl)ethan-1-one |
| 21 | 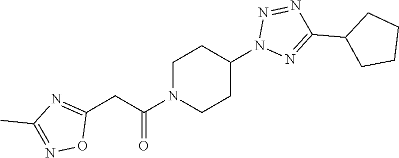 [SMILES: Cc1noc(CC(=O)N2CCC(n3nnc(C4CCCC4)n3)CC2)n1] | 7.35 | 1-(4-(5-cyclopentyl-2H- tetrazol-2-yl)piperidin-1-yl)- 2-(3-methyl-1,2,4-oxadia- zol-5-yl)ethan-1-one |
| 22 | 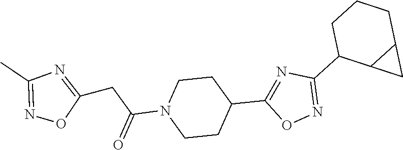 [SMILES: Cc1noc(CC(=O)N2CCC(c3nc(C4CCCC5CC54)no3)CC2)n1] | 0.89 | 1-(4-(3-(bicyclo[4.1.0] heptan-2-yl)-1,2,4-oxadia- zol-5-yl)piperidin-1-yl)-2- (3-methyl-1,2,4-oxadia- zol-5-yl)ethan-1-one |
| 23 | 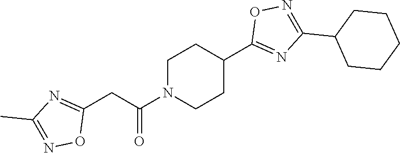 [SMILES: Cc1noc(CC(=O)N2CCC(c3nc(C4CCCCC4)no3)CC2)n1] | >10 | 1-(4-(3-cyclohexyl-1,2,4- oxadiazol-5-yl)piperidin-1- yl)-2-(3-methyl-1,2,4-oxa- diazol-5-yl)ethan-1-one |
| 24 | 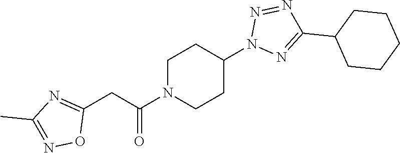 [SMILES: Cc1noc(CC(=O)N2CCC(n3nnc(C4CCCCC4)n3)CC2)n1] | >25 | 1-(4-(5-cyclohexyl-2H- tetrazol-2-yl)piperidin-1-yl)- 2-(3-methyl-1,2,4-oxadia- zol-5-yl)ethan-1-one |
| 25 | 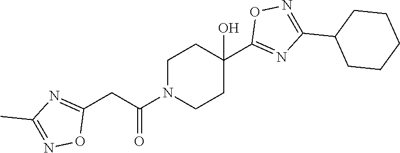 [SMILES: Cc1noc(CC(=O)N2CCC(O)(c3nc(C4CCCCC4)no3)CC2)n1] | >10 | 1-(4-(3-cyclohexyl-1,2,4- oxadiazol-5-yl)-4-hydroxy- piperidin-1-yl)-2-(3-meth- yl-1,2,4-oxadiazol-5-yl)- ethan-1-one |
| 26 | 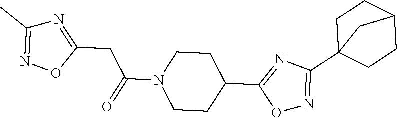 [SMILES: Cc1noc(CC(=O)N2CCC(c3nc(C45CCC(CC4)C5)no3)CC2)n1] | >25 | 1-(4-(3-(bicyclo[2.2.1] heptan-1-yl)-1,2,4-oxadia- zol-5-yl)piperidin-1-yl)-2- (3-methyl-1,2,4-oxadiazol- 5-yl)ethan-1-one |
| 27 | 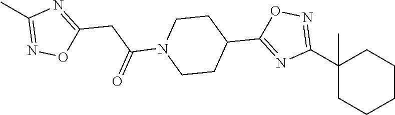 [SMILES: Cc1noc(CC(=O)N2CCC(c3nc(C4(C)CCCCC4)no3)CC2)n1] | >25 | 2-(3-methyl-1,2,4-oxadia- zol-5-yl)-1-(4-(3-(1-meth- ylcyclohexyl)-1,2,4-oxadia- zol-5-yl)piperidin-1-yl)- ethan-1-one |
| 28 | 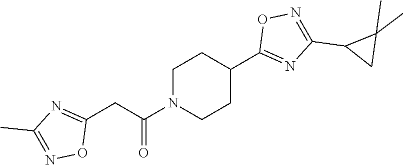 | >25 | 1-(4-(3-(2,2-dimethylcyclo- propyl)-1,2,4-oxadiazol-5- yl)piperidin-1-yl)-2-(3- methyl-1,2,4-oxadiazol-5- yl)ethan-1-one |
| 29 | 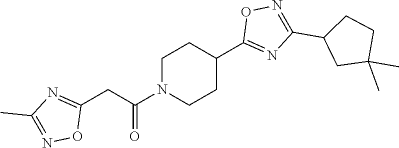 | 3.73 | 1-(4-(3-(3,3-dimethylcyclo- pentyl)-1,2,4-oxadiazol-5- yl)piperidin-1-yl)-2-(3- methyl-1,2,4-oxadiazol-5- yl)ethan-1-one |
| 30 | 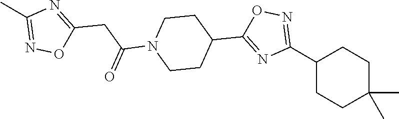 | 1.14 | 1-(4-(3-(4,4-dimethylcyclo- hexyl)-1,2,4-oxadiazol-5- yl)piperidin-1-yl)-2-(3- methyl-1,2,4-oxadiazol-5- yl)ethan-1-one |
| 31 | 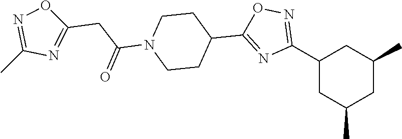 | >25 | 1-(4-(3-((3R,5S)-3,5- dimethylcyclohexyl)-1,2,4- oxadiazol-5-yl)piperidin-1- yl)-2-(3-methyl-1,2,4-oxa- diazol-5-yl)ethan-1-one |
| 32 | 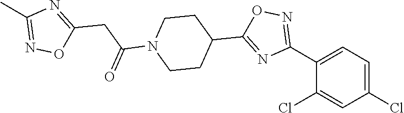 [SMILES: Cc1noc(CC(=O)N2CCC(c3nc(-c4ccc(Cl)cc4Cl)no3)CC2)n1] | 0.718 | 1-(4-(3-(2,4-dichlorophen- yl)-1,2,4-oxadiazol-5-yl)- piperidin-1-yl)-2-(3-methyl- 1,2,4-oxadiazol-5-yl)ethan- 1-one |
| 33 | 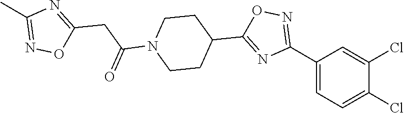 [SMILES: Cc1noc(CC(=O)N2CCC(c3nc(-c4ccc(Cl)c(Cl)c4)no3)CC2)n1] | 0.14 | 1-(4-(3-(3,4-dichlorophen- yl)-1,2,4-oxadiazol-5-yl)- piperidin-1-yl)-2-(3-methyl- 1,2,4-oxadiazol-5-yl)ethan- 1-one |
| 34 | 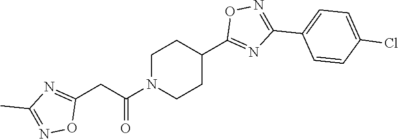 [SMILES: Cc1noc(CC(=O)N2CCC(c3nc(-c4ccc(Cl)cc4)no3)CC2)n1] | 0.399 | 1-(4-(3-(4-chlorophenyl)- 1,2,4-oxadiazol-5-yl)piperi- din-1-yl)-2-(3-methyl-1,2,4- oxadiazol-5-yl)ethan-1-one |
| 35 | 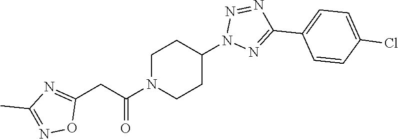 [SMILES: Cc1noc(CC(=O)N2CCC(n3nnc(-c4ccc(Cl)cc4)n3)CC2)n1] | 1.08 | 1-(4-(5-(4-chlorophenyl)- 2H-tetrazol-2-yl)piperidin- 1-yl)-2-(3-methyl-1,2,4-oxa- diazol-5-yl)ethan-1-one |
| 36 | 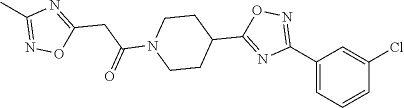 [SMILES: Cc1noc(CC(=O)N2CCC(c3nc(-c4cccc(Cl)c4)no3)CC2)n1] | 1.63 | 1-(4-(3-(3-chlorophenyl)- 1,2,4-oxadiazol-5-yl)piperi- din-1-yl)-2-(3-methyl-1,2,4- oxadiazol-5-yl)ethan-1-one |
| 37 | 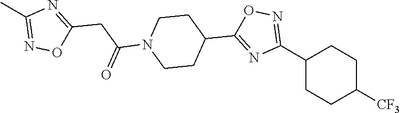 [SMILES: Cc1noc(CC(=O)N2CCC(c3nc(C4CCC(C(F)(F)F)CC4)no3)CC2)n1] | 7.02 | 2-(3-methyl-1,2,4-oxadia- zol-5-yl)-1-(4-(3-(4-(tri- fluoromethyl)cyclohexyl)- 1,2,4-oxadiazol-5-yl)piperi- din-1-yl)ethan-1-one |
| 38 | 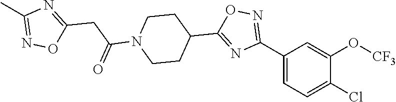 [SMILES: Cc1noc(CC(=O)N2CCC(c3nc(-c4ccc(Cl)c(OC(F)(F)F)c4)no3)CC2)n1] | 1.05 | 1-(4-(3-(4-chloro-3-tri- fluoromethoxy)phenyl)- 1,2,4-oxadiazol-5-yl)piperi- din-1-yl)-2-(3-methyl-1,2,4- oxadiazol-5-yl)ethan-1-one |
| 39 | 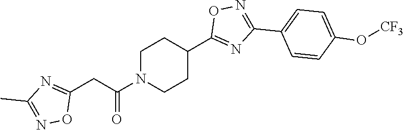 [SMILES: Cc1noc(CC(=O)N2CCC(c3nc(-c4ccc(OC(F)(F)F)cc4)no3)CC2)n1] | 0.657 | 2-(3-methyl-1,2,4-oxadia- zol-5-yl)-1-(4-(3-(4-(tri- fluoromethoxy)phenyl)- 1,2,4-oxadiazol-5-yl)piperi- din-1-yl)ethan-1-one |
| 40 | 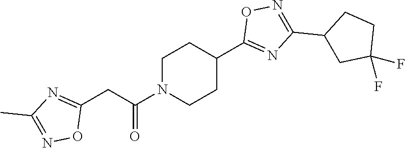 [SMILES: Cc1noc(CC(=O)N2CCC(c3nc(C4CCC(F)(F)C4)no3)CC2)n1] | >25 | 1-(4-(3-(3,3-difluorocyclo- pentyl)-1,2,4-oxadiazol-5- yl)piperidin-1-yl)-2-(3- methyl-1,2,4-oxadiazol-5- yl)ethan-1-one |
| 41 | 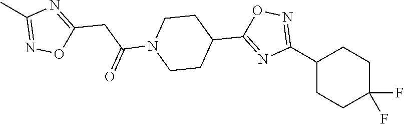 [SMILES: Cc1noc(CC(=O)N2CCC(c3nc(C4CCC(F)(F)CC4)no3)CC2)n1] | >10 | 1-(4-(3-(4,4-difluorocyclo- hexyl)-1,2,4-oxadiazol-5- yl)piperidin-1-yl)-2-(3- methyl-1,2,4-oxadiazol-5- yl)ethan-1-one |
| 42 | 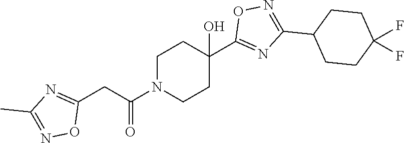 [SMILES: Cc1noc(CC(=O)N2CCC(O)(c3nc(C4CCC(F)(F)CC4)no3)CC2)n1] | >10 | 1-(4-(3-(4,4-difluorocyclo- hexyl)-1,2,4-oxadiazol-5- yl)-4-hydroxypiperidin-1- yl)-2-(3-methyl-1,2,4-oxa- diazol-5-yl)ethan-1-one |
| 43 | 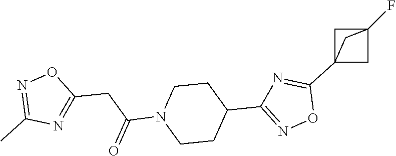 [SMILES: Cc1noc(CC(=O)N2CCC(c3noc(C45CC(F)(C4)C5)n3)CC2)n1] | >25 | 1-(4-(5-(3-fluorobicyclo [1.1.1]pentan-1-yl)-1,2,4- oxadiazol-3-yl)piperidin-1- yl)-2-(3-methyl-1,2,4-oxa- diazol-5-yl)ethan-1-one |
| 44 | 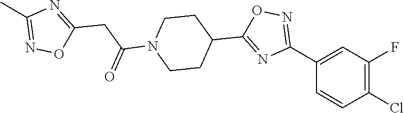 [SMILES: Cc1noc(CC(=O)N2CCC(c3nc(-c4ccc(Cl)c(F)c4)no3)CC2)n1] | 0.229 | 1-(4-(3-(4-chloro-3-fluoro- phenyl)-1,2,4-oxadiazol-5- yl)piperidin-1-yl)-2-(3- methyl-1,2,4-oxadiazol-5- yl)ethan-1-one |
| 45 | 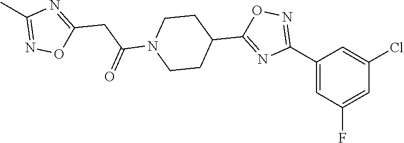 [SMILES: Cc1noc(CC(=O)N2CCC(c3nc(-c4cc(F)cc(Cl)c4)no3)CC2)n1] | 2.19 | 1-(4-(3-(3-chloro-5-fluoro- phenyl)-1,2,4-oxadiazol-5- yl)piperidin-1-yl)-2-(3- methyl-1,2,4-oxadiazol-5- yl)ethan-1-one |
| 46 | 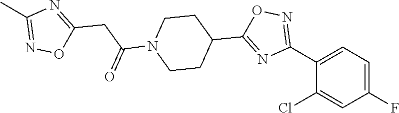 [SMILES: Cc1noc(CC(=O)N2CCC(c3nc(-c4ccc(F)cc4Cl)no3)CC2)n1] | 2.97 | 1-(4-(3-(2-chloro-4-fluoro- phenyl)-1,2,4-oxadiazol-5- yl)piperidin-1-yl)-2-(3- methyl-1,2,4-oxadiazol-5- yl)ethan-1-one |
| 47 | 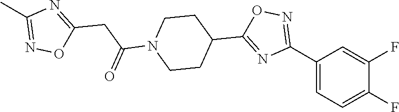 [SMILES: Cc1noc(CC(=O)N2CCC(c3nc(-c4ccc(F)c(F)c4)no3)CC2)n1] | 1.45 | 1-(4-(3-(3,4-difluorophen- yl)-1,2,4-oxadiazol-5-yl)- piperidin-1-yl)-2-(3-meth- yl-1,2,4-oxadiazol-5-yl)- ethan-1-one |
| 48 | 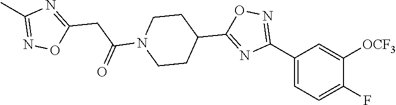 [SMILES: Cc1noc(CC(=O)N2CCC(c3nc(-c4ccc(F)c(OC(F)(F)F)c4)no3)CC2)n1] | 0.73 | 1-(4-(3-(4-fluoro-3-(tri- fluoromethoxy)phenyl)- 1,2,4-oxadiazol-5-yl)piperi- din-1-yl)-2-(3-methyl-1,2,4- oxadiazol-5-yl)ethan-1-one |
| 49 | 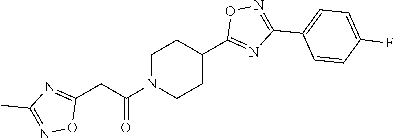 [SMILES: Cc1noc(CC(=O)N2CCC(c3nc(-c4ccc(F)cc4)no3)CC2)n1] | 1.44 | 1-(4-(3-(4-fluorophenyl)- 1,2,4-oxadiazol-5-yl)piperi- din-1-yl)-2-(3-methyl-1,2,4- oxadiazol-5-yl)ethan-1-one |
| 50 | 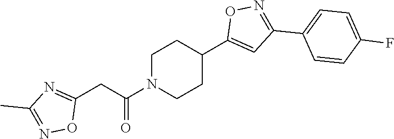 [SMILES: Cc1noc(CC(=O)N2CCC(c3cc(-c4ccc(F)cc4)no3)CC2)n1] | >10 | 1-(4-(3-(4-fluorophenyl)- isoxazol-5-yl)piperidin-1- yl)-2-(3-methyl-1,2,4-oxa- diazol-5-yl)ethan-1-one |
| 51 | 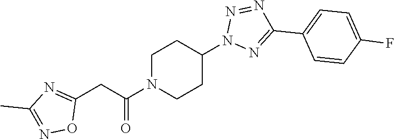 [SMILES: Cc1noc(CC(=O)N2CCC(n3nnc(-c4ccc(F)cc4)n3)CC2)n1] | 7.64 | 1-(4-(5-(4-fluorophenyl)- 2H-tetrazol-2-yl)piperidin- 1-yl)-2-(3-methyl-1,2,4- oxadiazol-5-yl)ethan-1-one |
| 52 | 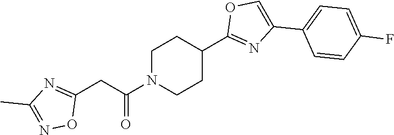 [SMILES: Cc1noc(CC(=O)N2CCC(c3nc(-c4ccc(F)cc4)co3)CC2)n1] | >10 | 1-(4-(4-(4-fluorophenyl)- oxazol-2-yl)piperidin-1-yl)- 2-(3-methyl-1,2,4-oxadia- zol-5-yl)ethan-1-one |
| 53 | 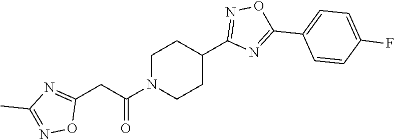 [SMILES: Cc1noc(CC(=O)N2CCC(c3noc(-c4ccc(F)cc4)n3)CC2)n1] | >10 | 1-(4-(5-(4-fluorophenyl)- 1,2,4-oxadiazol-3-yl)piperi- din-1-yl)-2-(3-methyl-1,2,4- oxadiazol-5-yl)ethan-1-one |
| 54 | 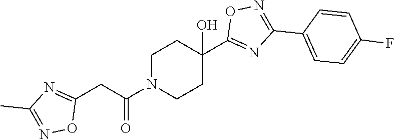 [SMILES: Cc1noc(CC(=O)N2CCC(O)(c3nc(-c4ccc(F)cc4)no3)CC2)n1] | >10 | 1-(4-(3-(4-fluorophenyl)- 1,2,4-oxadiazol-5-yl)-4- hydroxypiperidin-1-yl)-2- (3-methyl-1,2,4-oxadiazol- 5-yl)ethan-1-one |
| 55 | 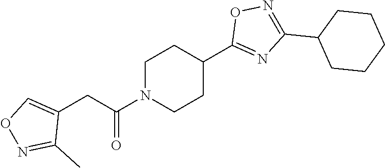 [SMILES: Cc1nocc1CC(=O)N1CCC(c2nc(C3CCCCC3)no2)CC1] | >25 | 1-(4-(3-cyclohexyl-1,2,4- oxadiazol-5-yl)piperidin-1- yl)-2-(3-methylisoxazol-4- yl)ethan-1-one |
| 56 | 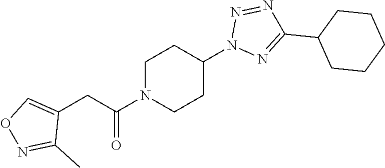 [SMILES: Cc1nocc1CC(=O)N1CCC(n2nnc(C3CCCCC3)n2)CC1] | >25 | 1-(4-(5-cyclohexyl-2H- tetrazol-2-yl)piperidin-1-yl)- 2-(3-methylisoxazol-4-yl)- ethan-1-one |
| 57 | 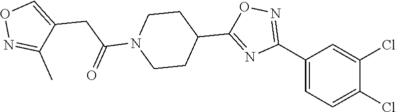 [SMILES: Cc1nocc1CC(=O)N1CCC(c2nc(-c3ccc(Cl)c(Cl)c3)no2)CC1] | 0.691 | 1-(4-(3-(3,4-dichlorophen- yl)-1,2,4-oxadiazol-5-yl)- piperidin-1-yl)-2-(3-meth- ylisoxazol-4-yl)ethan-1-one |
| 58 | 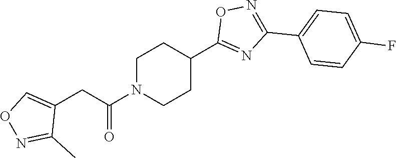 [SMILES: Cc1nocc1CC(=O)N1CCC(c2nc(-c3ccc(F)cc3)no2)CC1] | 2.15 | 1-(4-(3-(4-fluorophenyl)- 1,2,4-oxadiazol-5-yl)piperi- din-1-yl)-2-(3-methylisox- azol-4-yl)ethan-1-one |
| 59 | 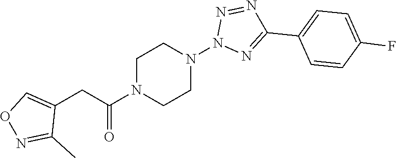 [SMILES: Cc1nocc1CC(=O)N1CCN(n2nnc(-c3ccc(F)cc3)n2)CC1] | >25 | 1-(4-(5-(4-fluorophenyl)- 2H-tetrazol-2-yl)piperidin- 1-yl)-2-(3-methylisoxazol- 4-yl)ethan-1-one |
| 60 | 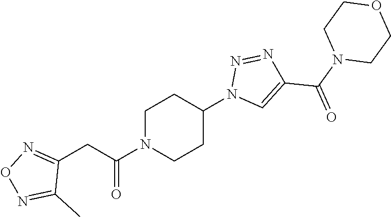 [SMILES: Cc1nonc1CC(=O)N1CCC(n2cc(C(=O)N3CCOCC3)nn2)CC1] | >10 | 2-(4-methyl-1,2,5-oxadia- zol-3-yl)-1-(4-(4-(morph- oline-4-carbonyl)-1H-1,2,3- triazol-1-yl)piperidin-1-yl)- ethan-1-one |
| 61 | 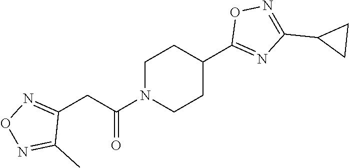 [SMILES: Cc1nonc1CC(=O)N1CCC(c2nc(C3CC3)no2)CC1] | >10 | 1-(4-(3-cyclopropyl-1,2,4- oxadiazol-5-yl)piperidin-1- yl)-2-(4-methyl-1,2,5-oxa- diazol-3-yl)ethan-1-one |
| 62 | 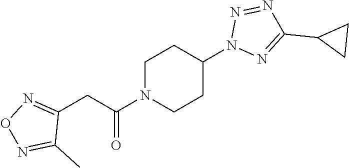 [SMILES: Cc1nonc1CC(=O)N1CCC(n2nnc(C3CC3)n2)CC1] | 10 | 1-(4-(5-cyclopropyl-2H- tetrazol-2-yl)piperidin-1- yl)-2-(4-methyl-1,2,5-oxa- diazol-3-yl)ethan-1-one |
| 63 | 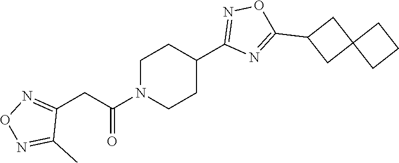 [SMILES: Cc1nonc1CC(=O)N1CCC(c2noc(C3CC4(CCC4)C3)n2)CC1] | | 2-(4-methyl-1,2,5-oxadia- zol-3-yl)-1-(4-(5-(spiro [3.3]heptan-2-yl)-1,2,4-oxa- diazol-3-yl)piperidin-1-yl)- ethan-1-one |
| 64 | 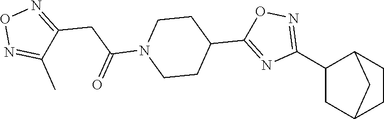 [SMILES: Cc1nonc1CC(=O)N1CCC(c2nc(C3CC4CCC3C4)no2)CC1] | 0.447 | 1-(4-(3-(bicyclo[2.2.1] heptan-2-yl)-1,2,4-oxadia- zol-5-yl)piperidin-1-yl)-2- (4-methyl-1,2,5-oxadiazol- 3-yl)ethan-1-one |
| 65 | 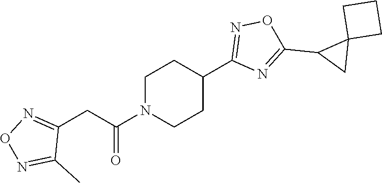 [SMILES: Cc1nonc1CC(=O)N1CCC(c2noc(C3CC34CCC4)n2)CC1] | 14.99 | 2-(4-methyl-1,2,5-oxadia- zol-3-yl)-1-(4-(5-(spiro [2.3]hexan-1-yl)-1,2,4-oxa- diazol-3-yl)piperidin-1-yl)- ethan-1-one |
| 66 | 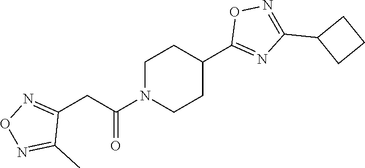 [SMILES: Cc1nonc1CC(=O)N1CCC(c2nc(C3CCC3)no2)CC1] | >25 | 1-(4-(3-cyclobutyl-1,2,4- oxadiazol-5-yl)piperidin-1- yl)-2-(4-methyl-1,2,5-oxa- diazol-3-yl)ethan-1-one |
| 67 | 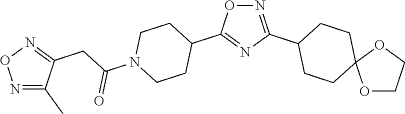 [SMILES: Cc1nonc1CC(=O)N1CCC(c2nc(C3CCC4(CC3)OCCO4)no2)CC1] | >10 | 1-(4-(3-(1,4-dioxaspiro[4.5] decan-8-yl)-1,2,4-oxadiazol- 5-yl)piperidin-1-yl)-2-(4- methyl-1,2,5-oxadiazol-3- yl)ethan-1-one |
| 68 | 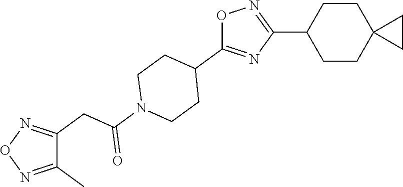 [SMILES: Cc1nonc1CC(=O)N1CCC(c2nc(C3CCC4(CC3)CC4)no2)CC1] | 1.86 | 2-(4-methyl-1,2,5-oxadia- zol-3-yl)-1-(4-(3-(spiro [2.5]octan-6-yl)-1,2,4-oxa- diazol-5-yl)piperidin-1-yl)- ethan-1-one |
| 69 | 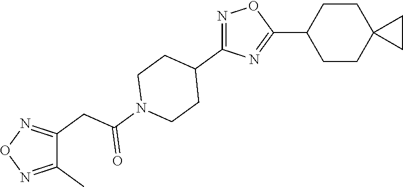 [SMILES: Cc1nonc1CC(=O)N1CCC(c2noc(C3CCC4(CC3)CC4)n2)CC1] | >25 | 2-(4-methyl-1,2,5-oxadia- zol-3-yl)-1-(4-(5-(spiro [2.5]octan-6-yl)-1,2,4-oxa- diazol-3-yl)piperidin-1-yl)- ethan-1-one |
| 70 | 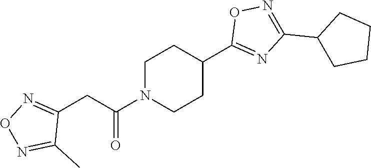 [SMILES: Cc1nonc1CC(=O)N1CCC(c2nc(C3CCCC3)no2)CC1] | 3.68 | 1-(4-(3-cyclopentyl-1,2,4- oxadiazol-5-yl)piperidin-1- yl)-2-(4-methyl-1,2,5-oxa- diazol-3-yl)ethan-1-one |
| 71 | 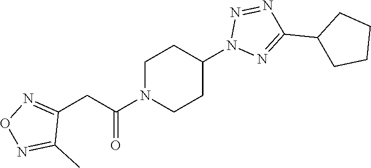 [SMILES: Cc1nonc1CC(=O)N1CCC(n2nnc(C3CCCC3)n2)CC1] | >25 | 1-(4-(5-cyclopentyl-2H- tetrazol-2-yl)piperidin-1- yl)-2-(4-methyl-1,2,5-oxa- diazol-3-yl)ethan-1-one |
| 72 | 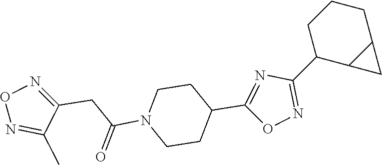 [SMILES: Cc1nonc1CC(=O)N1CCC(c2nc(C3CCCC4CC43)no2)CC1] | >10 | 1-(4-(3-(bicyclo[4.1.0] heptan-2-yl)-1,2,4-oxadia- zol-5-yl)piperidin-1-yl)-2- (4-methyl-1,2,5-oxadiazol- 3-yl)ethan-1-one |
| 73 | 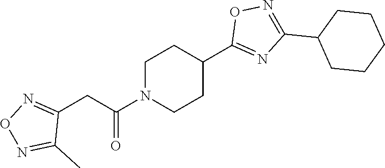 [SMILES: Cc1nonc1CC(=O)N1CCC(c2nc(C3CCCCC3)no2)CC1] | 0.674 | 1-(4-(3-cyclohexyl-1,2,4- oxadiazol-5-yl)piperidin-1- yl)-2-(4-methyl-1,2,5-oxa- diazol-3-yl)ethan-1-one |
| 74 | 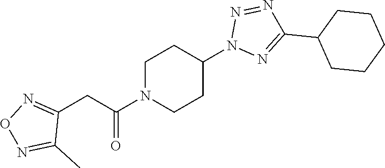 [SMILES: Cc1nonc1CC(=O)N1CCC(n2nnc(C3CCCCC3)n2)CC1] | 2.07 | 1-(4-(5-cyclohexyl-2H- tetrazol-2-yl)piperidin-1- yl)-2-(4-methyl-1,2,5-oxa- diazol-3-yl)ethan-1-one |
| 75 | 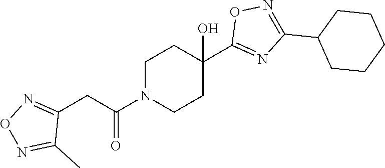 [SMILES: Cc1nonc1CC(=O)N1CCC(O)(c2nc(C3CCCCC3)no2)CC1] | >10 | 1-(4-(3-cyclohexyl-1,2,4- oxadiazol-5-yl)-4-hydroxy- piperidin-1-yl)-2-(4-meth- yl-1,2,5-oxadiazol-3-yl)- ethan-1-one |
| 76 | 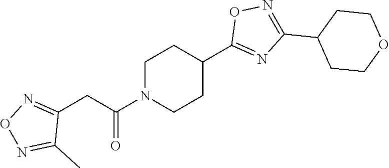 [SMILES: Cc1nonc1CC(=O)N1CCC(c2nc(C3CCOCC3)no2)CC1] | >10 | 2-(4-methyl-1,2,5-oxadia- zol-3-yl)-1-(4-(3-(tetra- hydro-2H-pyran-4-yl)-1,2,4- oxadiazol-5-yl)piperidin-1- yl)ethan-1-one |
| 77 | 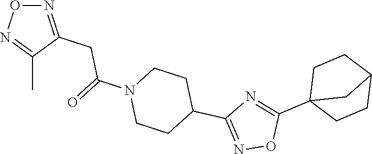 [SMILES: Cc1nonc1CC(=O)N1CCC(c2noc(C34CCC(CC3)C4)n2)CC1] | >25 | 1-(4-(5-(bicyclo[2.2.1] heptan-1-yl)-1,2,4-oxadia- zol-3-yl)piperidin-1-yl)-2- (4-methyl-1,2,5-oxadiazol- 3-yl)ethan-1-one |
| 78 | 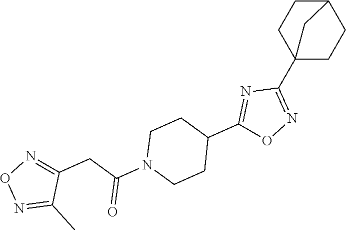 [SMILES: Cc1nonc1CC(=O)N1CCC(c2nc(C34CCC(CC3)C4)no2)CC1] | 3.76 | 1-(4-(3-(bicyclo[2.2.1] heptan-1-yl)-1,2,4-oxadia- zol-5-yl)piperidin-1-yl)-2- (4-methyl-1,2,5-oxadiazol- 3-yl)ethan-1-one |
| 79 | 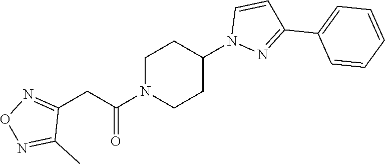 [SMILES: Cc1nonc1CC(=O)N1CCC(n2ccc(-c3ccccc3)n2)CC1] | >10 | 2-(4-methyl-1,2,5-oxadia- zol-3-yl)-1-(4-(3-phenyl- 1H-pyrazol-1-yl)piperidin- 1-yl)ethan-1-one |
| 80 | 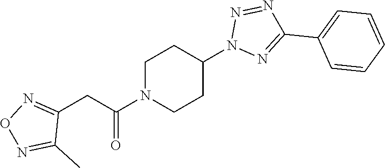 [SMILES: Cc1nonc1CC(=O)N1CCC(n2nnc(-c3ccccc3)n2)CC1] | 1.58 | 2-(4-methyl-1,2,5-oxadia- zol-3-yl)-1-(4-(5-phenyl- 2H-tetrazol-2-yl)piperidin- 1-yl)ethan-1-one |
| 81 | 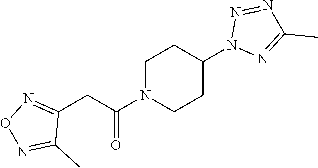 [SMILES: Cc1nnn(C2CCN(C(=O)Cc3nonc3C)CC2)n1] | >10 | 2-(4-methyl-1,2,5-oxadia- zol-3-yl)-1-(4-(5-methyl- 2H-tetrazol-2-yl)piperidin- 1-yl)ethan-1-one |
| 82 | 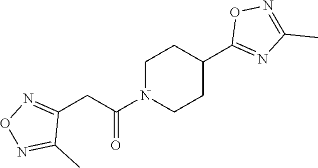 [SMILES: Cc1noc(C2CCN(C(=O)Cc3nonc3C)CC2)n1] | >10 | 1-(4-(3-methyl-1,2,4-oxa- diazol-5-yl)piperidin-1-yl)- 2-(4-methyl-1,2,5-oxadia- zol-3-yl)ethan-1-one |
| 83 | 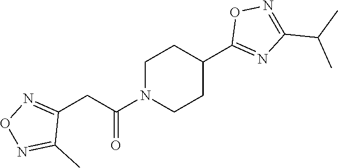 [SMILES: Cc1nonc1CC(=O)N1CCC(c2nc(C(C)C)no2)CC1] | >10 | 1-(4-(3-isopropyl-1,2,4-oxa- diazol-5-yl)piperidin-1-yl)- 2-(4-methyl-1,2,5-oxadia- zol-3-yl)ethan-1-one |
| 84 | 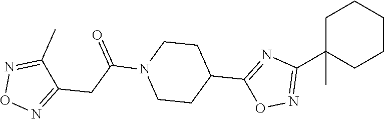 [SMILES: Cc1nonc1CC(=O)N1CCC(c2nc(C3(C)CCCCC3)no2)CC1] | 2.13 | 2-(4-methyl-1,2,5-oxadia- zol-3-yl)-1-(4-(3-(1-meth- ylcyclohexyl)-1,2,4-oxadia- zol-5-yl)piperidin-1-yl)- ethan-1-one |
| 85 | 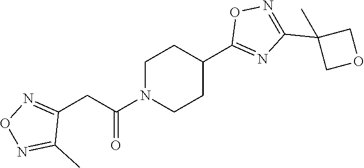 [SMILES: Cc1nonc1CC(=O)N1CCC(c2nc(C3(C)COC3)no2)CC1] | >10 | 2-(4-methyl-1,2,5-oxadia- zol-3-yl)-1-(4-(3-(3-meth- yloxetan-3-yl)-1,2,4-oxa- diazol-5-yl)piperidin-1-yl)- ethan-1-one |
| 86 | 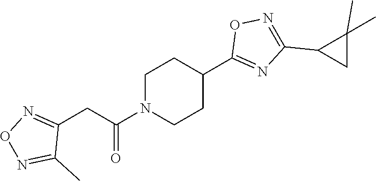 [SMILES: Cc1nonc1CC(=O)N1CCC(c2nc(C3CC3(C)C)no2)CC1] | 1.81 | 1-(4-(3-(2,2-dimethylcyclo- propyl)-1,2,4-oxadiazol-5- yl)piperidin-1-yl)-2-(4- methyl-1,2,5-oxadiazol-3- yl)ethan-1-one |
| 87 | 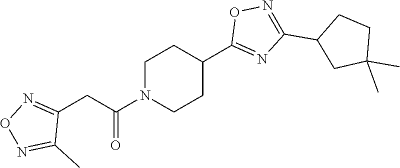 [SMILES: Cc1nonc1CC(=O)N1CCC(c2nc(C3CCC(C)(C)C3)no2)CC1] | 0.249 | 1-(4-(3-(3,3-dimethylcyclo- pentyl)-1,2,4-oxadiazol-5- yl)piperidin-1-yl)-2-(4- methyl-1,2,5-oxadiazol-3- yl)ethan-1-one |
| 88 | 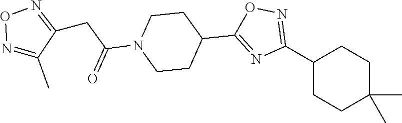 [SMILES: Cc1nonc1CC(=O)N1CCC(c2nc(C3CCC(C)(C)CC3)no2)CC1] | 0.181 | 1-(4-(3-(4,4-dimethylcyclo- hexyl)-1,2,4-oxadiazol-5- yl)piperidin-1-yl)-2-(4- methyl-1,2,5-oxadiazol-3- yl)ethan-1-one |
| 89 | 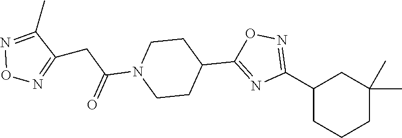 [SMILES: Cc1nonc1CC(=O)N1CCC(c2nc(C3CCCC(C)(C)C3)no2)CC1] | 0.512 | 1-(4-(3-(3,3-dimethylcyclo- hexyl)-1,2,4-oxadiazol-5- yl)piperidin-1-yl)-2-(4- methyl-1,2,5-oxadiazol-3- yl)ethan-1-one |
| 90 | 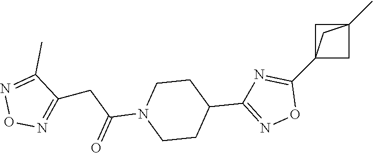 [SMILES: Cc1nonc1CC(=O)N1CCC(c2noc(C34CC(C)(C3)C4)n2)CC1] | 2.05 | 2-(4-methyl-1,2,5-oxadia- zol-3-yl)-1-(4-(5-(3-methyl- bicyclo[1.1.1]pentan-1-yl)- 1,2,4-oxadiazol-3-yl)piperi- din-1-yl)ethan-1-one |
| 91 | 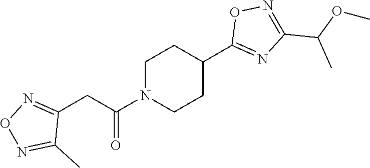 [SMILES: COC(C)c1noc(C2CCN(C(=O)Cc3nonc3C)CC2)n1] | >10 | 1-(4-(3-(1-methoxyethyl)- 1,2,4-oxadiazol-5-yl)piperi- din-1-yl)-2-(4-methyl-1,2,5- oxadiazol-3-yl)ethan-1-one |
| 92 | 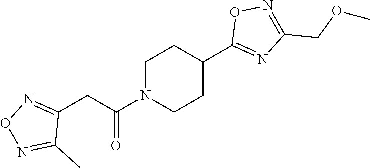 [SMILES: COCc1noc(C2CCN(C(=O)Cc3nonc3C)CC2)n1] | >10 | 1-(4-(3-(methoxymethyl)- 1,2,4-oxadiazol-5-yl)piperi- din-1-yl)-2-(4-methyl-1,2,5- oxadiazol-3-yl)ethan-1-one |
| 93 | 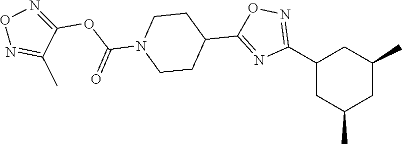 [SMILES: Cc1nonc1OC(=O)N1CCC(c2nc(C3C[C@@H](C)C[C@@H](C)C3)no2)CC1] | 2.34 | 1-(4-(3-((3R,5S)-3,5-dimeth- ylcyclohexyl)-1,2,4-oxadia- zol-5-yl)piperidin-1-yl)-2- (4-methyl-1,2,5-oxadiazol- 3-yl)ethan-1-one |
| 94 | 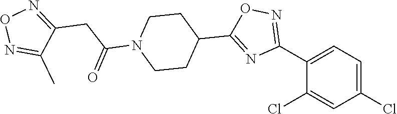 [SMILES: Cc1nonc1CC(=O)N1CCC(c2nc(-c3ccc(Cl)cc3Cl)no2)CC1] | 0.146 | 1-(4-(3-(2,4-dichlorophen- yl)-1,2,4-oxadiazol-5-yl)- piperidin-1-yl)-2-(4-methyl- 1,2,5-oxadiazol-3-yl)ethan- 1-one |
| 95 | 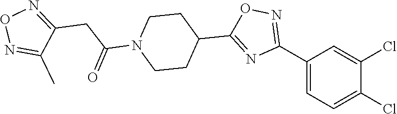 [SMILES: Cc1nonc1CC(=O)N1CCC(c2nc(-c3ccc(Cl)c(Cl)c3)no2)CC1] | 0.0644 | 1-(4-(3-(3,4-dichlorophen- yl)-1,2,4-oxadiazol-5-yl)- piperidin-1-yl)-2-(4-methyl- 1,2,5-oxadiazol-3-yl)ethan- 1-one |
| 96 | 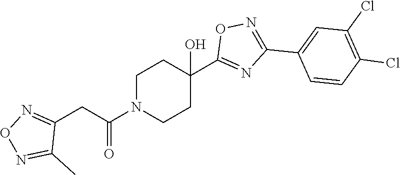 [SMILES: Cc1nonc1CC(=O)N1CCC(O)(c2nc(-c3ccc(Cl)c(Cl)c3)no2)CC1] | 0.61 | 1-(4-(3-(3,4-dichlorophen- yl)-1,2,4-oxadiazol-5-yl)-4- hydroxypiperidin-1-yl)-2- (4-methyl-1,2,5-oxadiazol- 3-yl)ethan-1-one |
| 97 | 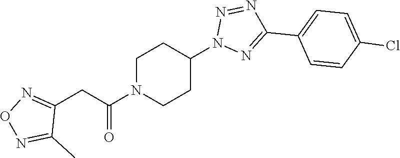 [SMILES: Cc1nonc1CC(=O)N1CCC(n2nnc(-c3ccc(Cl)cc3)n2)CC1] | 0.149 | 1-(4-(5-(4-chlorophenyl)- 2H-tetrazol-2-yl)piperidin- 1-yl)-2-(4-methyl-1,2,5-oxa- diazol-3-yl)ethan-1-one |
| 98 | 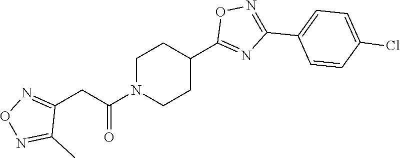 [SMILES: Cc1nonc1CC(=O)N1CCC(c2nc(-c3ccc(Cl)cc3)no2)CC1] | 0.114 | 1-(4-(3-(4-chlorophenyl)- 1,2,4-oxadiazol-5-yl)piperi- din-1-yl)-2-(4-methyl-1,2,5- oxadiazol-3-yl)ethan-1-one |
| 99 | 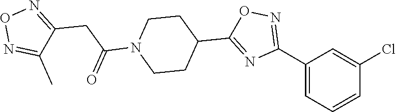 [SMILES: Cc1nonc1CC(=O)N1CCC(c2nc(-c3cccc(Cl)c3)no2)CC1] | 0.53 | 1-(4-(3-(3-chlorophenyl)- 1,2,4-oxadiazol-5-yl)piperi- din-1-yl)-2-(4-methyl-1,2,5- oxadiazol-3-yl)ethan-1-one |
| 100 | 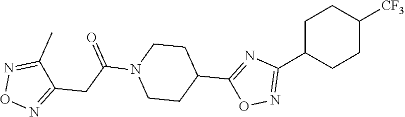 [SMILES: Cc1nonc1CC(=O)N1CCC(c2nc(C3CCC(C(F)(F)F)CC3)no2)CC1] | 0.688 | 2-(4-methyl-1,2,5-oxadia- zol-3-yl)-1-(4-(3-(4-(tri- fluoromethyl)cyclohexyl)- 1,2,4-oxadiazol-5-yl)piperi- din-1-yl)ethan-1-one |
| 101 | 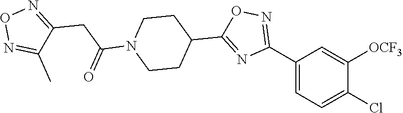 [SMILES: Cc1nonc1CC(=O)N1CCC(c2nc(-c3ccc(Cl)c(OC(F)(F)F)c3)no2)CC1] | 0.731 | 1-(4-(3-(4-chloro-3-tri- fluoromethoxy)phenyl)- 1,2,4-oxadiazol-5-yl)piperi- din-1-yl)-2-(4-methyl-1,2,5- oxadiazol-3-yl)ethan-1-one |
| 102 | 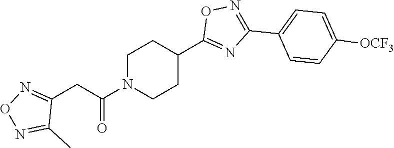 [SMILES: Cc1nonc1CC(=O)N1CCC(c2nc(-c3ccc(OC(F)(F)F)cc3)no2)CC1] | 0.167 | 2-(4-methyl-1,2,5-oxadia- zol-3-yl)-1-(4-(3-(4-(tri- fluoromethoxy)phenyl)- 1,2,4-oxadiazol-5-yl)- piperidin-1-yl)ethan-1-one |
| 103 | 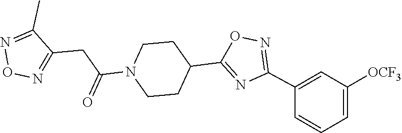 [SMILES: Cc1nonc1CC(=O)N1CCC(c2nc(-c3cccc(OC(F)(F)F)c3)no2)CC1] | 0.383 | 2-(4-methyl-1,2,5-oxadia- zol-3-yl)-1-(4-(3-(3-(tri- fluoromethoxy)phenyl)- 1,2,4-oxadiazol-5-yl)- piperidin-1-yl)ethan-1-one |
| 104 | 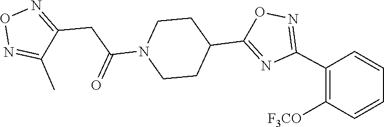 [SMILES: Cc1nonc1CC(=O)N1CCC(c2nc(-c3ccccc3OC(F)(F)F)no2)CC1] | 2.96 | 2-(4-methyl-1,2,5-oxadia- zol-3-yl)-1-(4-(3-(2-(tri- fluoromethoxy)phenyl)- 1,2,4-oxadiazol-5-yl)- piperidin-1-yl)ethan-1-one |
| 105 | 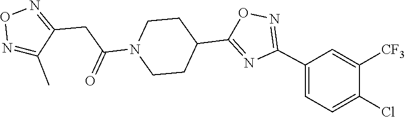 [SMILES: Cc1nonc1CC(=O)N1CCC(c2nc(-c3ccc(Cl)c(C(F)(F)F)c3)no2)CC1] | 0.744 | 1-(4-(3-(4-chloro-3-(tri- fluoromethyl)phenyl)-1,2,4- oxadiazol-5-yl)piperidin-1- yl)-2-(4-methyl-1,2,5-oxa- diazol-3-yl)ethan-1-one |
| 106 | 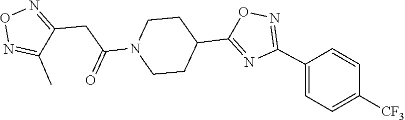 [SMILES: Cc1nonc1CC(=O)N1CCC(c2nc(-c3ccc(C(F)(F)F)cc3)no2)CC1] | 0.754 | 2-(4-methyl-1,2,5-oxadia- zol-3-yl)-1-(4-(3-(4-(tri- fluoromethyl)phenyl)-1,2,4- oxadiazol-5-yl)piperidin-1- yl)ethan-1-one |
| 107 | 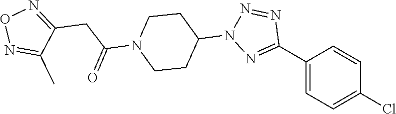 [SMILES: Cc1nonc1CC(=O)N1CCC(n2nnc(-c3ccc(Cl)cc3)n2)CC1] | 0.765 | 2-(4-methyl-1,2,5-oxadia- zol-3-yl)-1-(4-(5-(4-(tri- fluoromethyl)phenyl)-2H- tetrazol-2-yl)piperidin-1- yl)ethan-1-one |
| 108 | 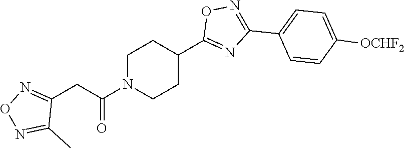 [SMILES: Cc1nonc1CC(=O)N1CCC(c2nc(-c3ccc(OC(F)F)cc3)no2)CC1] | 0.716 | 1-(4-(3-(4-(difluorometh- oxy)phenyl)-1,2,4-oxadia- zol-5-yl)piperidin-1-yl)-2- (4-methyl-1,2,5-oxadiazol- 3-yl)ethan-1-one |
| 109 | 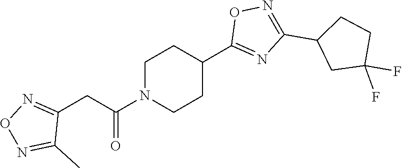 [SMILES: Cc1nonc1CC(=O)N1CCC(c2nc(C3CCC(F)(F)C3)no2)CC1] | 3.25 | 1-(4-(3-(3,3-difluorocyclo- pentyl)-1,2,4-oxadiazol-5- yl)piperidin-1-yl)-2-(4- methyl-1,2,5-oxadiazol-3- yl)ethan-1-one |
| 110 | 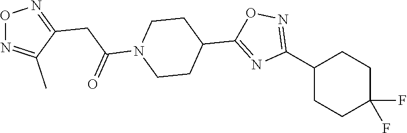 [SMILES: Cc1nonc1CC(=O)N1CCC(c2nc(C3CCC(F)(F)CC3)no2)CC1] | 0.969 | 1-(4-(3-(4,4-difluorocyclo- hexyl)-1,2,4-oxadiazol-5- yl)piperidin-1-yl)-2-(4- methyl-1,2,5-oxadiazol-3- yl)ethan-1-one |
| 111 | 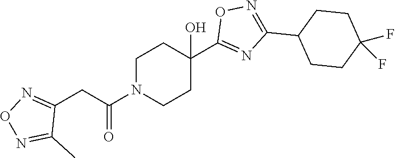 [SMILES: Cc1nonc1CC(=O)N1CCC(O)(c2nc(C3CCC(F)(F)CC3)no2)CC1] | >10 | 1-(4-(3-(4,4-difluorocyclo- hexyl)-1,2,4-oxadiazol-5- yl)-4-hydroxypiperidin-1- yl)-2-(4-methyl-1,2,5-oxa- diazol-3-yl)ethan-1-one |
| 112 | 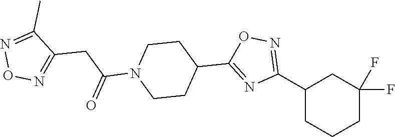 [SMILES: Cc1nonc1CC(=O)N1CCC(c2nc(C3CCCC(F)(F)C3)no2)CC1] | 2.67 | 1-(4-(3-(3,3-difluorocyclo- hexyl)-1,2,4-oxadiazol-5- yl)piperidin-1-yl)-2-(4- methyl-1,2,5-oxadiazol-3- yl)ethan-1-one |
| 113 | 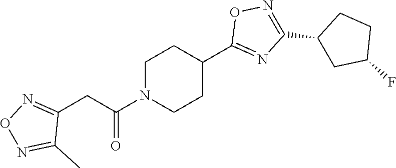 [SMILES: Cc1nonc1CC(=O)N1CCC(c2nc([C@@H]3CC[C@H](F)C3)no2)CC1] | >25 | 1-(4-(3-((1R,3S)-3-fluoro- cyclopentyl)-1,2,4-oxadia- zol-5-yl)piperidin-1-yl)-2- (4-methyl-1,2,5-oxadiazol- 3-yl)ethan-1-one |
| 114 | 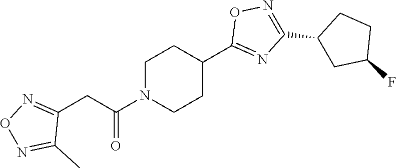 [SMILES: Cc1nonc1CC(=O)N1CCC(c2nc([C@@H]3CC[C@@H](F)C3)no2)CC1] | 7.04 | 1-(4-(3-((1R,3R)-3-fluoro- cyclopentyl)-1,2,4-oxadia- zol-5-yl)piperidin-1-yl)-2- (4-methyl-1,2,5-oxadiazol- 3-yl)ethan-1-one |
| 115 | 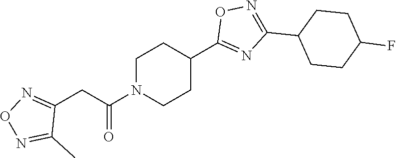 [SMILES: Cc1nonc1CC(=O)N1CCC(c2nc(C3CCC(F)CC3)no2)CC1] | >25 | 1-(4-(3-(4-fluorocyclohex- yl)-1,2,4-oxadiazol-5-yl)- piperidin-1-yl)-2-(4-methyl- 1,2,5-oxadiazol-3-yl)ethan- 1-one |
| 116 | 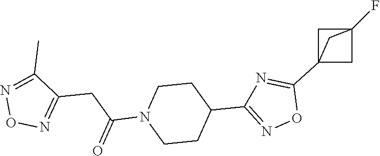 [SMILES: Cc1nonc1CC(=O)N1CCC(c2noc(C34CC(F)(C3)C4)n2)CC1] | 9.23 | 1-(4-(5-(3-fluorobicyclo [1.1.1]pentan-1-yl)-1,2,4- oxadiazol-3-yl)piperidin-1- yl)-2-(4-methyl-1,2,5-oxa- diazol-3-yl)ethan-1-one |
| 117 | 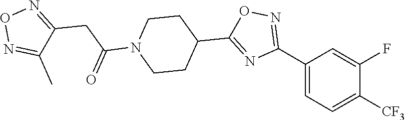 [SMILES: Cc1nonc1CC(=O)N1CCC(c2nc(-c3ccc(C(F)(F)F)c(F)c3)no2)CC1] | 0.278 | 1-(4-(3-(3-fluoro-4-trifluoro- methyl)phenyl)-1,2,4-oxa- diazol-5-yl)piperidin-1-yl)- 2-(4-methyl-1,2,5-oxadia- zol-3-yl)ethan-1-one |
| 118 | 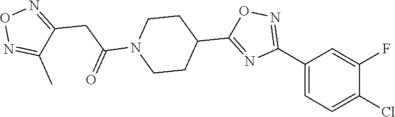 [SMILES: Cc1nonc1CC(=O)N1CCC(c2nc(-c3ccc(Cl)c(F)c3)no2)CC1] | 0.0805 | 1-(4-(3-(4-chloro-3-fluoro- phenyl)-1,2,4-oxadiazol-5- yl)piperidin-1-yl)-2-(4- methyl-1,2,5-oxadiazol-3- yl)ethan-1-one |
| 119 | 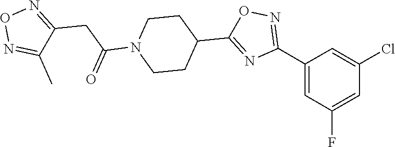 [SMILES: Cc1nonc1CC(=O)N1CCC(c2nc(-c3cc(F)cc(Cl)c3)no2)CC1] | 0.47 | 1-(4-(3-(3-chloro-5-fluoro- phenyl)-1,2,4-oxadiazol-5- yl)piperidin-1-yl)-2-(4- methyl-1,2,5-oxadiazol-3- yl)ethan-1-one |
| 120 | 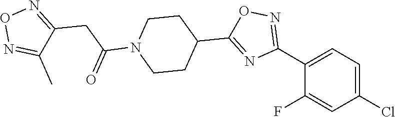 [SMILES: Cc1nonc1CC(=O)N1CCC(c2nc(-c3ccc(Cl)cc3F)no2)CC1] | 0.37 | 1-(4-(3-(4-chloro-2-fluoro- phenyl)-1,2,4-oxadiazol-5- yl)piperidin-1-yl)-2-(4- methyl-1,2,5-oxadiazol-3- yl)ethan-1-one |
| 121 | 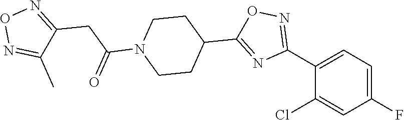 [SMILES: Cc1nonc1CC(=O)N1CCC(c2nc(-c3ccc(F)cc3Cl)no2)CC1] | 0.38 | 1-(4-(3-(2-chloro-4-fluoro- phenyl)-1,2,4-oxadiazol-5- yl)piperidin-1-yl)-2-(4- methyl-1,2,5-oxadiazol-3- yl)ethan-1-one |
| 122 | 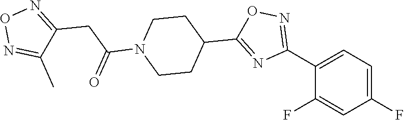 [SMILES: Cc1nonc1CC(=O)N1CCC(c2nc(-c3ccc(F)cc3F)no2)CC1] | 0.39 | 1-(4-(3-(2,4-difluorophen- yl)-1,2,4-oxadiazol-5-yl)- piperidin-1-yl)-2-(4-methyl- 1,2,5-oxadiazol-3-yl)ethan- 1-one |
| 123 | 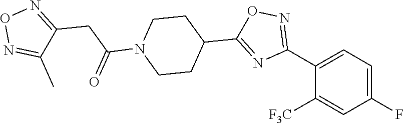 [SMILES: Cc1nonc1CC(=O)N1CCC(c2nc(-c3ccc(F)cc3C(F)(F)F)no2)CC1] | 1.48 | 1-(4-(3-(4-fluoro-2-trifluoro- methyl)phenyl)-1,2,4-oxa- diazol-5-yl)piperidin-1-yl)- 2-(4-methyl-1,2,5-oxadiazol- 3-yl)ethan-1-one |
| 124 | 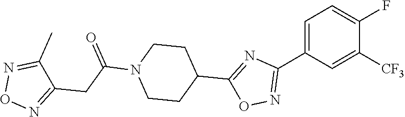 [SMILES: Cc1nonc1CC(=O)N1CCC(c2nc(-c3ccc(F)c(C(F)(F)F)c3)no2)CC1] | 0.334 | 1-(4-(3-(4-fluoro-3-trifluoro- methyl)phenyl)-1,2,4-oxa- diazol-5-yl)piperidin-1-yl)- 2-(4-methyl-1,2,5-oxadia- zol-3-yl)ethan-1-one |
| 125 | 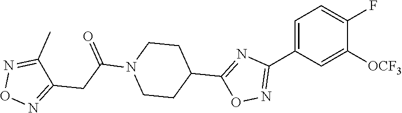 [SMILES: Cc1nonc1CC(=O)N1CCC(c2nc(-c3ccc(F)c(OC(F)(F)F)c3)no2)CC1] | 0.288 | 1-(4-(3-(4-fluoro-3-trifluoro- methoxy)phenyl)-1,2,4- oxadiazol-5-yl)piperidin-1- yl)-2-(4-methyl-1,2,5-oxa- diazol-3-yl)ethan-1-one |
| 126 | 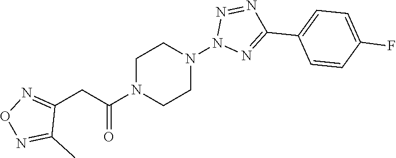 [SMILES: Cc1nonc1CC(=O)N1CCN(n2nnc(-c3ccc(F)cc3)n2)CC1] | 0.519 | 1-(4-(5-(4-fluorophenyl)- 2H-tetrazol-2-yl)piperidin- 1-yl)-2-(4-methyl-1,2,5-oxa- diazol-3-yl)ethan-1-one |
| 127 | 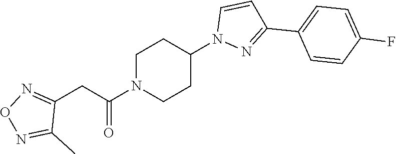 [SMILES: Cc1nonc1CC(=O)N1CCC(n2ccc(-c3ccc(F)cc3)n2)CC1] | 2.97 | 1-(4-(3-(4-fluorophenyl)- 1H-pyrazol-1-yl)piperidin- 1-yl)-2-(4-methyl-1,2,5-oxa- diazol-3-yl)ethan-1-one |
| 128 | 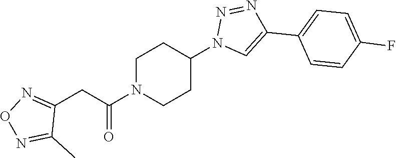 [SMILES: Cc1nonc1CC(=O)N1CCC(n2cc(-c3ccc(F)cc3)nn2)CC1] | >10 | 1-(4-(4-(4-fluorophenyl)- 1H-1,2,3-triazol-1-yl)piperi- din-1-yl)-2-(4-methyl-1,2,5- oxadiazol-3-yl)ethan-1-one |
| 129 | 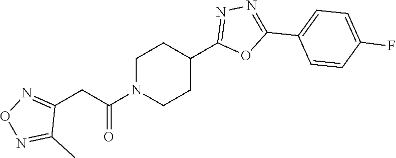 [SMILES: Cc1nonc1CC(=O)N1CCC(c2nnc(-c3ccc(F)cc3)o2)CC1] | >10 | 1-(4-(5-(4-fluorophenyl)- 1,3,4-oxadiazol-2-yl)piperi- din-1-yl)-2-(4-methyl-1,2,5- oxadiazol-3-yl)ethan-1-one |
| 130 | 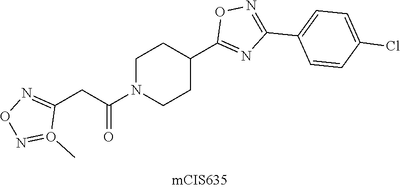 | 0.275 | 1-(4-(3-(4-fluorophenyl)- 1,2,4-oxadiazol-5-yl)piperi- din-1-yl)-2-(4-methyl-1,2,5- oxadiazol-3-yl)ethan-1-one |
| 131 | 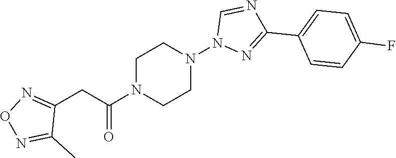 [SMILES: Cc1nonc1CC(=O)N1CCN(n2cnc(-c3ccc(F)cc3)n2)CC1] | 8 | 1-(4-(3-(4-fluorophenyl)- 1H-1,2,4-triazol-1-yl)piperi- din-1-yl)-2-(4-methyl-1,2,5- oxadiazol-3-yl)ethan-1-one |
| 132 | 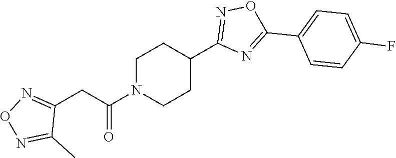 [SMILES: Cc1nonc1CC(=O)N1CCC(c2noc(-c3ccc(F)cc3)n2)CC1] | 1.07 | 1-(4-(5-(4-fluorophenyl)- 1,2,4-oxadiazol-3-yl)piperi- din-1-yl)-2-(4-methyl-1,2,5- oxadiazol-3-yl)ethan-1-one |
| 133 | 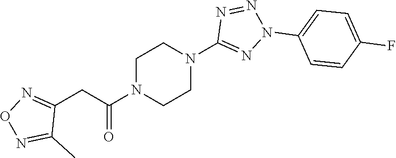 [SMILES: Cc1nonc1CC(=O)N1CCN(c2nnn(-c3ccc(F)cc3)n2)CC1] | 1.29 | 1-(4-(2-(4-fluorophenyl)- 2H-tetrazol-5-yl)piperidin- 1-yl)-2-(4-methyl-1,2,5- oxadiazol-3-yl)ethan-1-one |
| 134 | 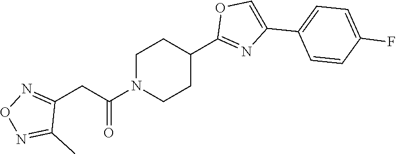 [SMILES: Cc1nonc1CC(=O)N1CCC(c2nc(-c3ccc(F)cc3)co2)CC1] | 1.43 | 1-(4-(4-(4-fluorophenyl)- oxazol-2-yl)piperidin-1-yl)- 2-(4-methyl-1,2,5-oxadia- zol-3-yl)ethan-1-one |
| 135 | 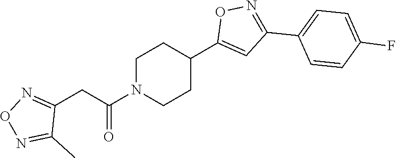 [SMILES: Cc1nonc1CC(=O)N1CCC(c2cc(-c3ccc(F)cc3)no2)CC1] | 1.58 | 1-(4-(3-(4-fluorophenyl)- isoxazol-5-yl)piperidin-1- yl)-2-(4-methyl-1,2,5-oxa- diazol-3-yl)ethan-1-one |
| 136 | 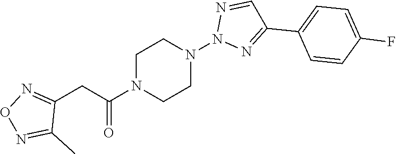 [SMILES: Cc1nonc1CC(=O)N1CCN(n2ncc(-c3ccc(F)cc3)n2)CC1] | 1.58 | 1-(4-(4-(4-fluorophenyl)- 2H-1,2,3-triazol-2-yl)piperi- din-1-yl)-2-(4-methyl-1,2,5- oxadiazol-3-yl)ethan-1-one |
| 137 | 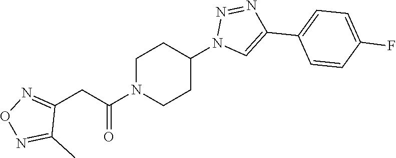 [SMILES: Cc1nonc1CC(=O)N1CCC(n2cc(-c3ccc(F)cc3)nn2)CC1] | >10 | 1-(4-(4-(4-fluorophenyl)- 1H-1,2,3-triazol-1-yl)piperi- din-1-yl)-2-(4-methyl-1,2,5- oxadiazol-3-yl)ethan-1-one |
| 138 | 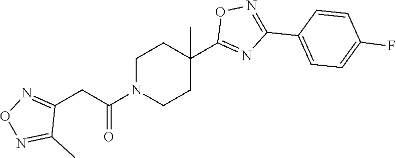 [SMILES: Cc1nonc1CC(=O)N1CCC(C)(c2nc(-c3ccc(F)cc3)no2)CC1] | 1.47 | 1-(4-(3-(4-fluorophenyl)- 1,2,4-oxadiazol-5-yl)-4- methylpiperidin-1-yl)-2-(4- methyl-1,2,5-oxadiazol-3- yl)ethan-1-one |
| 139 | 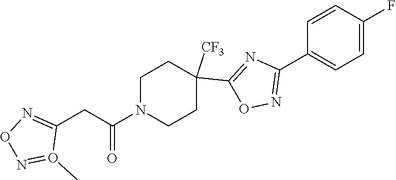 | >10 | 1-(4-(3-(4-fluorophenyl)- 1,2,4-oxadiazol-5-yl)-4- (trifluoromethyl)piperidin- 1-yl)-2-(4-methyl-1,2,5- oxadiazol-3-yl)ethan-1-one |
| 140 | 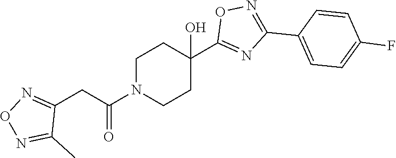 [SMILES: Cc1nonc1CC(=O)N1CCC(O)(c2nc(-c3ccc(F)cc3)no2)CC1] | 0.685 | 1-(4-(3-(4-fluorophenyl)- 1,2,4-oxadiazol-5-yl)-4- hydroxypiperidin-1-yl)-2- (4-methyl-1,2,5-oxadiazol- 3-yl)ethan-1-one |
| 141 | 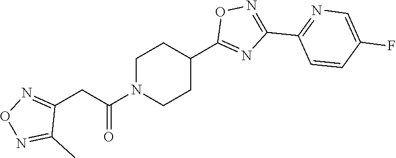 [SMILES: Cc1nonc1CC(=O)N1CCC(c2nc(-c3ccc(F)cn3)no2)CC1] | >10 | 1-(4-(3-(5-fluoropyridin-2- yl)-1,2,4-oxadiazol-5-yl)- piperidin-1-yl)-2-(4-methyl- 1,2,5-oxadiazol-3-yl)ethan- 1-one |
| 142 | 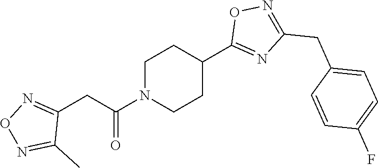 [SMILES: Cc1nonc1CC(=O)N1CCC(c2nc(Cc3ccc(F)cc3)no2)CC1] | 6.64 | 1-(4-(3-(4-fluorobenzyl)- 1,2,4-oxadiazol-5-yl)piperi- din-1-yl)-2-(4-methyl-1,2,5- oxadiazol-3-yl)ethan-1-one |
| 143 | 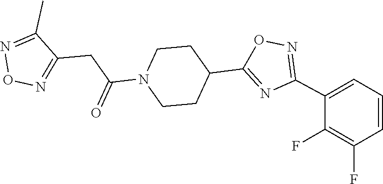 [SMILES: Cc1nonc1CC(=O)N1CCC(c2nc(-c3cccc(F)c3F)no2)CC1] | 1.2 | 1-(4-(3-(2,3-difluorophen- yl)-1,2,4-oxadiazol-5-yl)- piperidin-1-yl)-2-(4-methyl- 1,2,5-oxadiazol-3-yl)ethan- 1-one |
| 144 | 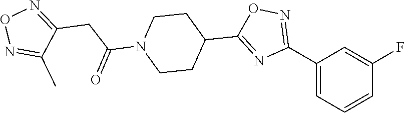 [SMILES: Cc1nonc1CC(=O)N1CCC(c2nc(-c3cccc(F)c3)no2)CC1] | 0.59 | 1-(4-(3-(3-fluorophenyl)- 1,2,4-oxadiazol-5-yl)piperi- din-1-yl)-2-(4-methyl-1,2,5- oxadiazol-3-yl)ethan-1-one |
| 145 | 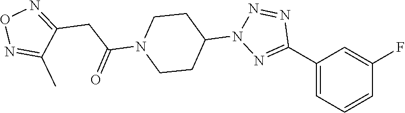 [SMILES: Cc1nonc1CC(=O)N1CCC(n2nnc(-c3cccc(F)c3)n2)CC1] | 0.963 | 1-(4-(5-(3-fluorophenyl)- 2H-tetrazol-2-yl)piperidin- 1-yl)-2-(4-methyl-1,2,5-oxa- diazol-3-yl)ethan-1-one |
| 146 | 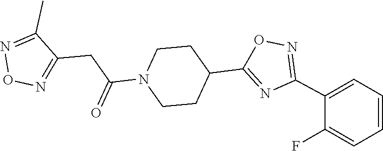 [SMILES: Cc1nonc1CC(=O)N1CCC(c2nc(-c3ccccc3F)no2)CC1] | 1.36 | 1-(4-(3-(2-fluorophenyl)- 1,2,4-oxadiazol-5-yl)piperi- din-1-yl)-2-(4-methyl-1,2,5- oxadiazol-3-yl)ethan-1-one |
| 147 | 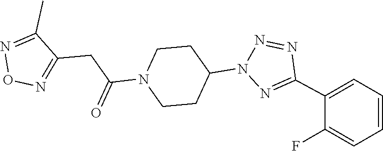 [SMILES: Cc1nonc1CC(=O)N1CCC(n2nnc(-c3ccccc3F)n2)CC1] | 1.45 | 1-(4-(5-(2-fluorophenyl)- 2H-tetrazol-2-yl)piperidin- 1-yl)-2-(4-methyl-1,2,5-oxa- diazol-3-yl)ethan-1-one |
| 148 | 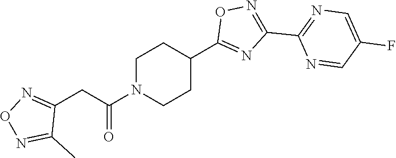 [SMILES: Cc1nonc1CC(=O)N1CCC(c2nc(-c3ncc(F)cn3)no2)CC1] | >10 | 1-(4-(3-(5-fluoropyrimidin- 2-yl)-1,2,4-oxadiazol-5-yl)- piperidin-1-yl)-2-(4-methyl- 1,2,5-oxadiazol-3-yl)ethan- 1-one |
| 149 | 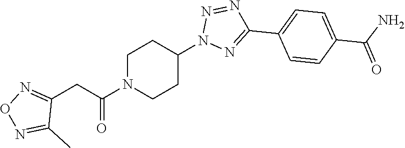 [SMILES: Cc1nonc1CC(=O)N1CCC(n2nnc(-c3ccc(C(N)=O)cc3)n2)CC1] | >10 | 4-(2-(1-(2-(4-methyl-1,2,5- oxadiazol-3-yl)acetyl)piperi- din-4-yl)-2H-tetrazol-5-yl)- benzamide |
| 150 | 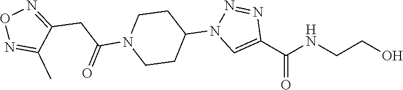 [SMILES: Cc1nonc1CC(=O)N1CCC(n2cc(C(=O)NCCO)nn2)CC1] | >10 | N-(2-hydroxyethyl)-1-(1-(2- (4-methyl-1,2,5-oxadiazol-3- yl)acetyl)piperidin-4-yl)-1H- 1,2,3-triazole-4-carboxamide |
| 151 | 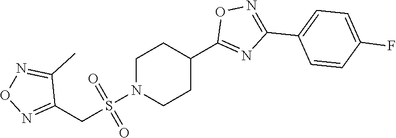 [SMILES: Cc1nonc1CS(=O)(=O)N1CCC(c2nc(-c3ccc(F)cc3)no2)CC1] | >10 | 3-(4-fluorophenyl)-5-(1-(((4- methyl-1,2,5-oxadiazol-3- yl)methyl)sulfonyl)piperidin- 4-yl)-1,2,4-oxadiazole |
| 152 | 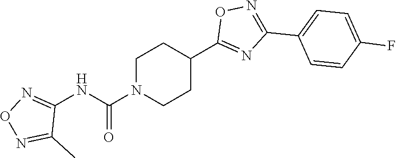 [SMILES: Cc1nonc1NC(=O)N1CCC(c2nc(-c3ccc(F)cc3)no2)CC1] | 0.653 | 4-(3-(4-fluorophenyl)-1,2,4- oxadiazol-5-yl)-N-(4-methyl- 1,2,5-oxadiazol-3-yl)piperi- dine-1-carboxamide |
| 153 | 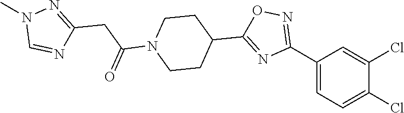 [SMILES: Cn1cnc(CC(=O)N2CCC(c3nc(-c4ccc(Cl)c(Cl)c4)no3)CC2)n1] | >10 | 1-(4-(3-(3,4-dichlorophen- yl)-1,2,4-oxadiazol-5-yl)- piperidin-1-yl)-2-(1-methyl- 1H-1,2,4-triazol-3-yl)ethan- 1-one |
| 154 | 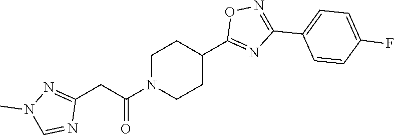 [SMILES: Cn1cnc(CC(=O)N2CCC(c3nc(-c4ccc(F)cc4)no3)CC2)n1] | >10 | 1-(4-(3-(4-fluorophenyl)- 1,2,4-oxadiazol-5-yl)piperi- din-1-yl)-2-(1-methyl-1H- 1,2,4-triazol-3-yl)ethan-1- one |
| 155 | 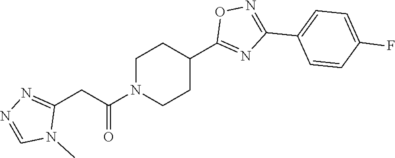 [SMILES: Cn1cnnc1CC(=O)N1CCC(c2nc(-c3ccc(F)cc3)no2)CC1] | >10 | 1-(4-(3-(4-fluorophenyl)- 1,2,4-oxadiazol-5-yl)piperi- din-1-yl)-2-(4-methyl-4H- 1,2,4-triazol-3-yl)ethan-1- one |
| 156 | 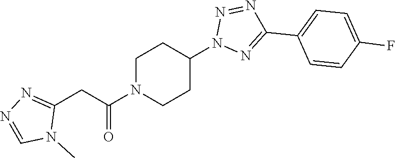 [SMILES: Cn1cnnc1CC(=O)N1CCC(n2nnc(-c3ccc(F)cc3)n2)CC1] | >10 | 1-(4-(5-(4-fluorophenyl)- 2H-tetrazol-2-yl)piperidin- 1-yl)-2-(4-methyl-4H-1,2,4- triazol-3-yl)ethan-1-one |
| 157 | 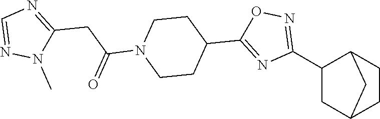 [SMILES: Cn1ncnc1CC(=O)N1CCC(c2nc(C3CC4CCC3C4)no2)CC1] | 1.86 | 1-(4-(3-(bicyclo[2.2.1] heptan-2-yl)-1,2,4-oxadia- zol-5-yl)piperidin-1-yl)-2- (1-methyl-1H-1,2,4-triazol- 5-yl)ethan-1-one |
| 158 | 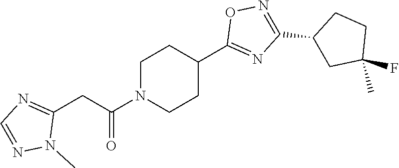 [SMILES: Cn1ncnc1CC(=O)N1CCC(c2nc([C@@H]3CC[C@@](C)(F)C3)no2)CC1] | >10 | 1-(4-(3-((1R,3R)-3-fluoro-3- methylcyclopentyl)-1,2,4- oxadiazol-5-yl)piperidin-1- yl)-2-(1-methyl-1H-1,2,4- triazol-5-yl)ethan-1-one |
| 159 | 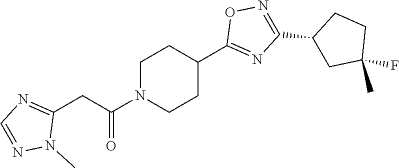 [SMILES: Cn1ncnc1CC(=O)N1CCC(c2nc([C@@H]3CC[C@](C)(F)C3)no2)CC1] | >10 | 1-(4-(3-((1R,3S)-3-fluoro-3- methylcyclopentyl)-1,2,4- oxadiazol-5-yl)piperidin-1- yl)-2-(1-methyl-1H-1,2,4- triazol-5-yl)ethan-1-one |
| 160 | 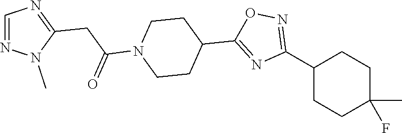 [SMILES: Cn1ncnc1CC(=O)N1CCC(c2nc(C3CCC(C)(F)CC3)no2)CC1] | >10 | 1-(4-(3-(4-fluoro-4-meth- ylcyclohexyl)-1,2,4-oxadia- zol-5-yl)piperidin-1-yl)-2- (1-methyl-1H-1,2,4-triazol- 5-yl)ethan-1-one |
| 161 | 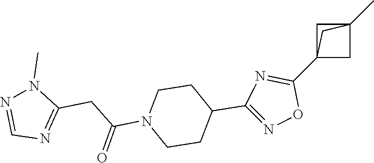 [SMILES: Cn1ncnc1CC(=O)N1CCC(c2noc(C34CC(C)(C3)C4)n2)CC1] | >10 | 2-(1-methyl-1H-1,2,4-tria- zol-5-yl)-1-(4-(5-(3-methyl- bicyclo[1.1.1]pentan-1-yl)- 1,2,4-oxadiazol-3-yl)piperi- din-1-yl)ethan-1-one |
| 162 | 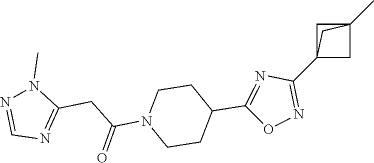 [SMILES: Cn1ncnc1CC(=O)N1CCC(c2nc(C34CC(C)(C3)C4)no2)CC1] | >10 | 2-(1-methyl-1H-1,2,4-tria- zol-5-yl)-1-(4-(3-(3-methyl- bicyclo[1.1.1]pentan-1-yl)- 1,2,4-oxadiazol-5-yl)piperi- din-1-yl)ethan-1-one |
| 163 | 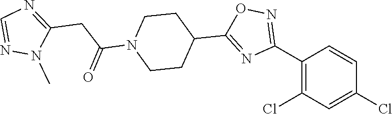 [SMILES: Cn1ncnc1CC(=O)N1CCC(c2nc(-c3ccc(Cl)cc3Cl)no2)CC1] | 0.2 | 1-(4-(3-(2,4-dichlorophen- yl)-1,2,4-oxadiazol-5-yl)- piperidin-1-yl)-2-(1-methyl- 1H-1,2,4-triazol-5-yl)ethan- 1-one |
| 164 | 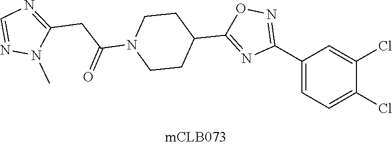 [SMILES: Cn1ncnc1CC(=O)N1CCC(c2nc(-c3ccc(Cl)c(Cl)c3)no2)CC1] | 0.0647 | 1-(4-(3-(3,4-dichlorophen- yl)-1,2,4-oxadiazol-5-yl)- piperidin-1-yl)-2-(1-methyl- 1H-1,2,4-triazol-5-yl)ethan- 1-one |
| 165 | 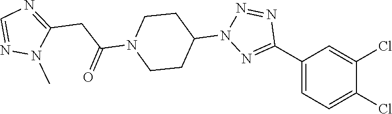 [SMILES: Cn1ncnc1CC(=O)N1CCC(n2nnc(-c3ccc(Cl)c(Cl)c3)n2)CC1] | 0.1 | 1-(4-(5-(3,4-dichlorophen- yl)-2H-tetrazol-2-yl)piperi- din-1-yl)-2-(1-methyl-1H- 1,2,4-triazol-5-yl)ethan-1- one |
| 166 | 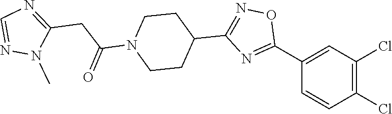 [SMILES: Cn1ncnc1CC(=O)N1CCC(c2noc(-c3ccc(Cl)c(Cl)c3)n2)CC1] | 0.71 | 1-(4-(5-(3,4-dichlorophen- yl)-1,2,4-oxadiazol-3-yl)- piperidin-1-yl)-2-(1-methyl- 1H-1,2,4-triazol-5-yl)ethan- 1-one |
| 167 | 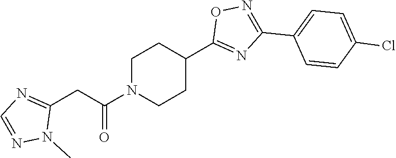 [SMILES: Cn1ncnc1CC(=O)N1CCC(c2nc(-c3ccc(Cl)cc3)no2)CC1] | 0.221 | 1-(4-(3-(4-chlorophenyl)- 1,2,4-oxadiazol-5-yl)piperi- din-1-yl)-2-(1-methyl-1H- 1,2,4-triazol-5-yl)ethan-1- one |
| 168 | 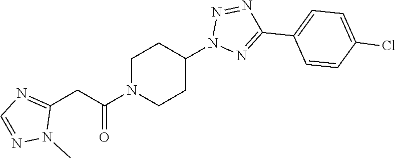 [SMILES: Cn1ncnc1CC(=O)N1CCC(n2nnc(-c3ccc(Cl)cc3)n2)CC1] | 0.472 | 1-(4-(5-(4-chlorophenyl)- 2H-tetrazol-2-yl)piperidin- 1-yl)-2-(1-methyl-1H-1,2,4- triazol-5-yl)ethan-1-one |
| 169 | 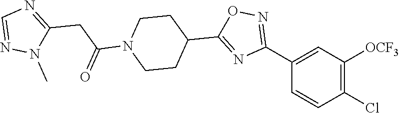 [SMILES: Cn1ncnc1CC(=O)N1CCC(c2nc(-c3ccc(Cl)c(OC(F)(F)F)c3)no2)CC1] | 0.33 | 1-(4-(3-(4-chloro-3-(tri- fluoromethoxy)phenyl)- 1,2,4-oxadiazol-5-yl)- piperidin-1-yl)-2-(1-meth- yl-1H-1,2,4-triazol-5-yl)- ethan-1-one |
| 170 | 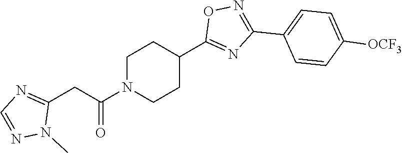 [SMILES: Cn1ncnc1CC(=O)N1CCC(c2nc(-c3ccc(OC(F)(F)F)cc3)no2)CC1] | 0.13 | 2-(1-methyl-1H-1,2,4-tria- zol-5-yl)-1-(4-(3-(4-(tri- fluoromethoxy)phenyl)- 1,2,4-oxadiazol-5-yl)piperi- din-1-yl)ethan-1-one |
| 171 | 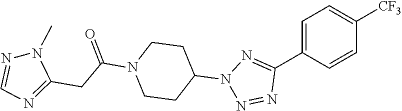 [SMILES: Cn1ncnc1CC(=O)N1CCC(n2nnc(-c3ccc(C(F)(F)F)cc3)n2)CC1] | 0.963 | 2-(1-methyl-1H-1,2,4- triazol-5-yl)-1-(4-(5-(4- (trifluoromethyl)phenyl)- 2H-tetrazol-2-yl)piperidin- 1-yl)ethan-1-one |
| 172 | 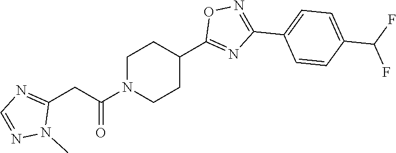 [SMILES: Cn1ncnc1CC(=O)N1CCC(c2nc(-c3ccc(C(F)F)cc3)no2)CC1] | 2.07 | 1-(4-(3-(4-(difluorometh- yl)phenyl)-1,2,4-oxadiazol- 5-yl)piperidin-1-yl)-2-(1- methyl-1H-1,2,4-triazol-5- yl)ethan-1-one |
| 173 | 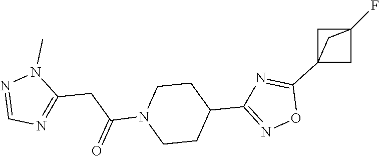 [SMILES: Cn1ncnc1CC(=O)N1CCC(c2noc(C34CC(F)(C3)C4)n2)CC1] | >10 | 1-(4-(5-(3-fluorobicyclo [1.1.1]pentan-1-yl)-1,2,4- oxadiazol-3-yl)piperidin-1- yl)-2-(1-methyl-1H-1,2,4- triazol-5-yl)ethan-1-one |
| 174 | 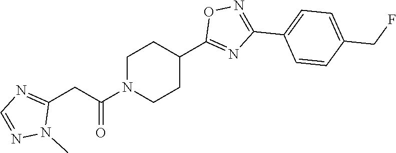 [SMILES: Cn1ncnc1CC(=O)N1CCC(c2nc(-c3ccc(CF)cc3)no2)CC1] | 2.26 | 1-(4-(3-(4-(fluoromethyl)- phenyl)-1,2,4-oxadiazol- 5-yl)piperidin-1-yl)-2-(1- methyl-1H-1,2,4-triazol- 5-yl)ethan-1-one |
| 175 | 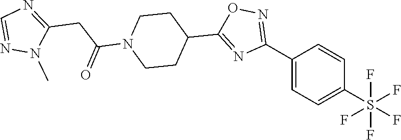 [SMILES: Cn1ncnc1CC(=O)N1CCC(c2nc(-c3ccc(S(F)(F)(F)(F)F)cc3)no2)CC1] | 1.63 | 2-(1-methyl-1H-1,2,4-tria- zol-5-yl)-1-(4-(3-(4-(penta- fluoro-16-sulfaneyl)phen- yl)-1,2,4-oxadiazol-5-yl)- piperidin-1-yl)ethan-1-one |
| 176 | 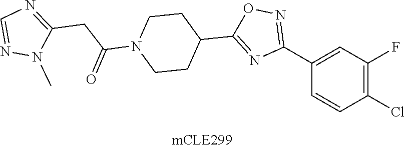 [SMILES: Cn1ncnc1CC(=O)N1CCC(c2nc(-c3ccc(Cl)c(F)c3)no2)CC1] | 0.115 | 1-(4-(3-(4-chloro-3-fluoro- phenyl)-1,2,4-oxadiazol- 5-yl)piperidin-1-yl)-2-(1- methyl-1H-1,2,4-triazol- 5-yl)ethan-1-one |
| 177 | 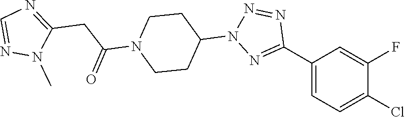 [SMILES: Cn1ncnc1CC(=O)N1CCC(n2nnc(-c3ccc(Cl)c(F)c3)n2)CC1] | 0.17 | 1-(4-(5-(4-chloro-3-fluoro- phenyl)-2H-tetrazol-2-yl)- piperidin-1-yl)-2-(1-methyl- 1H-1,2,4-triazol-5-yl)ethan- 1-one |
| 178 | 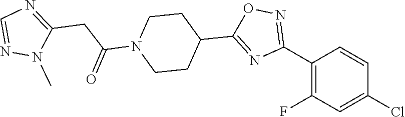 [SMILES: Cn1ncnc1CC(=O)N1CCC(c2nc(-c3ccc(Cl)cc3F)no2)CC1] | 0.2 | 1-(4-(3-(4-chloro-2-fluoro- phenyl)-1,2,4-oxadiazol-5- yl)piperidin-1-yl)-2-(1- methyl-1H-1,2,4-triazol-5- yl)ethan-1-one |
| 179 | 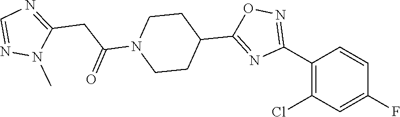 [SMILES: Cn1ncnc1CC(=O)N1CCC(c2nc(-c3ccc(F)cc3Cl)no2)CC1] | 1.97 | 1-(4-(3-(2-chloro-4-fluoro- phenyl)-1,2,4-oxadiazol-5- yl)piperidin-1-yl)-2-(1- methyl-1H-1,2,4-triazol- 5-yl)ethan-1-one |
| 180 | 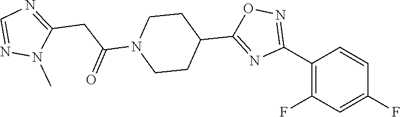 [SMILES: Cn1ncnc1CC(=O)N1CCC(c2nc(-c3ccc(F)cc3F)no2)CC1] | 0.48 | 1-(4-(3-(2,4-difluorophen- yl)-1,2,4-oxadiazol-5-yl)- piperidin-1-yl)-2-(1-methyl- 1H-1,2,4-triazol-5-yl)ethan- 1-one |
| 181 | 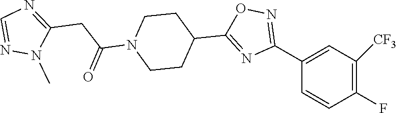 [SMILES: Cn1ncnc1CC(=O)N1CCC(c2nc(-c3ccc(F)c(C(F)(F)F)c3)no2)CC1] | 0.467 | 1-(4-(3-(4-fluoro-3-trifluoro- methyl)phenyl)-1,2,4-oxa- diazol-5-yl)piperidin-1-yl)- 2-(1-methyl-1H-1,2,4-tria- zol-5-yl)ethan-1-one |
| 182 | 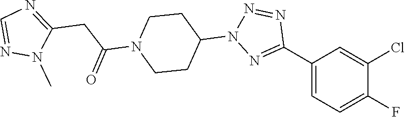 [SMILES: Cn1ncnc1CC(=O)N1CCC(n2nnc(-c3ccc(F)c(Cl)c3)n2)CC1] | 0.37 | 1-(4-(5-(3-chloro-4-fluoro- phenyl)-2H-tetrazol-2-yl)- piperidin-1-yl)-2-(1-meth- yl-1H-1,2,4-triazol-5-yl)- ethan-1-one |
| 183 | 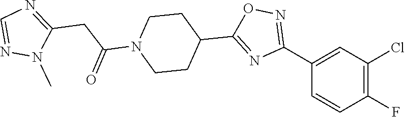 [SMILES: Cn1ncnc1CC(=O)N1CCC(c2nc(-c3ccc(F)c(Cl)c3)no2)CC1] | 0.21 | 1-(4-(3-(3-chloro-4-fluoro- phenyl)-1,2,4-oxadiazol-5- yl)piperidin-1-yl)-2-(1- methyl-1H-1,2,4-triazol- 5-yl)ethan-1-one |
| 184 | 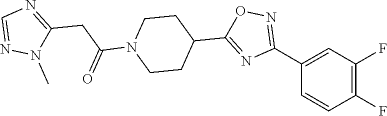 [SMILES: Cn1ncnc1CC(=O)N1CCC(c2nc(-c3ccc(F)c(F)c3)no2)CC1] | 0.458 | 1-(4-(3-(3,4-difluorophen- yl)-1,2,4-oxadiazol-5-yl)- piperidin-1-yl)-2-(1-methyl- 1H-1,2,4-triazol-5-yl)ethan- 1-one |
| 185 | 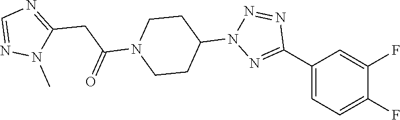 [SMILES: Cn1ncnc1CC(=O)N1CCC(n2nnc(-c3ccc(F)c(F)c3)n2)CC1] | 0.74 | 1-(4-(5-(3,4-difluorophen- yl)-2H-tetrazol-2-yl)piperi- din-1-yl)-2-(1-methyl-1H- 1,2,4-triazol-5-yl)ethan-1- one |
| 186 | 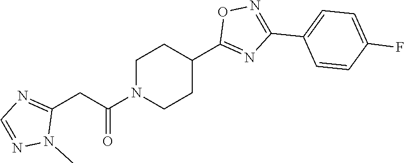 [SMILES: Cn1ncnc1CC(=O)N1CCC(c2nc(-c3ccc(F)cc3)no2)CC1] | 0.48 | 1-(4-(3-(4-fluorophenyl)- 1,2,4-oxadiazol-5-yl)piperi- din-1-yl)-2-(1-methyl-1H- 1,2,4-triazol-5-yl)ethan-1- one |
| 187 | 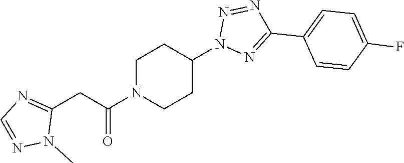 [SMILES: Cn1ncnc1CC(=O)N1CCC(n2nnc(-c3ccc(F)cc3)n2)CC1] | 3.07 | 1-(4-(5-(4-fluorophenyl)- 2H-tetrazol-2-yl)piperidin- 1-yl)-2-(1-methyl-1H-1,2,4- triazol-5-yl)ethan-1-one |
| 188 | 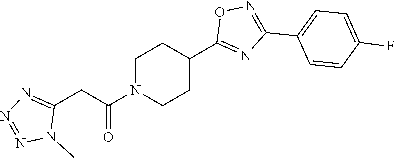 [SMILES: Cn1nnnc1CC(=O)N1CCC(c2nc(-c3ccc(F)cc3)no2)CC1] | 6.33 | 1-(4-(3-(4-fluorophenyl)- 1,2,4-oxadiazol-5-yl)piperi- din-1-yl)-2-(1-methyl-1H- tetrazol-5-yl)ethan-1-one |
| 189 | 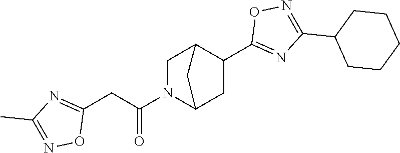 [SMILES: Cc1noc(CC(=O)N2CC3CC2CC3c2nc(C3CCCCC3)no2)n1] | >10 | 1-(5-(3-cyclohexyl-1,2,4- oxadiazol-5-yl)-2-aza- bicyclo[2.2.1]heptan-2-yl)- 2-(3-methyl-1,2,4-oxadia- zol-5-yl)ethan-1-one |
| 190 | 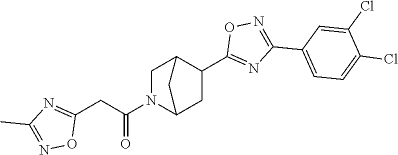 [SMILES: Cc1noc(CC(=O)N2CC3CC2CC3c2nc(-c3ccc(Cl)c(Cl)c3)no2)n1] | 2.9 | 1-(5-(3-(3,4-dichlorophen- yl)-1,2,4-oxadiazol-5-yl)-2- azabicyclo[2.2.1]heptan-2- yl)-2-(3-methyl-1,2,4-oxa- diazol-5-yl)ethan-1-one |
| 191 | 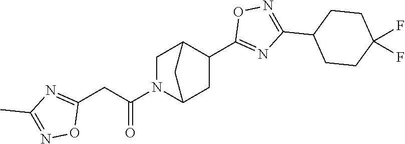 [SMILES: Cc1noc(CC(=O)N2CC3CC2CC3c2nc(C3CCC(F)(F)CC3)no2)n1] | >10 | 1-(5-(3-(4,4-difluorocyclo- hexyl)-1,2,4-oxadiazol-5- yl)-2-azabicyclo[2.2.1] heptan-2-yl)-2-(3-methyl- 1,2,4-oxadiazol-5-yl)ethan- 1-one |
| 192 | 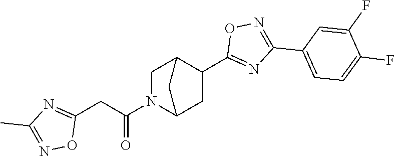 [SMILES: Cc1noc(CC(=O)N2CC3CC2CC3c2nc(-c3ccc(F)c(F)c3)no2)n1] | >10 | 1-(5-(3-(3,4-difluorophen- yl)-1,2,4-oxadiazol-5-yl)-2- azabicyclo[2.2.1]heptan-2- yl)-2-(3-methyl-1,2,4-oxa- diazol-5-yl)ethan-1-one |
| 193 | 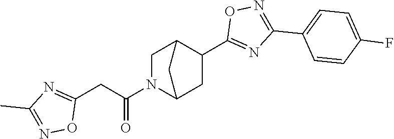 [SMILES: Cc1noc(CC(=O)N2CC3CC2CC3c2nc(-c3ccc(F)cc3)no2)n1] | >10 | 1-(5-(3-(4-fluorophenyl)- 1,2,4-oxadiazol-5-yl)-2- azabicyclo[2.2.1]heptan-2- yl)-2-(3-methyl-1,2,4-oxa- diazol-5-yl)ethan-1-one |
| 194 | 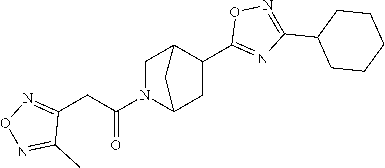 [SMILES: Cc1nonc1CC(=O)N1CC2CC1CC2c1nc(C2CCCCC2)no1] | 0.79 | 1-(5-(3-cyclohexyl-1,2,4- oxadiazol-5-yl)-2-aza- bicyclo[2.2.1]heptan-2-yl)- 2-(4-methyl-1,2,5-oxadia- zol-3-yl)ethan-1-one |
| 195 | 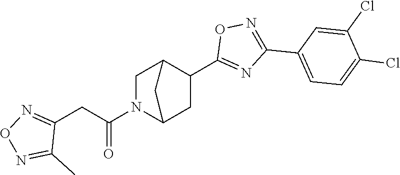 [SMILES: Cc1nonc1CC(=O)N1CC2CC1CC2c1nc(-c2ccc(Cl)c(Cl)c2)no1] | 0.094 | 1-(5-(3-(3,4-dichlorophen- yl)-1,2,4-oxadiazol-5-yl)-2- azabicyclo[2.2.1]heptan-2- yl)-2-(4-methyl-1,2,5-oxa- diazol-3-yl)ethan-1-one |
| 196 | 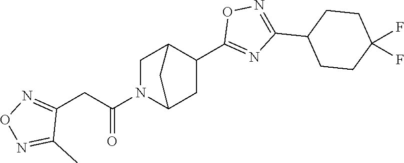 [SMILES: Cc1nonc1CC(=O)N1CC2CC1CC2c1nc(C2CCC(F)(F)CC2)no1] | 1.25 | 1-(5-(3-(4,4-difluorocyclo- hexyl)-1,2,4-oxadiazol-5- yl)-2-azabicyclo[2.2.1] heptan-2-yl)-2-(4-methyl- 1,2,5-oxadiazol-3-yl)ethan- 1-one |
| 197 | 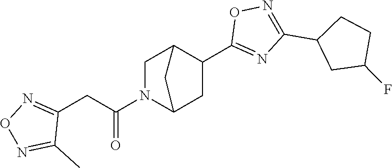 [SMILES: Cc1nonc1CC(=O)N1CC2CC1CC2c1nc(C2CCC(F)C2)no1] | 15.46 | 1-(5-(3-(3-fluorocyclopent- yl)-1,2,4-oxadiazol-5-yl)-2- azabicyclo[2.2.1]heptan-2- yl)-2-(4-methyl-1,2,5-oxa- diazol-3-yl)ethan-1-one |
| 198 | 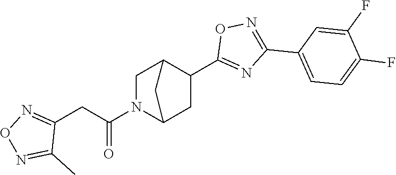 [SMILES: Cc1nonc1CC(=O)N1CC2CC1CC2c1nc(-c2ccc(F)c(F)c2)no1] | 0.247 | 1-(5-(3-(3,4-difluorophen- yl)-1,2,4-oxadiazol-5-yl)-2- azabicyclo[2.2.1]heptan-2- yl)-2-(4-methyl-1,2,5-oxa- diazol-3-yl)ethan-1-one |
| 199 | 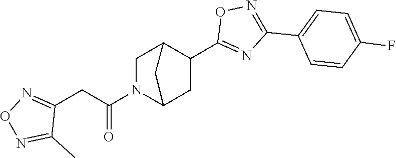 [SMILES: Cc1nonc1CC(=O)N1CC2CC1CC2c1nc(-c2ccc(F)cc2)no1] | 0.324 | 1-(5-(3-(4-fluorophenyl)- 1,2,4-oxadiazol-5-yl)-2- azabicyclo[2.2.1]heptan-2- yl)-2-(4-methyl-1,2,5-oxa- diazol-3-yl)ethan-1-one |
| 200 | 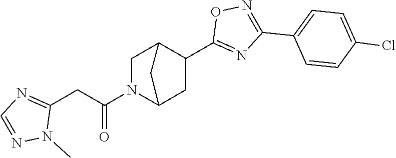 [SMILES: Cn1ncnc1CC(=O)N1CC2CC1CC2c1nc(-c2ccc(Cl)cc2)no1] | 0.37 | 1-(5-(3-(4-chlorophenyl)- 1,2,4-oxadiazol-5-yl)-2- azabicyclo[2.2.1]heptan-2- yl)-2-(1-methyl-1H-1,2,4- triazol-5-yl)ethan-1-one |
| 201 | 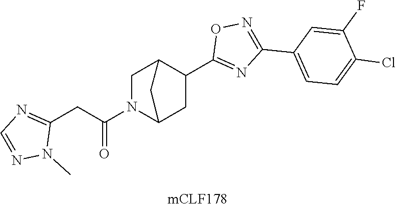 [SMILES: Cn1ncnc1CC(=O)N1CC2CC1CC2c1nc(-c2ccc(Cl)c(F)c2)no1] | 0.2 | 1-(5-(3-(4-chloro-3-fluoro- phenyl)-1,2,4-oxadiazol-5- yl)-2-azabicyclo[2.2.1] heptan-2-yl)-2-(1-methyl- 1H-1,2,4-triazol-5-yl)ethan- 1-one |
| 202 | 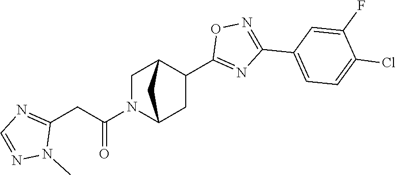 [SMILES: Cn1ncnc1CC(=O)N1C[C@H]2C[C@@H]1CC2c1nc(-c2ccc(Cl)c(F)c2)no1] | | 1-((1R,4S)-5-(3-(4-chloro- 3-fluorophenyl)-1,2,4-oxa- diazol-5-yl)-2-azabicyclo [2.2.1]heptan-2-yl)-2-(1- methyl-1H-1,2,4-triazol-5- yl)ethan-1-one |
| 203 | 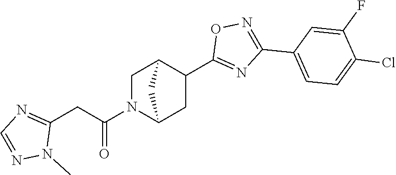 [SMILES: Cn1ncnc1CC(=O)N1C[C@@H]2C[C@H]1CC2c1nc(-c2ccc(Cl)c(F)c2)no1] | | 1-((1S,4R)-5-(3-(4-chloro- 3-fluorophenyl)-1,2,4-oxa- diazol-5-yl)-2-azabicyclo [2.2.1]heptan-2-yl)-2-(1- methyl-1H-1,2,4-triazol-5- yl)ethan-1-one |
| 204 | 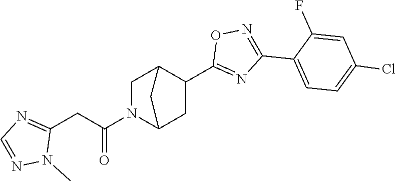 [SMILES: Cn1ncnc1CC(=O)N1CC2CC1CC2c1nc(-c2ccc(Cl)cc2F)no1] | 0.74 | 1-(5-(3-(4-chloro-2-fluoro- phenyl)-1,2,4-oxadiazol-5- yl)-2-azabicyclo[2.2.1] heptan-2-yl)-2-(1-methyl- 1H-1,2,4-triazol-5-yl)ethan- 1-one |
| 205 | 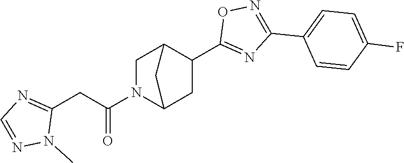 [SMILES: Cn1ncnc1CC(=O)N1CC2CC1CC2c1nc(-c2ccc(F)cc2)no1] | 1.6 | 1-(5-(3-(4-fluorophenyl)- 1,2,4-oxadiazol-5-yl)-2- azabicyclo[2.2.1]heptan-2- yl)-2-(1-methyl-1H-1,2,4- triazol-5-yl)ethan-1-one |
| 206 | 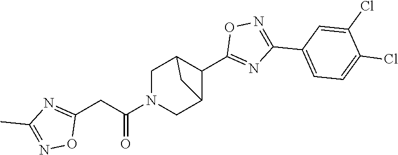 [SMILES: Cc1noc(CC(=O)N2CC3CC(C2)C3c2nc(-c3ccc(Cl)c(Cl)c3)no2)n1] | >10 | 1-(6-(3-(3,4-dichlorophen- yl)-1,2,4-oxadiazol-5-yl)-3- azabicyclo[3.1.1]heptan-3- yl)-2-(3-methyl-1,2,4-oxa- diazol-5-yl)ethan-1-one |
| 207 | 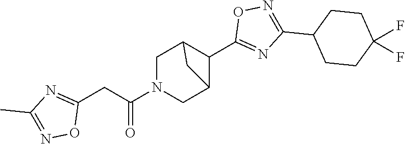 [SMILES: Cc1noc(CC(=O)N2CC3CC(C2)C3c2nc(C3CCC(F)(F)CC3)no2)n1] | >10 | 1-(6-(3-(4,4-difluorocyclo- hexyl)-1,2,4-oxadiazol-5- yl)-3-azabicyclo[3.1.1] heptan-3-yl)-2-(3-methyl- 1,2,4-oxadiazol-5-yl)ethan- 1-one |
| 208 | 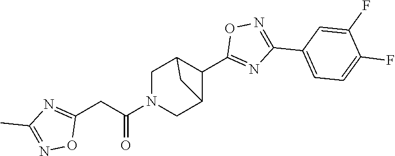 [SMILES: Cc1noc(CC(=O)N2CC3CC(C2)C3c2nc(-c3ccc(F)c(F)c3)no2)n1] | 10 | 1-(6-(3-(3,4-difluorophen- yl)-1,2,4-oxadiazol-5-yl)-3- azabicyclo[3.1.1]heptan-3- yl)-2-(3-methyl-1,2,4-oxa- diazol-5-yl)ethan-1-one |
| 209 | 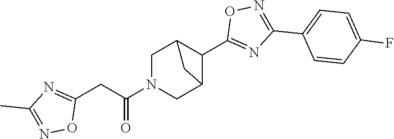 [SMILES: Cc1noc(CC(=O)N2CC3CC(C2)C3c2nc(-c3ccc(F)cc3)no2)n1] | 10 | 1-(6-(3-(4-fluorophenyl)- 1,2,4-oxadiazol-5-yl)-3- azabicyclo[3.1.1]heptan-3- yl)-2-(3-methyl-1,2,4-oxa- diazol-5-yl)ethan-1-one |
| 210 | 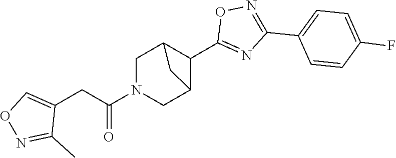 [SMILES: Cc1nocc1CC(=O)N1CC2CC(C1)C2c1nc(-c2ccc(F)cc2)no1] | 25 | 1-(6-(3-(4-fluorophenyl)- 1,2,4-oxadiazol-5-yl)-3- azabicyclo[3.1.1]heptan-3- yl)-2-(3-methylisoxazol-4- yl)ethan-1-one |
| 211 | 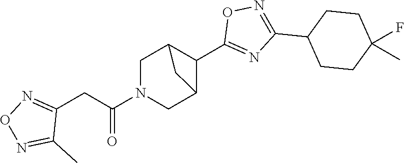 [SMILES: Cc1nonc1CC(=O)N1CC2CC(C1)C2c1nc(C2CCC(C)(F)CC2)no1] | 3.76 | 1-(6-(3-(4-fluoro-4-methyl- cyclohexyl)-1,2,4-oxadia- zol-5-yl)-3-azabicyclo[3.1.1] heptan-3-yl)-2-(4-methyl- 1,2,5-oxadiazol-3-yl)ethan- 1-one |
| 212 | 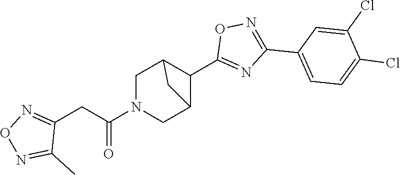 [SMILES: Cc1nonc1CC(=O)N1CC2CC(C1)C2c1nc(-c2ccc(Cl)c(Cl)c2)no1] | 5.01 | 1-(6-(3-(3,4-dichlorophen- yl)-1,2,4-oxadiazol-5-yl)-3- azabicyclo[3.1.1]heptan-3- yl)-2-(4-methyl-1,2,5-oxa- diazol-3-yl)ethan-1-one |
| 213 | 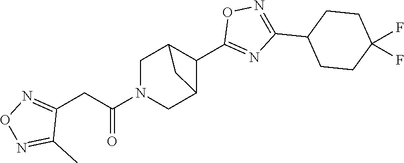 [SMILES: Cc1nonc1CC(=O)N1CC2CC(C1)C2c1nc(C2CCC(F)(F)CC2)no1] | 0.488 | 1-(6-(3-(4,4-difluorocyclo- hexyl)-1,2,4-oxadiazol-5- yl)-3-azabicyclo[3.1.1] heptan-3-yl)-2-(4-methyl- 1,2,5-oxadiazol-3-yl)ethan- 1-one |
| 214 | 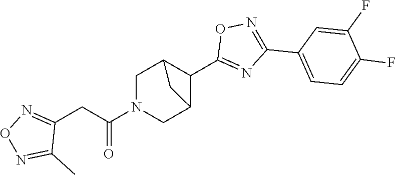 [SMILES: Cc1nonc1CC(=O)N1CC2CC(C1)C2c1nc(-c2ccc(F)c(F)c2)no1] | 5.05 | 1-(6-(3-(3,4-difluorophen- yl)-1,2,4-oxadiazol-5-yl)-3- azabicyclo[3.1.1]heptan-3- yl)-2-(4-methyl-1,2,5-oxa- diazol-3-yl)ethan-1-one |
| 215 | 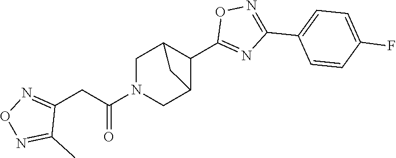 [SMILES: Cc1nonc1CC(=O)N1CC2CC(C1)C2c1nc(-c2ccc(F)cc2)no1] | 0.132 | 1-(6-(3-(4-fluorophenyl)- 1,2,4-oxadiazol-5-yl)-3- azabicyclo[3.1.1]heptan-3- yl)-2-(4-methyl-1,2,5-oxa- diazol-3-yl)ethan-1-one |
| 216 | 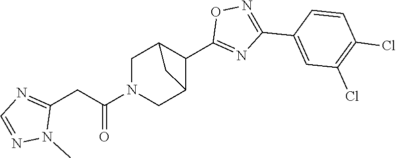 [SMILES: Cn1ncnc1CC(=O)N1CC2CC(C1)C2c1nc(-c2ccc(Cl)c(Cl)c2)no1] | >10 | 1-(6-(3-(3,4-dichlorophen- yl)-1,2,4-oxadiazol-5-yl)-3- azabicyclo[3.1.1]heptan-3- yl)-2-(1-methyl-1H-1,2,4- triazol-5-yl)ethan-1-one |
| 217 | 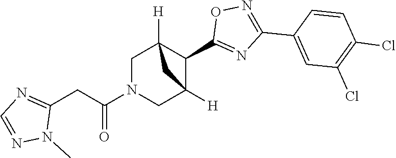 [SMILES: Cn1ncnc1CC(=O)N1C[C@H]2C[C@@H](C1)[C@@H]2c1nc(-c2ccc(Cl)c(Cl)c2)no1] | 0.1 | 1-((1R,5S,6r)-6-(3-(3,4- dichlorophenyl)-1,2,4- oxadiazol-5-yl)-3-aza- bicyclo[3.1.1]heptan-3- yl)-2-(1-methyl-1H-1,2,4- triazol-5-yl)ethan-1-one |
| 218 | 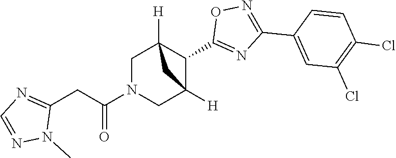 [SMILES: Cn1ncnc1CC(=O)N1C[C@H]2C[C@@H](C1)[C@H]2c1nc(-c2ccc(Cl)c(Cl)c2)no1] | >10 | 1-((1R,5S,6s)-6-(3-(3,4- dichlorophenyl)-1,2,4-oxa- diazol-5-yl)-3-azabicyclo [3.1.1]heptan-3-yl)-2-(1- methyl-1H-1,2,4-triazol-5- yl)ethan-1-one |
| 219 | 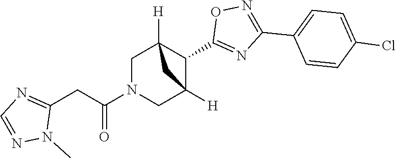 [SMILES: Cn1ncnc1CC(=O)N1C[C@H]2C[C@@H](C1)[C@H]2c1nc(-c2ccc(Cl)cc2)no1] | >10 | 1-((1R,5S,6s)-6-(3-(4- chlorophenyl)-1,2,4-oxa- diazol-5-yl)-3-azabicyclo [3.1.1]heptan-3-yl)-2-(1- methyl-1H-1,2,4-triazol- 5-yl)ethan-1-one |
| 220 | 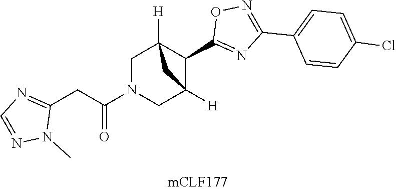 [SMILES: Cn1ncnc1CC(=O)N1C[C@H]2C[C@@H](C1)[C@@H]2c1nc(-c2ccc(Cl)cc2)no1] | 0.14 | 1-((1R,5S,6r)-6-(3-(4- chlorophenyl)-1,2,4-oxa- diazol-5-yl)-3-azabicyclo [3.1.1]heptan-3-yl)-2-(1- methyl-1H-1,2,4-triazol- 5-yl)ethan-1-one |
| 221 | 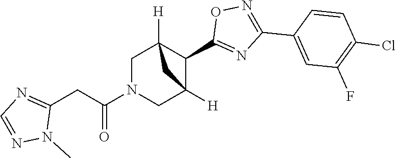 [SMILES: Cn1ncnc1CC(=O)N1C[C@H]2C[C@@H](C1)[C@@H]2c1nc(-c2ccc(Cl)c(F)c2)no1] | 0.1 | 1-((1R,5S,6r)-6-(3-(4-chloro- 3-fluorophenyl)-1,2,4-oxa- diazol-5-yl)-3-azabicyclo [3.1.1]heptan-3-yl)-2-(1- methyl-1H-1,2,4-triazol-5- yl)ethan-1-one |
| 222 | 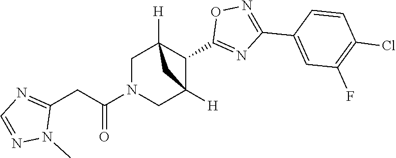 [SMILES: Cn1ncnc1CC(=O)N1C[C@H]2C[C@@H](C1)[C@H]2c1nc(-c2ccc(Cl)c(F)c2)no1] | >10 | 1-((1R,5S,6s)-6-(3-(4-chloro- 3-fluorophenyl)-1,2,4-oxa- diazol-5-yl)-3-azabicyclo [3.1.1]heptan-3-yl)-2-(1- methyl-1H-1,2,4-triazol- 5-yl)ethan-1-one |
| 223 | 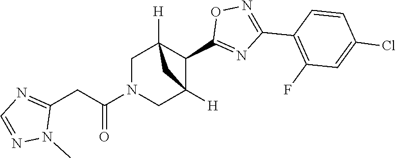 [SMILES: Cn1ncnc1CC(=O)N1C[C@H]2C[C@@H](C1)[C@@H]2c1nc(-c2ccc(Cl)cc2F)no1] | 0.29 | 1-((1R,5S,6r)-6-(3-(4-chloro- 2-fluorophenyl)-1,2,4-oxa- diazol-5-yl)-3-azabicyclo [3.1.1]heptan-3-yl)-2-(1- methyl-1H-1,2,4-triazol- 5-yl)ethan-1-one |
| 224 | 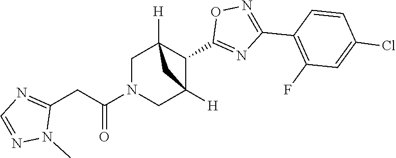 [SMILES: Cn1ncnc1CC(=O)N1C[C@H]2C[C@@H](C1)[C@H]2c1nc(-c2ccc(Cl)cc2F)no1] | >10 | 1-((1R,5S,6s)-6-(3-(4-chloro- 2-fluorophenyl)-1,2,4-oxa- diazol-5-yl)-3-azabicyclo [3.1.1]heptan-3-yl)-2-(1- methyl-1H-1,2,4-triazol- 5-yl)ethan-1-one |
| 225 | 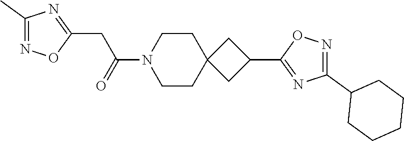 [SMILES: Cc1noc(CC(=O)N2CCC3(CC2)CC(c2nc(C4CCCCC4)no2)C3)n1] | >10 | 1-(2-(3-cyclohexyl-1,2,4- oxadiazol-5-yl)-7-azaspiro [3.5]nonan-7-yl)-2-(3-meth- yl-1,2,4-oxadiazol-5-yl)- ethan-1-one |
| 226 | 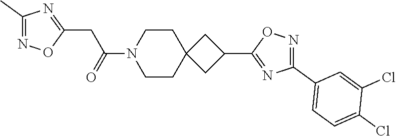 [SMILES: Cc1noc(CC(=O)N2CCC3(CC2)CC(c2nc(-c4ccc(Cl)c(Cl)c4)no2)C3)n1] | 0.109 | 1-(2-(3-(3,4-dichlorophenyl)- 1,2,4-oxadiazol-5-yl)-7-aza- spiro[3.5]nonan-7-yl)-2-(3- methyl-1,2,4-oxadiazol-5- yl)ethan-1-one |
| 227 | 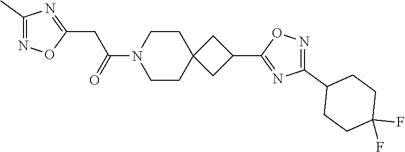 [SMILES: Cc1noc(CC(=O)N2CCC3(CC2)CC(c2nc(C4CCC(F)(F)CC4)no2)C3)n1] | >10 | 1-(2-(3-(4,4-difluorocyclo- hexyl)-1,2,4-oxadiazol-5- yl)-7-azaspiro[3.5]nonan-7- yl)-2-(3-methyl-1,2,4-oxa- diazol-5-yl)ethan-1-one |
| 228 | 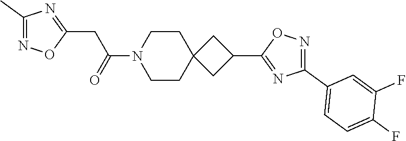 [SMILES: Cc1noc(CC(=O)N2CCC3(CC2)CC(c2nc(-c4ccc(F)c(F)c4)no2)C3)n1] | 0.294 | 1-(2-(3-(3,4-difluorophen- yl)-1,2,4-oxadiazol-5-yl)-7- azaspiro[3.5]nonan-7-yl)-2- (3-methyl-1,2,4-oxadiazol- 5-yl)ethan-1-one |
| 229 | 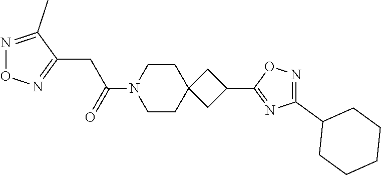 [SMILES: Cc1nonc1CC(=O)N1CCC2(CC1)CC(c1nc(C3CCCCC3)no1)C2] | 3.06 | 1-(2-(3-cyclohexyl-1,2,4- oxadiazol-5-yl)-7-azaspiro [3.5]nonan-7-yl)-2-(4-meth- yl-1,2,5-oxadiazol-3-yl)- ethan-1-one |
| 230 | 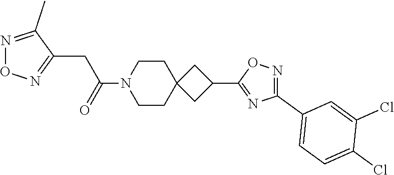 [SMILES: Cc1nonc1CC(=O)N1CCC2(CC1)CC(c1nc(-c3ccc(Cl)c(Cl)c3)no1)C2] | 0.176 | 1-(2-(3-(3,4-dichlorophen- yl)-1,2,4-oxadiazol-5-yl)-7- azaspiro[3.5]nonan-7-yl)-2- (4-methyl-1,2,5-oxadiazol- 3-yl)ethan-1-one |
| 231 | 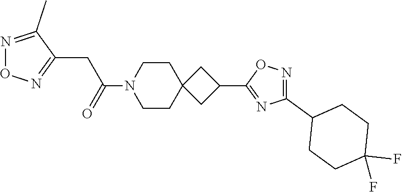 [SMILES: Cc1nonc1CC(=O)N1CCC2(CC1)CC(c1nc(C3CCC(F)(F)CC3)no1)C2] | >10 | 1-(2-(3-(4,4-difluorocyclo- hexyl)-1,2,4-oxadiazol-5- yl)-7-azaspiro[3.5]nonan-7- yl)-2-(4-methyl-1,2,5-oxa- diazol-3-yl)ethan-1-one |
| 232 | 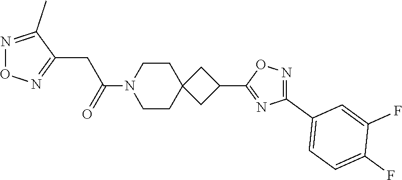 [SMILES: Cc1nonc1CC(=O)N1CCC2(CC1)CC(c1nc(-c3ccc(F)c(F)c3)no1)C2] | 0.257 | 1-(2-(3-(3,4-difluorophen- yl)-1,2,4-oxadiazol-5-yl)-7- azaspiro[3.5]nonan-7-yl)-2- (4-methyl-1,2,5-oxadiazol- 3-yl)ethan-1-one |
| 233 | 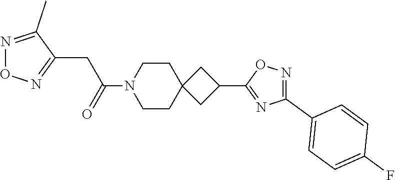 [SMILES: Cc1nonc1CC(=O)N1CCC2(CC1)CC(c1nc(-c3ccc(F)cc3)no1)C2] | 0.45 | 1-(2-(3-(4-fluorophenyl)- 1,2,4-oxadiazol-5-yl)-7- azaspiro[3.5]nonan-7-yl)-2- (4-methyl-1,2,5-oxadiazol- 3-yl)ethan-1-one |
| 234 | 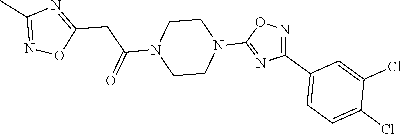 [SMILES: Cc1noc(CC(=O)N2CCN(c3nc(-c4ccc(Cl)c(Cl)c4)no3)CC2)n1] | >10 | 1-(4-(3-(3,4-dichlorophen- yl)-1,2,4-oxadiazol-5-yl)- piperazin-1-yl)-2-(3-methyl- 1,2,4-oxadiazol-5-yl)ethan- 1-one |
| 235 | 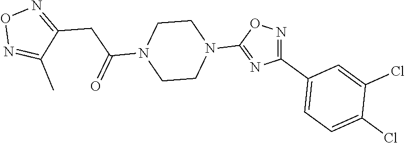 [SMILES: Cc1nonc1CC(=O)N1CCN(c2nc(-c3ccc(Cl)c(Cl)c3)no2)CC1] | 0.27 | 1-(4-(3-(3,4-dichlorophen- yl)-1,2,4-oxadiazol-5-yl)- piperazin-1-yl)-2-(4-methyl- 1,2,5-oxadiazol-3-yl)ethan- 1-one |
| 236 | 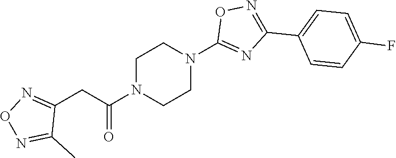 [SMILES: Cc1nonc1CC(=O)N1CCN(c2nc(-c3ccc(F)cc3)no2)CC1] | 0.649 | 1-(4-(3-(4-fluorophenyl)- 1,2,4-oxadiazol-5-yl)piper- azin-1-yl)-2-(4-methyl- 1,2,5-oxadiazol-3-yl)ethan- 1-one |
| 237 | 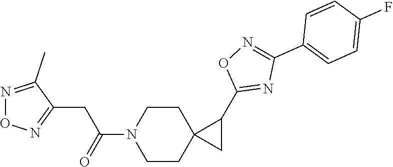 [SMILES: Cc1nonc1CC(=O)N1CCC2(CC1)CC2c1nc(-c2ccc(F)cc2)no1] | >10 | 1-(1-(3-(4-fluorophenyl)- 1,2,4-oxadiazol-5-yl)-6- azaspiro[2.5]octan-6-yl)-2- (4-methyl-1,2,5-oxadiazol- 3-yl)ethan-1-one |
| 238 | 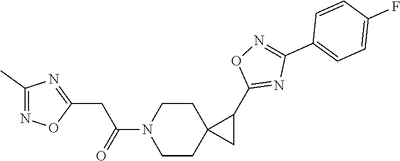 [SMILES: Cc1noc(CC(=O)N2CCC3(CC2)CC3c2nc(-c3ccc(F)cc3)no2)n1] | >10 | 1-(1-(3-(4-fluorophenyl)- 1,2,4-oxadiazol-5-yl)-6- azaspiro[2.5]octan-6-yl)-2- (3-methyl-1,2,4-oxadiazol- 5-yl)ethan-1-one |
| 239 | 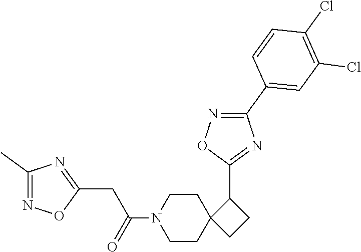 [SMILES: Cc1noc(CC(=O)N2CCC3(CCC3c3nc(-c4ccc(Cl)c(Cl)c4)no3)CC2)n1] | >25 | 1-(1-(3-(3,4-dichlorophen- yl)-1,2,4-oxadiazol-5-yl)-7- azaspiro[3.5]nonan-7-yl)-2- (3-methyl-1,2,4-oxadiazol- 5-yl)ethan-1-one |
| 240 | 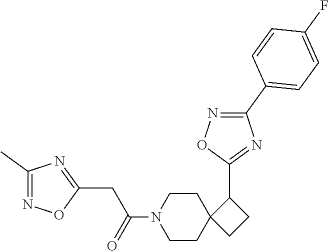 [SMILES: Cc1noc(CC(=O)N2CCC3(CCC3c3nc(-c4ccc(F)cc4)no3)CC2)n1] | >10 | 1-(1-(3-(4-fluorophenyl)- 1,2,4-oxadiazol-5-yl)-7-aza- spiro[3.5]nonan-7-yl)-2-(3- methyl-1,2,4-oxadiazol-5- yl)ethan-1-one |
| 241 | 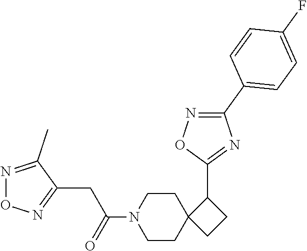 [SMILES: Cc1nonc1CC(=O)N1CCC2(CCC2c2nc(-c3ccc(F)cc3)no2)CC1] | >10 | 1-(1-(3-(4-fluorophenyl)- 1,2,4-oxadiazol-5-yl)-7- azaspiro[3.5]nonan-7-yl)-2- (4-methyl-1,2,5-oxadiazol- 3-yl)ethan-1-one |
| 242 | 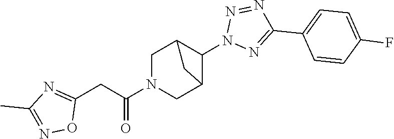 [SMILES: Cc1noc(CC(=O)N2CC3CC(C2)C3n2nnc(-c3ccc(F)cc3)n2)n1] | >25 | 1-(6-(5-(4-fluorophenyl)- 2H-tetrazol-2-yl)-3-aza- bicyclo[3.1.1]heptan-3-yl)- 2-(3-methyl-1,2,4-oxadia- zol-5-yl)ethan-1-one |
| 243 | 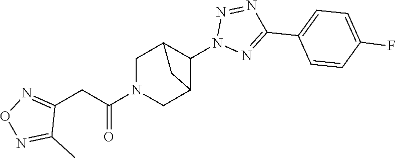 [SMILES: Cc1nonc1CC(=O)N1CC2CC(C1)C2n1nnc(-c2ccc(F)cc2)n1] | >10 | 1-(6-(5-(4-fluorophenyl)- 2H-tetrazol-2-yl)-3-aza- bicyclo[3.1.1]heptan-3-yl)- 2-(4-methyl-1,2,5-oxadia- zol-3-yl)ethan-1-one |
| 244 | 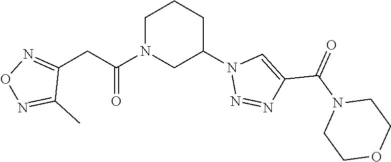 [SMILES: Cc1nonc1CC(=O)N1CCCC(n2cc(C(=O)N3CCOCC3)nn2)C1] | >10 | 2-(4-methyl-1,2,5-oxadia- zol-3-yl)-1-(3-(4-(morph- oline-4-carbonyl)-1H-1,2,3- triazol-1-yl)piperidin-1-yl)- ethan-1-one |
| 245 | 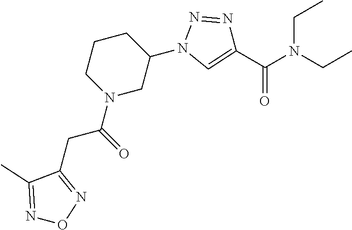 [SMILES: CCN(CC)C(=O)c1cn(C2CCCN(C(=O)Cc3nonc3C)C2)nn1] | >10 | N,N-diethyl-1-(1-(2-(4- methyl-1,2,5-oxadiazol-3- yl)acetyl)piperidin-3-yl)- 1H-1,2,3-triazole-4-carbox- amide |
| 246 | 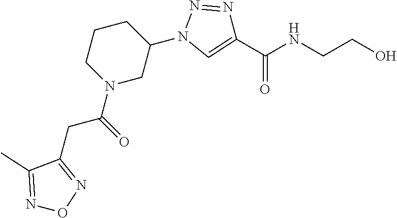 [SMILES: Cc1nonc1CC(=O)N1CCCC(n2cc(C(=O)NCCO)nn2)C1] | >10 | N-(2-hydroxyethyl)-1-(1-(2- (4-methyl-1,2,5-oxadiazol- 3-yl)acetyl)piperidin-3-yl)- 1H-1,2,3-triazole-4-carbox- amide |
| 247 | 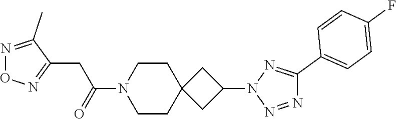 [SMILES: Cc1nonc1CC(=O)N1CCC2(CC1)CC(n1nnc(-c3ccc(F)cc3)n1)C2] | 1.25 | 1-(2-(5-(4-fluorophenyl)- 2H-tetrazol-2-yl)-7-aza- spiro[3.5]nonan-7-yl)-2- (4-methyl-1,2,5-oxadiazol- 3-yl)ethan-1-one |
| 248 | 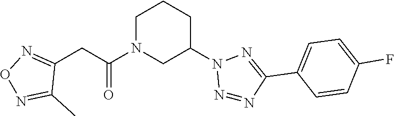 [SMILES: Cc1nonc1CC(=O)N1CCCC(n2nnc(-c3ccc(F)cc3)n2)C1] | >10 | 1-(3-(5-(4-fluorophenyl)- 2H-tetrazol-2-yl)piperidin- 1-yl)-2-(4-methyl-1,2,5- oxadiazol-3-yl)ethan-1-one |
| 249 | 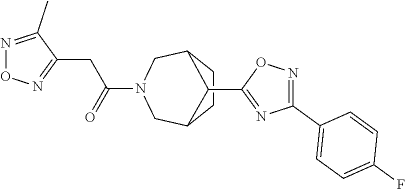 [SMILES: Cc1nonc1CC(=O)N1CC2CCC(C1)C2c1nc(-c2ccc(F)cc2)no1] | 1.29 | 1-(8-(3-(4-fluorophenyl)- 1,2,4-oxadiazol-5-yl)-3- azabicyclo[3.2.1]octan-3- yl)-2-(4-methyl-1,2,5-oxa- diazol-3-yl)ethan-1-one |
| 250 | 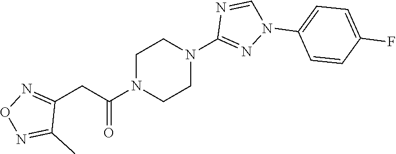 [SMILES: Cc1nonc1CC(=O)N1CCN(c2ncn(-c3ccc(F)cc3)n2)CC1] | >10 | 1-(4-(1-(4-fluorophenyl)- 1H-1,2,4-triazol-3-yl)piper- azin-1-yl)-2-(4-methyl- 1,2,5-oxadiazol-3-yl)ethan- 1-one |
| 251 | 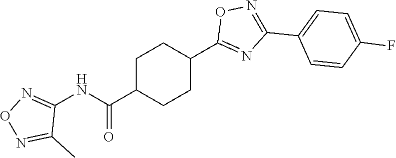 [SMILES: Cc1nonc1NC(=O)C1CCC(c2nc(-c3ccc(F)cc3)no2)CC1] | >10 | 4-(3-(4-fluorophenyl)-1,2,4- oxadiazol-5-yl)-N-(4-meth- yl-1,2,5-oxadiazol-3-yl)- cyclohexane-1-carboxamide |
| 252 | 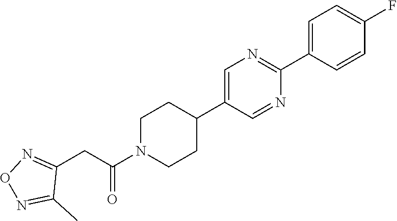 [SMILES: Cc1nonc1CC(=O)N1CCC(c2cnc(-c3ccc(F)cc3)nc2)CC1] | 2.91 | 1-(4-(2-(4-fluorophenyl)- pyrimidin-5-yl)piperidin-1- yl)-2-(4-methyl-1,2,5-oxa- diazol-3-yl)ethan-1-one |
| 253 | 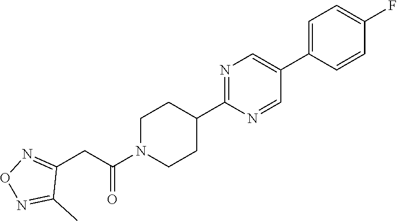 [SMILES: Cc1nonc1CC(=O)N1CCC(c2ncc(-c3ccc(F)cc3)cn2)CC1] | >10 | 1-(4-(5-(4-fluorophenyl)- pyrimidin-2-yl)piperidin-1- yl)-2-(4-methyl-1,2,5-oxa- diazol-3-yl)ethan-1-one |
| 254 | 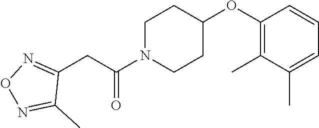 [SMILES: Cc1cccc(OC2CCN(C(=O)Cc3nonc3C)CC2)c1C] | >10 | 1-(4-(2,3-dimethylphen- oxy)piperidin-1-yl)-2-(4- methyl-1,2,5-oxadiazol-3- yl)ethan-1-one |
| 255 | 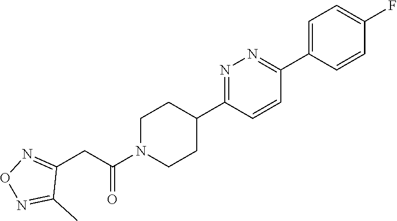 [SMILES: Cc1nonc1CC(=O)N1CCC(c2ccc(-c3ccc(F)cc3)nn2)CC1] | >10 | 1-(4-(6-(4-fluorophenyl)- pyridazin-3-yl)piperidin-1- yl)-2-(4-methyl-1,2,5-oxa- diazol-3-yl)ethan-1-one |
| 256 | 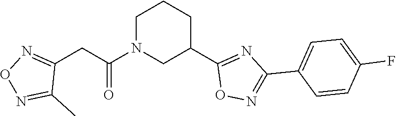 [SMILES: Cc1nonc1CC(=O)N1CCCC(c2nc(-c3ccc(F)cc3)no2)C1] | >10 | 1-(3-(3-(4-fluorophenyl)- 1,2,4-oxadiazol-5-yl)piperi- din-1-yl)-2-(4-methyl-1,2,5- oxadiazol-3-yl)ethan-1-one |
| 257 | 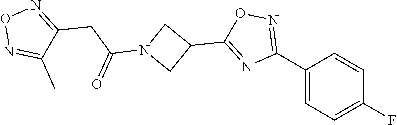 [SMILES: Cc1nonc1CC(=O)N1CC(c2nc(-c3ccc(F)cc3)no2)C1] | >10 | 1-(3-(3-(4-fluorophenyl)- 1,2,4-oxadiazol-5-yl)azeti- din-1-yl)-2-(4-methyl-1,2,5- oxadiazol-3-yl)ethan-1-one |
| 258 | 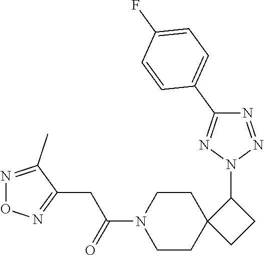 [SMILES: Cc1nonc1CC(=O)N1CCC2(CCC2n2nnc(-c3ccc(F)cc3)n2)CC1] | >25 | 1-(1-(5-(4-fluorophenyl)- 2H-tetrazol-2-yl)-7-azaspiro [3.5]nonan-7-yl)-2-(4-meth- yl-1,2,5-oxadiazol-3-yl)- ethan-1-one |

### EXAMPLES

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. It should be appreciated that the synthetic reaction schemes shown in the Examples section are merely illustrative of some methods by which the compounds of the invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data. Unless specified to the contrary, the reactions described herein are typically conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

Various substituents on the compounds of the invention can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups are known in the art, and can be employed. Examples of many of the possible groups can be found in “Protective Groups in Organic Synthesis” by Green et al., John Wiley and Sons, 1999. For example, nitro groups can be added by nitration and the nitro group can be converted to other groups, such as amino by reduction, and halogen by diazotization of the amino group and replacement of the diazo group with halogen. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), 1,3-Diisopropylcarbodiimide (DIC), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

Additional embodiments of the disclosure reside in specific examples and data described in more detail herein. See also: Wilburn, K. M., et al. (2022). PLoS pathogens, 18(2), e1009862. https://doi.org/10.1371/journal.ppat.1009862, which is herein incorporated by reference).

Synthesis of Compounds. The compounds of the present disclosure are made according to, and by adaptation of, the following exemplary procedures.

### Example 1. Synthesis of 1-{4-[5-(p-Fluorophenyl)-2H-1,2,3,4-tetraazol-2-yl]-1-piperidyl}-2-(4-methyl-3-furazanyl)-1-ethanone (mCED315)

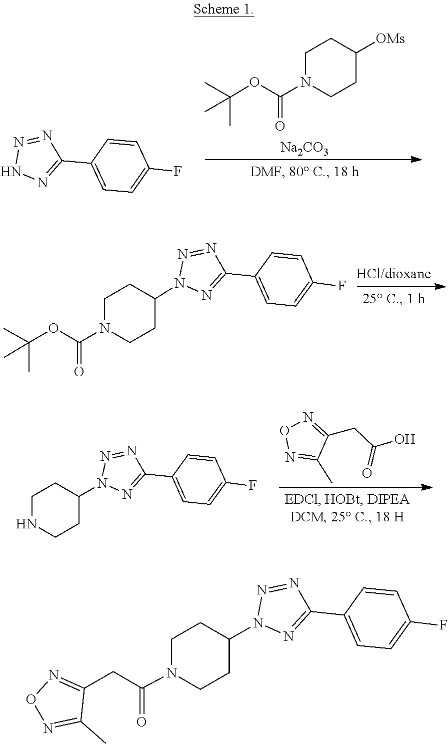

### tert-Butyl 4-(5-(4-fluorophenyl)-2H-tetrazol-2-yl)piperidine-1-carboxylate

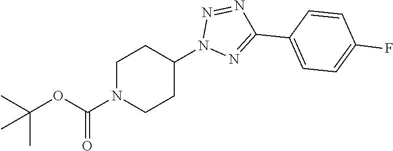 [SMILES: CC(C)(C)OC(=O)N1CCC(n2nnc(-c3ccc(F)cc3)n2)CC1]

To a solution of 5-(4-fluorophenyl)-2H-tetrazole (492 mg, 4.0 mmol, 1.0 eq) and tert-butyl 4-((methylsulfonyl)oxy)piperidine-1-carboxylate (977 mg, 3.5 mmol, 1.17 eq) in DMF (10 mL) was added Na₂CO₃ (848 mg, 8 mmol, 2.0 eq). The mixture was stirred and heated at 80° C. for 18 hours. LC/MS showed the starting material was consumed completely and the desired compound was detected. The reaction mixture was filtered and concentrated. The residue was purified by silica gel chromatography eluted with 0-30% ethyl acetate in hexane to afford tert-butyl 4-(5-(4-fluorophenyl)-2H-tetrazol-2-yl)piperidine-1-carboxylate (580 mg, 1.67 mmol, 56% yield) as a white solid.

### 4-(5-(4-Fluorophenyl)-2H-tetrazol-2-yl)piperidine

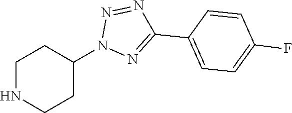 [SMILES: Fc1ccc(-c2nnn(C3CCNCC3)n2)cc1]

A solution of tert-butyl tert-butyl 4-(5-(4-fluorophenyl)-2H-tetrazol-2-yl)piperidine-1-carboxylate (580 mg, 1.67 mmol) in HCl/dioxane (4N, 2.0 mL) was stirred at 25° C. for 1 hr. LC/MS showed the starting material was consumed completely and the desired compound was detected. The mixture reaction was concentrated in vacuum to give 4-(5-(4-fluorophenyl)-2H-tetrazol-2-yl)piperidine (409 mg, 99% yield, HCl) as a white solid.

### 1-{4-[5-(p-Fluorophenyl)-2H-1,2,3,4-tetraazol-2-yl]-1-piperidyl}-2-(4-methyl-3-furazanyl)-1-ethanone (mCED315)

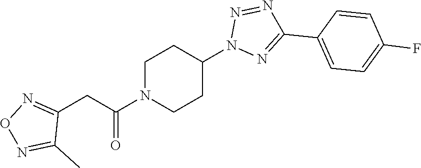 [SMILES: Cc1nonc1CC(=O)N1CCC(n2nnc(-c3ccc(F)cc3)n2)CC1]

A solution of 4-(5-(4-fluorophenyl)-2H-tetrazol-2-yl)piperidine (247 mg, 1.0 mmol, HCl, 1.0 eq), 2-(4-methyl-1,2,5-oxadiazol-3-yl)acetic acid (170 mg, 1.2 mmol, 1.2 eq) and EDCI (155 mg, 1.0 mmol, 1.0 eq), HOBt (135 mg, 1.0 mmol, 1.0 eq), DIPEA (13.2 g, 2.0 mmol, 2.0 eq) in DCM (5 mL) was stirred at 25° C. for 16 hrs. LC/MS showed the starting material was consumed completely and the desired compound was detected. The reaction mixture was extracted with aq NaHCO₃ (2×50 mL). The combined organic layer was washed with saline solution (30 mL) and dried over Na₂SO₄, and the filtrate was concentrated. The residue was purified by silica gel chromatography eluted with 0-10% MeOH in DCM to 1-{4-[5-(p-fluorophenyl)-2H-1,2,3,4-tetraazol-2-yl]-1-piperidyl}-2-(4-methyl-3-furazanyl)-1-ethanone (148.4 mg, 40% yield) as white solid.

LC/MS [ESI, M+1]: m/z 372.3.

¹H NMR (500 MHz, DMSO-d₆) δ 8.13-8.10 (m, 2H), 7.43-7.39 (m, 2H), 5.28-5.22 (m, 1H), 4.38-4.36 (m, 1H), 4.18 (q, J=2.0, 2H), 4.08-4.05 (m, 1H), 3.48-3.42 (m, 1H), 3.08-3.02 (m, 1H), 2.36-2.32 (m, 2H), 2.30 (s, 3H), 2.25-2.17 (m, 1H), 2.01-1.93 (m, 1H); ¹³C NMR (500 MHz, DMSO-d₆) δ 166.10, 164.87, 163.61, 162.90, 153.05, 152.05, 129.31, 129.24, 124.07, 116.96, 116.81, 60.50, 43.93, 31.81, 31.92, 28.07, 8.41; ¹⁹F NMR (500 MHz, DMSO-d₆) δ −110.33.

### Example 2. Synthesis of 1-(4-(3-(4-fluorophenyl)-1,2,4-oxadiazol-5-yl)piperidin-1-yl)-2-(4-methyl-1,2,5-oxadiazol-3-yl)ethan-1-one (mCIS635)

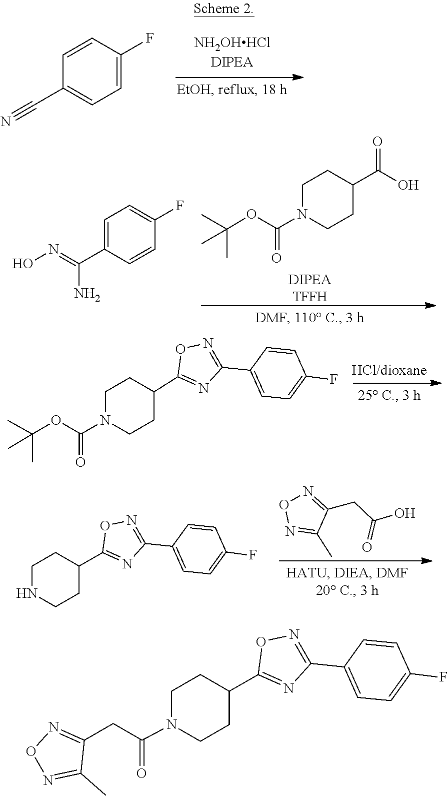

### 4-Fluoro-N′-hydroxy-benzamidine

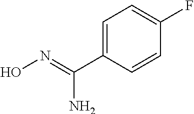 [SMILES: N/C(=N\O)c1ccc(F)cc1]

To a solution of 4-Fluorobenzonitrile (4.8 g, 40 mmol, 1.0 eq) in EtOH (20 mL) was added NH₂OH·HCl (2.6 g, 40 mmol, 1.0 eq) and DIPEA (8.3 g, 64 mmol, 1.6 eq) at 0° C. The mixture was stirred and refluxed for 18 hours. LC/MS showed the starting material was consumed completely and the desired compound was detected. The reaction mixture was concentrated, and dissolved in ethyl acetate (80 mL), and extracted with water (2×100 mL). Then the organic layer was washed with brine (80 mL) and dried over Na₂SO₄. Then the mixture was filtered, and the filtrate was concentrated to afford 4-fluoro-N′-hydroxy-benzamidine (5.7 g, 36.9 mmol, 92% yield) as a white solid.

### Tert-butyl 4-(3-(4-fluorophenyl)-1,2,4-oxadiazol-5-yl)piperidine-1-carboxylate

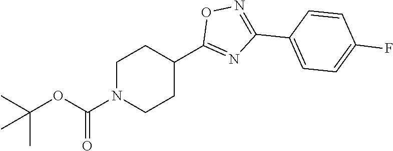 [SMILES: CC(C)(C)OC(=O)N1CCC(c2nc(-c3ccc(F)cc3)no2)CC1]

To a solution of 1-(tert-butoxycarbonyl)piperidine-4-carboxylic acid in DMF (30 mL) was added DIPEA (2.6 g, 20 mmol, 1.0 eq) and TFFH (5.3 g, 20 mmol, 1.0 eq). The mixture was stirred at 25° C. for 30 min, then 4-fluoro-N′-hydroxy-benzamidine (3.1 g, 20 mmol, 1.0 eq) was added. The mixture was stirred at 110° C. for 3 hours. LC/MS showed the starting material was completely consumed and the desired compound was detected. The reaction mixture was concentrated, and then dissolved in ethyl acetate (50 mL). The organic phase was washed with 1 N aqueous HCl solution (2×50 mL) and then the organic phase was dried over Na₂SO₄, and the filtrate was concentrated to afford tert-butyl 4-(3-(4-fluorophenyl)-1,2,4-oxadiazol-5-yl)piperidine-1-carboxylate (5.2 g, 15 mmol, 75% yield) as a white solid.

### 3-(4-Fluorophenyl)-5-(piperidin-4-yl)-1,2,4-oxadiazole

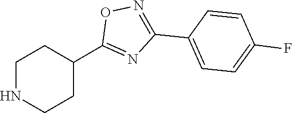 [SMILES: Fc1ccc(-c2noc(C3CCNCC3)n2)cc1]

HCl/dioxane (4.0 M, 20 mL, 5.3 eq) was added to 4-(3-(4-fluorophenyl)-1,2,4-oxadiazol-5-yl)piperidine-1-carboxylate (5.2 g, 15 mmol, 1.0 eq). The mixture was stirred at 25° C. for 3 hours. LC/MS showed the starting material was consumed completely and the desired compound was detected. The mixture was concentrated under vacuum to give 3-(4-fluorophenyl)-5-(piperidin-4-yl)-1,2,4-oxadiazole (HCl salt, 3.5 g, 14.1 mmol, 94% yield) as light brown solid.

### 1-(4-(3-(4-fluorophenyl)-1,2,4-oxadiazol-5-yl)piperidin-1-yl)-2-(4-methyl-1,2,5-oxadiazol-3-yl)ethan-1-one (mCIS635)

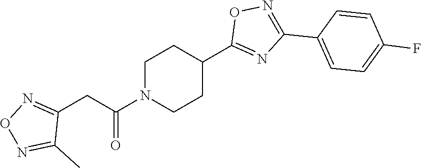 [SMILES: Cc1nonc1CC(=O)N1CCC(c2nc(-c3ccc(F)cc3)no2)CC1]

To a solution of 3-(4-fluorophenyl)-5-(piperidin-4-yl)-1,2,4-oxadiazole (HCl salt, 1.4 g, 10 mmol, 1.0 eq) in DMF (20 mL) was added HATU (5.7 g, 15 mmol, 1.5 eq), 2-(4-methyl-1,2,5-oxadiazol-3-yl)acetic acid (1.7 g, 12 mmol, 1.2 eq) and DIPEA (3.9 g, 30 mmol, 5.2 mL, 3.0 eq). The mixture was stirred at 20° C. for 18 hours. The mixture was concentrated under vacuum and diluted with ethyl acetate (50 mL) and washed with water (2×100 mL). Then the organic layer was washed with brine (80 mL), dried over Na₂SO₄, and the mixture was filtered, and the filtrate was concentrated. The residue was purified by silica gel chromatography eluted with 0-50% ethyl acetate in hexane to afford 1-(4-(3-(4-fluorophenyl)-1,2,4-oxadiazol-5-yl)piperidin-1-yl)-2-(4-methyl-1,2,5-oxadiazol-3-yl)ethan-1-one (1.3 g, 3.5 mmol, 35% yield) as white solid.

LC/MS [ESI, M+1]: m/z 372.2.

¹H NMR (500 MHz, CDCl3) δ 8.08-8.05 (m, 2H), 7.18-7.15 (m, 2H), 4.44-4.41 (m, 1H), 4.05-4.02 (m, 1H), 3.89 (s, 2H), 3.44-3.38 (m, 1H), 3.32-3.26 (m, 1H), 3.11-3.05 (m, 1H), 2.41 (s, 3H), 2.25-2.17 (m, 2H), 2.02-1.88 (m, 2H); ¹³C NMR (500 MHz, CDCl₃) δ 180.66, 167.62, 165.71, 165.02, 163.70, 151.67, 149.85, 129.72, 129.65, 129.65, 116.25, 116.07, 45.27, 41.20, 34.05, 29.52, 28.91, 8.51; ¹⁹F NMR (400 MHz, CDCl₃) δ −108.48.

### Example 3. Synthesis of 1-(4-(3-(3,4-Dichlorophenyl)-1,2,4-oxadiazol-5-yl)piperidin-1-yl)-2-(1-methyl-1H-1,2,4-triazol-5-yl)ethan-1-one (mCLB073)

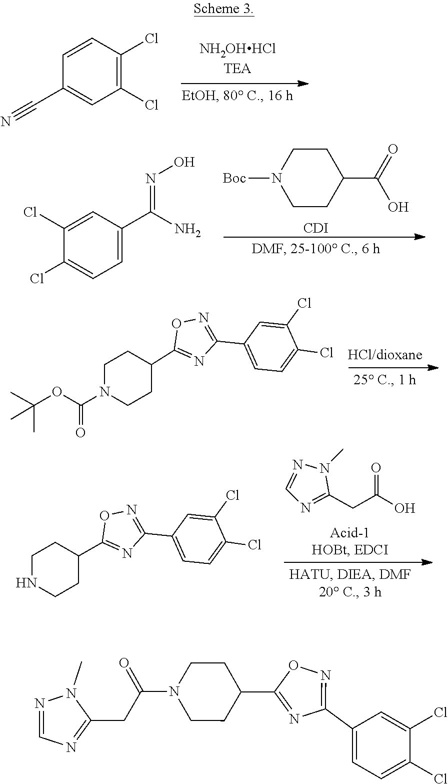

### 2-(1-Methyl-1H-1,2,4-triazol-5-yl)acetic acid (Acid-1)

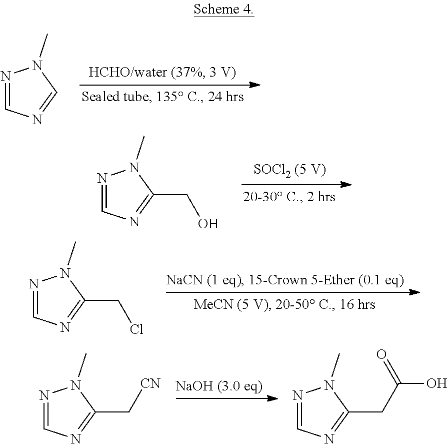

### (1-Methyl-1H-1,2,4-triazol-5-yl)methanol

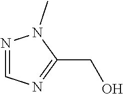

The mixture of 1-methyl-1H-1,2,4-triazole (1.70 kg, 20.5 mol, 1.00 eq) in HCHO/water (5.10 L, 37%) was stirred in a closed container at 135° C. for 24 hrs. TLC (DCM:MeOH=10:1) showed 1-methyl-1H-1,2,4-triazole (Rf=0.5) was consumed, and a new point (Rf=0.45) was appeared. After cooling to room temperature, the solvent was evaporated in vacuum to give (1-methyl-1H-1,2,4-triazol-5-yl)methanol (2.96 kg, crude) as a yellow oil.

### 5-(Chloromethyl)-1-methyl-1H-1,2,4-triazole

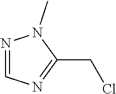

3 batches in parallel: To the solution of SOCl₂ (4.73 kg, 39.8 mol, 2.89 L, 3.00 eq) was added (1-Methyl-1H-1,2,4-triazol-5-yl)methanol (1.50 kg, 13.3 mol, 1.00 eq) slowly at 20-30° C., then the mixture was stirred at 20° C. for 2 hrs. TLC (DCM:MeOH=10:1) showed (1-methyl-1H-1,2,4-triazol-5-yl)methanol (Rf=0.45) was consumed and two new points (Rf=0.2, 0.5) were appeared. The mixture was combined and concentrated under vacuum. The residue was diluted with EtOAc (10.0 L) and washed with NaHCO₃ solution (10.0 L) and brine (5.00 L). The separated organic layer was concentrated to give 5-(chloromethyl)-1-methyl-1H-1,2,4-triazole (2.37 kg, 37.3% yield) as a brown oil.

### 2-(1-methyl-1H-1,2,4-triazol-5-yl)acetonitrile

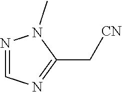

7 batches in parallel: To the solution of 5-(Chloromethyl)-1-methyl-1H-1,2,4-triazole (300 g, 2.28 mol, 1.00 eq) and 15-Crown 5-Ether (50.3 g, 228 mmol, 45.3 mL, 0.10 eq) in MeCN (1.50 L) was added NaCN (112 g, 2.28 mol, 1.00 eq) slowly at 25° C., then the mixture was stirred at 50° C. for 16 hrs. TLC (PE:EA=0:1) showed 5-(chloromethyl)-1-methyl-1H-1,2,4-triazole (R_{f}=0.7) was consumed and a new spot (R_{f}=0.3) was detected. The mixture was concentrated under vacuum. The residue was diluted with EtOAc (500 mL) and filtered. The filtrate was combined and concentrated to give 2-(1-methyl-1H-1,2,4-triazol-5-yl)acetonitrile (1.70 kg, crude) as a black brown oil.

### 2-(1-methyl-11H-1,2,4-triazol-5-yl)acetic Acid (Acid-1)

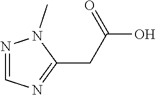

4 batches in parallel: The mixture of 2-(1-methyl-1H-1,2,4-triazol-5-yl)acetonitrile (425 g, 3.48 mol, 1.00 eq) and NaOH (418 g, 10.4 mol, 3.00 eq) in water (1.00 L) and EtOH (1.00 L) was stirred at 70° C. for 2 hrs. The mixture was combined and concentrated to remove EtOH, the result solution was washed with MTBE (2.00 L), then the aqueous phase was adjusted to pH=5 with 6 N HCl and concentrated to give a residue. The residue was triturated with EtOAc/EtOH (10.0 L, 5/1) at 20° C. for 1 hr, after filtered, the filtrate was concentrated to give 2-(1-methyl-1H-1,2,4-triazol-5-yl)acetic acid (752 g, crude) as a black brown gum.

### 3,4-Dichloro-N′-hydroxybenzimidamide

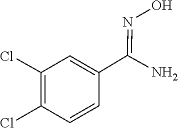 [SMILES: N/C(=N\O)c1ccc(Cl)c(Cl)c1]

To the mixture of 3,4-dichlorobenzonitrile (300 g, 1.74 mol, 1.0 eq) in EtOH (1.5 L) was added hydroxylamine (133 g, 1.92 mol, 1.10 eq, HCl) and DIEA (270 g, 2.09 mol, 365 mL, 1.20 eq) at 20-30° C., then the mixture was stirred at 20° C. for 3 hours. TLC (PE:EA=5:1, R_{f}=0.3) showed the reaction was complete. The mixture was concentrated under vacuum. The residue was diluted with EtOAC (2.00 L) and washed with water (2.00 L) and brine (2.00 L). The organic layer was concentrated to afford 3,4-dichloro-N′-hydroxybenzimidamide (357 g, 99.8% yield) as an off-white solid.

### Tert-butyl 4-(3-(3,4-dichlorophenyl)-1,2,4-oxadiazol-5-yl)piperidine-1-carboxylate

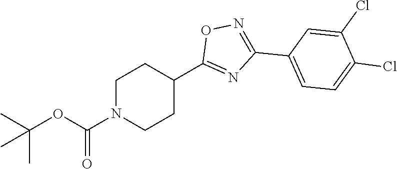 [SMILES: CC(C)(C)OC(=O)N1CCC(c2nc(-c3ccc(Cl)c(Cl)c3)no2)CC1]

To a solution of 1-(tert-butoxycarbonyl)piperidine-4-carboxylic acid (399 g, 1.74 mol, 1.0 eq) in DMF (1.6 L) was added CDI (296 g, 1.83 mol, 1.05 eq). The mixture was stirred at 25° C. for 1 hour, then 3,4-dichloro-N′-hydroxybenzimidamide (357 g, 1.74 mol, 1.0 eq) was added. The mixture was stirred at 25° C. for 2 hours, and then stirred at 100° C. for another 3 hours. TLC (PE:EA=5:1, R_{f}=0.7) showed the reaction was complete. The reaction mixture was poured into H₂O (5.0 L), after stirred at 20° C. for 2 hrs, the mixture was filtered to give compound tert-butyl 4-(3-(3,4-dichlorophenyl)-1,2,4-oxadiazol-5-yl)piperidine-1-carboxylate (512 g, 70.7% yield) as a white solid.

### 3-(3,4-Dichlorophenyl)-5-(piperidin-4-yl)-1,2,4-oxadiazole

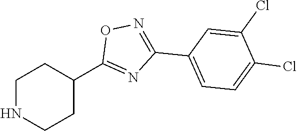 [SMILES: Clc1ccc(-c2noc(C3CCNCC3)n2)cc1Cl]

To a solution of 4-(3-(3,4-dichlorophenyl)-1,2,4-oxadiazol-5-yl)piperidine-1-carboxylate (252 g, 605 mmol, 1.0 eq) was added HCl/dioxane (4 M, 605 mL, 4.0 eq). The mixture was stirred at 25° C. for 1 hour. LC/MS showed 3-(3,4-dichlorophenyl)-5-(piperidin-4-yl)-1,2,4-oxadiazole was consumed. The mixture was filtered under vacuum to give 3-(3,4-dichlorophenyl)-5-(piperidin-4-yl)-1,2,4-oxadiazole (HCl, 411 g, crude) as a white solid.

### 1-(4-(3-(3,4-Dichlorophenyl)-1,2,4-oxadiazol-5-yl)piperidin-1-yl)-2-(1-methyl-1H-1,2,4-triazol-5-yl)ethan-1-one (mCLB073)

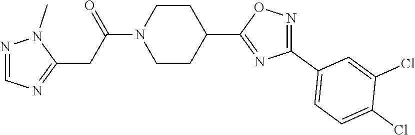 [SMILES: Cn1ncnc1CC(=O)N1CCC(c2nc(-c3ccc(Cl)c(Cl)c3)no2)CC1]

The mixture of compound 3-(3,4-dichlorophenyl)-5-(piperidin-4-yl)-1,2,4-oxadiazole (411 g, 1.23 mol, 1.0 eq, HCl), 2-(1-methyl-1H-1,2,4-triazol-5-yl)acetic acid (347 g, 2.46 mol, 2.0 eq), EDCI (471 g, 2.46 mol, 2.0 eq), DIEA (794 g, 6.14 mol, 1.07 L, 5.0 eq) and HOBt (332 g, 2.46 mol, 2.00 eq) in DCM (2.45 L) was stirred at 20° C. for 20 hrs. TLC (EA: MeOH=10:1, R_{f}=0.3) showed the reaction was complete. The mixture was washed with water (3.0 L), brine (2.0 L), then concentrated to give a residue. The residue was purified by silica gel chromatography (EtOAc/EtOH=1/0˜10/1), then slurried in n-hexane (1.00 L) and EtOH (1.00 L) at 60° C. for 2 hours to afford 1-(4-(3-(3,4-dichlorophenyl)-1,2,4-oxadiazol-5-yl)piperidin-1-yl)-2-(1-methyl-1H-1,2,4-triazol-5-yl)ethan-1-one (502 g, 39.4% yield) as an off-white solid.

LC/MS [ESI, M+1]: 421.1.

¹H NMR (500 MHz, CDCl₃) δ 8.16 (d, J=3.0 Hz, 1H), 8.02 (s, 1H), 7.89 (dd, J=2.0, 8.2 Hz, 1H), 7.55 (d, J=8.3 Hz, 1H), 4.37 (dt, J=4.0, 13.3 Hz, 1H), 4.26-4.17 (m, 3H), 3.99 (s, 3H), 3.48-3.43 (m, 1H), 3.32-3.26 (m, 1H), 3.13-3.07 (m, 1H), 2.28-2.24 (m, 1H), 2.21-2.16 (m, 1H), 2.02-1.87 (m, 2H); ¹³C NMR (500 MHz, CDCl₃) δ 18.55, 166.24, 163.27, 148.76, 146.24, 135.13, 132.90, 130.55, 128.85, 126.10, 126.00, 45.02, 40.49, 36.19, 33.43, 30.83, 28.95, 28.18.

### Example 4. 1-(4-(3-(4-fluorophenyl)isoxazol-5-yl)piperidin-1-yl)-2-(4-methyl-1,2,5-oxadiazol-3-yl)ethan-1-one (mCIV896)

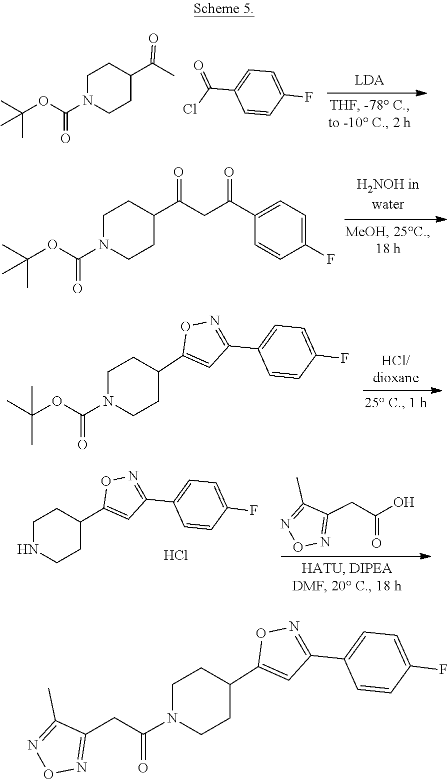

### tert-butyl 4-(3-(4-fluorophenyl)-3-oxopropanoyl)piperidine-1-carboxylate

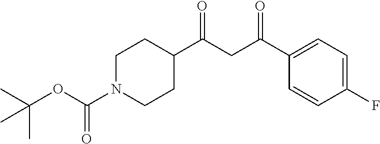 [SMILES: CC(C)(C)OC(=O)N1CCC(C(=O)CC(=O)c2ccc(F)cc2)CC1]

To a solution of tert-butyl 4-acetylpiperidine-1-carboxylate (250 mg, 1.1 mmol) in anhydrous THE (5 mL) at −78° C. under argon was added lithium diisopropylamide (2.0 M in heptane, 0.6 mL, 1.2 mmol) dropwise. The mixture was stirred at −78° C. for 30 minutes, and then 4-fluorobenzoyl chloride (180 mg, 1.2 mmol) in THE (0.5 mL) was added dropwise. Then, the reaction mixture was warmed to around −10° C., stirred at −10° C. for 2 h, and then the mixture was quenched with water (5 mL). The aqueous layer was extracted with ethyl acetate (3×10 mL); the combined organic layer was washed with 1.0 M HCl (5 mL), saturated NaHCO₃ (5 mL) and dried over Na₂SO₄. The solvent was removed under reduced pressure and the crude material was purified by flash chromatography to afford tert-butyl 4-(3-(4-fluorophenyl)-3-oxopropanoyl)piperidine-1-carboxylate (127 mg, 36% yield).

### 3-(4-Fluorophenyl)-5-(piperidin-4-yl)isoxazole

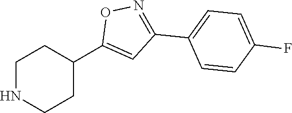 [SMILES: Fc1ccc(-c2cc(C3CCNCC3)on2)cc1]

To the mixture of tert-butyl 4-(3-(4-fluorophenyl)-3-oxopropanoyl)piperidine-1-carboxylate (105 mg, 0.3 mmol, 1.0 eq) in MeOH (2 mL) was added hydroxylamine (16M in H2O, 63 μL, 1.0 mmol). The reaction mixture was refluxed for 4 h and the solvent was removed under reduced pressure. The resulting residue was partitioned between ethyl acetate (10 mL) and water (10 mL). The aqueous layer was extracted with ethyl acetate (3×10 mL); the combined organic layer was washed with 1M HCl (10 mL) and dried over Na₂SO₄. The residue was purified by silica gel chromatography to afford tert-butyl 4-(3-(4-fluorophenyl)isoxazol-5-yl)piperidine-1-carboxylate. Then, the Boc protected isoxazole was dissolved in HCl/dioxane (4.0 M, 2 mL) and stirred at room temperature for 30 min. The solvent was removed under vacuum to afford 3-(4-fluorophenyl)-5-(piperidin-4-yl)isoxazole (HCl, 250 mg, crude) as a white solid.

### 1-(4-(3-(4-fluorophenyl)isoxazol-5-yl)piperidin-1-yl)-2-(4-methyl-1,2,5-oxadiazol-3-yl)ethan-1-one

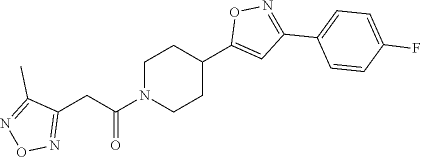 [SMILES: Cc1nonc1CC(=O)N1CCC(c2cc(-c3ccc(F)cc3)no2)CC1]

To a solution of 3-(4-Fluorophenyl)-5-(piperidin-4-yl)isoxazole (HCl salt, 56 mg, 0.2 mmol, 1.0 eq) in DMF (4 mL) was added HATU (114 mg, 0.3 mmol, 1.5 eq), 2-(4-methyl-1,2,5-oxadiazol-3-yl)acetic acid (34 mg, 0.24 mmol, 1.2 eq) and DIPEA (77.6 mg, 0.6 mmol, 104 μL, 3.0 eq). The mixture was stirred at 20° C. for 18 hours. The mixture was concentrated under vacuum and diluted with ethyl acetate (10 mL) and washed with water (2×10 mL). Then the organic layer was washed with brine (10 mL), dried over Na₂SO₄, and the mixture was filtered, and the filtrate was concentrated. The residue was purified by silica gel chromatography to afford 1-(4-(3-(4-fluorophenyl)-1,2,4-oxadiazol-5-yl)piperidin-1-yl)-2-(4-methyl-1,2,5-oxadiazol-3-yl)ethan-1-one (30 mg, 0.08 mmol, 40% yield) as white solid.

LC/MS [ESI, M+1]: m/z 371.46.

1H NMR (400 MHz, DMSO-D6) δ7.93-7.88 (m, 2H), 7.41-7.33 (m, 2H), 4.40-4.37 (m, 1H), 4.14-4.11 (m, 2H), 4.03-4.00 (m, 1H), 3.33-3.20 (m, 2H), 3.11-3.05 (m, 1H), 2.89-2.80 (m, 1H), 2.29 (s, 3H), 2.11-1.95 (m, 2H), 1.78-1.69 (m, 1H), 1.58-1.49 (m, 1H).

### Example 5. Synthesis of 1-(4-(2-(4-fluorophenyl)pyrimidin-5-yl)piperidin-1-yl)-2-(4-methyl-1,2,5-oxadiazol-3-yl)ethan-1-one (mCIV115)

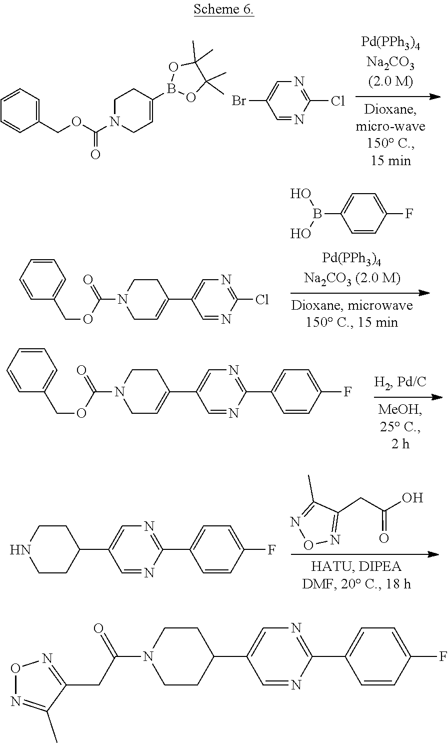

### Benzyl 4-(2-chloropyrimidin-5-yl)-3,6-dihydropyridine-1(2H)-carboxylate

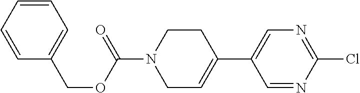 [SMILES: O=C(OCc1ccccc1)N1CC=C(c2cnc(Cl)nc2)CC1]

Benzyl 4-(4,4,5,5-tetramethyl-1,3,2-dioxaborolan-2-yl)-3,6-dihydropyridine-1(2H)-carboxylate (343 mg, 1.0 mmol, 1.0 eq), 5-bromo-2-chloropyrimidine (193 mg, 1.0 mmol, 1.0 eq), Pd(PPh₃)₄ (289 mg, 0.25 mmol, 0.25 eq), Na₂CO₃ (2.0 M in water, 0.6 mL, 1.2 mmol, 1.2 eq) and dioxane (10 ml) were added to microware vial with small stir bar. The reaction mixture was reacted at 150° C. in microwave reactor for 15 min. The reaction mixture was combined and filtered by syringe filter. Dilute with EtOAc and washed with brine, dry over Na₂SO₄, Evaporation. The residue was purified by silica gel chromatography to afford benzyl 4-(2-chloropyrimidin-5-yl)-3,6-dihydropyridine-1(2H)-carboxylate (236 mg, 0.72 mmol, 72% yield) as pale yellow solid.

### Benzyl 4-(2-(4-fluorophenyl)pyrimidin-5-yl)-3,6-dihydropyridine-1(2H)-carboxylate

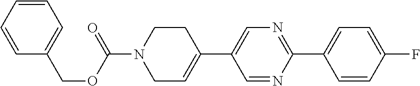 [SMILES: O=C(OCc1ccccc1)N1CC=C(c2cnc(-c3ccc(F)cc3)nc2)CC1]

Benzyl 4-(2-chloropyrimidin-5-yl)-3,6-dihydropyridine-1(2H)-carboxylate (66 mg, 0.2 mmol, 1.0 eq), (4-fluorophenyl)boronic acid (28 mg, 0.2 mmol, 1.0 eq), Pd(PPh₃)₄ (58 mg, 0.05 mmol, 0.25 eq), Na₂CO₃ (2.0 M in water, 0.12 mL, 0.24 mmol, 1.2 eq) and dioxane (3 ml) were added to microware vial with small stir bar. The reaction mixture was reacted at 150° C. in microwave reactor for 15 min. The reaction mixture was combined and filtered by syringe filter. Dilute with EtOAc and washed with brine, dry over Na₂SO₄, Evaporation. The residue was purified by silica gel chromatography to afford benzyl 4-(2-(4-fluorophenyl)pyrimidin-5-yl)-3,6-dihydropyridine-1(2H)-carboxylate (56 mg, 0.14 mmol, 72% yield) as pale yellow solid.

### 2-(4-Fluorophenyl)-5-(piperidin-4-yl)pyrimidine

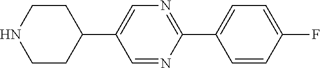 [SMILES: Fc1ccc(-c2ncc(C3CCNCC3)cn2)cc1]

To the mixture of benzyl 4-(2-(4-fluorophenyl)pyrimidin-5-yl)-3,6-dihydropyridine-1(2H)-carboxylate (56 mg, 0.14 mmol, 1.0 eq) in MeOH (5 mL) was added Palladium on carbon (Pd/C, 30 mg, 0.28 mmol, 2.0 eq). The reaction vial was sealed and bubbled with H2 gas for 2 hours at 25° C. The reaction mixture was filtered by syringe filter and evaporation. The crude of 2-(4-fluorophenyl)-5-(piperidin-4-yl)pyrimidine (34 mg, 0.13 mmol, 95%) yield was used directly for next step. 1-(4-(2-(4-fluorophenyl)pyrimidin-5-yl)piperidin-1-yl)-2-(4-methyl-1,2,5-oxadiazol-3-yl)ethan-1-one

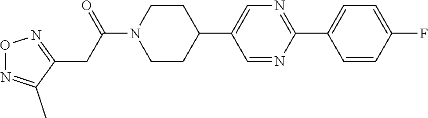 [SMILES: Cc1nonc1CC(=O)N1CCC(c2cnc(-c3ccc(F)cc3)nc2)CC1]

To a solution of 2-(4-Fluorophenyl)-5-(piperidin-4-yl)pyrimidine (26 mg, 0.1 mmol, 1.0 eq) in DMF (4 mL) was added HATU (57 mg, 0.15 mmol, 1.5 eq), 2-(4-methyl-1,2,5-oxadiazol-3-yl)acetic acid (17 mg, 0.12 mmol, 1.2 eq) and DIPEA (38.8 mg, 0.3 mmol, 52 μL, 3.0 eq). The mixture was stirred at 20° C. for 18 hours. The mixture was concentrated under vacuum and diluted with ethyl acetate (10 mL) and washed with water (2×10 mL). Then the organic layer was washed with brine (10 mL), dried over Na₂SO₄, and the mixture was filtered, and the filtrate was concentrated. The residue was purified by silica gel chromatography to afford 1-(4-(2-(4-fluorophenyl)pyrimidin-5-yl)piperidin-1-yl)-2-(4-methyl-1,2,5-oxadiazol-3-yl)ethan-1-one (19 mg, 0.05 mmol, 50% yield) as white solid.

LC/MS [ESI, M+1]: m/z 382.45.

¹H NMR (400 MHz, DMSO-D6) δ 8.84 (s, 2H), 8.43-8.40 (m, 2H), 7.37-7.32 (m, 2H), 4.55-4.51 (m, 1H), 4.15-4.13 (m, 3H), 3.28-3.20 (m, 1H), 2.97-2.91 (m, 1H), 2.75-2.67 (m, 1H), 2.29 (s, 3H), 1.95-1.86 (m, 2H), 1.84-1.74 (m, 1H), 1.67-1.58 (m, 1H).

### Bioassays

Extracellular Activity Against M. tuberculosis in Cholesterol Media (Alamar Blue Reduction Assay)

To determine compound potency against Mtb (Erdman wild type, CDC1551 wild type, or Rv1625c transposon mutant (Tn::rv1625c)) in liquid culture an Alamar Blue reduction assay was used as described. For inhibition assays in media containing cholesterol as the carbon source (7H12-Cholesterol) Mtb was first cultured to an (OD600 of 0.4) in 7H12 media (7H9 base, 0.1% casamino acids, 100 mM 2-morpholinoethanesulfonic acid pH 6.6) and 0.1% (wt/vol) acetate as the carbon source and 0.05% tyloxapol. Cholesterol was added to the assay culture media at a final concentration of 100 μM as ethanol/tyloxapol micelles according to Lee, W., et al. J Biol Chem, 2013. 288(10): p. 6788-800. For the inhibition assay, bacteria were washed in PBS tyloxapol 0.05% twice and 1.0×10⁶ bacteria were added to 96-well microplates containing 7H12-Cholesterol media to a final volume of 200 μl containing the experimental compounds or controls. Compounds were tested in a 12-point dose-titration, serially diluted 1:2 starting at either 25 μM or 10 μM. The microplates were incubated for 10 days in humidified, sealed plastic bags at 37° C. To quantify bacterial proliferation 40 μl of an Alamar Blue solution 50% was added to each well and the plates were re-incubated at 37° C. for 16 hr. Alamar Blue reduction was quantified using an Envision Multilabel plate reader (Perkin Elmer) with λex=492 nm and λem=595 nm. All assay plates contained DMSO and 10 μM rifampicin control wells and percent inhibition for the experimental compounds was calculated. EC₅₀ values were determined by fitting the percent inhibition dose response curves in Prism (GraphPad Software), using a sigmoidal variable slope fit with the maximum % activity and the minimum % activity fixed at 100% and 0%, respectively. Corresponding data are reported in Table 1 above.

Intramacrophage Activity of mCLB073 in THP-1 Cells

THP-1 macrophages were obtained from ATCC TIB202 and maintained at 37° C. with 5% CO₂. The Cells were passaged in RPMI 1640 supplemented with 10% fetal bovine serum (FBS) and 50 μM 2-Mercaptoethanol (Gibco). 100 mL of 10⁵ cells/mL were differentiated by resuspending them in propagation media plus 50 nM of Phorbol myristate acetate (PMA) and incubating at 37° C. with 5% CO₂ overnight. The mid-log phase Mtb H37Rv strain carrying the lux-CDABE operon (Mtb-lux) was then diluted in propagation media and added to flask containing differentiated THP-1 at MOI of 1:1 for 24 h. The infected cells were washed twice with PBS and then resuspended in fresh media. The cells were then scrapped from the flask counted and added to drug spotted plates at final concentration of 10⁵ cells/well. Luminescence was recorded in CLARIOstar Plus (BMG Labtech) microplate reader at day 3 and data normalized to negative control (DMSO) minus inhibitor (Rifampicin 10 μM) on Genedata Screener Analyzer (v16.0.8.) Each drug concentration was tested in triplicates.

mCLB073 was tested for its ability to inhibit growth of Mtb residing inside PMA-differentiated THP-1 cells. An EC₅₀ value of 0.04 μM was determined for mCLB073. There was no significant variation in activity between replicates (FIG. 10). The recently FDA-approved anti-TB drug Pretomanid was used as a positive control. Representative curves are shown in FIG. 10 of the non-linear regression curve fit of the drug concentration versus the normalized luminescence signal. Of note, mCLB073 demonstrated a partial response based on S_{inf} values (indicating maximum inhibition). The S_{inf} for mCLB073 was 76.8%, whereas Pretomanid showed the maximal response.

Intramacrophage Activity of mCLB073 in J774.1 Cells

The intracellular activity was evaluated in J774.1 (ATCC 113-67TM) cells as previously described (Sukheja et al., 2017). J774A.1 (ATCC) were grown in DMEM (Gibco) supplemented with 10% heat inactivated FBS, HEPES 10 mM (Gibco) and Sodium pyruvate 1 mM (Gibco) in a 5% CO₂ humidified atmosphere at 37-C. For macrophage infection, cells were washed once in PBS (Gibco), scraped, and counted with Trypan Blue and adjusted to final density of 10⁵ cells/ml The mid-log phase Mtb-expressing luciferase strain was then diluted in DMEM and added to the J774.1 flask at MOI of 1:1 for 24 h. The infected cells were washed twice with PBS and then resuspended in DMEM media. The cells were then scrapped from the flask and were counted and added to drug spotted plates at final concentration of 10⁵ cells/well. On day 3 luminescence was recorded in CLARIOstar Plus (BMG Labtech) microplate reader and data normalized to negative control (DMSO) minus inhibitor (Rifampicin 10 uM) on Genedata Screener Analyzer (v16.0.8). Each drug concentration was tested in triplicates.

mCLB073 was tested for its ability to inhibit growth of Mtb residing inside J774.1 macrophages. An EC₅₀ value of 0.05 μM was determined for mCLB073. There was no significant variation in activity between replicates (FIG. 11). The recently FDA-approved anti-TB drug Pretomanid was used as a positive control. Representative curves are shown in FIG. 11 of the non-linear regression curve fit of the drug concentration versus the normalized luminescence signal. Of note, mCLB073 demonstrated a partial response based on S_{inf} values (indicating maximum inhibition). The S_{inf} for mCLB073 was 42.1%, whereas Pretomanid showed the maximal response.

### MIC Determination in Mtb H37Rv

The minimum inhibitory concentration (MIC) of the test compounds were determined using ten-point, two-fold serial dilutions in 7H12-Cholesterol media using Microdilution Alamar Blue Assay (MABA) as previously described (Cho et al., 2015; VanderVen et al., 2015). Briefly, Mycobacterial cells at the mid-logarithmic phase of growth were diluted (1:1,000). Twenty μl of this dilution was added to each well containing 2-fold serially diluted test compounds and incubated for 7 days at 37° C. Alamar Blue (Invitrogen) reagent (4 μL per well) was added along with 20% Tween 80 (2.5 μL per well, Sigma Aldrich) to evaluate bacterial cell viability. Plates were read 24 h after adding Alamar Blue at absorbance 570 nm with a reference wavelength of 600 nm.

Two batches of mCLB073 were tested for in vitro inhibition of Mtb in cholesterol media. MIC90 values ranged from 0.19 μM to 0.31 μM with an average value at 0.28 μM. There was no significant variation in activity between replicates (FIG. 12). Rifampicin, the most potent first-line anti-TB drug, was used as a positive control. A representative dose-response curve is shown in FIG. 12.

In a surrogate assay of intramacrophage activity, mCLB073 demonstrated an MIC90 of 0.31 mM against H37Rv, a virulent strain of Mtb, and an IC50 value of 0.03 mM against the attenuated strain H37Ra.

### IC50 Determination in Mtb H37Ra

To determine compound potency against Mtb H37Ra in liquid culture an Alamar Blue reduction assay was used as described (VanderVen et al., 2015). For inhibition assays in media containing cholesterol as the carbon source (7H12-Cholesterol) Mtb was first cultured to an (OD600 of 0.4) in 7H12 media (7H9 base, 0.1% casamino acids, 100 mM 2-morpholinoethanesulfonic acid pH 6.6) and 0.1% (wt/vol) acetate as the carbon source and 0.05% tyloxapol. Cholesterol was added to the assay culture media at a final concentration of 100 μM as ethanol/tyloxapol micelles according to Lee et al., 2013. For the inhibition assay, bacteria were washed in PBS tyloxapol 0.05% twice and 1.0′10⁶ bacteria were added to 384-well microplates containing 7H12-Cholesterol media to a final volume of 20 L containing the experimental compounds or controls. Compounds were tested in a 9-point dose-titration, serially diluted 1:3 starting at 30 μM. The microplates were incubated for 10 days in humidified, sealed plastic bags at 37° C. To quantify bacterial proliferation 10 L of an Alamar Blue solution 50% was added to each well and the plates were re-incubated at 37° C. for 16 hr. Alamar Blue reduction was quantified using a PHERAstar plate reader (BMG Labtech) with λex=492 nm and λem=595 nm. All assay plates contained DMSO and 10 μM rifampicin control wells and percent inhibition for the experimental compounds was calculated. IC₅₀and IC₉₀ values were determined by using the Smart Fit function in Genedata Screener Analyzer (v16.0.8) with the maximum % activity and the minimum % activity fixed at 100% and 0%, respectively.

mCLB073, related compound mCIS635, and control compounds Rifampicin and Isoniazid were tested against the attenuated BSL2 Mtb strain H37Ra in cholesterol media. The measured half maximal concentration (IC50) of mCLB073 was 30.1 nM (FIG. 13).

Cytotoxicity of mCLB073 and Related Compounds

Four batches of mCLB073 were tested in two separate assays for in vitro growth inhibition of two mammalian cell lines: HEK293T and HepG2. The half maximal cytotoxic concentration (CC₅₀) is reported for each compound or batch in both cell lines. Related compounds and controls were also included in each assay.

### HEK293T Cytotoxicity Assay

HEK293T (ATCC) were grown in DMEM (Gibco Cat #11965-092) supplemented with 10% heat-inactivated FBS, Pen Strep (Gibco) 1× in a 5% CO₂ humidified atmosphere at 37° C. Cells were washed once with DPBS 1× (Gibco), incubated for less than 5 minutes at room temperature in TrypLE solution (Gibco), washed once in propagation medium to remove trypsin, and counted with Trypan Blue. 5 μL of cell suspension (7.5×10⁴ cells/well) was dispensed into 1536-well plates containing serial 3-fold drug dilution ranging from 40 μM to 2 nM and incubated for 3 days in a 5% CO₂ humidified atmosphere at 37° C. Cell Titer-Glo® Luminescent Cell Viability Assay (Promega) reagent was diluted 1:1 with water and then 2 μL were added to plates. Luminescence was measured in a PHERAStar plate reader (BMG Labtech) and data normalized to negative control (DMSO) minus inhibitor (Puromycin 10 μM). Dose-response curves were fit using the Smart Fit function in Genedata Screener Analyzer (v16.0.8).

### HEPG2 Cytotoxicity Assay

HepG2 (ATCC) were grown in DMEM (Gibco Cat #31053-028) supplemented with 10% heat-inactivated FBS, Pen Strep (Gibco) 1×, Glutamax 1× (Gibco), Sodium pyruvate 1 mM (Gibco) in a 5% CO₂ humidified atmosphere at 37° C. Cells were washed twice with DPBS 1× (Gibco), incubated ten minutes at 37° C. in TrypLE solution (Gibco), washed once in propagation medium to remove trypsin and counted with Trypan Blue. 5 μL of cell suspension (5.0×10⁴ cells/well) was dispensed into 1536-well plates, containing serial 3-fold drug dilution ranging from 40 μM to 2 nM and incubated for 3 days in a 5% CO₂ humidified atmosphere at 37° C. Cell Titer-Glo® Luminescent Cell Viability Assay (Promega) reagent was diluted 1:1 with water and then 2 μL were added to plates. Luminescence was measured in a PHERAStar plate reader (BMG Labtech) and data normalized to negative control (DMSO) minus inhibitor (Puromycin 10 μM). Dose-response curves were fit using the Smart Fit function in Genedata Screener Analyzer (v16.0.8).

Determining Extracellular Activity Against M. tuberculosis in Cholesterol Media

To determine compound potency against Mtb (Erdman wild type, CDC1551 wild type, or Rv1625c transposon mutant (Tn::rv1625c)[1]) in liquid culture an Alamar Blue reduction assay was used as described [2, 3]. For inhibition assays in media containing cholesterol as the carbon source (7H12-Cholesterol)Mtb was first cultured to an (OD600 of 0.4) in 7H12 media (7H9 base, 0.1% casamino acids, 100 mM 2-morpholinoethanesulfonic acid pH 6.6) and 0.1% (wt/vol) acetate as the carbon source and 0.05% tyloxapol [4]. Cholesterol was added to the assay culture media at a final concentration of 100 μM as ethanol/tyloxapol micelles according to [5]. For the inhibition assay, bacteria were washed in PBS tyloxapol 0.05% twice and 1.0×10⁶ bacteria were added to 96-well microplates containing 7H12-Cholesterol media to a final volume of 200 μl containing the experimental compounds or controls. Compounds were tested in a 12-point dose-titration, serially diluted 1:2 starting at either 25 μM or 10 μM. The microplates were incubated for 10 days in humidified, sealed plastic bags at 37° C. To quantify bacterial proliferation 40 μl of an Alamar Blue solution 50% was added to each well and the plates were re-incubated at 37° C. for 16 hr. Alamar Blue reduction was quantified using an Envision Multilabel plate reader (Perkin Elmer) with λex=492 nm and λem=595 nm. All assay plates contained DMSO and 10 μM rifampicin control wells and percent inhibition for the experimental compounds was calculated. IC50 values were determined by fitting the percent inhibition dose response curves in Prism (GraphPad Software), using a sigmoidal variable slope fit with the maximum % activity and the minimum % activity fixed at 100% and 0%, respectively.

Summary of activity of mCLB073 in 7H12-Cholesterol media against Mtb Erdman wild-type, CDC1551 wild-type, or the Rv1625c transposon mutant (CDC1551 Tn::rv1625c) is shown in FIG. 14. For each experiment, the compound batch is identified, and n=1 for all values.

Determining Activity of mCLB073 in a Hu Coats 100-Day Old Culture Model

Test the potency of mCLB073 as a monotherapy or in combination with pretomanid and bedaquiline in an advanced in vitro model of non-replicating M. tuberculosis.

M. tuberculosis was grown in 10 mL of 7H9 medium supplemented with OADC and 0.025% Tween 80 in 25-mL screw cap Nunc tubes in a static incubator for 100 days. To disperse clumps, the cultures were sonicated using a low-intensity water bath sonicator (Branson 1800) by applying 3 pulses of 2 min of sonication and 1 min of rest followed by vortex mixing. The cidal activity of each compound alone and each combination against 100-day-old bacilli was determined by exposing each tube to the respective compounds for 5 days at 37° C. followed by three washes with PBS to remove drug carryover and then measuring CFU by plating appropriate dilutions on 7H11-OADC-agar plates.

Efficacy of NixTB combination (Pretomanid, P; Bedaquiline, B; Linezolid, L) either alone or by replacing Linezolid with mCLB073 at 20 μM and 50 μM was tested in the Hu Coats 100-day-old culture model. Rv1625c activator mCLB073 had no efficacy when tested individually in this model (FIG. 15A). However, combination of mCLB073 with Pretomanid and Bedaquiline (with all compounds at 20 μM) produced a one-log improvement in cidal activity compared to the NixTB combination. Moreover, when mCLB073 combination with Pretomanid and Bedaquiline was increased to 50 μM, it led to complete sterilization of non-replicating Mtb, which was two log units better than the NixTB combination (FIG. 15B).

Testing was repeated using a fixed concentrations of Pretomanid and Bedaquiline at their estimated physiological concentration in human dosing (Pretomanid=20.87 μM and Bedaquiline=2.25 μM). The PB combination was tested either with mCLB073 or Linezolid at three concentrations (20 μM, 8 μM and 1 μM). It was observed that mCLB073 in combination was comparable in activity to NixTB combination (FIG. 15C).

This was an exploratory study to investigate whether the in vivo potentiation of Rv1625c activators with Pretomanid and Bedaquiline could be recapitulated in vitro. The Hu Coates model deprives Mtb of oxygen and nutrients for a prolonged period to induce the culture to enter into a non-replicating state that is presumed to mimic the Mtb persisters present in human disease. This method is more time consuming and lower throughput than other in vitro models, so the number of combinations and repeats were limited. However, the team was surprised to see the Rv1625c activator mCLB073 show potentiation when combined with PB.

The initial study blindly dosed all the compounds at a single concentration: 1, 5, 20 and 50 μM. At 50 μM, the addition of LIN or mCLB073 showed superior CFU reduction to PB (FIG. 15B; black bar).

A more thoughtful study was repeated in which the estimated plasma concentrations of P and Ba were used in combination with variable concentrations (1, 8, and 20 μM) of LIN or mCLB073. Even at 1 μM, the Rv1625c activators show a 3-10× reduction in CFU compared to PBL. Interestingly, this model may be predictive of in vivo potentiation of compounds in the chronic Balb/c infection model.

Combination Testing of mCLB073 with Known Anti-TB Drugs

To test for interactions between mCLB073 and known anti-tubercular drugs in combination in an in vitro growth assay, a checkerboard assay to test for interactions between mCLB073 and drugs with known anti-TB activity using Alamar Blue as a viability marker (Lechartier et al., 2012) was used. Drug combinations were categorized based on fractional inhibitory concentration (FIC) index of FIC≤0.5 as synergistic, 0.5<FIC≤1 as additive, 1<FIC≤4 as no interaction and FIC ≥4 as antagonism (Hall et al., 1983).

FIG. 16A shows a schematic representation of checkerboard assay. FIG. 16B shows the formula for calculating FIC index. FIG. 16C shows a table with interaction category based on FIC index value (adapted from Emery Pharma).

Performed were synergy studies between mCLB073 and nine anti-TB drugs: Moxifloxacin (MOXI), Isoniazid (INH), Rifampicin (RIF), Streptomycin (STR), Levofloxacin (LEV), Linezolid (LIN), Ethionamide (ETH), Pretomanid (PA-824), and Bedaquiline (BDQ). A checkerboard titration in a microtiter plate was used as previously described (Hsieh et al., 1993). FIGS. 16A-C shows the FIC index and interpretation of each combination. FIG. 16 D shows the synergy between mCLB073 and anti-tubercular drugs.

It was observed that mCLB073 was well-tolerated with known anti-TB drugs and did not display any antagonism. In addition, an additive effect of mCLB073 with RIF, STR, PA-824, and BDQ was observed.

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

1. A compound of Formula I or Formula II, 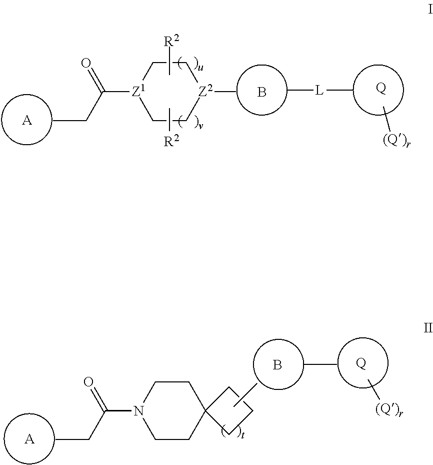
wherein:
A is selected from the group consisting of: 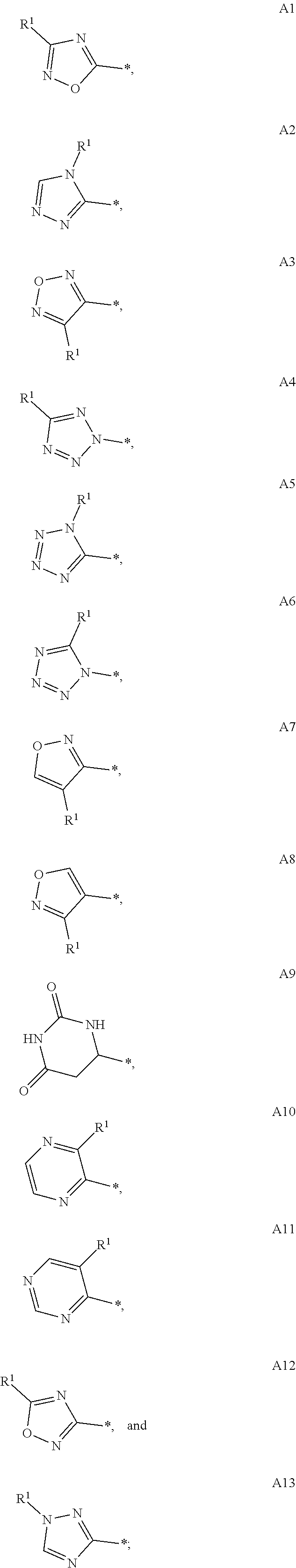
each R¹ is independently H or (C₁-C₆)alkyl;
Z¹ is CH or N;
u is 0 to 2;
v is 0 to 2;
both R² are absent or both R² together form a (C₁-C₂)alkylenyl bridge between two ring carbon atoms, provided that u and v are not both 0;
Z² is CH, CR³, or N;
R³ is OH, CF₃, or (C₁-C₆)alkyl;
t is 0 or 1;
B is selected from the group consisting of: 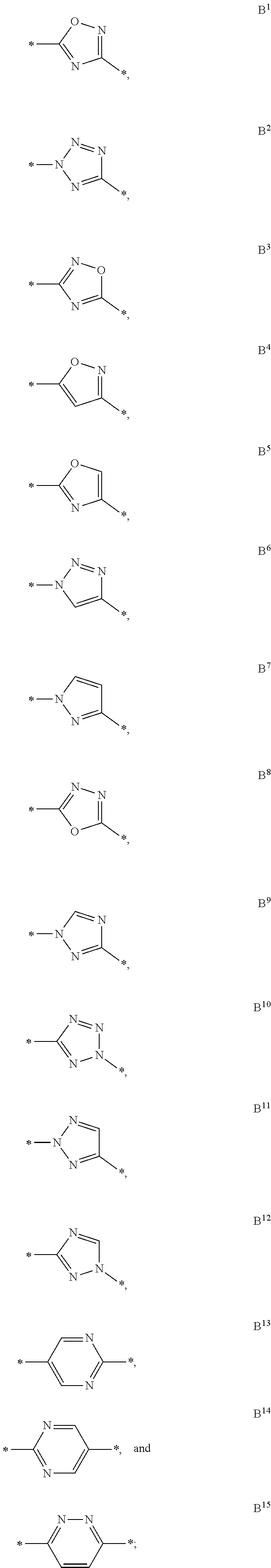
L is a bond;
or if A is A³ and B is B¹, B², or B⁶, L may be —C(═O)—, —C(═O)N(R⁹)—, —CH₂—, or —O—:
R⁹ is H, (C₁-C₆)alkyl, or (C₁-C₆)heteroalkyl;
Q is 3- to 8-membered monocyclic, 6- to 16-membered bicyclic, 4- to 14-membered fused bicyclic, or 5- to 11-membered spirocyclic cycloalkyl or heterocycloalkyl, Ph, or 5- to 8-membered heteroaryl, each optionally substituted with (Q′)r;
or if A is A³ and B is Bi, B², or B⁶, Q may be (C₁-C₆)alkyl or (C₁-C₆)heteroalkyl;
each Q′ is independently halo, halo (C₁-C₆) alkyl, haloalkoxy, —CN, (C₁-C₆)alkyl, (C₂-C₆)alkenyl, (C₂-C₆)alkynyl, (C₁-C₆)heteroalkyl, OH, oxo, amino, amido, imino, (C₁-C₆)alkyl ester, carboxyl, —SF₅, 3- to 8-membered cycloalkyl, 3- to 8-membered heterocycloalkyl, 3- to 7-membered spirocyclic cycloalkyl or heterocycloalkyl, aryl, or 5- to 6-membered heteroaryl; and
r is 0-5;
with the proviso that the compound of Formula I is not V-9, 2-(4-methyl-1,2,5-oxadiazol-3-yl)-1-(4-(5-(p-tolyl)-2H-tetrazol-2-yl)piperidin-1-yl)ethan-1-one; 2-(4-methyl-1,2,5-oxadiazol-3-yl)-1-(4-(5-(pyridin-4-yl)-1,2,4-oxadiazol-3-yl)piperidin-1-yl)ethan-1-one; 2-(4-methyl-1,2,5-oxadiazol-3-yl)-1-(4-(5-(6-methylpyridin-3-yl)-1,2,4-oxadiazol-3-yl)piperidin-1-yl)ethan-1-one; 2-(4-methyl-1,2,5-oxadiazol-3-yl)-1-(3-(3-phenyl-1,2,4-oxadiazol-5-yl)piperidin-1-yl)ethan-1-one; 1-(3-(5-cyclopropylpyrimidin-2-yl)azetidin-1-yl)-2-(4-methyl-1,2,5-oxadiazol-3-yl)ethan-1-one; 8) 1-(4-(5-(furan-3-yl)-1,3,4-oxadiazol-2-yl)piperidin-1-yl)-2-(2H-tetrazol-2-yl)ethan-1-one; 2-(2H-tetrazol-2-yl)-1-(3-(5-(thiophen-3-yl)-1,3,4-oxadiazol-2-yl)piperidin-1-yl)ethan-1-one; 2-(1H-tetrazol-5-yl)-1-(4-(3-(thiophen-2-yl)-1,2,4-oxadiazol-5-yl)piperidin-1-yl)ethan-1-one; 2-(5-isopropyl-1H-tetrazol-1-yl)-1-(4-(3-phenyl-1,2,4-oxadiazol-5-yl)piperidin-1-yl)ethan-1-one; 1-(3-(3-phenyl-1,2,4-oxadiazol-5-yl)piperidin-1-yl)-2-(1H-tetrazol-1-yl)ethan-1-one; 1-(3-(5-(1-(cyclopropylmethyl)pyrrolidin-2-yl)-1,2,4-oxadiazol-3-yl)azetidin-1-yl)-2-(1H-tetrazol-1-yl)ethan-1-one; 1-(3-(4-phenyl-1H-1,2,3-triazol-1-yl)azetidin-1-yl)-2-(1H-tetrazol-1-yl)ethan-1-one; 1-(3-(4-phenyl-1H-1,2,3-triazol-1-yl)pyrrolidin-1-yl)-2-(1H-tetrazol-1-yl)ethan-1-one; 1-(3-(5-cyclopropylpyrimidin-2-yl)azetidin-1-yl)-2-(1H-tetrazol-1-yl)ethan-1-one; 1-(4-(6-cyclopropylpyridazin-3-yl)piperazin-1-yl)-2-(1H-tetrazol-1-yl)ethan-1-one; 1-(4-(6-(1H-1,2,4-triazol-1-yl)pyridazin-3-yl)piperazin-1-yl)-2-(1H-tetrazol-1-yl)ethan-1-one; 1-(3-(3-cyclopropyl-1,2,4-oxadiazol-5-yl)pyrrolidin-1-yl)-2-(isoxazol-3-yl)ethan-1-one; 2-(isoxazol-3-yl)-1-(3-(3-phenyl-1,2,4-oxadiazol-5-yl)azetidin-1-yl)ethan-1-one; or 1-(3-(3-isobutyl-1,2,4-oxadiazol-5-yl)piperidin-1-yl)-2-(4-methyl-1,2,5-oxadiazol-3-yl)ethan-1-one; or any pharmaceutically acceptable salts or any stereochemically isomeric forms thereof.

2-60. (canceled)

61. A compound having any one of the formulae selected from the group consisting of:
6-(2-(4-(3-(4-fluorophenyl)-1,2,4-oxadiazol-5-yl)piperidin-1-yl)-2-oxoethyl)pyrimidine-2,4(1H,3H)-dione;
1-(4-(5-(4-fluorophenyl)-2H-tetrazol-2-yl)piperidin-1-yl)-2-(pyrazin-2-yl)ethan-1-one;
6-((4-(3-(3,4-dichlorophenyl)-1,2,4-oxadiazol-5-yl)piperidin-1-yl)methyl)pyrimidine-2,4(1H,3H)-dione;
1-(4-(5-(4-fluorophenyl)-2H-tetrazol-2-yl)piperidin-1-yl)-2-(pyrazin-2-yl)ethan-1-one;
1-(4-(3-(3,4-dichlorophenyl)-1,2,4-oxadiazol-5-yl)piperidin-1-yl)-2-(3-isopropyl-1,2,4-oxadiazol-5-yl)ethan-1-one;
1-(4-(3-(4-fluorophenyl)-1,2,4-oxadiazol-5-yl)piperidin-1-yl)-2-(3-isopropyl-1,2,4-oxadiazol-5-yl)ethan-1-one;
1-(4-(3-cyclohexyl-1,2,4-oxadiazol-5-yl)piperidin-1-yl)-2-(5-methylpyrimidin-4-yl)ethan-1-one;
1-(4-(3-(3,4-dichlorophenyl)-1,2,4-oxadiazol-5-yl)piperidin-1-yl)-2-(5-methylpyrimidin-4-yl)ethan-1-one;
1-(4-(3-(4-fluorophenyl)-1,2,4-oxadiazol-5-yl)piperidin-1-yl)-2-(5-methylpyrimidin-4-yl)ethan-1-one;
1-(4-(3-(3,4-dichlorophenyl)-1,2,4-oxadiazol-5-yl)piperidin-1-yl)-2-(4-methylisoxazol-3-yl)ethan-1-one;
1-(4-(3-(4-fluorophenyl)-1,2,4-oxadiazol-5-yl)piperidin-1-yl)-2-(4-methylisoxazol-3-yl)ethan-1-one;
1-(4-(3-(4-fluorophenyl)-1,2,4-oxadiazol-5-yl)piperidin-1-yl)-2-(5-methyl-1,2,4-oxadiazol-3-yl)ethan-1-one;
1-(4-(3-cyclohexyl-1,2,4-oxadiazol-5-yl)piperidin-1-yl)-2-(3-methylpyrazin-2-yl)ethan-1-one;
1-(4-(3-(3,4-dichlorophenyl)-1,2,4-oxadiazol-5-yl)piperidin-1-yl)-2-(3-methylpyrazin-2-yl)ethan-1-one;
1-(4-(3-(4-fluorophenyl)-1,2,4-oxadiazol-5-yl)piperidin-1-yl)-2-(3-methylpyrazin-2-yl)ethan-1-one;
1-(4-(3-(4-chloro-3-fluorophenyl)-1,2,4-oxadiazol-5-yl)piperidin-1-yl)-2-(5-methyl-2H-tetrazol-2-yl)ethan-1-one;
1-(4-(3-(4-chloro-3-fluorophenyl)-1,2,4-oxadiazol-5-yl)piperidin-1-yl)-2-(5-methyl-1H-tetrazol-1-yl)ethan-1-one;
1-(4-(3-(bicyclo[2.2.1]heptan-2-yl)-1,2,4-oxadiazol-5-yl)piperidin-1-yl)-2-(3-methyl-1,2,4-oxadiazol-5-yl)ethan-1-one;
1-(4-(3-(1,4-dioxaspiro[4.5]decan-8-yl)-1,2,4-oxadiazol-5-yl)piperidin-1-yl)-2-(3-methyl-1,2,4-oxadiazol-5-yl)ethan-1-one;
1-(4-(3-cyclopentyl-1,2,4-oxadiazol-5-yl)piperidin-1-yl)-2-(3-methyl-1,2,4-oxadiazol-5-yl)ethan-1-one;
1-(4-(5-cyclopentyl-2H-tetrazol-2-yl)piperidin-1-yl)-2-(3-methyl-1,2,4-oxadiazol-5-yl)ethan-1-one;
1-(4-(3-(bicyclo[4.1.0]heptan-2-yl)-1,2,4-oxadiazol-5-yl)piperidin-1-yl)-2-(3-methyl-1,2,4-oxadiazol-5-yl)ethan-1-one;
1-(4-(3-cyclohexyl-1,2,4-oxadiazol-5-yl)piperidin-1-yl)-2-(3-methyl-1,2,4-oxadiazol-5-yl)ethan-1-one;
1-(4-(5-cyclohexyl-2H-tetrazol-2-yl)piperidin-1-yl)-2-(3-methyl-1,2,4-oxadiazol-5-yl)ethan-1-one;
1-(4-(3-cyclohexyl-1,2,4-oxadiazol-5-yl)-4-hydroxypiperidin-1-yl)-2-(3-methyl-1,2,4-oxadiazol-5-yl)ethan-1-one;
1-(4-(3-(bicyclo[2.2.1]heptan-1-yl)-1,2,4-oxadiazol-5-yl)piperidin-1-yl)-2-(3-methyl-1,2,4-oxadiazol-5-yl)ethan-1-one;
2-(3-methyl-1,2,4-oxadiazol-5-yl)-1-(4-(3-(1-methylcyclohexyl)-1,2,4-oxadiazol-5-yl)piperidin-1-yl)ethan-1-one;
1-(4-(3-(2,2-dimethylcyclopropyl)-1,2,4-oxadiazol-5-yl)piperidin-1-yl)-2-(3-methyl-1,2,4-oxadiazol-5-yl)ethan-1-one;
1-(4-(3-(3,3-dimethylcyclopentyl)-1,2,4-oxadiazol-5-yl)piperidin-1-yl)-2-(3-methyl-1,2,4-oxadiazol-5-yl)ethan-1-one;
1-(4-(3-(4,4-dimethylcyclohexyl)-1,2,4-oxadiazol-5-yl)piperidin-1-yl)-2-(3-methyl-1,2,4-oxadiazol-5-yl)ethan-1-one;
1-(4-(3-((3R,5S)-3, 5-dimethylcyclohexyl)-1,2,4-oxadiazol-5-yl)piperidin-1-yl)-2-(3-methyl-1,2,4-oxadiazol-5-yl)ethan-1-one;
1-(4-(3-(2,4-dichlorophenyl)-1,2,4-oxadiazol-5-yl)piperidin-1-yl)-2-(3-methyl-1,2,4-oxadiazol-5-yl)ethan-1-one;
1-(4-(3-(3,4-dichlorophenyl)-1,2,4-oxadiazol-5-yl)piperidin-1-yl)-2-(3-methyl-1,2,4-oxadiazol-5-yl)ethan-1-one;
1-(4-(3-(4-chlorophenyl)-1,2,4-oxadiazol-5-yl)piperidin-1-yl)-2-(3-methyl-1,2,4-oxadiazol-5-yl)ethan-1-one;
1-(4-(5-(4-chlorophenyl)-2H-tetrazol-2-yl)piperidin-1-yl)-2-(3-methyl-1,2,4-oxadiazol-5-yl)ethan-1-one;
1-(4-(3-(3-chlorophenyl)-1,2,4-oxadiazol-5-yl)piperidin-1-yl)-2-(3-methyl-1,2,4-oxadiazol-5-yl)ethan-1-one;
2-(3-methyl-1,2,4-oxadiazol-5-yl)-1-(4-(3-(4-(trifluoromethyl)cyclohexyl)-1,2,4-oxadiazol-5-yl)piperidin-1-yl)ethan-1-one;
1-(4-(3-(4-chloro-3-(trifluoromethoxy)phenyl)-1,2,4-oxadiazol-5-yl)piperidin-1-yl)-2-(3-methyl-1,2,4-oxadiazol-5-yl)ethan-1-one;
2-(3-methyl-1,2,4-oxadiazol-5-yl)-1-(4-(3-(4-(trifluoromethoxy)phenyl)-1,2,4-oxadiazol-5-yl)piperidin-1-yl)ethan-1-one;
1-(4-(3-(3,3-difluorocyclopentyl)-1,2,4-oxadiazol-5-yl)piperidin-1-yl)-2-(3-methyl-1,2,4-oxadiazol-5-yl)ethan-1-one;
1-(4-(3-(4,4-difluorocyclohexyl)-1,2,4-oxadiazol-5-yl)piperidin-1-yl)-2-(3-methyl-1,2,4-oxadiazol-5-yl)ethan-1-one;
1-(4-(3-(4,4-difluorocyclohexyl)-1,2,4-oxadiazol-5-yl)-4-hydroxypiperidin-1-yl)-2-(3-methyl-1,2,4-oxadiazol-5-yl)ethan-1-one;
1-(4-(5-(3-fluorobicyclo[1.1.1]pentan-1-yl)-1,2,4-oxadiazol-3-yl)piperidin-1-yl)-2-(3-methyl-1,2,4-oxadiazol-5-yl)ethan-1-one;
1-(4-(3-(4-chloro-3-fluorophenyl)-1,2,4-oxadiazol-5-yl)piperidin-1-yl)-2-(3-methyl-1,2,4-oxadiazol-5-yl)ethan-1-one;
1-(4-(3-(3-chloro-5-fluorophenyl)-1,2,4-oxadiazol-5-yl)piperidin-1-yl)-2-(3-methyl-1,2,4-oxadiazol-5-yl)ethan-1-one;
1-(4-(3-(2-chloro-4-fluorophenyl)-1,2,4-oxadiazol-5-yl)piperidin-1-yl)-2-(3-methyl-1,2,4-oxadiazol-5-yl)ethan-1-one;
1-(4-(3-(3,4-difluorophenyl)-1,2,4-oxadiazol-5-yl)piperidin-1-yl)-2-(3-methyl-1,2,4-oxadiazol-5-yl)ethan-1-one;
1-(4-(3-(4-fluoro-3-(trifluoromethoxy)phenyl)-1,2,4-oxadiazol-5-yl)piperidin-1-yl)-2-(3-methyl-1,2,4-oxadiazol-5-yl)ethan-1-one;
1-(4-(3-(4-fluorophenyl)-1,2,4-oxadiazol-5-yl)piperidin-1-yl)-2-(3-methyl-1,2,4-oxadiazol-5-yl)ethan-1-one;
1-(4-(3-(4-fluorophenyl)isoxazol-5-yl)piperidin-1-yl)-2-(3-methyl-1,2,4-oxadiazol-5-yl)ethan-1-one;
1-(4-(5-(4-fluorophenyl)-2H-tetrazol-2-yl)piperidin-1-yl)-2-(3-methyl-1,2,4-oxadiazol-5-yl)ethan-1-one;
1-(4-(4-(4-fluorophenyl)oxazol-2-yl)piperidin-1-yl)-2-(3-methyl-1,2,4-oxadiazol-5-yl)ethan-1-one;
1-(4-(5-(4-fluorophenyl)-1,2,4-oxadiazol-3-yl)piperidin-1-yl)-2-(3-methyl-1,2,4-oxadiazol-5-yl)ethan-1-one;
1-(4-(3-(4-fluorophenyl)-1,2,4-oxadiazol-5-yl)-4-hydroxypiperidin-1-yl)-2-(3-methyl-1,2,4-oxadiazol-5-yl)ethan-1-one;
1-(4-(3-cyclohexyl-1,2,4-oxadiazol-5-yl)piperidin-1-yl)-2-(3-methylisoxazol-4-yl)ethan-1-one;
1-(4-(5-cyclohexyl-2H-tetrazol-2-yl)piperidin-1-yl)-2-(3-methylisoxazol-4-yl)ethan-1-one;
1-(4-(3-(3,4-dichlorophenyl)-1,2,4-oxadiazol-5-yl)piperidin-1-yl)-2-(3-methylisoxazol-4-yl)ethan-1-one;
1-(4-(3-(4-fluorophenyl)-1,2,4-oxadiazol-5-yl)piperidin-1-yl)-2-(3-methylisoxazol-4-yl)ethan-1-one;
1-(4-(5-(4-fluorophenyl)-2H-tetrazol-2-yl)piperidin-1-yl)-2-(3-methylisoxazol-4-yl)ethan-1-one;
2-(4-methyl-1,2,5-oxadiazol-3-yl)-1-(4-(4-(morpholine-4-carbonyl)-1H-1,2,3-triazol-1-yl)piperidin-1-yl)ethan-1-one;
1-(4-(3-cyclopropyl-1,2,4-oxadiazol-5-yl)piperidin-1-yl)-2-(4-methyl-1,2,5-oxadiazol-3-yl)ethan-1-one;
1-(4-(5-cyclopropyl-2H-tetrazol-2-yl)piperidin-1-yl)-2-(4-methyl-1,2,5-oxadiazol-3-yl)ethan-1-one;
2-(4-methyl-1,2,5-oxadiazol-3-yl)-1-(4-(5-(spiro[3.3]heptan-2-yl)-1,2,4-oxadiazol-3-yl)piperidin-1-yl)ethan-1-one;
1-(4-(3-(bicyclo[2.2.1]heptan-2-yl)-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(5-(spiro[2.3]hexan-1-yl)-1,2,4-oxadiazol-3-yl)piperidin-1-yl)ethan-1-one;
1-(4-(3-cyclobutyl-1,2,4-oxadiazol-5-yl)piperidin-1-yl)-2-(4-methyl-1,2,5-oxadiazol-3-yl)ethan-1-one;
1-(4-(3-(1,4-dioxaspiro[4.5]decan-8-yl)-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(3-(spiro[2.5]octan-6-yl)-1,2,4-oxadiazol-5-yl)piperidin-1-yl)ethan-1-one;
2-(4-methyl-1,2,5-oxadiazol-3-yl)-1-(4-(5-(spiro[2.5]octan-6-yl)-1,2,4-oxadiazol-3-yl)piperidin-1-yl)ethan-1-one;
1-(4-(3-cyclopentyl-1,2,4-oxadiazol-5-yl)piperidin-1-yl)-2-(4-methyl-1,2,5-oxadiazol-3-yl)ethan-1-one;
1-(4-(5-cyclopentyl-2H-tetrazol-2-yl)piperidin-1-yl)-2-(4-methyl-1,2,5-oxadiazol-3-yl)ethan-1-one;
1-(4-(3-(bicyclo[4.1.0]heptan-2-yl)-1,2,4-oxadiazol-5-yl)piperidin-1-yl)-2-(4-methyl-1,2,5-oxadiazol-3-yl)ethan-1-one;
1-(4-(3-cyclohexyl-1,2,4-oxadiazol-5-yl)piperidin-1-yl)-2-(4-methyl-1,2,5-oxadiazol-3-yl)ethan-1-one;
1-(4-(5-cyclohexyl-2H-tetrazol-2-yl)piperidin-1-yl)-2-(4-methyl-1,2,5-oxadiazol-3-yl)ethan-1-one;
1-(4-(3-cyclohexyl-1,2,4-oxadiazol-5-yl)-4-hydroxypiperidin-1-yl)-2-(4-methyl-1,2,5-oxadiazol-3-yl)ethan-1-one;
2-(4-methyl-1,2,5-oxadiazol-3-yl)-1-(4-(3-(tetrahydro-2H-pyran-4-yl)-1,2,4-oxadiazol-5-yl)piperidin-1-yl)ethan-1-one;
1-(4-(5-(bicyclo[2.2.1]heptan-1-yl)-1,2,4-oxadiazol-3-yl)piperidin-1-yl)-2-(4-methyl-1,2,5-oxadiazol-3-yl)ethan-1-one;
1-(4-(3-(bicyclo[2.2.1]heptan-1-yl)-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(3-phenyl-1H-pyrazol-1-yl)piperidin-1-yl)ethan-1-one;
2-(4-methyl-1,2,5-oxadiazol-3-yl)-1-(4-(5-methyl-2H-tetrazol-2-yl)piperidin-1-yl)ethan-1-one;
1-(4-(3-methyl-1,2,4-oxadiazol-5-yl)piperidin-1-yl)-2-(4-methyl-1,2,5-oxadiazol-3-yl)ethan-1-one;
1-(4-(3-isopropyl-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(3-(1-methylcyclohexyl)-1,2,4-oxadiazol-5-yl)piperidin-1-yl)ethan-1-one;
2-(4-methyl-1,2,5-oxadiazol-3-yl)-1-(4-(3-(3-methyloxetan-3-yl)-1,2,4-oxadiazol-5-yl)piperidin-1-yl)ethan-1-one;
1-(4-(3-(2,2-dimethylcyclopropyl)-1,2,4-oxadiazol-5-yl)piperidin-1-yl)-2-(4-methyl-1,2,5-oxadiazol-3-yl)ethan-1-one;
1-(4-(3-(3,3-dimethylcyclopentyl)-1,2,4-oxadiazol-5-yl)piperidin-1-yl)-2-(4-methyl-1,2,5-oxadiazol-3-yl)ethan-1-one;
1-(4-(3-(4,4-dimethylcyclohexyl)-1,2,4-oxadiazol-5-yl)piperidin-1-yl)-2-(4-methyl-1,2,5-oxadiazol-3-yl)ethan-1-one;
1-(4-(3-(3,3-dimethylcyclohexyl)-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(5-(3-methylbicyclo[1.1.1]pentan-1-yl)-1,2,4-oxadiazol-3-yl)piperidin-1-yl)ethan-1-one;
1-(4-(3-(1-methoxyethyl)-1,2,4-oxadiazol-5-yl)piperidin-1-yl)-2-(4-methyl-1,2,5-oxadiazol-3-yl)ethan-1-one;
1-(4-(3-(methoxymethyl)-1,2,4-oxadiazol-5-yl)piperidin-1-yl)-2-(4-methyl-1,2,5-oxadiazol-3-yl)ethan-1-one;
1-(4-(3-((3R,5S)-3,5-dimethylcyclohexyl)-1,2,4-oxadiazol-5-yl)piperidin-1-yl)-2-(4-methyl-1,2,5-oxadiazol-3-yl)ethan-1-one;
1-(4-(3-(2,4-dichlorophenyl)-1,2,4-oxadiazol-5-yl)piperidin-1-yl)-2-(4-methyl-1,2,5-oxadiazol-3-yl)ethan-1-one;
1-(4-(3-(3,4-dichlorophenyl)-1,2,4-oxadiazol-5-yl)piperidin-1-yl)-2-(4-methyl-1,2,5-oxadiazol-3-yl)ethan-1-one;
1-(4-(3-(3,4-dichlorophenyl)-1,2,4-oxadiazol-5-yl)-4-hydroxypiperidin-1-yl)-2-(4-methyl-1,2,5-oxadiazol-3-yl)ethan-1-one;
1-(4-(5-(4-chlorophenyl)-2H-tetrazol-2-yl)piperidin-1-yl)-2-(4-methyl-1,2,5-oxadiazol-3-yl)ethan-1-one;
1-(4-(3-(4-chlorophenyl)-1,2,4-oxadiazol-5-yl)piperidin-1-yl)-2-(4-methyl-1,2,5-oxadiazol-3-yl)ethan-1-one;
1-(4-(3-(3-chlorophenyl)-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(3-(4-(trifluoromethyl)cyclohexyl)-1,2,4-oxadiazol-5-yl)piperidin-1-yl)ethan-1-one;
1-(4-(3-(4-chloro-3-(trifluoromethoxy)phenyl)-1,2,4-oxadiazol-5-yl)piperidin-1-yl)-2-(4-methyl-1,2,5-oxadiazol-3-yl)ethan-1-one;
2-(4-methyl-1,2,5-oxadiazol-3-yl)-1-(4-(3-(4-(trifluoromethoxy)phenyl)-1,2,4-oxadiazol-5-yl)piperidin-1-yl)ethan-1-one;
2-(4-methyl-1,2,5-oxadiazol-3-yl)-1-(4-(3-(3-(trifluoromethoxy)phenyl)-1,2,4-oxadiazol-5-yl)piperidin-1-yl)ethan-1-one;
2-(4-methyl-1,2,5-oxadiazol-3-yl)-1-(4-(3-(2-(trifluoromethoxy)phenyl)-1,2,4-oxadiazol-5-yl)piperidin-1-yl)ethan-1-one;
1-(4-(3-(4-chloro-3-(trifluoromethyl)phenyl)-1,2,4-oxadiazol-5-yl)piperidin-1-yl)-2-(4-methyl-1,2,5-oxadiazol-3-yl)ethan-1-one:
2-(4-methyl-1,2,5-oxadiazol-3-yl)-1-(4-(3-(4-(trifluoromethyl)phenyl)-1,2,4-oxadiazol-5-yl)piperidin-1-yl)ethan-1-one;
2-(4-methyl-1,2,5-oxadiazol-3-yl)-1-(4-(5-(4-(trifluoromethyl)phenyl)-2H-tetrazol-2-yl)piperidin-1-yl)ethan-1-one;
1-(4-(3-(4-(difluoromethoxy)phenyl)-1,2,4-oxadiazol-5-yl)piperidin-1-yl)-2-(4-methyl-1,2,5-oxadiazol-3-yl)ethan-1-one;
1-(4-(3-(3,3-difluorocyclopentyl)-1,2,4-oxadiazol-5-yl)piperidin-1-yl)-2-(4-methyl-1,2,5-oxadiazol-3-yl)ethan-1-one;
1-(4-(3-(4,4-difluorocyclohexyl)-1,2,4-oxadiazol-5-yl)piperidin-1-yl)-2-(4-methyl-1,2,5-oxadiazol-3-yl)ethan-1-one;
1-(4-(3-(4,4-difluorocyclohexyl)-1,2,4-oxadiazol-5-yl)-4-hydroxypiperidin-1-yl)-2-(4-methyl-1,2,5-oxadiazol-3-yl)ethan-1-one;
1-(4-(3-(3,3-difluorocyclohexyl)-1,2,4-oxadiazol-5-yl)piperidin-1-yl)-2-(4-methyl-1,2,5-oxadiazol-3-yl)ethan-1-one;
1-(4-(3-((1R,3S)-3-fluorocyclopentyl)-1,2,4-oxadiazol-5-yl)piperidin-1-yl)-2-(4-methyl-1,2,5-oxadiazol-3-yl)ethan-1-one;
1-(4-(3-((1R,3R)-3-fluorocyclopentyl)-1,2,4-oxadiazol-5-yl)piperidin-1-yl)-2-(4-methyl-1,2,5-oxadiazol-3-yl)ethan-1-one;
1-(4-(3-(4-fluorocyclohexyl)-1,2,4-oxadiazol-5-yl)piperidin-1-yl)-2-(4-methyl-1,2,5-oxadiazol-3-yl)ethan-1-one;
1-(4-(5-(3-fluorobicyclo[1.1.1]pentan-1-yl)-1,2,4-oxadiazol-3-yl)piperidin-1-yl)-2-(4-methyl-1,2,5-oxadiazol-3-yl)ethan-1-one;
1-(4-(3-(3-fluoro-4-(trifluoromethyl)phenyl)-1,2,4-oxadiazol-5-yl)piperidin-1-yl)-2-(4-methyl-1,2,5-oxadiazol-3-yl)ethan-1-one;
1-(4-(3-(4-chloro-3-fluorophenyl)-1,2,4-oxadiazol-5-yl)piperidin-1-yl)-2-(4-methyl-1,2,5-oxadiazol-3-yl)ethan-1-one;
1-(4-(3-(3-chloro-5-fluorophenyl)-1,2,4-oxadiazol-5-yl)piperidin-1-yl)-2-(4-methyl-1,2,5-oxadiazol-3-yl)ethan-1-one;
1-(4-(3-(4-chloro-2-fluorophenyl)-1,2,4-oxadiazol-5-yl)piperidin-1-yl)-2-(4-methyl-1,2,5-oxadiazol-3-yl)ethan-1-one;
1-(4-(3-(2-chloro-4-fluorophenyl)-1,2,4-oxadiazol-5-yl)piperidin-1-yl)-2-(4-methyl-1,2,5-oxadiazol-3-yl)ethan-1-one;
1-(4-(3-(2,4-difluorophenyl)-1,2,4-oxadiazol-5-yl)piperidin-1-yl)-2-(4-methyl-1,2,5-oxadiazol-3-yl)ethan-1-one;
1-(4-(3-(4-fluoro-2-(trifluoromethyl)phenyl)-1,2,4-oxadiazol-5-yl)piperidin-1-yl)-2-(4-methyl-1,2,5-oxadiazol-3-yl)ethan-1-one;
1-(4-(3-(4-fluoro-3-(trifluoromethyl)phenyl)-1,2,4-oxadiazol-5-yl)piperidin-1-yl)-2-(4-methyl-1,2,5-oxadiazol-3-yl)ethan-1-one;
1-(4-(3-(4-fluoro-3-(trifluoromethoxy)phenyl)-1,2,4-oxadiazol-5-yl)piperidin-1-yl)-2-(4-methyl-1,2,5-oxadiazol-3-yl)ethan-1-one;
1-(4-(3-(4-fluorophenyl)-1H-pyrazol-1-yl)piperidin-1-yl)-2-(4-methyl-1,2,5-oxadiazol-3-yl)ethan-1-one;
1-(4-(4-(4-fluorophenyl)-1H-1,2,3-triazol-1-yl)piperidin-1-yl)-2-(4-methyl-1,2,5-oxadiazol-3-yl)ethan-1-one;
1-(4-(5-(4-fluorophenyl)-1,3,4-oxadiazol-2-yl)piperidin-1-yl)-2-(4-methyl-1,2,5-oxadiazol-3-yl)ethan-1-one;
1-(4-(3-(4-fluorophenyl)-1H-1,2,4-triazol-1-yl)piperidin-1-yl)-2-(4-methyl-1,2,5-oxadiazol-3-yl)ethan-1-one;
1-(4-(5-(4-fluorophenyl)-1,2,4-oxadiazol-3-yl)piperidin-1-yl)-2-(4-methyl-1,2,5-oxadiazol-3-yl)ethan-1-one;
1-(4-(2-(4-fluorophenyl)-2H-tetrazol-5-yl)piperidin-1-yl)-2-(4-methyl-1,2,5-oxadiazol-3-yl)ethan-1-one;
1-(4-(4-(4-fluorophenyl)oxazol-2-yl)piperidin-1-yl)-2-(4-methyl-1,2,5-oxadiazol-3-yl)ethan-1-one;
1-(4-(3-(4-fluorophenyl)isoxazol-5-yl)piperidin-1-yl)-2-(4-methyl-1,2,5-oxadiazol-3-yl)ethan-1-one;
1-(4-(4-(4-fluorophenyl)-2H-1,2,3-triazol-2-yl)piperidin-1-yl)-2-(4-methyl-1,2,5-oxadiazol-3-yl)ethan-1-one;
1-(4-(4-(4-fluorophenyl)-1H-1,2,3-triazol-1-yl)piperidin-1-yl)-2-(4-methyl-1,2,5-oxadiazol-3-yl)ethan-1-one;
1-(4-(3-(4-fluorophenyl)-1,2,4-oxadiazol-5-yl)-4-methylpiperidin-1-yl)-2-(4-methyl-1,2,5-oxadiazol-3-yl)ethan-1-one;
1-(4-(3-(4-fluorophenyl)-1,2,4-oxadiazol-5-yl)-4-(trifluoromethyl)piperidin-1-yl)-2-(4-methyl-1,2,5-oxadiazol-3-yl)ethan-1-one;
1-(4-(3-(4-fluorophenyl)-1,2,4-oxadiazol-5-yl)-4-hydroxypiperidin-1-yl)-2-(4-methyl-1,2,5-oxadiazol-3-yl)ethan-1-one;
1-(4-(3-(5-fluoropyridin-2-yl)-1,2,4-oxadiazol-5-yl)piperidin-1-yl)-2-(4-methyl-1,2,5-oxadiazol-3-yl)ethan-1-one;
1-(4-(3-(4-fluorobenzyl)-1,2,4-oxadiazol-5-yl)piperidin-1-yl)-2-(4-methyl-1,2,5-oxadiazol-3-yl)ethan-1-one;
1-(4-(3-(2,3-difluorophenyl)-1,2,4-oxadiazol-5-yl)piperidin-1-yl)-2-(4-methyl-1,2,5-oxadiazol-3-yl)ethan-1-one;
1-(4-(3-(3-fluorophenyl)-1,2,4-oxadiazol-5-yl)piperidin-1-yl)-2-(4-methyl-1,2,5-oxadiazol-3-yl)ethan-1-one;
1-(4-(5-(3-fluorophenyl)-2H-tetrazol-2-yl)piperidin-1-yl)-2-(4-methyl-1,2,5-oxadiazol-3-yl)ethan-1-one;
1-(4-(3-(2-fluorophenyl)-1,2,4-oxadiazol-5-yl)piperidin-1-yl)-2-(4-methyl-1,2,5-oxadiazol-3-yl)ethan-1-one;
1-(4-(5-(2-fluorophenyl)-2H-tetrazol-2-yl)piperidin-1-yl)-2-(4-methyl-1,2,5-oxadiazol-3-yl)ethan-1-one;
1-(4-(3-(5-fluoropyrimidin-2-yl)-1,2,4-oxadiazol-5-yl)piperidin-1-yl)-2-(4-methyl-1,2,5-oxadiazol-3-yl)ethan-1-one;
4-(2-(1-(2-(4-methyl-1,2,5-oxadiazol-3-yl)acetyl)piperidin-4-yl)-2H-tetrazol-5-yl)benzamide;
N-(2-hydroxyethyl)-1-(1-(2-(4-methyl-1,2,5-oxadiazol-3-yl)acetyl)piperidin-4-yl)-1H-1,2,3-triazole-4-carboxamide;
3-(4-fluorophenyl)-5-(I-(((4-methyl-1,2,5-oxadiazol-3-yl)methyl)sulfonyl)piperidin-4-yl)-1,2,4-oxadiazole;
4-(3-(4-fluorophenyl)-1,2,4-oxadiazol-5-yl)-N-(4-methyl-1,2,5-oxadiazol-3-yl)piperidine-1-carboxamide;
1-(4-(3-(3,4-dichlorophenyl)-1,2,4-oxadiazol-5-yl)piperidin-1-yl)-2-(1-methyl-H-1,2,4-triazol-3-yl)ethan-1-one;
1-(4-(3-(4-fluorophenyl)-1,2,4-oxadiazol-5-yl)piperidin-1-yl)-2-(1-methyl-1H-1,2,4-triazol-3-yl)ethan-1-one;
1-(4-(3-(4-fluorophenyl)-1,2,4-oxadiazol-5-yl)piperidin-1-yl)-2-(4-methyl-4H-1,2,4-triazol-3-yl)ethan-1-one;
1-(4-(5-(4-fluorophenyl)-2H-tetrazol-2-yl)piperidin-1-yl)-2-(4-methyl-4H-1,2,4-triazol-3-yl)ethan-1-one;
1-(4-(3-(bicyclo[2.2.1]heptan-2-yl)-1,2,4-oxadiazol-5-yl)piperidin-1-yl)-2-(1-methyl-1H-1,2,4-triazol-5-yl)ethan-1-one;
1-(4-(3-((1R,3R)-3-fluoro-3-methylcyclopentyl)-1,2,4-oxadiazol-5-yl)piperidin-1-yl)-2-(1-methyl-1H-1,2,4-triazol-5-yl)ethan-1-one;
1-(4-(3-((1R,3S)-3-fluoro-3-methylcyclopentyl)-1,2,4-oxadiazol-5-yl)piperidin-1-yl)-2-(1-methyl-1H-1,2,4-triazol-5-yl)ethan-1-one;
1-(4-(3-(4-fluoro-4-methylcyclohexyl)-1,2,4-oxadiazol-5-yl)piperidin-1-yl)-2-(1-methyl-1H-1,2,4-triazol-5-yl)ethan-1-one;
2-(I-methyl-1H-1,2,4-triazol-5-yl)-1-(4-(5-(3-methylbicyclo[1.1.1]pentan-1-yl)-1,2,4-oxadiazol-3-yl)piperidin-1-yl)ethan-1-one;
2-(1-methyl-1H-1,2,4-triazol-5-yl)-1-(4-(3-(3-methylbicyclo[1.1.1]pentan-1-yl)-1,2,4-oxadiazol-5-yl)piperidin-1-yl)ethan-1-one;
1-(4-(3-(2,4-dichlorophenyl)-1,2,4-oxadiazol-5-yl)piperidin-1-yl)-2-(1-methyl-1H-1,2,4-triazol-5-yl)ethan-1-one;
1-(4-(5-(3,4-dichlorophenyl)-2H-tetrazol-2-yl)piperidin-1-yl)-2-(1-methyl-1H-1,2,4-triazol-5-yl)ethan-1-one;
1-(4-(5-(3,4-dichlorophenyl)-1,2,4-oxadiazol-3-yl)piperidin-1-yl)-2-(1-methyl-1H-1,2,4-triazol-5-yl)ethan-1-one;
1-(4-(3-(4-chlorophenyl)-1,2,4-oxadiazol-5-yl)piperidin-1-yl)-2-(1-methyl-1H-1,2,4-triazol-5-yl)ethan-1-one;
1-(4-(5-(4-chlorophenyl)-2H-tetrazol-2-yl)piperidin-1-yl)-2-(1-methyl-1H-1,2,4-triazol-5-yl)ethan-1-one;
1-(4-(3-(4-chloro-3-(trifluoromethoxy)phenyl)-1,2,4-oxadiazol-5-yl)piperidin-1-yl)-2-(1-methyl-1H-1,2,4-triazol-5-yl)ethan-1-one;
2-(1-methyl-1H-1,2,4-triazol-5-yl)-1-(4-(3-(4-(trifluoromethoxy)phenyl)-1,2,4-oxadiazol-5-yl)piperidin-1-yl)ethan-1-one;
2-(1-methyl-1H-1,2,4-triazol-5-yl)-1-(4-(5-(4-(trifluoromethyl)phenyl)-2H-tetrazol-2-yl)piperidin-1-yl)ethan-1-one;
1-(4-(3-(4-(difluoromethyl)phenyl)-1,2,4-oxadiazol-5-yl)piperidin-1-yl)-2-(1-methyl-1H-1,2,4-triazol-5-yl)ethan-1-one;
1-(4-(5-(3-fluorobicyclo[1.1.1]pentan-1-yl)-1,2,4-oxadiazol-3-yl)piperidin-1-yl)-2-(1-methyl-1H-1,2,4-triazol-5-yl)ethan-1-one;
1-(4-(3-(4-(fluoromethyl)phenyl)-1,2,4-oxadiazol-5-yl)piperidin-1-yl)-2-(l-methyl-1H-1,2,4-triazol-5-yl)ethan-1-one;
2-(1-methyl-1H-1,2,4-triazol-5-yl)-1-(4-(3-(4-(pentafluoro-16-sulfaneyl)phenyl)-1,2,4-oxadiazol-5-yl)piperidin-1-yl)ethan-1-one;
1-(4-(5-(4-chloro-3-fluorophenyl)-2H-tetrazol-2-yl)piperidin-1-yl)-2-(1-methyl-1H-1,2,4-triazol-5-yl)ethan-1-one;
1-(4-(3-(4-chloro-2-fluorophenyl)-1,2,4-oxadiazol-5-yl)piperidin-1-yl)-2-(1-methyl-1H-1,2,4-triazol-5-yl)ethan-1-one;
1-(4-(3-(2-chloro-4-fluorophenyl)-1,2,4-oxadiazol-5-yl)piperidin-1-yl)-2-(1-methyl-1H-1,2,4-triazol-5-yl)ethan-1-one;
1-(4-(3-(2,4-difluorophenyl)-1,2,4-oxadiazol-5-yl)piperidin-1-yl)-2-(1-methyl-1H-1,2,4-triazol-5-yl)ethan-1-one;
1-(4-(3-(4-fluoro-3-(trifluoromethyl)phenyl)-1,2,4-oxadiazol-5-yl)piperidin-1-yl)-2-(1-methyl-1H-1,2,4-triazol-5-yl)ethan-1-one;
1-(4-(5-(3-chloro-4-fluorophenyl)-2H-tetrazol-2-yl)piperidin-1-yl)-2-(1-methyl-1H-1,2,4-triazol-5-yl)ethan-1-one;
1-(4-(3-(3-chloro-4-fluorophenyl)-1,2,4-oxadiazol-5-yl)piperidin-1-yl)-2-(1-methyl-1H-1,2,4-triazol-5-yl)ethan-1-one;
1-(4-(3-(3,4-difluorophenyl)-1,2,4-oxadiazol-5-yl)piperidin-1-yl)-2-(l-methyl-1H-1,2,4-triazol-5-yl)ethan-1-one;
1-(4-(5-(3,4-difluorophenyl)-2H-tetrazol-2-yl)piperidin-1-yl)-2-(I-methyl-1H-1,2,4-triazol-5-yl)ethan-1-one;
1-(4-(3-(4-fluorophenyl)-1,2,4-oxadiazol-5-yl)piperidin-1-yl)-2-(1-methyl-1H-1,2,4-triazol-5-yl)ethan-1-one;
1-(4-(5-(4-fluorophenyl)-2H-tetrazol-2-yl)piperidin-1-yl)-2-(1-methyl-1H-1,2,4-triazol-5-yl)ethan-1-one;
1-(4-(3-(4-fluorophenyl)-1,2,4-oxadiazol-5-yl)piperidin-1-yl)-2-(1-methyl-1H-tetrazol-5-yl)ethan-1-one;
1-(5-(3-cyclohexyl-1,2,4-oxadiazol-5-yl)-2-azabicyclo[2.2.1]heptan-2-yl)-2-(3-methyl-1,2,4-oxadiazol-5-yl)ethan-1-one;
1-(5-(3-(3,4-dichlorophenyl)-1,2,4-oxadiazol-5-yl)-2-azabicyclo[2.2.1]heptan-2-yl)-2-(3-methyl-1,2,4-oxadiazol-5-yl)ethan-1-one;
1-(5-(3-(4,4-difluorocyclohexyl)-1,2,4-oxadiazol-5-yl)-2-azabicyclo[2.2.1]heptan-2-yl)-2-(3-methyl-1,2,4-oxadiazol-5-yl)ethan-1-one;
1-(5-(3-(3,4-difluorophenyl)-1,2,4-oxadiazol-5-yl)-2-azabicyclo[2.2.1]heptan-2-yl)-2-(3-methyl-1,2,4-oxadiazol-5-yl)ethan-1-one;
1-(5-(3-(4-fluorophenyl)-1,2,4-oxadiazol-5-yl)-2-azabicyclo[2.2.1]heptan-2-yl)-2-(3-methyl-1,2,4-oxadiazol-5-yl)ethan-1-one;
1-(5-(3-cyclohexyl-1,2,4-oxadiazol-5-yl)-2-azabicyclo[2.2.1]heptan-2-yl)-2-(4-methyl-1,2,5-oxadiazol-3-yl)ethan-1-one;
1-(5-(3-(3,4-dichlorophenyl)-1,2,4-oxadiazol-5-yl)-2-azabicyclo[2.2.1]heptan-2-yl)-2-(4-methyl-1,2,5-oxadiazol-3-yl)ethan-1-one;
1-(5-(3-(4,4-difluorocyclohexyl)-1,2,4-oxadiazol-5-yl)-2-azabicyclo[2.2.1]heptan-2-yl)-2-(4-methyl-1,2,5-oxadiazol-3-yl)ethan-1-one;
1-(5-(3-(3-fluorocyclopentyl)-1,2,4-oxadiazol-5-yl)-2-azabicyclo[2.2.1]heptan-2-yl)-2-(4-methyl-1,2,5-oxadiazol-3-yl)ethan-1-one;
1-(5-(3-(3,4-difluorophenyl)-1,2,4-oxadiazol-5-yl)-2-azabicyclo[2.2.1]heptan-2-yl)-2-(4-methyl-1,2,5-oxadiazol-3-yl)ethan-1-one;
1-(5-(3-(4-fluorophenyl)-1,2,4-oxadiazol-5-yl)-2-azabicyclo[2.2.1]heptan-2-yl)-2-(4-methyl-1,2,5-oxadiazol-3-yl)ethan-1-one;
1-(5-(3-(4-chlorophenyl)-1,2,4-oxadiazol-5-yl)-2-azabicyclo[2.2.1]heptan-2-yl)-2-(1-methyl-1H-1,2,4-triazol-5-yl)ethan-1-one;
1-((1R,4S)-5-(3-(4-chloro-3-fluorophenyl)-1,2,4-oxadiazol-5-yl)-2-azabicyclo[2.2.1]heptan-2-yl)-2-(1-methyl-1H-1,2,4-triazol-5-yl)ethan-1-one;
1-((1S,4R)-5-(3-(4-chloro-3-fluorophenyl)-1,2,4-oxadiazol-5-yl)-2-azabicyclo[2.2.1]heptan-2-yl)-2-(i-methyl-1H-1,2,4-triazol-5-yl)ethan-1-one;
1-(5-(3-(4-chloro-2-fluorophenyl)-1,2,4-oxadiazol-5-yl)-2-azabicyclo[2.2.1]heptan-2-yl)-2-(1-methyl-1H-1,2,4-triazol-5-yl)ethan-1-one;
1-(5-(3-(4-fluorophenyl)-1,2,4-oxadiazol-5-yl)-2-azabicyclo[2.2.1]heptan-2-yl)-2-(1-methyl-1H-1,2,4-triazol-5-yl)ethan-1-one;
1-(6-(3-(3,4-dichlorophenyl)-1,2,4-oxadiazol-5-yl)-3-azabicyclo[3.1.1]heptan-3-yl)-2-(3-methyl-1,2,4-oxadiazol-5-yl)ethan-1-one;
1-(6-(3-(4,4-difluorocyclohexyl)-1,2,4-oxadiazol-5-yl)-3-azabicyclo[3.1.1]heptan-3-yl)-2-(3-methyl-1,2,4-oxadiazol-5-yl)ethan-1-one;
1-(6-(3-(3,4-difluorophenyl)-1,2,4-oxadiazol-5-yl)-3-azabicyclo[3.1.1]heptan-3-yl)-2-(3-methyl-1,2,4-oxadiazol-5-yl)ethan-1-one:
1-(6-(3-(4-fluorophenyl)-1,2,4-oxadiazol-5-yl)-3-azabicyclo[3.1.1]heptan-3-yl)-2-(3-methyl-1,2,4-oxadiazol-5-yl)ethan-1-one;
1-(6-(3-(4-fluorophenyl)-1,2,4-oxadiazol-5-yl)-3-azabicyclo[3.1.1]heptan-3-yl)-2-(3-methylisoxazol-4-yl)ethan-1-one;
1-(6-(3-(4-fluoro-4-methylcyclohexyl)-1,2,4-oxadiazol-5-yl)-3-azabicyclo[3.1.1]heptan-3-yl)-2-(4-methyl-1,2,5-oxadiazol-3-yl)ethan-1-one;
1-(6-(3-(3,4-dichlorophenyl)-1,2,4-oxadiazol-5-yl)-3-azabicyclo[3.1.1]heptan-3-yl)-2-(4-methyl-1,2,5-oxadiazol-3-yl)ethan-1-one;
1-(6-(3-(4,4-difluorocyclohexyl)-1,2,4-oxadiazol-5-yl)-3-azabicyclo[3.1.1]heptan-3-yl)-2-(4-methyl-1,2,5-oxadiazol-3-yl)ethan-1-one;
1_-(6-(3-(3,4-difluorophenyl)-1,2,4-oxadiazol-5-yl)-3-azabicyclo[3.1.1]heptan-3-yl)-2-(4-methyl-1,2,5-oxadiazol-3-yl)ethan-1-one;
1-(6-(3-(4-fluorophenyl)-1,2,4-oxadiazol-5-yl)-3-azabicyclo[3.1.1]heptan-3-yl)-2-(4-methyl-1,2,5-oxadiazol-3-yl)ethan-1-one;
1-(6-(3-(3,4-dichlorophenyl)-1,2,4-oxadiazol-5-yl)-3-azabicyclo[3.1.1]heptan-3-yl)-2-(I-methyl-1H-1,2,4-triazol-5-yl)ethan-1-one;
1-((1R,5S,6r)-6-(3-(3,4-dichlorophenyl)-1,2,4-oxadiazol-5-yl)-3-azabicyclo[3.1.1]heptan-3-yl)-2-(1-methyl-1H-1,2,4-triazol-5-yl)ethan-1-one;
1-((1R,5S,6s)-6-(3-(3,4-dichlorophenyl)-1,2,4-oxadiazol-5-yl)-3-azabicyclo[3.1.1]heptan-3-yl)-2-(1-methyl-1H-1,2,4-triazol-5-yl)ethan-1-one;
1-((1R,5S,6s)-6-(3-(4-chlorophenyl)-1,2,4-oxadiazol-5-yl)-3-azabicyclo[3.1.1]heptan-3-yl)-2-(1-methyl-1H-1,2,4-triazol-5-yl)ethan-1-one;
1-((1R,5S,6r)-6-(3-(4-chloro-3-fluorophenyl)-1,2,4-oxadiazol-5-yl)-3-azabicyclo[3.1.1]heptan-3-yl)-2-(1-methyl-1H-1,2,4-triazol-5-yl)ethan-1-one;
1-((1R,5S,6s)-6-(3-(4-chloro-3-fluorophenyl)-1,2,4-oxadiazol-5-yl)-3-azabicyclo[3.1.1]heptan-3-yl)-2-(1-methyl-1H-1,2,4-triazol-5-yl)ethan-1-one;
1-((1R,5S,6r)-6-(3-(4-chloro-2-fluorophenyl)-1,2,4-oxadiazol-5-yl)-3-azabicyclo[3.1.1]heptan-3-yl)-2-(1-methyl-1H-1,2,4-triazol-5-yl)ethan-1-one;
1-((1R,5S,6s)-6-(3-(4-chloro-2-fluorophenyl)-1,2,4-oxadiazol-5-yl)-3-azabicyclo[3.1.1]heptan-3-yl)-2-(1-methyl-1H-1,2,4-triazol-5-yl)ethan-1-one;
1-(2-(3-cyclohexyl-1,2,4-oxadiazol-5-yl)-7-azaspiro[3.5]nonan-7-yl)-2-(3-methyl-1,2,4-oxadiazol-5-yl)ethan-1-one;
1-(2-(3-(3,4-dichlorophenyl)-1,2,4-oxadiazol-5-yl)-7-azaspiro[3.5]nonan-7-yl)-2-(3-methyl-1,2,4-oxadiazol-5-yl)ethan-1-one;
1-(2-(3-(4,4-difluorocyclohexyl)-1,2,4-oxadiazol-5-yl)-7-azaspiro[3.5]nonan-7-yl)-2-(3-methyl-1,2,4-oxadiazol-5-yl)ethan-1-one;
1-(2-(3-(3,4-difluorophenyl)-1,2,4-oxadiazol-5-yl)-7-azaspiro[3.5]nonan-7-yl)-2-(3-methyl-1,2,4-oxadiazol-5-yl)ethan-1-one;
1-(2-(3-cyclohexyl-1,2,4-oxadiazol-5-yl)-7-azaspiro[3.5]nonan-7-yl)-2-(4-methyl-1,2,5-oxadiazol-3-yl)ethan-1-one;
1-(2-(3-(3,4-dichlorophenyl)-1,2,4-oxadiazol-5-yl)-7-azaspiro[3.5]nonan-7-yl)-2-(4-methyl-1,2,5-oxadiazol-3-yl)ethan-1-one;
1-(2-(3-(4,4-difluorocyclohexyl)-1,2,4-oxadiazol-5-yl)-7-azaspiro[3.5]nonan-7-yl)-2-(4-methyl-1,2,5-oxadiazol-3-yl)ethan-1-one:
1-(2-(3-(3,4-difluorophenyl)-1,2,4-oxadiazol-5-yl)-7-azaspiro[3.5]nonan-7-yl)-2-(4-methyl-1,2,5-oxadiazol-3-yl)ethan-1-one;
1-(2-(3-(4-fluorophenyl)-1,2,4-oxadiazol-5-yl)-7-azaspiro[3.5]nonan-7-yl)-2-(4-methyl-1,2,5-oxadiazol-3-yl)ethan-1-one;
1-(4-(3-(3,4-dichlorophenyl)-1,2,4-oxadiazol-5-yl)piperazin-1-yl)-2-(3-methyl-1,2,4-oxadiazol-5-yl)ethan-1-one;
1-(4-(3-(3,4-dichlorophenyl)-1,2,4-oxadiazol-5-yl)piperazin-1-yl)-2-(4-methyl-1,2,5-oxadiazol-3-yl)ethan-1-one;
1-(4-(3-(4-fluorophenyl)-1,2,4-oxadiazol-5-yl)piperazin-1-yl)-2-(4-methyl-1,2,5-oxadiazol-3-yl)ethan-1-one;
1-(1-(3-(4-fluorophenyl)-1,2,4-oxadiazol-5-yl)-6-azaspiro[2.5]octan-6-yl)-2-(4-methyl-1,2,5-oxadiazol-3-yl)ethan-1-one;
1-(1-(3-(4-fluorophenyl)-1,2,4-oxadiazol-5-yl)-6-azaspiro[2.5]octan-6-yl)-2-(3-methyl-1,2,4-oxadiazol-5-yl)ethan-1-one;
1-(1-(3-(3,4-dichlorophenyl)-1,2,4-oxadiazol-5-yl)-7-azaspiro[3.5]nonan-7-yl)-2-(3-methyl-1,2,4-oxadiazol-5-yl)ethan-1-one;
1-(1-(3-(4-fluorophenyl)-1,2,4-oxadiazol-5-yl)-7-azaspiro[3.5]nonan-7-yl)-2-(3-methyl-1,2,4-oxadiazol-5-yl)ethan-1-one;
1-(1-(3-(4-fluorophenyl)-1,2,4-oxadiazol-5-yl)-7-azaspiro[3.5]nonan-7-yl)-2-(4-methyl-1,2,5-oxadiazol-3-yl)ethan-1-one;
1-(6-(5-(4-fluorophenyl)-2H-tetrazol-2-yl)-3-azabicyclo[3.1.1]heptan-3-yl)-2-(3-methyl-1,2,4-oxadiazol-5-yl)ethan-1-one;
1-(6-(5-(4-fluorophenyl)-2H-tetrazol-2-yl)-3-azabicyclo[3.1.1]heptan-3-yl)-2-(4-methyl-1,2,5-oxadiazol-3-yl)ethan-1-one;
2-(4-methyl-1,2,5-oxadiazol-3-yl)-1-(3-(4-(morpholine-4-carbonyl)-1H-1,2,3-triazol-1-yl)piperidin-1-yl)ethan-1-one;
N,N-diethyl-1-(1-(2-(4-methyl-1,2,5-oxadiazol-3-yl)acetyl)piperidin-3-yl)-1H-1,2,3-triazole-4-carboxamide;
N-(2-hydroxyethyl)-1-(1-(2-(4-methyl-1,2,5-oxadiazol-3-yl)acetyl)piperidin-3-yl)-1H-1,2,3-triazole-4-carboxamide;
1-(2-(5-(4-fluorophenyl)-2H-tetrazol-2-yl)-7-azaspiro[3.5]nonan-7-yl)-2-(4-methyl-1,2,5-oxadiazol-3-yl)ethan-1-one;
1-(3-(5-(4-fluorophenyl)-2H-tetrazol-2-yl)piperidin-1-yl)-2-(4-methyl-1,2,5-oxadiazol-3-yl)ethan-1-one;
1-(8-(3-(4-fluorophenyl)-1,2,4-oxadiazol-5-yl)-3-azabicyclo[3.2.1]octan-3-yl)-2-(4-methyl-1,2,5-oxadiazol-3-yl)ethan-1-one;
1-(4-(1-(4-fluorophenyl)-1H-1,2,4-triazol-3-yl)piperazin-1-yl)-2-(4-methyl-1,2,5-oxadiazol-3-yl)ethan-1-one;
4-(3-(4-fluorophenyl)-1,2,4-oxadiazol-5-yl)-N-(4-methyl-1,2,5-oxadiazol-3-yl)cyclohexane-1-carboxamide;
1-(4-(2-(4-fluorophenyl)pyrimidin-5-yl)piperidin-1-yl)-2-(4-methyl-1,2,5-oxadiazol-3-yl)ethan-1-one;
1-(4-(5-(4-fluorophenyl)pyrinidin-2-yl)piperidin-1-yl)-2-(4-methyl-1,2,5-oxadiazol-3-yl)ethan-1-one;
1-(4-(2,3-dimethylphenoxy)piperidin-1-yl)-2-(4-methyl-1,2,5-oxadiazol-3-yl)ethan-1-one;
1-(4-(6-(4-fluorophenyl)pyridazin-3-yl)piperidin-1-yl)-2-(4-methyl-1,2,5-oxadiazol-3-yl)ethan-1-one;
1-(3-(3-(4-fluorophenyl)-1,2,4-oxadiazol-5-yl)piperidin-1-yl)-2-(4-methyl-1,2,5-oxadiazol-3-yl)ethan-1-one;
1-(3-(3-(4-fluorophenyl)-1,2,4-oxadiazol-5-yl)azetidin-1-yl)-2-(4-methyl-1,2,5-oxadiazol-3-yl)ethan-1-one; and
1-(1-(5-(4-fluorophenyl)-2H-tetrazol-2-yl)-7-azaspiro[3.5]nonan-7-yl)-2-(4-methyl-1,2,5-oxadiazol-3-yl)ethan-1-one.

62. The compound of claim 61 having the formula:
1-(4-(3-(3,4-dichlorophenyl)-1,2,4-oxadiazol-5-yl)piperidin-1-yl)-2-(1-methyl-1H-1,2,4-triazol-5-yl)ethan-1-one.

63. The compound of claim 61 having the formula:
1-(4-(3-(4-fluorophenyl)-1,2,4-oxadiazol-5-yl)piperidin-1-yl)-2-(4-methyl-1,2,5-oxadiazol-3-yl)ethan-1-one.

64. The compound of claim 61 having the formula:
1-(4-(3-(4-chloro-3-fluorophenyl)-1,2,4-oxadiazol-5-yl)piperidin-1-yl)-2-(1-methyl-1H-1,2,4-triazol-5-yl)ethan-1-one.

65. The compound of claim 61 having the formula:
1-((1R,5S,6r)-6-(3-(4-chlorophenyl)-1,2,4-oxadiazol-5-yl)-3-azabicyclo[3.1.1]heptan-3-yl)-2-(1-methyl-1H-1,2,4-triazol-5-yl)ethan-1-one.

66. The compound of claim 61 having the formula:
1-(5-(3-(4-chloro-3-fluorophenyl)-1,2,4-oxadiazol-5-yl)-2-azabicyclo[2.2.1]heptan-2-yl)-2-(1-methyl-1H-1,2,4-triazol-5-yl)ethan-1-one.

67. A composition comprising the compound of claim 1, admixed with a pharmaceutically acceptable carrier, diluent, or excipient.

68. The composition of claim 67, further comprising one or more therapeutic compounds or compositions used for the treatment of a mycobacterial disease.

69. The composition of claim 67, further comprising one or more therapeutic compounds or compositions used for the treatment of a TB infection.

70. The composition of claim 69, wherein the one or more therapeutic compounds or compositions is selected from bedaquiline, pretomanid and linezolid.

71. The composition of claim 69, wherein the one or more therapeutic compounds or compositions is Nix-TB.

72. The composition of claim 69, wherein the one or more therapeutic compounds or compositions is selected from bedaquiline, pretonanid, linezolid, isoniazid, rifampin, rifabutin, rifapentine, pyrazinamide, ethambutol, ethionamide, moxifloxacin, tedizolid, radezolid, sutezolid, posizolid clofazimine, gatifloxacin, kanamycin, nitroimidazo-oxazine, delamanid, OPC-167832, streptomycin, prednisolone, an oxazolidinone, a fluoroquinolone, a corticosteroid, EMB analogue SQ109, a benzothiazinone, a dinitrobenzamide, and an antiviral agent including an antiretroviral agent.

73. The composition of claim 69, wherein the one or more therapeutic compounds or compositions is a therapeutic agent approved or recommended for the treatment of tuberculosis.

74. The composition of claim 67, wherein the composition is suitable for oral administration.

75. A method of activating Mycobacterium tuberculosis (Mtb) adenylyl cyclase encoded by Rv1625c, comprising treating intracellular and/or extracellular Mtb with the compound of claim 1.

76. A method of inhibiting the cholesterol degradation pathway during intramacrophage Mtb infection or non-replicating extracellular Mtb, comprising treating Mtb with the compound of claim 1.

77. A method of preventing, ameliorating, or treating a mycobacterial disease, comprising administering to a subject in need thereof a therapeutically effective amount of the composition of claim 67.

78. A method of preventing, ameliorating, or treating a TB infection, comprising administering to a subject in need thereof a therapeutically effective amount of the composition of claim 67.

79. (canceled)
